(19) United States
(12) Patent Application Publication
(10) Pub. No.: US 2008/0279812 A1
(43) Pub. Date: Nov. 13, 2008

(54) Disease Prevention and Vaccination Prior to Thymic Reactivation

(75) Inventors: Richard Lennox Boyd, Hampton (AU); Gabrielle Lianne Goldberg, Brighton (AU); Ann Patricia Chidgey, Black Rock (AU); Jayne Suzanne Sutherland, Port Melbourne (AU)

Correspondence Address: WILMERHALE/BOSTON, 60 STATE STREET, BOSTON, MA, 02109, US

(73) Assignees: Norwood Immunology, Ltd., Chelsea Heights (AU); Monash University, Clayton (AU)

(21) Appl. No.: 10/553,594

(22) PCT Filed: Apr. 19, 2004
(86) PCT No.: PCT/US04/11921

§ 371 Date: Jul. 30, 2007

Related U.S. Application Data
(60) Provisional application No. 60/527,001, filed on Dec. 5, 2003.

(51) Int. Cl. A61K 38/20; A61K 39/00; A61K 31/57; A61K 38/00; A61K 38/08; A61K 31/496; A61K 31/167; A61K 38/21; A61D 1/06; A61K 38/18; A61P 33/00; A61K 35/00; A61P 31/00; A61P 35/00; A61P 37/08; A61K 38/22

(52) U.S. Cl. 424/85.2; 424/184.1; 514/177; 514/12; 514/15; 514/254.07; 514/629; 514/178; 424/93.7; 424/85.1; 424/85.5; 606/135

(57) ABSTRACT

The present disclosure provides methods for preventing or treating illness, improving responsiveness to immunization, and improving the efficacy of gene therapy in a patient by disrupting sex steroid signalling in the patient, wherein the bone marrow and other immune cell functionality is improved without, prior to, or concurrently with, thymic regeneration. In some embodiments, sex steroid signalling is interrupted or ablated in the patient by the administration of LHRH agonists, LHRH antagonists, anti-LHRH receptor antibodies, anti-LHRH vaccines, anti-androgens, anti-estrogens, selective estrogen receptor modulators (SERMs), selective androgen receptor modulators (SARMs), selective progesterone response modulators (SPRMs), ERDs, aromatase inhibitors, or various combinations thereof.

### FIELD OF THE INVENTION

The present invention is in the fields of cellular immunology, disease prevention, vaccination, and gene therapy. More specifically, the invention is directed to enhancing bone marrow (BM) hematopoiesis and functionality, enhancing BM engraftment following hematopoietic stem cell transplant (HSCT), and increasing the functionality of new and pre-existing T cells and other cells of the immune system.

### BACKGROUND

### The Immune System

The major function of the immune system is to distinguish “foreign” (i.e., derived from any source outside the body) antigens from “self” (i.e., derived from within the body) and respond accordingly to protect the body against infection. In more practical terms, the immune response has also been described as responding to danger signals. These danger signals may be any change in the property of a cell or tissue which alerts cells of the immune system that this cell/tissue in question is no longer “normal.” Such alerts may be very important in causing, for example, rejection of foreign agents such as viral, bacterial, parasitic, and fungal infections; they may also be used to induce anti-tumor responses. However, such danger signals may also be the reason why some autoimmune diseases start, due to either inappropriate cell changes in the self cells which then become targeted by the immune system (e.g., the pancreatic β-islet cells in diabetes mellitus). Alternatively, inappropriate stimulation of the immune cells themselves can lead to the destruction of normal self cells, in addition to the foreign cell or microorganism which induced the initial response.

In normal immune responses, the sequence of events involves dedicated antigen presenting cells (APC) capturing foreign antigen and processing it into small peptide fragments which are then presented in clefts of major histocompatibility complex (MHC) molecules on the APC surface. The MHC molecules can either be of class I expressed on all nucleated cells (recognized by cytotoxic T cells (Tc or CTL)) or of class II expressed primarily by cells of the immune system (recognized by helper T cells (Th)). Th cells recognize the MHC II/peptide complexes on APC and respond. Factors released by these cells then promote the activation of either, or both, of Tc cells or antibody producing B cells, which are specific for the particular antigen. The importance of Th cells in virtually all immune responses is best illustrated in HIV/AIDS where their absence through destruction by the virus causes severe immune deficiency, which eventually leads to death due to opportunistic infections. Inappropriate development of Th (and to a lesser extent Tc) cells can also lead to a variety of other conditions such as allergies, cancer, and autoimmunity.

The inappropriate development of such cells may be due to an abnormal thymus in which the structural organization is markedly altered, e.g., in many autoimmune diseases, the medullary epithelial cells, which are required for development of mature thymocytes, are ectopically expressed in the cortex where immature T cells normally reside. This could mean that the developing immature T cells prematurely receive late stage maturation signals and, in doing so, become insensitive to the negative selection signals that would normally delete potentially autoreactive cells. Indeed, this type of thymic abnormality has been found in NZB mice, which develop Lupus-like symptoms (Takeoka et al., (1999) Clin. Immunol. 90:388), and more recently in NOD mice, which develop type I diabetes (Thomas-Vaslin et al., (1997) P.N.A.S. USA 94:4598; Atlan-Gepner et al., (1999) Autoimmunity 3:249-260). It is not known how or when these forms of thymic abnormality develop, but it could be through the natural aging process or from destructive agents such as viral infections (changes in the thymus have been described in AIDS patients), stress, chemotherapy, and radiation therapy (Mackall et al., (1995) N. Eng. J. Med. 332:143; Heitger et al., (1997) Blood 99:4053; Mackall and Gress, (1997) Immunol. Rev. 160:91). It is also possible that the defects are present at birth.

The ability to recognize antigen is encompassed in a plasma membrane receptor in T and B lymphocytes. These receptors are randomly generated by a complex series of rearrangements of many possible genes, such that each individual T or B cell has a unique antigen receptor. This enormous potential diversity means that for any single antigen the body might encounter, multiple lymphocytes will be able to recognize it with varying degrees of binding strength (affinity) and elicit varying degrees of responses. Since antigen receptor specificity arises by chance, the problem thus arises as to why the body does not self destruct through lymphocytes reacting against self antigens. Fortunately, there are several mechanisms which prevent the T and B cells from doing so. Collectively, these mechanisms create a situation where the immune system is tolerant to self.

The most efficient form of self tolerance is to physically remove or kill any potentially reactive lymphocytes at the sites where they are produced. These sites include the thymus for T cells and the BM for B cells. This is called central tolerance. An important, additional method of tolerance is through regulatory Th cells which inhibit autoreactive cells either directly or via the production of cytokines. Given that virtually all immune responses require initiation and regulation by T helper cells, a major aim of any tolerance induction regime would be to target these T helper cells. Similarly, since Tc's are very important effector cells, their production is a major aim of strategies for, e.g., anti-cancer and anti-viral therapy. In addition, T regulatory cells (Tregs), such as CD4⁺CD25⁺ and NKT cells, provide a means whereby they can suppress potentially autoreactive cells.

The Thymus

The thymus essentially consists of developing thymocytes (T lymphocytes within the thymus) interspersed within the diverse stromal cells (predominantly epithelial cell subsets) which constitute the microenvironment and provide the growth factors (GF) and cellular interactions necessary for the optimal development of the T cells.

The thymus is an important organ in the immune system because it is the primary site of production of T lymphocytes. The role of the thymus is to attract appropriate BM-derived precursor cells from the blood, as described below, and induce their commitment to the T cell lineage, including the gene rearrangements necessary for the production of the T cell receptor (TCR) for antigen. Each T cell has a single TCR type and is unique in its specificity. Associated with this TCR production is cell division, which expands the number of T cells with that TCR type and, hence, increases the likelihood that every foreign antigen will be recognized and eliminated. However, a unique feature of T cell recognition of antigen is that, unlike B cells, the TCR only recognizes peptide fragments physically associated with MHC molecules. Normally, this is self MHC, and the ability of a TCR to recognize the self MHC/peptide complex is selected for in the thymus. This process is called positive selection and is an exclusive feature of cortical epithelial cells. If the TCR fails to bind to the self MHC/peptide complexes, the T cell dies by “neglect” because the T cells need some degree of signalling through the TCR for its continued survival and maturation.

Since the outcome of the TCR gene rearrangements is a random event, some T cells will develop which, by chance, can recognize self MHC/peptide complexes with high affinity. Such T cells are, thus, potentially self-reactive and could be involved in autoimmune diseases, such as multiple sclerosis (MS), rheumatoid arthritis (RA), diabetes, thyroiditis, and systemic lupus erythematosus (SLE). Fortunately, if the affinity of the TCR to self MHC/peptide complexes is too high, and the T cell encounters this specific complex in the thymus, the developing thymocyte is induced to undergo a suicidal activation and dies by apoptosis, a process called negative selection. This process is also called central tolerance. Such “high affinity for self” T cells die rather than respond because in the thymus they are still immature. The most potent inducers of this negative selection in the thymus are APC called dendritic cells (DC). DC deliver the strongest signal to the T cells, which causes deletion in the thymus. However, in the peripheral lymphoid organs where the T cells are more mature, the DC presenting the same MHC/peptide complex to the same TCR would cause activation of that T cell bearing the TCR.

Thymus Atrophy and Age

While the thymus is fundamental for a functional immune system, releasing about 1% of its T cell content into the bloodstream per day, one of the apparent anomalies of mammals and other animals is that this organ undergoes severe atrophy as a result of sex steroid production. This atrophy occurs gradually over a period of about 5-7 years, with the nadir level of T cell output being reached around 20 years of age (Douek et al., Nature (1998) 396:690-695) and is in contrast to the reversible atrophy induced during a stress response to corticosteroids. Structurally, the thymic atrophy involves a progressive loss of lymphocyte content, a collapse of the cortical epithelial network, an increase in extracellular matrix material, and an infiltration of the gland with fat cells (adipocytes) and lipid deposits (Haynes et al., (1999) J. Clin. Invest. 103: 453). This process may even begin in young children (e.g., around five years of age); (Mackall et al., (1995) N. Eng. J. Med. 332:143), but it is profound from the time of puberty when sex steroid levels reach a maximum.

The impact of thymus atrophy is reflected in the periphery, with reduced thymic input to the T cell pool, which results in a less diverse TCR repertoire (as this can only be provided by the new naïve T cells). Altered cytokine profiles (Hobbs et al., (1993) J. Immunol. 150:3602; Kurashima et al., (1995) Int. Immunol. 7:97), changes in CD4⁺ and CD8⁺ subsets, biases towards memory as opposed to naïve T cells (Mackall et al., (1995) N. Engl. J. Med. 332:143), and a reduced ability to respond to antigenic or mitogenic stimulation are also observed.

Since the thymus is the primary site for the production and maintenance of the peripheral T cell pool, this atrophy has been widely postulated as being the primary cause of the increased incidence of immune-based disorders in the elderly. In particular, conditions, such as general immunodeficiency, poor responsiveness to opportunistic infections and vaccines, and an increase in the frequency of autoimmune diseases, such as multiple sclerosis, rheumatoid arthritis, and lupus (Doria et al., (1997) Mech. Age. Dev. 95: 131-142; Weyand et al., (1998) Mech. Age. Dev. 102: 131-147; Castle, (2000) Clin Infect Dis 31(2): 578-585; Murasko et al., (2002) Exp. Gerontol. 37:427-439), increase in incidence and severity with age. Such deficiencies of the immune system are often illustrated by a decrease in T cell dependent immune functions (e.g., cytolytic T cell activity and mitogenic responses). While homeostatic mechanisms maintain T cell numbers in healthy individuals, when there is a major loss of T cells, e.g., in AIDS, and following chemotherapy or radiotherapy, adult patients are highly susceptible to opportunistic infections because all these conditions involve a loss of T cells and/or other blood cells (see below). Lymphocyte recovery is also severely retarded. The atrophic thymus is unable to reconstitute CD4⁺ T cells that are lost during HIV infection (Douek et al., Nature (1998) 396:690-695) and CD4⁺ T cells take three to four times longer to return to normal levels following chemotherapy in post-pubertal patients as compared to pre-pubertal patients (Mackall et al., (1995) N. Engl. J. Med. 332:143-149). As a consequence, these patients lack the cells needed to respond to infections, and they become severely immune suppressed (Mackall et al., (1995) N. Eng. J. Med. 332:143; Heitger et al., (2002) Blood 99:4053). There is also an increase in cancers and tumor load in later life (Hirokawa, (1998) “Immunity and Ageing,” in PRINCIPLES AND PRACTICE OF GERIATRIC MEDICINE, (M. Pathy, ed.) John Wiley and Sons Ltd; Doria et al., (1997) Mech. Age. Dev. 95: 131; Castle, (2000) Clin. Infect. Dis. 31:578).

However, recent work by Douek et al., ((1998) Nature 396:690) has shown thymic output occurs even if only very slight (about 5% of the young levels), in older humans (e.g., even sixty-five years old and above, and after anti-retroviral treatment in older HIV patients). This was exemplified by the presence of T cells with T Cell Receptor Excision Circles (TRECs); TRECs are formed as part of the generation of the TCR for antigen and are only found in newly produced T cells. Furthermore, Timm and Thoman ((1999) J. Immunol. 162:711) have shown that although CD4⁺ T cells are regenerated in old mice post-bone marrow transplant (BMT), they appear to show a bias towards memory cells due to the aged peripheral microenvironment coupled with poor thymic production of naïve T cells. TREC levels have also been analyzed following hematopoietic stem cell transplantation (Douek et al., (2000) Lancet 355:1875).

Thymus and the Neuroendocrine Axis

The thymus is influenced to a great extent by its bidirectional communication with the neuroendocrine system (Kendall, (1988) “Anatomical and physiological factors influencing the thymic microenvironment,” in THYMUS UPDATE I, Vol. 1. (M. D. Kendall, and M. A. Ritter, eds.) Harwood Academic Publishers, p. 27). Of particular importance is the interplay between the pituitary, adrenals, and gonads on thymic function, including both trophic (thyroid stimulating hormone or TSH, and growth hormone or GH) and atrophic effects (luteinizing hormone or LH, follicle stimulating hormone or FSH, and adrenocorticotropic hormone or ACTH) (Kendall, (1988) “Anatomical and physiological factors influencing the thymic microenvironment,” in THYMUS UPDATE I, Vol. 1. (M. D. Kendall, and M. A. Ritter, eds.) Harwood Academic Publishers, p. 27; Homo-Delarche et al., (1993) Springer Sem. Immunopathol. 14:221) Indeed, one of the characteristic features of thymic physiology is the progressive decline in structure and function, which is commensurate with the increase in circulating sex steroid production around puberty which, in humans, generally, occurs from the age of 12-14 onwards (Hirokawa and Makinodan, (1975) J. Immunol. 114:1659; Tosi et al., (1982) Clin. Exp. Immunol. 47:497; and Hirokawa, et al., (1994) Immunol. Lett. 40:269).

The thymus essentially consists of developing thymocytes interspersed within the diverse stromal cells (predominantly epithelial cell subsets) which constitute the microenvironment and provide the growth factors and cellular interactions necessary for the optimal development of the T cells. The precise target of the hormones, as well as the mechanism by which they induce thymus atrophy and improved immune responses, has yet to be determined. Examination of testicular feminized mutant mice, however, indicates that functional sex steroid receptors must be expressed on the stromal cells of the thymus for atrophy to occur. The symbiotic developmental relationship between thymocytes and the epithelial subsets that control their differentiation and maturation (Boyd et al., (1993) Immunol. Today 14:445) means that sex-steroid inhibition could occur at the level of either cell type, which would then influence the status of the other cell type. Bone marrow stem cells are reduced in number and are qualitatively different in aged patients. HSC are able to repopulate the thymus, although to a lesser degree than in the young. Thus, the major factor influencing thymic atrophy appears to be intrathymic. Furthermore, thymocytes in older aged animals (e.g., those ≧18 months) retain their ability to differentiate to at least some degree (George and Ritter, (1996) Immunol. Today 17:267; Hirokawa et al., (1994) Immunology Leters 40:269; Mackall et al., (1998) Eur. J. Immunol. 28: 1886). However, recent work by Aspinall has shown that in aged mice there is a defect in thymocyte production, which is manifested as a block within the precursor triple negative population, namely, the CD44+CD25+ (TN2) stage. (Aspinall et al., (1997) J. Immunol. 158:3037).

In the particular case for AIDS, the primary defect in the immune system is the destruction of CD4+ cells and, to a lesser extent, the cells of the myeloid lineages of macrophages and dendritic cells (DC). Without these, the immune system is paralysed and the patient is extremely susceptible to opportunistic infection with death a common consequence. The present treatment for AIDS is based on a multitude of anti-viral drugs to kill or deplete the HIV virus. Such therapies are now becoming more effective with viral loads being reduced dramatically to the point where the patient can be deemed as being in remission. The major problem of immune deficiency still exists, however, because there are still very few functional T cells, and those which do recover, do so very slowly. The period of immune deficiency is thus still a very long time and in some cases immune defense mechanisms may never recover sufficiently.

Hematopoiesis

Bone Marrow and Hematopoietic Stem Cells

The major cells of the immune system are the B and T lymphocytes (a major class of white blood cells), and the antigen presenting cells (APC). All immune cells are basically derived from hematopoietic stem cells (HSC) and their progeny, the Common Lymphoid Progenitor (CLP) and the Common Myeloid Progenitor (CMP), which are produced in the BM. Some of the precursor cells migrate to the thymus and are converted into T cells and thymic DC. DC play a role in inducing self-tolerance.

Only a small proportion (approximately 0.01% in young animals) of the HSC are released from the BM and find their way to the thymus via the blood supply, where they undergo division and maturation to form T cells, which are then returned into the general circulation. These new recent thymic emigrant (RTE) cells form a major part of the immune system, and are primarily Th and Tc and are important in maintaining a constant supply of new T cells with a diverse TCR repertoire for the initiation of almost all immune responses. Prior to leaving the thymus, Th and Tc cells can effectively distinguish foreign antigen because, as described above, T cells are “selected” in the thymus so that those T cells that leave recognize all of the cells in the body as self and, under normal circumstances, do not respond against them.

B cells are also ultimately derived from HSC and develop in the BM before exiting into the peripheral immune system. Following interactions with T cells and other cells of the immune system, B cells develop into plasma cells that produce and release large amounts of antibodies, which help the body destroy infective organisms and abnormal cells.

Other HSC produced by the BM are also utilized for the production of all other blood cells, such as NK cells, regulatory cells, common myeloid progenitor derived cells, neutrophils, basophils and eosinophils, dendritic cells, monocytes, macrophages, platelets, and red blood cells.

Hematopoietic Stem Cell Transplantation

Hematopoietic stem cell transplantation (HSCT)—also commonly known as bone marrow transplantation (BMT)—is a treatment used to enhance the recovery of the immune system in, e.g., certain critical cancer conditions. “HSCT” and “BMT” and “transplant” are used interchangeably and are herein defined as a transplant into a recipitent, containing or enriched for HSC, BM cells, stem cells, and/or any other cells which gives rise to blood, thymus, BM and/or any other immune cells, including, but not limited to, HSC, epithelial cells, common lymphoid progenitors (CLP), common myelolymphoid progenitors (CMLP), multilineage progenitors (MLP), and/or mesenchymal stem cells in the BM. In some embodiments, the transplant may be a peripheral blood stem cell transplant (PBSCT). The HSC maybe be mobilized from the BM and then harvested from the blood, or contained within BM physically extracted from the donor. The HSC may be either purified, enriched, or simply part of the collected BM or blood, and are then injected into a recipient. Transplants may be allogeneic, autologous, syngeneic, or xenogenic, and may involve the transplant of any number of cells, including “mini-transplants,” which involve smaller numbers of cells. In some embodiments, HSCT is given prior to, concurrently with, or after sex steroid inhibition.

HSC is a nonlimiting exemplary type of cell, which may be transplanted and/or genetically modified, as used throughout this application. However, as will be readily understood by one skilled in the art, in practicing the inventions provided herein, HSC may be replaced with any one (or more) of a number of substitute cell types without undue experimentation, including, but not limited to, BM cells, stem cells, and/or any other cell which gives rise to blood, thymus, BM and/or any other immune cells, including, but not limited to, HSC, epithelial stem cells, CLP, CMLP, MLP, and/or mesenchymal stem cells in the BM. In some embodiments, HSC are derived from a fetal liver and/or spleen.

Both chemotherapy and radiation therapy can destroy cancer cells. However, because of the lack of specificity, these therapies also kill healthy cells, including virtually all white blood cells (WBC), as well as the HSC in the BM. It is this destruction of WBC and HSC that leads to the patient's need for HSCT. HSCT allows, for example, stem cells and their progeny cells that were damaged by, e.g., chemotherapy or radiation treatment, to be replaced with healthy stem cells that can ultimately produce the blood cells that the patient needs.

HSCT is the basic treatment for a number of hematological cancers, such as leukemias and lymphomas (cancers of the blood and immune system cells), as well as non-malignant immune disorders such as severe combined immunodeficiency, Fanconi's anemia, myelodysplastic syndromes, amyloidosis, aplastic anemia, Diamond Blackfan anemia, hemophagocytic lymphohitiocytosis, Kostmann syndrome, Wiskott-Aldrich syndrome, thrombocytopenias, and hemoglobinopathologies, such as sickle cell disease and thalassemia. Leukemia and lymphoma, which are commonly treated by myeloablation or myelodepletion to rid the body of cancerous cells, are commonly followed by HSCT to recover immune function. The ability of the HSC to first colonize the BM and convert to blood cells (engraftment) is directly linked to the absolute number and quality of the HSC injected, and the functional capacity of the recipient bone marrow microenvironment and the HSC niches. The methods of the present invention either alone or in combination (concurrently or sequentially) with the administration of HSC mobilizing agents, such as cytokines (e.g., G-CSF or GM-CSF), or drugs (e.g., cyclophosphamide), allow faster and/or better engraftment and may also allow chemotherapy and radiation therapy to be given at higher doses and/or more frequently.

Modern clinical medical procedures often employ a transplantation of HSC derived from another donor's blood (an allogeneic HSCT), where advantage is taken of donor T cells reacting against the host cancer cells (graft versus leukemia (GVL)) but this is counterbalanced by other T donor T cells reacting against the host in general (graft versus host (GVH) disease) which can be fatal. Since the success of HSCT and, hence, patient survival, is directly related to the number of HSC injected and the speed of engraftment, using the methods of the present invention means that current HSCT programs will be more successful and that many more patients will be able to receive HSCT than is currently possible.

Mechanisms of enhancing HSC mobilization from the BM are important in ensuring that as many HSC as possible are available for collection from a donor. GM-CSF and G-CSF are presently used for this purpose, but other agents, such as chemotherapy and cytokines have also been shown to be effective. The ability to more effectively mobilize HSC has application beyond hematological repair. Recent studies have shown that HSC are multipotent and may be utilized for repair of damaged tissues, e.g., cardiac muscle, skeletal muscle, liver, bone, connective tissue, epithelial tissue, pancreas, and vasculature.

One limitation of current HSCT strategies is associated with infection, particularly viral, fungal, and encapsulated bacteria, due to prolonged immunodeficiency, and this remains a significant cause of post-transplantal morbidity and mortality in adults. The infections associated with HSCT are generally very difficult to control, even with modern antimicrobial reagents. Children generally recover immune capacity within months after HSCT (Parkman et al., (1997) Immunol. Rev. 157:73), in contrast to the delay in lymphoid recovery in adult recipients which may last years and even then be a very poor reflection of the young optimum. This delay in adults is dependent on a variety of factors, but the susceptibility to infection is primarily due to the well-recognized decline in T and B cell production with age (Parkman et al., (1997) Immunol. Rev. 157:73).

Additionally, the rate of engraftment plays a role, wherein the longer the rate of engraftment, the more likely opportunistic infection will occur.

A second limitation of current HSCT strategies occurs when the grafted cells “reject” the recipient of the cells. This is known clinically as “graft versus host disease” (GVHD). An autologous transplant may avoid GVHD. However, the overall anti-cancer success rates of autologous transplants are lower as compared to allogeneic transplants. In cancer patients, autologous transplants have the disadvantage that they do not produce a Graft Versus Tumor (GVT) effect (which is similar to the GVH effect), and there is the risk that cancerous cells may be returned to the patient with the transplant. It has been discovered that sex steroid inhibition in murine allogeneic HSCT models and castrated recipients of allogeneic HSCT improves post-transplant reconstitution of cells of both the myeloid and lymphoid lineages. Data presented herein shows a significant increase in T and B cell reconstitution without an exacerbation of GVHD or loss of GVT activity (see, e.g., Example 19).

A further limitation of HSCT treatments is the lack of donors to treat all the potential candidates. Although umbilical cord blood (UCB) has been utilized to a fairly limited extent, there are few cells from each donor and, as a consequence, this has been mainly used in children where the total number of HSC required is lower (HSC number required is linked to patient body weight). Other than UCB, donors are in limited supply, and there must be an acceptable MHC match or the risk of GVH is high. If less cells were required, as a result of improved engraftment or a less rigorous match was required, thus reducing the risk of rejection or GVH, potentially HSCT could be used more widely, for example, to treat autoimmune disease, and sources such as cord blood could be utilized (e.g., 1.5×10⁷ cells/kg for recipient engraftment).

T Cells

T cells are the major component of the immune system and are produced in the thymus. The most important T cells are Th cells because these are the cells that initiate virtually all immune responses. The absence of these Th cells (e.g., caused by HIV infection, chemotherapy, radiation, etc.) directly results in immunosuppression and the consequent susceptibility to infections and tumors, and death occurs quickly. An important role of a subset of Th cells is to regulate immune responses. The balance between enhancement and suppression of T and B cell function has a major effect on e.g., whether a vaccine is efficacious, whether a cancer or tumor is attacked, or whether a transplant is tolerated or rejected.

The thymus, while being very active in the young, progressively declines in both size and functional output with age. This is particularly evident at the onset of puberty. Since thymocyte export is directly related to the cellularity in the thymus (Scollay et al., (1980) Eur. J. Immunol. 10:210; Berzins et al., (1998) J. Exp. Med. 187:1839), age-related thymic atrophy results in a gradual decrease in recent thymic emigrants (RTEs) (Steffens et al., (2000) Clin. Immunol. 97:95; Sempowski et al., (2002) Mol. Immunol. 38:841-848; Sutherland et al., (submitted)) and a decrease in the naïve to memory T cell ratio (Ernst et al., (1990) J. Immunol. 145:1295); Kurashima et al., (1995) Int. Immunol. 7:97; Utsuyama et al., (1992) Mech. Ageing Dev. 63:57) resulting in a restricted TCR repertoire in both CD4⁺ and CD8+ T cells (Mosley et al., (1998) Cell. Immunol. 189: 10; LeMaoult et al., (2000) J. Immunol. 165:2367). Consequently, T cell proliferation in response to non-specific and receptor-mediated (CD3/TCR) stimulation is severely compromised with age (Hertogh-Huijbregts et al., (1990) Mech. Ageing Dev. 53:141-155; Flurkey et al., (1992) J. Gerontol. 47:B115; Kirschmann et al., (1992) Cell. Immunol. 139:426).

With increasing age there is a gradual decline in the immune function of humans; children respond very well, younger adults reasonably so, but from middle age and older, this response can be very poor. This decline indicates the presence of deficiencies or alterations in one of more of the three major cell types involved in virtually all responses: (i) antigen presenting cells (which capture antigen and present it to, and thereby activate, T lymphocytes); (ii) T lymphocytes, and (iii) B lymphocytes. Deficiencies or alterations in any one of these three cell types may explain why the immune response to stimulation against antigens may be suboptimal. The deficiency or alterations may be at the level of the cell or may refer to quantity or functionality. Of these, the most likely defect is encompassed within the T cell compartment because of the dramatic decline in thymus function with age primarily due to the impact of sex steroids. This leads to a loss of new or “naïve” T cells exported into the bloodstream, which are needed for responses to new antigens. In addition to the numerical loss of potential responding T cells, the pre-existing T cells may be suppressed to some degree by the presence of sex steroids.

Cancer Therapy

As indicated above, chemotherapy and radiotherapy used to treat cancers are often deleterious to the patient's non-cancerous cells, particularly the blood cells. The major limitation to increasing frequency and dose of such treatments is the ability of the patient to survive the treatment and avoid susceptibility to opportunistic infection as a result of the compromised immune system. Thus, it would greatly benefit the patient if the immune recovery was more rapid or the damage less severe.

### SUMMARY OF THE INVENTION

The present invention relates to methods for preventing illness or aiding recovery in a patient by enhancing BM haemopoieses and functionality, enhancing BM engraftment following HSCT, and increasing the functionality of pre-existing T cells and other immune cells by disrupting sex steroid and other hormonal signalling. Immune capacity will also be enhanced by increased levels of naïve T cells produced through renewed thymic function, and also B lymphocytes and other cells of the immune system produced via activated BM function.

It has been discovered that interruption of sex steroid and/or other hormonal signalling enhances the functionality of BM, HSC, T cells, and other cells of the immune system, either by direct effects or indirect effects. This discovery has been exploited to produce the present invention which, in one aspect of the invention, provides methods of enhancing BM haemopoiesis and/or functionality. In some embodiments, haemopoiesis and/or functionality of pre-existing BM is improved. In another embodiment, a patient receives an HSCT, and the HSC haemopoiesis and/or engraftment is improved in the patient.

In one aspect, the invention provides methods of enhancing engraftment following HSCT. In one embodiment, engraftment is enhanced in the BM. In another embodiment, engraftment and/or reconstitution is enhanced in the thymus, whereby thymic recovery is ultimately induced. In yet another embodiment, engraftment and/or reconstitution is enhanced in the spleen and/or other lymphoid organs, tissues, and/or blood. In some embodiments, the HSC are allogeneic, and in other embodiments, the HSC are autologous. In one embodiment, the number of T cell precursors is increased as compared to the number that would have been present in a patient that had HSCT without undergoing interruption of sex steroid signalling. In other embodiments, total white blood cells, donor-derived DC, BM precursors, HSC, CLP, MLP, lymphocytes, myeloid cells, granulocytes, neutrophils, macrophage, NK, NKT, platelets, naïve T cells, memory T cells, helper T cells, effector T cells, regulatory T cells, RBC, B cells, donor-, and/or host-derived peripheral T cells, APC, and/or donor-derived peripheral B cells are increased as compared to the number that would have been present in a patient that had HSCT without undergoing interruption of sex steroid signalling. In yet other embodiments, a patient is also treated with a cytokine (e.g., IL-7, SCF, IL-11, G-CSF, or GM-CSF) or hormone (e.g., growth hormone, or its mediator insulin dependent growth factor (IGF-1) or any member of the fibroblast growth factor family (e.g., FGF 7/Keratinocyte Growth Factor (KGF)) following HSCT to enhance immune recovery and/or engraftment. In another embodiment, the present invention either alone or in combination (concurrently or sequentially) with the administration of hemopoietic agents, such as cytokines (e.g., G-CSF or GM-CSF), allow faster and/or better engraftment and/or homing to the target tissue and/or enhance recovery of immune cells.

In a second aspect of the invention, methods of enhancing the functionality of immune cells in a patient following HSCT are provided. In one embodiment, the immune cells are T cells. In another embodiment, the T cell proliferative responsiveness to T cell receptor (TCR) stimulation is improved. In another embodiment, the T cell responsiveness to an antigen (e.g., tetanus toxoid (TT) or pokeweed mitogen (PWM), or Keyhole Limpet Hemocyanin (KLH)) stimulation is improved. In one embodiment, the T cell responsiveness is improved to a recall antigen (i.e., an improved T cell memory response). In yet another embodiment, the T cell proliferative responsiveness in respect of co-stimulatory or secondary signalling is improved. In some embodiments, the kinetics of T cell responsiveness is improved. In other embodiments, the T cell response to antigen presented by APC is improved. In some embodiments of the invention, immune cell responsiveness is improved within about five-, four-, three- or two months post-transplant. In certain embodiments of the invention, immune cell responsiveness is improved within about one month post-transplant. In other embodiments of the invention, immune cell responsiveness is improved within two weeks post-transplant. In another embodiment of the invention, immune cell responsiveness is improved within one week post-transplant. In yet other embodiments of the invention, immune cell responsiveness is improved within three days post-transplant. In other embodiments, the immune response is improved after three or more months post-treatment involving at this time input from newly-thymic-derived T cells in addition to pre-existing T cells.

In a third aspect, the invention provides methods of enhancing pre-existing immune cell functionality including, but not limited to, immune cells in the periphery. In one embodiment, the cells are T cells. In another embodiment, the cells are DC or other APC. In yet another embodiment, the cells are NK cells or regulatory cells, such as CD4+ CD25+ T cells and natural killer T (NKT) cells. In one embodiment of the invention, T cell proliferative responsiveness to TCR stimulation is improved in the patient. In another embodiment, the T cell responsiveness to antigen (e.g., TT, PWM, or KLH) stimulation is improved. In yet another embodiment, the T cell proliferative responsiveness to secondary or co-stimulatory signalling is improved. In other embodiments, the T cell response to antigen presented by APC is improved. In one specific embodiment, a LHRH/GnRH analog has a direct effect or indirect effect on the responsiveness of pre-existing immune cells. In some embodiments, sex steroid analogs (agonist and antagonists thereto), such as GnRH/LHRH analogs, are used in the methods of the invention to disrupt sex steroid-mediated signalling, immune cells, or BM. In other embodiments, sex steroid analogs directly stimulate (i.e., directly increase the functional activity of) the thymus, BM, and/or pre-existing cells of the immune system, such as T cells, B cells, and DC.

In a fourth aspect, the invention provides methods of enhancing HSC engraftment and mobilization in a patient, or in a blood, HSC, or BM donor. In one embodiment, disrupting sex steroid signalling increases the number and/or functionality of peripheral immune progenitor cells such as HSC, CD34+ cells, CLP, or CMP. One embodiment provides a method for enhancing HSC mobilization comprising disrupting sex steroid signalling, either alone or in combination with administration of an HSC mobilizing agent, for example, cytokines, GM-CSF, G-CSF, CSF, chemotherapeutics, cyclophosphamide, flt-3 ligand, KGF/FGF 7, or other members of the FGF family or IL-7.

In one embodiment, the present invention provides methods to allow chemotherapy and radiation therapy to be given at higher doses and/or more frequently and/or allow faster recovery of or less damage to the immune system after chemotherapy and radiation therapy.

In a fifth aspect, the invention provides methods to prevent or treat illness in a patient. One embodiment provides a method for preventing or diminishing the risk of an infection, illness, or disease in a patient, the method comprising disrupting sex steroid mediated signalling in the patient. In a certain embodiment, signalling is disrupted to the BM. In another embodiment, signalling is disrupted to the thymus. In yet another embodiment, signalling is disrupted to the spleen. In yet another embodiment, signalling is disrupted to the peripheral immune cells.

In some embodiments, the methods of the invention are used to prevent or treat viral infections, such as HIV, herpes, influenza, and hepatitis. In other embodiments, the methods of the invention are used to prevent or treat bacterial infections, such as pneumonia and tuberculosis (TB). In yet other embodiments, the methods of the invention are used to prevent or treat fungal infections, parasitic infections, allergies, and/or tumors, and other cancers, whether malignant or benign.

In other embodiments, the patient receives non-genetically modified HSC transplantation. In some embodiments, BM or HSC are transplanted into the patient to provide a reservoir of precursor cells, which may ultimately be used for the renewed thymic growth. Some of these HSC have the ability to turn into DC or other APC, which may have the effect of providing better antigen presentation to the T cells and, therefore, a better immune response (e.g., increased Ab production and effector T cell numbers and/or functions). In other embodiments, the atrophic thymus in an aged (post-pubertal) patient is in the process of being reactivated by disruption of sex-steroid signalling at the time of HSCT. The reactivating thymus becomes capable of taking up HSC, BM cells from the blood, and other appropriate progenitors, and converting them in the thymus to both new T cells and DC.

In a sixth aspect, the invention provides methods to improve the immune responsiveness of a patient to a vaccine.

In a seventh aspect, gene therapy utilizing genetically modified HSC, lymphoid progenitor, myeloid progenitor or epithelial stem cells, or combinations thereof (the group and each member herein referred to as “GM cells”), are delivered to the patient to create particular immunities useful in treating or preventing an illness.

In some embodiments of certain aspects of the invention, the illness is one that has a defined genetic basis, such as that caused by a genetic defect. These genetic diseases are well known to those in the art, and include autoimmune diseases, diseases resulting from the over- or under-production of certain proteins, tumors, and cancers, etc. The disease-causing genetic defect is repaired by insertion of the normal gene into the HSC and, using the methods of the invention, every cell produced from this HSC will then carry the gene correction.

In other embodiments, the disease is a T cell disorder selected from the group consisting of viral infections (such as human immunodeficiency virus (HIV)), T cell functional disorders, and any other disease or condition that reduces T cells numerically or functionally, either directly or indirectly, or causes T cells to function in a manner which is harmful to the individual.

In yet other embodiments, the present invention provides methods for treating or preventing infection by an infectious agent, such as HIV, by transplanting GM cells that have been genetically modified to resist or prevent infection, activity, replication, and the like, and combinations thereof into a patient prior to, or concurrently with, thymic reactivation. In one embodiment, the HSC are modified to include a gene whose product interferes with HIV infection, function, and/or replication in the T cells (and/or other HSC-derived cells) of the patient. In a particular embodiment, HSC are genetically modified with viral resistance gene, such as the RevM10 gene (see, e.g., Bonyhadi et al., (1997) J. Virol. 71:4707) or the CXCR4 or PolyTAR genes (Strayer et al., (2002) Mol. Ther. 5:33). This confers a degree of resistance to the virus, thereby preventing or treating disease caused by the virus.

In another aspect, the invention provides for disruption of sex steroid mediated signalling to, and subsequent reactivation of, the thymus. In one embodiment, castration is used to disrupt the sex steroid mediated signalling. In a particular embodiment, chemical castration is used. In another embodiment, surgical castration (e.g., by removal of the testes or by ovariectomy) is used. In some embodiments, complete inhibition of sex steroid signalling occurs. In another embodiment, partial disruption of sex steroid signalling occurs. In one embodiment, castration reverses the state of the thymus towards its pre-pubertal state, thereby reactivating it. In another embodiment, castration modifies the level of other molecules, which enhance immune cell responsiveness and/or proliferation and/or activation state by having, e.g., a direct effect on pre-existing immune cells.

In certain embodiments, sex steroid mediated signalling may be directly or indirectly blocked (e.g., inhibited, inactivated or made ineffectual) by the administration of modifiers of sex hormone production, action, binding or signalling including, but not limited to, agents which bind a sex hormone or its receptor, agonists or antagonists of sex hormones including, but not limited to, GnRH/LHRH, anti-estrogenic and anti-androgenic agents, SERMs, SARMs, anti-estrogen antibodies, anti-androgen ligands, anti-estrogen ligands, LHRH ligands, passive (antibody) or active (antigen) anti-LHRH (or other sex steroid) vaccinations, or combinations thereof (“blockers”). In one embodiment, one or more blocker is used. In some embodiments, the one or more blockers are administered by a sustained peptide-release formulation. Examples of sustained peptide-release formulations are provided in WO 98/08533, the entire contents of which are incorporated herein by reference.

### DESCRIPTION OF THE FIGURES

FIGS. 1A-B: Castration rapidly regenerates thymus cellularity. FIGS. 1A-B are graphic representations showing the changes in thymus weight and thymocyte number pre- and post-castration. Thymus atrophy results in a significant decrease in thymocyte numbers with age, as measured by thymus weight (FIG. 1A) or by the number of cells per thymus (FIG. 1B). For these studies, aged (i.e., 2-year old) male mice were surgically castrated. Thymus weight in relation to body weight (FIG. 1A) and thymus cellularity (FIG. 1B) were analyzed in aged (1 and 2 years) and at 2-4 weeks post-castration (post-cx) male mice. A significant decrease in thymus weight and cellularity was seen with age compared to young adult (2-month) mice. This decrease in thymus weight and cell number was restored by castration, although the decrease in cell number was still evident at 1 week post-castration (FIG. 1B). By 2 weeks post-castration, cell numbers were found to increase to approximately those levels seen in young adults (FIG. 1B). By 3 weeks post-castration, numbers have significantly increased from the young adult and these were stabilized by 4 weeks post-castration (FIG. 1B). Results are expressed as mean ±1 SD of 4-8 mice per group (FIG. 1A) or 8-12 mice per group (FIG. 1B). **=p≦0.01; ***=p≦0.001 compared to young adult (2 month) thymus and thymus of 2-6 weeks post-castrate mice.

FIGS. 2A-D: Castration restores the CD4:CD8 T cell ratio in the periphery. For these studies, aged (2-year old) mice were surgically castrated and analyzed at 2-6 weeks post-castration for peripheral lymphocyte populations. FIGS. 2A and 2B show the total lymphocyte numbers in the spleen. Spleen numbers remain constant with age and post-castration because homeostasis maintains total cell numbers within the spleen (FIGS. 2A and 2B). However, cell numbers in the lymph nodes in aged (18-24 months) mice were depleted (FIG. 2B). This decrease in lymph node cellularity was restored by castration (FIG. 2B). FIG. 2C shows that the ratio of B cells to T cells did not change with age or post-castration in either the spleen or lymph node, as no change in this ratio was seen with age or post-castration. However, a significant decrease (p<0.001) in the CD4⁺:CD8⁺ T cell ratio was seen with age in both the (pooled) lymph node and the spleen (FIG. 2D). This decrease was restored to young adult (i.e., 2 month) levels by 4-6 weeks post-castration (FIG. 2D).

Results are expressed as mean ±1 SD of 4-8 (FIGS. 2A, and 2C) or 8-10 (FIGS. 2B and 2D) mice per group. *=p≦0.05; **=p≦0.01; ***=p≦0.001 compared to young adult (2-month) and post-castrate mice.

FIG. 3: Thymocyte subpopulations are retained in similar proportions despite thymus atrophy or regeneration by castration. For these studies, aged (2-year old) mice were castrated and the thymocyte subsets analyzed based on the markers CD4 and CD8. Representative Fluorescence Activated Cell Sorter (FACS) profiles of CD4 (X-axis) vs. CD8 (Y-axis) for CD4⁻CD8⁻DN, CD4⁺CD8^{+DP, CD}4⁺CD8⁻ and CD4⁻CD8⁺ SP thymocyte populations are shown for young adult (2 months), aged (2 years) and aged, post-castrate animals (2 years, 4 weeks post-cx). Percentages for each quadrant are given above each plot. No difference was seen in the proportions of any CD4/CD8 defined subset with age or post-castration. Thus, subpopulations of thymocytes remain constant with age, and there was a synchronous expansion of thymocytes following castration.

FIGS. 4A-B: Regeneration of thymocyte proliferation by castration. Mice were injected with a pulse of BrdU and analyzed for proliferating (BrdU⁺) thymocytes. For these studies, aged (2-year old) mice were castrated and injected with a pulse of bromodeoxyuridine (BrdU) to determine levels of proliferation. Representative histogram profiles of the proportion of BrdU+ cells within the thymus with age and post-castration are shown (FIG. 4A). No difference was observed in the total proportion of proliferation within the thymus, as this proportion remains constant with age and following castration (FIG. 4A and left graph in FIG. 4B). However, a significant decrease in number of BrdU⁺ cells was seen with age (FIG. 4B, right graph). By 2 weeks post-castration, the number of BrdU⁺ cells increased to a number similar to that seen in young adults (i.e., 2 month) (FIG. 4B, right graph). Results are expressed as mean ±1 SD of 4-14 mice per group. ***=p≦0.001 compared to young adult (2-month) control mice and 2-6 weeks post-castration mice.

FIGS. 5A-H: Castration enhances proliferation within all thymocyte subsets. For these studies, aged (2-year old) mice were castrated and injected with a pulse of bromodeoxyuridine (BrdU) to determine levels of proliferation. Analysis of proliferation within the different subsets of thymocytes based on CD4 and CD8 expression within the thymus was performed. FIG. 5A shows that the proportion of each thymocyte subset within the BrdU+ population did not change with age or post-castration. However, as shown in FIG. 5B, a significant decrease in the proportion of DN (CD4⁻CD8⁻) thymocytes proliferating was seen with age. FIG. 5C shows that no change in the total proportion of BrdU+ cells (i.e., proliferating cells) within the TN subset was seen with age or post-castration. However, a significant decrease in proliferation within the TN1 (CD44⁺CD25⁻CD3⁻CD4⁻CD8⁻) subset (FIG. 5E) and significant increase in proliferation within TN2 (CD44+CD25+CD3−CD4−CD8⁻) subset (FIG. 5F) was seen with age. This was restored post-castration (FIGS. 5D-F). Results are expressed as mean ±1 SD of 4-17 mice per group. *=p<0.05; **=p≦0.01 (significant); ***=p≦0.001 (highly significant) compared to young adult (2-month) mice; ̂=significantly different from 2-6 weeks post-castrate mice (FIGS. 5E-H).

FIGS. 6A-C: Castration increases T cell export from the aged thymus. For these studies, aged (2-year old) mice were castrated and were injected intrathymically with FITC to determine thymic export rates. The number of FITC+ cells in the periphery was calculated 24 hours later. As shown in FIG. 6A, a significant decrease in recent thymic emigrant (RTE) cell numbers detected in the periphery over a 24 hour period was observed with age. Following castration, these values had significantly increased by 2 weeks post-cx. As shown in FIG. 6B, the rate of emigration (export/total thymus cellularity) remained constant with age, but was significantly reduced at 2 weeks post-castration. With age, a significant increase in the ratio of CD4⁻ to CD8⁺ RTE was seen; this was normalized by 1-week post-cx (FIG. 6C).

Results are expressed as mean ±1 SD of 4-8 mice per group. *=p≦0.05; **=p≦0.01; ***=p≦0.001 compared to young adult (2-month) mice for (FIG. 6A) and compared to all other groups (FIGS. 6B and 6C). ̂=p≦0.05 compared to aged (1- and 2-year old) non-cx mice and compared to 1-week post-cx, aged mice.

FIGS. 7A-B: Castration enhances thymocyte regeneration following T cell depletion. Three-month old mice were either treated with cyclophosphamide (intraperitoneal injection with 200 mg/kg body weight cyclophosphamide, twice over 2 days) (FIG. 7A) or exposed to sublethal irradiation (625 rads) (FIG. 7B). For both models of T cell depletion studied, castrated (Cx) mice showed a significant increase in the rate of thymus regeneration compared to their sham-castrated (ShCx) counterparts. Analysis of total thymocyte numbers at 1 and 2-weeks post-T cell depletion (TCD) showed that castration significantly increases thymus regeneration rates after treatment with either cyclophosphamide or sublethal irradiation (FIGS. 7A and 7B, respectively). Data is presented as mean ±1 SD of 4-8 mice per group. For FIG. 7A, ***=p≦0.001 compared to control (age-matched, untreated) mice; ̂=p≦0.001 compared to both groups of castrated mice. For FIG. 7B, ***=p≦0.001 compared to control mice; ̂=p≦:0.001 compared to mice castrated 1-week prior to treatment at 1-week post-irradiation and compared to both groups of castrated mice at 2-weeks post-irradiation.

FIGS. 8A-B: Total lymphocyte numbers within the spleen and lymph nodes post-cyclophosphamide treatment. For these studies, (3 month old) mice were depleted of lymphocytes using cyclophosphamide (intraperitoneal injection with 200 mg/kg body weight cyclophosphamide, twice over 2 days) and either surgically castrated or sham-castrated on the same day as the last cyclophosphamide injection. Thymus, spleen and lymph nodes (pooled) were isolated and total cellularity evaluated. Sham-castrated mice had significantly lower cell numbers in the spleen at 1 and 4-weeks post-treatment compared to control (age-matched, untreated) mice (FIG. 8A). A significant decrease in cell number was observed within the lymph nodes at 1 week post-treatment for both treatment groups (FIG. 8B). At 2-weeks post-treatment, Cx mice had significantly higher lymph node cell numbers compared to ShCx mice (FIG. 8B). Each bar represents the mean ±1 SD of 7-17 mice per group. *=p≦0-0.05; **=p≦0.01 compared to control (age-matched, untreated). ̂=p≦0.05 compared to castrated mice.

FIG. 9: Changes in thymus (open bars), spleen (gray bars) and lymph node (black bars) cell numbers following treatment with cyclophosphamide, a chemotherapy agent, and surgical or chemical castration performed on the same day. Note the rapid expansion of the thymus in castrated animals when compared to the non-castrate (cyclophosphamide alone) group at 1 and 2 weeks post-treatment. In addition, spleen and lymph node numbers of the castrate group were well increased compared to the cyclophosphamide alone group. (n=34 per treatment group and time-point). Chemical castration is comparable to surgical castration in regeneration of the immune system post-cyclophosphamide treatment.

FIGS. 10A-C: Changes in thymus (FIG. 10A), spleen (FIG. 10B) and lymph node (FIG. 10C) cell numbers following irradiation (625 rads) one week after surgical castration. For these studies, young (3-month old) mice were depleted of lymphocytes using sublethal (625 rads) irradiation. Mice were either sham-castrated or castrated 1-week prior to irradiation. A significant increase in thymus regeneration (i.e., faster rate of thymus regeneration) was observed with castration (FIG. 10A). Note the rapid expansion of the thymus in castrated animals when compared to the non-castrate (irradiation alone) group at 1 and 2 weeks post-treatment. (n=3-4 per treatment group and time-point). No difference in spleen (FIG. 10B) or lymph node (FIG. 10C) cell numbers was seen with castrated mice. Lymph node cell numbers were still chronically low at 2-weeks post-treatment compared to control mice (FIG. 10C). Results are expressed as mean ±1 SD of 4-8 mice per group. *=p≦0.05; **=p≦0.01 compared to control mice; ***=p≦0.001 compared to control and castrated mice.

FIGS. 11A-C: Changes in thymus (FIG. 11A), spleen (FIG. 11B) and lymph node (FIG. 11C) cell numbers following irradiation and castration on the same day. For these studies, young (3-month old) mice were depleted of lymphocytes using sublethal (625 rads) irradiation. Mice were either sham-castrated or castrated on the same day as irradiation. Castrated mice showed a significantly faster rate of thymus regeneration compared to sham-castrated counterparts (FIG. 11A). Note the rapid expansion of the thymus in castrated animals when compared to the non-castrated group at 2 weeks post-treatment. No difference in spleen (FIG. 11B) or lymph node (FIG. 11C) cell numbers was seen with castrated mice. Lymph node cell numbers were still chronically low at 2-weeks post-treatment compared to control mice (FIG. 11C). Results are expressed as mean ±1 SD of 4-8 mice per group. *=p≦0.05; **=p≦0.01 compared to control mice; ***=p≦0.001 compared to control and castrated mice.

FIG. 12A-B: Total lymphocyte numbers within the spleen and lymph nodes post-irradiation treatment. Three-month old mice were either castrated or sham-castrated 1-week prior to sublethal irradiation (625 rads). Severe lymphopenia was evident in both the spleen (FIG. 12A) and (pooled) lymph nodes (FIG. 12B) at 1-week post-treatment. Splenic lymphocyte numbers were returned to control levels by 2-weeks post-treatment (FIG. 12A), while lymph node cellularity was still significantly reduced compared to control (age-matched, untreated) mice (FIG. 12B). No differences were observed between the treatment groups. Each bar represents the mean ±1 SD of 6-8 mice per group. **=p≦0.01; ***=p≦0.001 compared to control mice.

FIGS. 13A-B: Castration restores responsiveness to HSV-1 immunization. Mice were immunized in the hind foot-hock with 4×10⁵ pfu of HSV. On Day 5 post-infection, the draining lymph nodes (popliteal) were analyzed for responding cells. FIG. 13A shows the lymph node cellularity following foot-pad immunization with Herpes Simplex Virus-1 (HSV-1). Note the increased cellularity in the aged post-castration as compared to the aged non-castrated group. FIG. 13B illustrates the overall activated cell number as gated on CD25 vs. CD8 cells by FACS (i.e., the activated cells are gated on CD8+ CD25+ cells). Castration of the aged mice restored the immune response to HSV-1 with CTL numbers equivalent to young mice. Results are expressed as mean ±1 SD of 8-12 mice. **=p≦0.01 compared to both young (2-month) and castrated mice

FIGS. 14A-C: Vβ10 expression (HSV-specific) on CTL (cytotoxic T lymphocytes) in activated LN (lymph nodes) following HSV-1 inoculation. Despite the normal Vβ10 responsiveness in aged (i.e., 18 months) mice overall, in some mice a complete loss of Vβ10 expression was observed. Representative histogram profiles are shown. Note the diminution of a clonal response in aged mice and the reinstatement of the expected response post-castration.

FIG. 15: Castration enhances activation following HSV-1 infection. FIG. 15 shows representative FACS profiles of activated (CD8⁺CD25⁺) cells in the LN of HSV-1 infected mice. No difference was seen in proportions of activated CTL with age or post-castration. As shown in castration of the aged mice restored the immune response to HSV-1 with CTL numbers equivalent to young mice. Results are expressed as mean ±1 SD of 8-12 mice. **=p≦0.01 compared to both young (2-month) and castrated mice.

FIG. 16: Specificity of the immune response to HSV-1. Popliteal lymph node cells were removed from mice immunized with HSV-1 (removed 5 days post-HSV-1 infection), cultured for 3-days, and then examined for their ability to lyse HSV peptide pulsed EL 4 target cells. CTL assays were performed with non-immunized mice as control for background levels of lysis (as determined by ⁵¹Cr-release). Aged mice showed a significant (p≦0.01, **) reduction in CTL activity at an E:T ratio of both 10:1 and 3:1 indicating a reduction in the percentage of specific CTL present within the lymph nodes. Castration of aged mice restored the CTL response to young adult levels since the castrated mice demonstrated a comparable response to HSV-1 as the young adult (2-month) mice. Results are expressed as mean of 8 mice, in triplicate ±1 SD. **=p≦0.01 compared to young adult mice; ̂=significantly different to aged control mice (p≦0.05 for E:T of 3:1; p≦0.01 for E:T of 0.3:1).

FIGS. 17A-B: Analysis of Vβ TCR expression and CD4⁺ T cells in the immune response to HSV-1. Popliteal lymph nodes were removed 5 days post-HSV-1 infection and analyzed ex-vivo for the expression of CD25, CD8 and specific TCR Vβ markers (FIG. 17A) and CD4/CD8 T cells (FIG. 17B). The percentage of activated (CD25⁺) CD8⁺ T cells expressing either Vβ10 or Vβ8.1 is shown as mean ±1 SD for 8 mice per group in FIG. 17A. No difference was observed with age or post-castration. However, a decrease in CD4/CD8 ratio in the resting LN population was seen with age (FIG. 17B). This decrease was restored post-castration. Results are expressed as mean ±1 SD of 8 mice per group. ***=p≦0.001 compared to young and post-castrated mice.

FIGS. 18A-D: Castration enhances regeneration of the thymus (FIG. 18A), spleen (FIG. 18B) and BM (FIG. 18D), but not lymph node (FIG. 18C) following BM transplantation (BMT) of Ly5 congenic mice. Three-month old, young adults, C57/BL6 Ly5.1+ (CD45.1+) mice were irradiated (at 6.25 Gy), castrated, or sham-castrated 1 day prior to transplantation with C57/BL6 Ly5.2+ (CD45.2+) adult BM cells (10⁶ cells). Mice were killed 2 and 4 weeks later and the thymus (FIG. 18A), spleen (FIG. 18B), lymph node (FIG. 18C) and BM (FIG. 18D) were analyzed for immune reconstitution. Donor/Host origin was determined with anti-CD45.2 (Ly5.2), which only reacts with leukocytes of donor origin. There were significantly more donor cells in the thymus of castrated mice 2 and 4 weeks after BMT compared to sham-castrated mice (FIG. 18A). Note the rapid expansion of the thymus in castrated animals when compared to the non-castrated group at all time-points post-treatment. There were significantly more cells in the spleen and BM of castrated mice 2 and 4 weeks after BMT compared to sham-castrated mice (FIGS. 18B and 18D). There was no significant difference in lymph node cellularity 2, 4, and 6 weeks after BMT (FIG. 18C). Castrated mice had significantly increased congenic (Ly5.2) cells compared to non-castrated animals. Data is expressed as mean ±1 SD of 4-5 mice per group. *=p≦0.05; **=p≦0.01.

FIGS. 19A-C: Castration increases BM and thymic cellularity following congenic BMT. As shown in FIG. 19A, there are significantly more cells in the BM of castrated mice 2 and 4 weeks after BMT. BM cellularity reached untreated control levels (1.5×10⁷±1.5×10⁶) in the sham-castrates by 2 weeks. BM cellularity is above control levels in castrated mice 2 and 4 weeks after congenic BMT. FIG. 19B shows that there are significantly more cells in the thymus of castrated mice 2 and 4 weeks after BMT. Thymus cellularity in the sham-castrated mice is below untreated control levels (7.6×10⁷ 5.2×10⁶) 2 and 4 weeks after congenics BMT. Four weeks after congenic BMT and castration thymic cellularity is increased above control levels. FIG. 19C shows that there is no significant difference in splenic cellularity 2 and 4 weeks after BMT. Spleen cellularity has reached control levels (8.5×10⁷±1.1×10⁷) in sham-castrated and castrated mice by 2 weeks. Each group contains 4 to 5 animals. Open bars indicate sham-castration; closed bars indicate castration.

FIG. 20: Castration increases the proportion of HSC following congenic BMT. Representative FACS dot plots illustrating c-kit (y-axis) versus sca-1 (x-axis) expression. HSC are c-kit^{hi}sca-1^{hi}. There is a significant increase in the proportion of donor-derived HSCs following castration, 2, and 4 weeks after BMT.

FIGS. 21A-B: Castration increases the proportion and number of HSC following congenic BMT. As shown in FIG. 21A, there was a significant increase in the proportion of HSCs following castration, 2 and 4 weeks after BMT (* p<0.05). FIG. 21B shows that the number of HSCs is significantly increased in castrated mice compared to sham-castrated controls, 2 and 4 weeks after BMT (* p<0.05 ** p<0.01). Each group contains 4 to 5 animals. Open bars indicate sham-castration; closed bars indicate castration.

FIGS. 22A-B: There are significantly more donor-derived B cell precursors and B cells in the BM of castrated mice following BMT. As shown in FIG. 22A, there were significantly more donor-derived CD45.1⁺B220⁺IgM⁻B cell precursors in the BM of castrated mice compared to the sham-castrated controls (* p<0.05). FIG. 22B shows that there were significantly more donor-derived B220⁺IgM⁺B cells in the BM of castrated mice compared to the sham-castrated controls (* p<0.05). Each group contains 4 to 5 animals. Open bars indicate sham-castration; closed bars indicate castration.

FIG. 23: Castration does not affect the donor-derived thymocyte proportions following congenic BMT. Two weeks after sham-castration and castration there is an increase in the proportion of donor-derived double negative (CD45.1⁺CD4⁻CD8⁻) early thymocytes. There are very few donor-derived (CD45.1⁺) CD4 and CD8 single positive cells at this early time-point. Four weeks after BMT, donor-derived thymocyte profiles of sham-castrated and castrated mice are similar to the untreated control.

FIG. 24: Castration does not increase peripheral B cell proportions following congenic BMT. There is no difference in splenic B220 expression comparing castrated and sham-castrated mice, 2 and 4 weeks after congenic BMT.

FIG. 25: Castration does not increase peripheral B cell numbers following congenic BMT. There is no significant difference in B cell numbers, 2 and 4 weeks after BMT. Two weeks after congenic BMT, B cell numbers in the spleen of sham-castrated and castrated mice approach untreated control levels (5.0×10⁷±4.5×10⁶). Each group contains 4 to 5 animals. Open bars indicate sham-castration; closed bars indicate castration.

FIGS. 26A-D: Donor-derived triple negative, double positive and CD4 and CD8 single positive thymocyte numbers are increased in castrated mice following BMT. FIG. 26A shows that there were significantly more donor-derived triple negative (CD45.1⁺CD3⁻CD4⁻CD8⁻) thymocytes in the castrated mice compared to the sham-castrated controls, 2 and 4 weeks after BMT (* p<0.05 **p<0.01). FIG. 26B shows that there were significantly more double positive (CD45.1⁺CD4⁺CD8⁺) thymocytes in the castrated mice compared to the sham-castrated controls, 2 and 4 weeks after BMT (* p<0.05 **p<0.01). As shown in FIG. 26C, that there were significantly more CD4 single positive (CD45.1+CD3⁺CD4⁺CD8⁻) thymocytes in the castrated mice compared to the sham-castrated controls 2 and 4 weeks after BMT (* p<0.05 **p<0.01). FIG. 26D shows there were significantly more CD8 single positive (CD45.1⁺CD3⁺CD4⁻CD8⁺) thymocytes in the castrated mice compared to the sham-castrated controls 4 weeks after BMT (* p<0.05 **p<0.01). Each group contains 4 to 5 animals. Open bars indicate sham-castration; closed bars indicate castration.

FIGS. 27A-B: There are very few donor-derived, peripheral T cells 2 and 4 weeks after congenic BMT. As shown in FIG. 27A, there was a very small proportion of donor-derived CD4⁺ and CD8⁺ T cells in the spleens of sham-castrated and castrated mice 2 and 4 weeks after congenic BMT. FIG. 27B shows that there was no significant difference in donor-derived T cell numbers 2 and 4 weeks after BMT. Four weeks after congenic BMT there are significantly less CD4⁺ and CD8⁺ T cells in both sham-castrated and castrated mice compared to untreated age-matched controls (CD4⁺−1.1×10⁷±1.4×10⁶, CD8⁺−6.0×10⁶±1.0×10⁵). Each group contains 4 to 5 animals. Open bars indicate sham-castration; closed bars indicate castration.

FIGS. 28A-B: Castration increases the number of donor-derived DC in the thymus 4 weeks after congenic BMT. As shown in FIG. 28A, donor-derived DC were CD45.1⁺CD11c⁺MHCII⁺. FIG. 28B shows that there were significantly more donor-derived thymic DC in the castrated mice 4 weeks after congenic BMT (* p<0.05). Dendritic cell numbers are at untreated control levels 2 weeks after congenic BMT (1.4×10⁵±2.8×10⁴). Four weeks after congenic BMT, dendritic cell numbers are above control levels in castrated mice. Each group contains 4 to 5 animals. Open bars indicate sham-castration; closed bars indicate castration.

FIGS. 29A-C: Castration enhances immune cell reconstitution in allogeneic HSCT recipients. Lethally irradiated (1300 cGy) CBA/J recipients (3 month old) received transplants with B10.BR TCD BM (5×10⁶). Recipients were either castrated or sham-castrated one day before transplant. Animals were humanely killed on days 14, 28, and 42 and BM (FIG. 29A), thymus (FIG. 29B), and spleen (FIG. 29C) organ cellularity were assessed * (p<0.05). Each group contained 4 to 5 animals.

FIGS. 30A-C: Castration enhances donor-derived HSC and B cells in allogeneic HSCT recipients. Castrated and sham-castrated recipients were transplanted as described in FIG. 29. As shown in FIG. 30A, 14 days after HSCT there were very few donor-derived HSCs (Ly9.1⁻Lin³¹ Sca-1⁺c-kit⁺) in both sham-castrated and castrated mice; however, by day 28, donor HSC numbers were 4-fold higher in the castrated group. Additionally, as shown in FIGS. 30B-C, there are significantly more donor-derived B cells in the BM (FIG. 30B) and spleen (FIG. 30C) of castrated mice. Central and peripheral B cell populations were analyzed using total BM or splenic cell counts and multicolor flow cytometry. B cells were separated into developmental stages based on CD45R, IgM, and CD43 expression: Total B cells (CD45R⁺), pro-B cells (CD43⁺CD45R⁺IgM⁻), pre-B cells (CD43⁻CD45R⁺IgM⁻), immature B cells (CD43⁻CD45R⁺IgM⁺). Donor/host origin was determined with anti-Ly9.1, which only reacts with leukocytes of host origin. Each group contained 4 to 5 animals. Open bars indicate sham-castrated animals; and closed bars represent castrated animals. * (p<0.05) represents a significant increase in cell number in the castrated group compared to the sham-castrated control.

FIGS. 31A-G: Castration enhances thymocyte and peripheral T cell reconstitution as well as the number of host and donor-derived DC in allogeneic HSCT recipients. Castrated and sham-castrated recipients were transplanted as described in FIG. 29. Animals were humanely killed on days 14, 28, and 42, and thymocyte and T cell populations were analyzed using total thymic (FIG. 31A-F) or splenic (FIG. 31G) cell counts and multicolor flow cytometry. DC were defined as CD11^{hi} Ia-k^{hi}. FIG. 31A depicts numbers of TN (CD3⁻CD4⁻CD8⁻) thymocytes. FIG. 31B depicts numbers of DP (CD4⁺CD8⁺) thymocytes. FIG. 31C depicts numbers of CD4⁺SP (CD3⁺CD4⁺CD8⁻) thymocytes. FIG. 31D depicts numbers of CD8+ SP (CD3⁺CD4⁻CD8⁺) thymocytes. As shown in FIG. 33E, there are significantly more host-derived DC in castrated recipients at both 14 and 28 days after allogeneic HSCT as compared to sham-castrated control recipients. Additionally, as shown in FIG. 31F, there are significantly more donor-derived DC in castrated recipients 28 days following allogeneic HSCT, as compared to sham-castrated controls. FIG. 31G depicts numbers of peripheral T cells, which were identified using anti-CD3, anti-CD4, and anti-CD8. Donor/host origin was determined with anti-Ly9.1, which only reacts with leukocytes of host origin. Donor CD4 T cells were Ly9.1⁻CD3⁺CD4⁺CD8⁻ and donor CD8 T cells were Ly9.1⁻CD3⁺CD4⁻CD8⁺. Each group contained 4 to 5 animals. Open bars indicate sham-castrated animals; closed bars represent castrated animals. * (p<0.05) and ** (p<0.01) represent a significant increase in cell number in the castrated group compared to the sham-castrated control.

FIGS. 32A-G: Castration does not alter the function of donor-derived T cells following allogeneic HSCT. Castrated and sham-castrated recipients were transplanted as in FIG. 29. T cell functionality was assessed 42 days after transplantation. FIG. 32A shows the number of donor-derived T cells (CD3⁺CD4⁺ and CD3⁺CD8⁺) six weeks after allo-HSCT.

FIG. 32B shows that castration has no effect on the proliferative capability of T cells after allogeneic HSCT. FIG. 32C shows no difference in alloreactive T-cell proliferation in an MLR. Splenic T cells (4×10⁵ cells/well) from each group (n=5) were incubated with irradiated (20 Gy) BALB/c splenic stimulator cells (2×10⁵ cells/well) in 96-well plates for 5 days and [3H]-thymidine was added during the final 20 hours of culture. FIG. 32D shows no difference in cytolytic activity of donor-derived T cells. FIG. 32E shows intracellular IFNγ expression of alloreactive T cells. Splenic B6 T cells were harvested on day 42 from sham-castrated or castrated recipients as described above and incubated with irradiated (20 Gy) (BALB/C—third party) splenic stimulator cells in 24-well plates for 5 days. Cells were harvested, and restimulated with TCD, irradiated (20 Gy) (BALB/C or B10.BR internal biological control) splenic stimulator cells for 16 hours. Brefeldin A (10 mg/mL) was added after the first hour of incubation. Intracellular IFNγ expression in donor-derived CD3⁺CD8⁺ cells was measured by flow cytometric analysis. Representative plots are shown in FIG. 32E and graphically represented as the percentage of donor-derived CD8⁺ T cells that express IFN-γ in FIG. 32F. FIG. 32G shows that T cell functionality was significantly enhanced 48 hours after challenge when mice were castrated at the time of allo-HSCT. The DTH assay was performed at week 6 following allogeneic HSCT in sham-castrated and castrated mice, and the swelling was measured by subtracting left hind footpad swell from right hind one. Open bars indicate sham-castrated animals; closed bars represent castrated animals.

FIGS. 33A-B: Castration does not aggravate GVHD or decrease GVL activity in allogeneic HSCT recipients. For the experiments depicted in FIG. 33A, lethally irradiated (1300 cGy) (B6×C3H) F1 recipients (3 months old) received transplants with B6 TCD BM cells (5×10⁶)+splenic T cells (0.5×10⁶). Survival is depicted as a Kaplan-Meier curve. Open circles represent a TCD-BM only (no T cells) control group (n=4). Closed circles represent sham-castrated recipients; open squares represent castrated recipients. Each group contained 8 animals. For the experiments depicted in FIG. 33B, lethally irradiated, 3 month old B6D2F1/J recipients received P815 (H-2d) cells (1×10³), C57/BL6 TCD BM cells (5×10⁶) and C57/BL6 T cells (5×10⁵). Survival is depicted as a Kaplan-Meier curve. Open circles indicate a TCD-BM only (no T cells) control group (n=4). Closed circles represent sham-castrated recipients; open squares represent castrated recipients. Each test group contained 8-9 animals.

FIGS. 34A-I: Castration and IL-7 treatment have an additive effect in the thymus following allogeneic HSCT. Castrated and sham-castrated recipients were transplanted as described in FIG. 29. Recipients killed on day 14 (FIG. 34A) received, in addition, 10 g/day IL-7 or PBS (control) by intraperitoneal injection from day 0 to day 13. Recipients killed on day 28 (FIG. 34B) received 10 g/day IL-7 or PBS (control) from day 21 to day 28. Thymic cellularity was calculated from total cell counts. * (p<0.05) represents a significant increase in cell number in the castrated group compared to the sham-castrated control. Control: sham-castrated, PBS injected recipients; CX: castrated, PBS injected recipients; IL-7: sham-castrated, IL-7 injected recipients; and IL-7 & CX: castrated, IL-7 injected recipients. Semiquantitative RT-PCR was performed on whole BM 2 weeks after allo-HSCT and castration. After HPRT equilibration templates from castrated and sham-castrated mice were compared for the expression of TGFβ₁ and KGF (FIG. 34C). KGF^{-/-} and IL7^{-/-} mice were castrated and 2 weeks later thymic, BM, and splenic cellularity were analyzed. FIG. 34D-F shows the results from the thymus (FIG. 34D), BM (FIG. 34E), spleen (FIG. 34F) of KGF−/− mice. FIG. 34G-I shows the results from the thymus (FIG. 34G), BM (FIG. 34H), and spleen (FIG. 34I) of IL7−/− mice.

FIGS. 35A-C: Castration enhances engraftment in the BM, thymus, and spleen following HSCT. Mice were castrated 1 day before congenic HSCT. 5×10⁶ Ly5.1+ BM cells were injected intravenously into irradiated (800 rads) C57/BL6 mice. The BM, spleen and thymus were analyzed by flow cytometry at various time-points (2-6 weeks) post-transplant. As shown in FIG. 35A, two weeks after castration and HSCT, there are significantly more cells in the BM of castrated mice as compared to sham-castrated controls. Similarly, as shown in FIG. 35B, there is a significant increase in thymic cell number 2, 4, and 6 weeks post-transplant as compared to sham-castrated controls. As shown in FIG. 35C, in the periphery, splenic cell numbers are also significantly higher than controls 4 and 6 weeks post-transplant in the castrated recipients. Gray bars represent castrated recipients; black bars represent sham-castrated controls.

FIGS. 36A-B: Castration enhances engraftment of HSC in the BM following congenic HSCT. Mice were castrated 1 day before congenic HSCT. 5×10 Ly5.1+ BM cells were injected intravenously into irradiated (800 rads) C57/BL6 mice. The BM was analyzed for lin-c-kit+sca-1+ HSC by flow cytometry at two weeks post-transplant (FIG. 36A). Two weeks after BMT transplantation and castration, there are significantly more donor-derived HSCs in the BM of castrated mice compared to sham-castrated controls (FIG. 36B).

FIGS. 37A-D: Castration enhances engraftment of HSC in the BM following congenic HSCT (2.5×10⁶ cells and 5×10⁶ cells). Mice were castrated 1 day before congenic HSCT. 2.5×10⁶ (FIG. 37A-B) or 5×10⁶ (FIG. 37C-D) Ly5.1+ BM cells were injected intravenously into irradiated (800 rads) C57/BL6 mice. The BM was analyzed for lin-c-kit+sca-1+ HSC by flow cytometry at two weeks post-transplant. FIGS. 37A-D depict percent of common lymphoid precursors in the BM. Two weeks after BMT transplantation and castration, there is a significantly increased proportion of donor-derived HSCs in the BM of castrated mice compared to sham-castrated controls.

FIGS. 38A-B: Castration enhances the rate of engraftment of donor-derived DC in the thymus following congenic HSCT (2.5×10⁶ cells and 5×10⁶ cells). 5×10⁶ Ly5.1+ BM cells were injected intravenously into irradiated (800 rads) C57/BL6 mice. Thymocytes were analyzed by flow cytometry at two weeks post-transplant (FIG. 38A). Donor-derived DC were defined as CD45.1⁺CD11c⁺MHC class II⁺ CD11b^{+or -}. Donor-derived CD11b⁺ and CD11b⁻ DC are significantly increased in the thymii of castrated mice compared to sham-castrated controls 2 weeks after BMT (FIG. 38B).

FIGS. 39A-D: Castration enhances the rate of engraftment of donor-derived B cells in the spleen following congenic HSCT. 5×10 Ly5.1⁺ BM cells were injected intravenously into irradiated (800 rads) C57/BL6 mice. Splenocytes were analyzed by flow cytometry at two weeks post-transplant (FIG. 39A-C). There are significantly more B220+B cells in the spleens of castrated mice, as compared to the sham-castrated controls, 2 weeks after congenics BMT (FIG. 39D).

FIG. 40: The phenotypic composition of peripheral blood lymphocytes was analyzed in human patients (all >60 years) undergoing LHRH agonist treatment for prostate cancer. Patient samples were analyzed before treatment and 4 months after beginning LHRH agonist treatment. Total lymphocyte cell numbers per ml of blood were at the lower end of control values before treatment in all patients. Following treatment, six out of nine patients showed substantial increases in total lymphocyte counts (in some cases a doubling of total cells was observed). Correlating with this was an increase in total T cell numbers in six out of nine patients. Within the CD4⁺ subset, this increase was even more pronounced with eight out of nine patients demonstrating increased levels of CD4 T cells. A less distinctive trend was seen within the CD8⁺ subset with four out of nine patients showing increased levels, albeit generally to a smaller extent than CD4⁺ T cells.

FIG. 41: Analysis of human patient blood before and after LHRH-agonist treatment demonstrated no substantial changes in the overall proportion of T cells, CD4 or CD8 T cells, and a variable change in the CD4:CD8 ratio following treatment. This indicates the minimal effect of treatment on the homeostatic maintenance of T cell subsets despite the substantial increase in overall T cell numbers following treatment. All values were comparative to control values.

FIG. 42: Analysis of the proportions of B cells and myeloid cells (NK, NKT, and macrophages) within the peripheral blood of human patients undergoing LHRH-agonist treatment demonstrated a varying degree of change within subsets. While NK, NKT, and macrophage proportions remained relatively constant following treatment, the proportion of B cells was decreased in four out of nine patients.

FIG. 43: Analysis of the total cell numbers of B and myeloid cells within the peripheral blood of human patients post-treatment showed clearly increased levels of NK (five out of nine patients), NKT (four out of nine patients), and macrophage (three out of nine patients) cell numbers post-treatment. B cell numbers showed no distinct trend with two out of nine patients showing increased levels; four out of nine patients showing no change, and three out of nine patients showing decreased levels.

FIGS. 44A-B: Chemical castration in humans enhances naïve and memory T cells. As shown in FIG. 44A, a significant increase in naïve (CD62L⁺CD45RA⁺CD45RO⁻) CD4⁺ T cells was observed following LHRH-A treatment. As shown in FIG. 44B, both naïve and memory (CD62L⁻CD45RA⁻CD45RO^{+) CD}8⁺ T cells numbers were enhanced following the LHRH agonist treatment. Each bar represents the mean ±1 SD of 16 patients. *=p≦0.05; **=p≦0.01 compared to pre-treatment values.

FIGS. 45A-B: Chemical castration in humans enhances peripheral blood lymphocyte numbers. The phenotypic composition of peripheral blood was analyzed in human patients (all >60 years of age) undergoing chemical castration with a LHRH-A as part of their routine treatment for prostate cancer. Patients were analyzed prior to treatment and at 4-months of treatment. As shown in FIG. 45A, total lymphocyte number per μl peripheral blood was significantly increased following LHRH-A treatment. This was reflected by a significant increase in total T cells, CD4⁺, and CD8⁺ T cells (FIG. 45B).

FIG. 46A-B: LHRH-A treatment effectively depletes serum testosterone and increases thymic function and T cell export. In the FIG. 46A experiment, prostate cancer patients were treated with LHRH-A for 4 months. Blood was analyzed by FACS and serum was analyzed by RIA both prior to treatment and following 4-months of LHRH-A treatment. As shown in FIG. 46A, no testosterone was detected in patient sera at 4-months of LHRH-A treatment. The bar represents the mean of 13 patients analyzed. In the FIG. 46B experiment, direct evidence for an increase in thymic function and T cell export was found following analysis of TREC levels in 10 patients. Within both the CD4⁺ and CD8⁺ T cell population, five out of ten patients showed an increase (>25% above initial presentation values) in absolute TREC levels (per ml of blood) by 4 months of LHRH-A treatment. This was also reflected in a proportional increase (per 1×10⁵ cells; data not shown). This correlated with six out of ten patients showing an overall increase in total TREC levels. Only 1 patient showed a decrease in total TRECs (about 30% decrease).

FIG. 47: Chemical castration in humans enhances NK numbers. Analysis was performed prior to LHRH-A treatment and at 4-months of treatment. A significant increase in NK cells, but not B cells, was observed with LHRH-A treatment. Results are presented as the mean ±1 SD of 13 patients. **=p≦0.01 compared to pre-treatment values.

FIGS. 48A-B: Chemical castration in humans does not increase proliferation of T cells. FIGS. 48A-B depict analyzes of cellular proliferation performed using Ki-67 antigen detection. In all patients, levels of proliferation within naïve, activated, and memory cell subsets for both CD4⁺ (FIG. 48A) and CD8⁺ T cells (FIG. 48B) was not altered with LHRH-A treatment.

FIGS. 49A-C: Analysis of natural killer (NK) cell recovery at various time-points (2-8 weeks) following HSCT in control patients and LHRH-A treated patients. As shown in FIGS. 49A-B, respectively, a similar trend was observed for both control allogeneic and autologous transplant recipients. In contrast, allogeneic patients who were given LHRH-A treatment 3 weeks prior to HSCT showed a significantly higher number of NKT cells from D14-5M post-transplant (FIG. 49C: data is expressed as mean ±1 SEM of 6-20 patients; *=p≦0.05).

FIG. 50: FACS analysis of NKT cell reconstitution at various time-points (day 14, 21, 28, and 35) following HSCT in control patients. An early recovery was observed in allogeneic patients, and was seen predominantly within the CD8⁺ population early post-transplant, which indicated extrathymic routes of regeneration. Also, CD4⁺ NKT cells were evident from 1 month post-transplant.

FIGS. 51A-B: B cell reconstitution following HSCT at various time-points (2-12 months) following HSCT in control patients. As shown in FIG. 51B, B cell regeneration occurs relatively faster in autologous transplant patients as compared to that of allogeneic patients (FIG. 51A). However, a return to control values (shaded) was not evident until at least 6 months post-transplant in both groups.

FIGS. 52A-B: CD4⁺ reconstitution following HSCT at various time-points (2-12 months) following HSCT in control patients. While B cell numbers were returning to control values by 6 months post-transplant (see FIGS. 51A-B), CD4⁺ T cell numbers were severely reduced, even at 12 months post-transplant, in both autologous (FIG. 52B) and allogeneic (FIG. 52A) recipients.

FIGS. 53A-C: CD8+ regeneration following HSCT at various time-points (2-12 months) following HSCT in control patients. As shown in FIG. 53A-B, CD8+ T cell numbers regenerated quite rapidly post-transplant in both allogeneic and autologous recipients, respectively. However, as shown in FIG. 53C, the CD8+ T cells are mainly of extrathymic origin as indicated by the increase in TCRγδ+ T CD8+ T cells, CD8αα T cells, and CD28⁻CD8⁺ T cells.

FIGS. 54A-B: FACS analysis of proliferation in various populations of CD4⁺ and CD8⁺ T cells before (FIG. 54A) and 28 days after (FIG. 54B) HSCT in control patients using the marker Ki-67. Cells were analyzed on the basis of naïve, memory, and activated phenotypes using the markers CD45RO and CD27. The majority of proliferation occurred in CD8⁺ T cell subset, which further indicated that these cells were extrathymically derived and that the predominance of proliferation occurred within peripheral T cell subsets.

FIGS. 55A-D: Naïve CD4⁺ T cell regeneration at various time-points (2-12 months) following HSCT in control patients and LHRH-A treated patients. FIG. 55A depicts FACS analysis of naïve CD4⁺ T cells (CD45RA+CD45RO-CD62L+) in control (no LHRH-A treatment) patients, and shows a severe loss of these cells throughout the study. As shown in FIGS. 55B-C, naïve CD4⁺ T cell began to regenerate in the control patients by 12 months post-HSCT in autologous transplant patients (FIG. 55C) but were still considerably lower than the control values in allogeneic control patients (FIG. 55B). These results indicated that the thymus was unable to restore adequate numbers of naïve T cells in control patients post-transplant due to the age of the patients. In contrast, in patients that were given LHRH-A 3-weeks prior to allogeneic HSCT showed a significantly higher number of naïve CD4⁺ T cells at both 9 and 12 months post-transplant compared to controls (FIG. 55D). This indicates enhanced regeneration of the thymic-dependent T cell pathway with sex steroid ablation therapy. Data is expressed as mean ±1 SEM of 6-20 patients. *=p≦0.05.

FIGS. 56A-D: TREC levels at various time-points (1-12 months) following HSCT in control patients and LHRH-A treated patients. Analysis of TREC levels, which are only seen in recent thymic emigrants (RTE), emphasized the inability of the thymus to restore levels following transplant in both allogeneic (FIG. 56A) and autologous (FIG. 56B) patients. Again, this was due to the age of the patients, as well as the lack of thymic function due to thymic atrophy, which has considerable implications in the morbidity and mortality of these patients. In contrast, patients undergoing allogeneic peripheral blood stem cell transplantation demonstrated a significant increase in CD4⁺TREC+ cells/ml blood when treated with an LHRH-A prior to allogeneic transplantation (p≦0.01 at 9 months post-transplant) compared to control (non-LHRH-A treated). Allogeneic patients who were given LHRH-A treatment showed a significantly higher number of CD4⁺TREC⁺ cells/ml blood at 9 months post-transplant (FIG. 56C) compared to controls. Autologous LHRH-A treated patients also showed significantly higher levels at 12 months post-transplant (FIG. 56D). This indicates enhanced regeneration of the thymus with sex steroid ablation therapy. Data is expressed as mean ±1 SEM of 5-18 patients. *=p≦0.01.

FIGS. 57A-C: LHRH-A administration enhances responsiveness to TCR specific stimulation following allogeneic (FIGS. 57A-B) and autologous (FIG. 57C) HSCT. Three weeks prior to HSCT, patients were given LHRH-A. Patients who did not receive the agonist were used as control patients. Analysis of TCR specific stimulation was performed using 5 μg anti-CD3 and 10 μg anti-CD28 cross-linking at various time-points (1-12 months) post-transplant. As shown in FIGS. 57A-B, allogeneic LHRH-A treated patients showed enhanced proliferative responses (assessed by ³H-Thymidine incorporation) compared to control patients at all time-points except 6 and 9 months (due to low patient numbers analyzed at this time). At 6 and 9 months post-transplant, control patients had similar responsiveness to pre-treatment values. However at all other time-points, they were considerably lower. In contrast, LHRH-A treated patients had equivalent responsiveness at all time-points except 6 months compared to pre-treatment. LHRH-A treated patients showed enhanced proliferative responses (assessed by ³H-Thymidine incorporation) compared to control patients at 1, 3, and 4 months post-transplant. This indicates a contribution of direct peripheral T cell effects, as new CD4⁺ T cells are not evident until at least 1-2 months post-transplant (FIG. 57B: Data is expressed as mean ±1 SEM of 5-12 patients. *=p≦0.05; **=p≦0.01). As shown in FIG. 57C, autologous LHRH-A treated patients showed enhanced proliferative responses (assessed by ³H-Thymidine incorporation) compared to control patients at all time-points except 5 months. Restoration to pre-treatment values was observed by 12 months post-transplant in both control and LHRH-A treated patients.

FIGS. 58A-B: LHRH-A administration enhances responsiveness to PWM and TT mitogenic stimulation following allogeneic HSCT. Three weeks prior to HSCT, patients were treated with LHRH-A. Patients who did not receive the agonist were used as control patients. Analysis of mitogenic responsiveness was performed using pokeweed mitogen (PWM) or tetanus toxoid (TT) at various time-points (1-12 months) post-transplant. Patients treated with LHRH-A prior to HSCT showed an enhanced responsiveness to PWM (FIG. 58A) and TT (FIG. 58B) stimulation at all time-points studied compared to control patients.

FIGS. 59A-B: LHRH-A administration enhances responsiveness to PWM and TT mitogenic stimulation following autologous HSCT. Three weeks prior to HSCT, patients were treated with LHRH-A. Patients who did not receive the agonist were used as control patients. Analysis of mitogenic responsiveness was performed using PWM or TT at various time-points (1-12 months) post-transplant. Patients treated with LHRH-A prior to HSCT showed an enhanced responsiveness to PWM (FIG. 59A) and TT (FIG. 59B) stimulation at the majority of time-points studied compared to control patients (p≦0.001 at 3 months). By 12-months post-transplant, LHRH-A treated patients had restored responsiveness.

FIGS. 60A-D: LHRH-A treatment enhances the rate of engraftment in autologous HSCT patients. Three weeks prior to HSCT, patients were treated with LHRH-A (FIGS. 60A, C and D). Patients who did not receive the agonist were used as control patients (FIG. 60B). Total white blood cell (WBC) counts and granulocyte (G) counts per μl of blood were determined at days 14, 28, and 35 post-transplant. As shown in FIG. 60A, autologous patients who were given LHRH-A treatment showed a significantly higher number of WBC at D14 post-transplant compared to controls (FIG. 60B) (p≦0.05), with 87% showing granulocyte engraftment (≧500 cells/μl blood) compared to 45% of controls (p≦0.05) at this time-point. Autologous patients who were given LHRH-A treatment also showed a significantly higher number of neutrophils at D10-12 post-transplant compared to controls (FIG. 60C: data is expressed as mean ±1 SEM of 8-20 patients. *=p≦0.05). In addition, although not significant, autologous patients had higher lymphocyte counts throughout the time-points analyzed in LHRH-A treated compared to control group (FIG. 60D).

FIGS. 61A-D: LHRH-A treatment enhances the rate of engraftment in allogeneic HSCT patients. Three weeks prior to HSCT, patients were treated with LHRH-A (FIGS. 61A, C, and D). Patients who did not receive the agonist were used as control patients (FIG. 61B). Total white blood cell (WBC) counts and granulocyte (G) counts per μl of blood were determined at day 14, 28, and 35 post-transplant. As shown in FIG. 61A, allogeneic patients who were given LHRH-A treatment showed a significantly higher number of WBC at D14 post-transplant compared to controls (FIG. 61B) (p≦0.05) with 64% showing granulocyte engraftment (≧500 cells/μl blood) compared to 44% of controls at this time-point. In addition, allogeneic patients who were given LHRH-A treatment showed a significantly higher number of neutrophils at D9, 12 and 19 post-transplant compared to controls (FIG. 61C: data is expressed as mean ±1 SEM of 8-20 patients. *=p≦0.05). Additionally, analysis of patients undergoing peripheral blood stem cell transplantation demonstrated a significant increase in lymphocyte counts when treated with an LHRH-A prior to allogeneic transplantation (p≦0.05 at days 10, 12, 13, and 17-21 post-transplant) (FIG. 61D).

FIGS. 62A-F. TCR specific peripheral T cell proliferative responses are enhanced within one week of castration. Eight week-old mice were castrated and analyzed for anti-CD3/anti-CD28 stimulated T cell proliferative response 3 days (FIGS. 62A, C, and E) and 7 days (FIGS. 62B, D, and F) after surgery. Peripheral (cervical, axillary, brachial, and inguinal) lymph node (FIGS. 62A and B), mesenteric lymph node (FIGS. 62C and D), and spleen cells (FIGS. 62E and F) were stimulated with varying concentrations of anti-CD3 and co-stimulated with anti-CD28 at a constant concentration of 10 μg/ml for 48 hours. Cells were then pulsed with tritiated thymidine for 18 hours and proliferation was measured as ³H-thymidine incorporation. Diamonds indicate castrated animals. Squares indicate sham-castrated control mice. n=4, *p≦0.05 (non-parametric, unpaired, Mann-Whitney statistical test).

FIG. 63: LHRH-A administration enhances responsiveness to TCR specific stimulation following treatment for chronic cancer sufferers. Patients with chronic malignancies were treated with LHRH-A. Analysis of TCR specific stimulation was performed using anti-CD3 and anti-CD28 cross-linking at various time-points (day 7-12 months) following LHRH-A administration. LHRH-A treated patients showed enhanced proliferative responses (assessed by ³H-Thymidine incorporation) compared to pre-treatment levels in a cyclical fashion. This reflected the administration of the agonist with monthly depot injections. These results indicate a direct influence on peripheral T cells. However, the enhanced response seen at 12-months post-treatment reflect changes in thymic-derived T cells as well, since agonist administration was ceased from 4-months for all patients.

FIG. 64 is a line graph showing that while 60% of the sham-operated mice had diabetes, fewer than 20% of the castrated group had diabetes.

FIG. 65 is a bar graph showing that castrated NOD mice had a marked increase in total thymocyte number but no differences in total spleen cells.

FIGS. 66A-C are bar graphs showing that there was a significant increase in all thymocyte subclasses (FIG. 66A) in castrated NOD mice. There was no change in B cells compared to sham-castrated NOD mice (FIG. 66C) nor in the total T or B cells in the spleen (FIG. 66B).

FIGS. 67A-B show a marked increase in total thymocytes (FIG. 67A) and spleen cells (FIG. 68B) in castrated NZB mice.

FIG. 68 is a graph showing decreased tumor incidence in mice that have been castrated and immunized as compared to controls.

FIGS. 69A-C are bar graphs showing that castrated and immunized mice have increased splenic cellularity as compared to controls.

FIGS. 70A-B are graphs showing increased γIFN production in mice that have been castrated and immunized as compared to controls.

FIGS. 71A-B are graphs showing that castrated and immunized mice exhibit enhanced antigen-specific CTL responses as compared to controls.

FIGS. 72A-E are graphs showing that thymectomy does not impact the effect of sex steroid inhibition/BMT on common lymphoid progenitors in the BM (FIG. 72A), total BM B cells (FIG. 72B), immature B cells in the BM (FIG. 64C), total cell numbers in the spleen (FIG. 72D), or on total B cells in the spleen (FIG. 72E).

### DETAILED DESCRIPTION OF THE INVENTION

The patent and scientific literature referred to herein establishes knowledge that is available to those with skill in the art. The issued U.S. patents, applications, published foreign applications, and references, including GenBank database sequences, that are cited herein are hereby incorporated by reference in its entirety to the same extent as if each was specifically and individually indicated to be incorporated by reference.

The present invention comprises methods for increasing the BM functionality following sex steroid ablation and/or interruption of sex steroid signalling, either without, prior to, or in combination with, thymus regeneration. “Increasing the function of BM” and “enhancing BM functionality” is herein defined as an improvement in the production and/or output of immune cells, including precursors, for example HSC (and consequent increases in blood cells) from the BM, including improvement in haemopoiesis and/or enhancement of engraftment following HSCT. An improvement in output may include, but is not limited to, an improved ability to mobilize immune cells, including HSC, into the periphery or to a target tissue, in particular, immune or damaged tissue. In one embodiment, HSC haemopoiesis is improved. In another embodiment, HSC output is improved. In certain embodiments, blood and/or immune cell numbers are increased. In yet another embodiment, HSC engraftment is improved following HSCT. In another embodiment, HSC mobilization into the periphery or homing to target tissue is improved. In yet another embodiment, proliferative ability and/or the ability to differentiate into haematopoietic or non-haemopoitetic progeny is improved.

The present invention also comprises methods for increasing the function of T cells and other immune cells following sex steroid ablation and/or interruption of sex steroid signalling, either without, prior to, or in combination with, thymus regeneration. The terms “immune cells” and “cells of the immune system” are used interchangeably and are herein defined as HSC, T cells, B cells, DC, and/or other blood cells, including, but not limited to, HSC progeny, CLP, MLP, lymphocytes, myeloid cells, neutrophils, granulocytes, basophils, eosinophils, NK, NKT, platelets, red blood cells, monocytes, macrophage, naïve T cells, and precursors of the aforementioned. The cells may or may not be peripheral, and the cells may be found in any one or more of the BM, blood, spleen, lymph nodes, thymus, mucosal membranes, skin, or other tissues.

“Increased” or “enhanced functionality” of immune cells means that the immune cells are more able to provide an adequate required immune response, when compared to the immune response normally expected without sex steroid ablation. In one case, the immune cells are T cells. In other examples, the immune cells are B cells, DC, and/or HSC. “Increased functionality” includes, but is not limited to, improved killing of target cells; increased lymphocyte proliferative response; improved signalling ability; improved homing ability; improved APC activation, increased levels or activity of receptors, cell adhesion molecules, or co-stimulatory molecules; decreased apoptosis; increased release of cytokines, interleukins, and other growth factors; increased levels of antibody (Ab) in the plasma; and increased levels of innate immunity (e.g., natural killer (NK) cells, DC, neutrophils, macrophages, etc.) in the blood and throughout the body. Each may either directly or indirectly assist in combating disease and infection, thereby increasing responsiveness to, resistance to, treatment for, and prevention of, e.g., infection by various foreign agents, and increasing immune responsiveness to vaccines.

The present invention further comprises methods for preventing, diminishing the risk, or treating illness or disease in a patient. In one embodiment, the disease is a T cell disorder. In another embodiment, the disease is an autoimmune disease or allergy. Additionally, the present disclosure also provides methods for improving a patient's immune response to a vaccine antigen (e.g., that of an agent) by disrupting sex steroid mediated signalling and causing the thymus to reactivate. In both cases, the functional status of the peripheral T cells may be improved and may be accomplished by quantitatively and qualitatively restoring the peripheral T cell pool, particularly at the level of naïve T cells. These naïve T cells are then able to respond to a greater degree to presented foreign antigen.

As described above, the aged (post-pubertal) thymus causes the body's immune system to function at less than peak levels (such as that found in the young, pre-pubertal thymus). “Post-pubertal” is herein defined as the period in which the thymus has reached substantial atrophy. In humans, this occurs by about 20-25 years of age, but may occur earlier or later in a given individual. “Pubertal” is herein defined as the time during which the thymus begins to atrophy, but may be before it is fully atrophied. In humans this occurs from about 10-20 years of age, but may occur earlier or later in a given individual. “Pre-pubertal” is herein defined as the time prior to the increase in sex steroids in an individual. In humans, this occurs at about 0-10 years of age, but may occur earlier or later in a given individual.

The terms “vaccinating,” “vaccination,” “vaccine,” “immunizing,” and “immunization,” are herein defined as administration to a patient of a preparation to elicit an immune response to an antigen. Vaccination may include both prophylactic and therapeutic vaccines. As will be understood by those skilled in the art, infection by, e.g., a virus or other agent, is also a method of vaccinating an individual.

The terms “improving,” “enhancing,” or “increasing” “vaccine responses” or “vaccine responsiveness” in a patient or “improving,” “enhancing,” or “increasing” the “immune responsiveness of a patient to a vaccine” and other similar language is used interchangeably and herein defined as meaning that a patient's immune response to the vaccine or vaccine antigen is improved compared to the immune response which would have otherwise occurred in a patient without disruption of sex steroid signalling.

“Illness” and “disease” are used interchangeably and are herein defined as any disease, infection, or medical condition (symptomatic or asymptomatic) in which an immune response, defense, or modified immune system would be beneficial to the patient. The illness may be caused by an infectious agent, cancer, drug treatment (e.g., chemotherapy), irradiation, chemical poisoning, genetic defect, or other disorder.

As herein defined, “prevention” of or “preventing” an illness is herein defined as complete as well as partial protection, including without limitation, reduced severity of clinical symptoms than would have otherwise occurred in the patient. With an improved or modified immune system the individual will have a reduced likelihood of succumbing to, or suffering from, a tumor or cancer, allergy, autoimmune diseases, a prevailing infection (e.g., viral, bacterial, fungal, or parasitic) or illness, and/or will show better responses to a vaccination (e.g., increased levels of antibody (Ab) specific to that vaccine or antigen, and development of effector T cells). Prevention of an illness may occur by activating or modifying immune defense mechanisms to inhibit or reduce the development of clinical symptoms, such as to a point where only reduced or minimal medical care is required. Preventing an infection also encompasses defending the body against infectious agents, such as viruses, bacteria, parasites, fungi, etc., or against non-infectious agents. This may take the form of preventing such agents from entering the cells in the body and/or the efficient removal of the agents by cells of the broad immune system (e.g., NK, DC, macrophages, neutrophils, etc.). In some instances, complete prevention of illness is not achieved and, instead, partial prevention is achieved in which a stronger, more resilient or more effective immune system will aid the body in decreasing the extent, severity, and duration of illness or clinical symptoms of illness or recovery time or delay the onset of clinical symptoms.

“Treatment” of or “treating” an illness encompasses completely or partially reducing the symptoms of the illness in the patients, as compared to those symptoms that would have otherwise occurred in the patient without sex steroid ablation or interruption of sex steroid mediated signalling. Treatment of an illness may occur by activating immune defense mechanisms to inhibit, delay, or reduce the development of clinical symptoms. In one example, the patient has already contacted the agent, or is at a high risk of doing so.

The ability to have improved response to, respond better to, or to overcome, a new (by prevention) or existing (by treatment) illness involves improving the immune system of the body, which includes increasing the number and/or functionality of the BM cells and/or thymic-derived factors, and/or increasing the number and/or functionality of immune cells. Activation of the immune system also increases the number of lymphocytes capable of responding to the antigen of the agent in question, which leads to the elimination (complete or partial) of the antigen and/or foreign agent creating a situation where the host is treated for or resistant to the infection or disease. With an improved or modified immune system the individual will have a reduced likelihood of succumbing to or suffering from a tumor or cancer, allergy, autoimmune diseases, a prevailing infection (e.g., viral, bacterial, fungal, or parasitic) or illness, and/or will show better responses to a vaccination (e.g., increased levels of antibody (Ab) specific to that vaccine or antigen, and development of effector T cells).

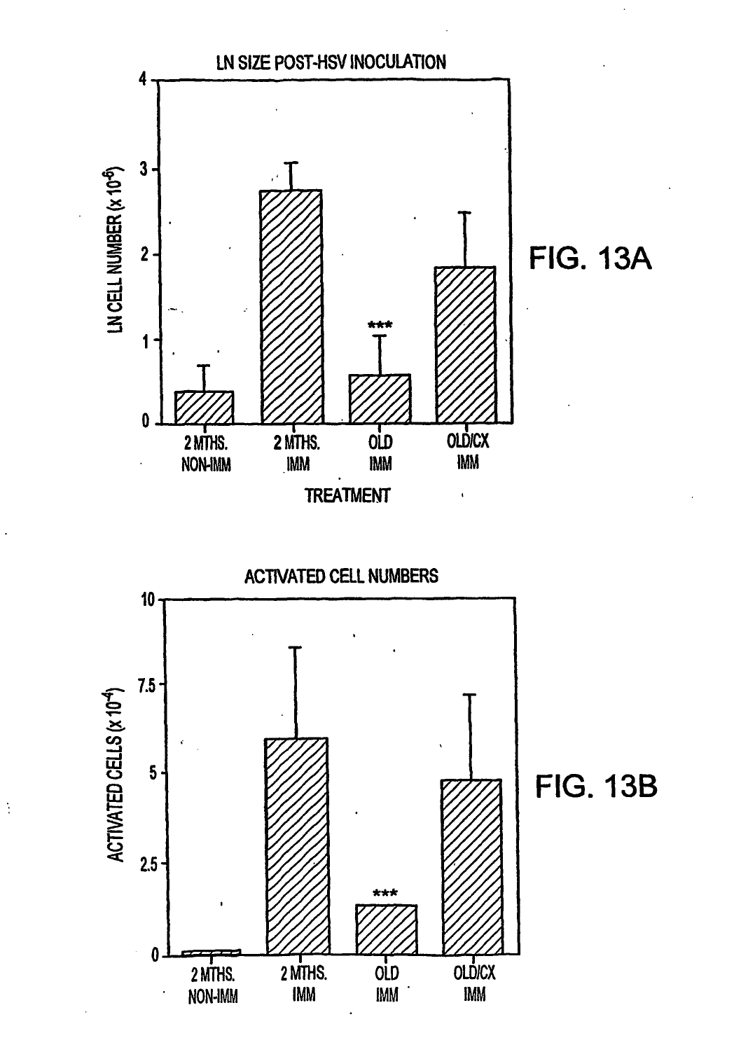

This increase in the immune defense was exemplified in the dramatic improvement of aged mice to the human herpes simplex virus infection (see Example 3, and FIGS. 13-17). The castrated aged mice initially showed a marked increase of lymphocyte infiltration into the draining lymph node. This infiltration is the first step in an immune response, and is generally required to increase the likelihood of an antigen-specific lymphocyte contacting the antigen. The next step is the activation of the lymphocytes by antigen and the development of Ab and/or CTL and release of cytokines from lymphocytes, all of which combine to destroy the agent.

“Infectious agents,” “foreign agents,” and “agents” are used interchangeably and include any cause of disease or illness in an individual. Agents include, but are not limited to, viruses, bacteria, fungi, parasites, prions, cancers, precancerous cells, chemical or biological toxins, allergens, asthma-inducing agents, self proteins, and antigens which contribute to autoimmune disease, etc.

In one case, the agent is a virus, bacteria, fungi, or parasite e.g., from the coat protein of a human papilloma virus (HPV), which causes uterine cancer; or an influenza peptide (e.g., hemagglutinin (HA), nucleoprotein (NP), or neuraminidase (N)).

Non-limiting examples of infectious viruses include: Retroviridae (e.g., human immunodeficiency viruses, such as HIV-1 (also referred to as HTLV-III, LAV or HTLV-II/LAV, or HIV-III) and other isolates, such as HIV-LP); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses, severe acute respiratory syndrome (SARS) virus); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arenaviridae (hemorrhagic fever viruses); Reoviridae (e.g., reoviruses, orbiviruses, and rotaviruses); Birnaviridae; Hepadnaviridae (e.g., Hepatitis B virus); Parvoviridae (parvoviruses); Papovaviridae (papilloma viruses, polyoma viruses); Adenoviridae (most adenoviruses); Herpesviridae (e.g., herpes simplex virus (HSV) 1 and 2, varicella zoster virus, cytomegalovirus (CMV), herpes viruses); Poxyiridae (e.g., variola viruses, vaccinia viruses, pox viruses); Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C)); Norwalk and related viruses, and astroviruses).

Non-limiting examples of infectious bacteria include: Helicobacter pyloris, Borelia burgdorferi, Legionella pneumophilia, Mycobacteria sporozoites (sp.) (e.g., M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniael, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelli.

Non-limiting examples of infectious fungi include: Cryptococcus neoformans, Histoplasma capsulatum, Coccidioides immitis, Blastomyces dermatitidis, Chlamydia trachomatis, Candida albicans.

Other infectious organisms (i.e., protists) include, but are not limited to, Plasmodium falciparum and Toxoplasma gondii.

In other embodiments, the agent is an allergen. Allergic conditions include, but are not limited to, eczema, allergic rhinitis or coryza, hay fever, bronchial asthma, urticaria (hives) and food allergies, and other atopic conditions.

In yet another embodiment, the agent is a cancer or tumor. The cancer or tumor may be malignant or non-malignant. As used herein, a tumor or cancer includes, e.g., tumors of the brain, lung (e.g., small cell and non-small cell), and pleura, gynecological, urogenital and endocrine system, (e.g., cervix, uterus, endometrium, bladder, renal organs, ovary, breast, and/or prostate), gastrointestinal tract (e.g., anal, bile duct, carcinoid tumor, gallbladder, gastric or stomach, liver, esophagus, pancreas, rectum, small intestine, and/or colon), as well as other carcinomas, and bone, skin and connective tissue (e.g., melanomas and/or sarcomas), and/or the hematological system (e.g., blood, myelodysplastic syndromes, myeloproliferative disorders, plasma cell neoplasm, lymphomas and/or leukemias).

This invention may be used with any animal species (including humans) having sex steroid driven maturation and an immune system, such as mammals and marsupials. In some examples, the invention is used with large mammals, such as humans.

The terms thymus “regeneration,” “reactivation” and “reconstitution” and their derivatives are used interchangeably herein, and are herein defined as the recovery of an atrophied or damaged (e.g., by chemicals, radiation, graft versus host disease, infections, genetic predisposition) thymus to its active state. “Active state” is herein defined as meaning a thymus in a patient whose sex steroid hormone mediated signalling has been disrupted, achieves an output of T cells that is at least 10%, or at least 20%, or at least 40%, or at least 60%, or at least 80%, or at least 90% of the output of a pre-pubertal thymus (i.e., a thymus in a patient who has not reached puberty).

“Recipient,” “patient” and “host” are used interchangeably and are herein defined as a subject receiving sex steroid ablation therapy and/or therapy to interrupt sex steroid mediated signalling and/or, when appropriate, the subject receiving the HSC transplant.

“Donor” is herein defined as the source of the transplant, which may be syngeneic, allogeneic or xenogeneic. In some instances, the patient may provide, e.g., his or her own autologous cells for transplant into the patient at a later time-point. Allogeneic HSC grafts may be used, and such allogeneic grafts are those that occur between unmatched members of the same species, while in xenogeneic HSC grafts the donor and recipient are of different species. Syngeneic HSC grafts between matched animals may also be used. The terms “matched,” “unmatched,” “mismatched,” and “non-identical” with reference to HSC grafts are herein defined as the MHC and/or minor histocompatibility markers of the donor, and the recipient are (matched) or are not (unmatched, mismatched and non-identical) the same.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

Disruption of Sex Steroid Mediated Signalling

The present invention further provides methods of disruption of sex steroid-mediated signalling in a patient, wherein the patient's thymus may or may not be subsequently reactivated. Additionally, the present invention provides methods of improving the functional status of immune cells (e.g., T cells) of the patient. With respect to T cells, the thymus begins to increase the rate of proliferation of the early precursor cells (CD3⁻CD4⁻CD8⁻ cells) and converts them into CD4⁺CD8⁺, and subsequently new mature CD3^{hi}hCD4⁺CD8⁻ (T helper (Th) lymphocytes) or CD3^{hi}CD4⁻CD8⁺ (cytotoxic T lymphocytes (CTL)). The rejuvenating thymus also increases its uptake of HSC, or other stem cells or progenitor cells capable of forming into T cells, from the blood stream and converts them into new T cells and intrathymic DC. The increased activity in the thymus resembles in many ways that found in a normal, younger thymus (e.g., a prepubertal patient). The result of this renewed thymic output is increased levels of naïve T cells (those T cells which have not yet encountered antigen) in the blood. There is also an increase in the ability of the peripheral T cells to respond to stimulation, e.g., by cross-linking with anti-CD28 Abs, or by TCR stimulation with, e.g., anti-CD3 antibodies, or stimulation with mitogens, such as pokeweed mitogen (PWM), and this increased T cell responsiveness can occur before thymic regeneration, such as within 2, 3, 4, 5, 6, 7, 14, or 21 days. This combination of events results in the body becoming better able to defend against infection and other immune system challenges (e.g., cancers), or recover from immune system challenges (e.g., becoming better able to recover from chemotherapy and radiotherapy). As a result, the methods of the invention may be used to prevent or treat an illness or infection, increase a patient's immune responsiveness to a vaccine, and for optional gene therapy.

As used herein, “sex steroid ablation,” “inhibition of sex steroid-mediated signalling,” “sex steroid disruption,” “interruption of sex steroid signaling,” and other similar terms are herein defined as at least partial disruption of sex steroid (and/or other hormonal) production and/or sex steroid (and/or other hormonal) signalling, whether by direct or indirect action. In one embodiment, sex steroid signalling to the thymus is interrupted. As will be readily understood, sex steroid-mediated signalling can be disrupted in a range of ways well known to those of skill in the art, some of which are described herein. For example, inhibition of sex hormone production or blocking of one or more sex hormone receptors will accomplish the desired disruption, as will administration of sex steroid agonists and/or antagonists, or active (antigen) or passive (antibody) anti-sex steroid vaccinations.

A non-limiting method for creating disruption of sex steroid-mediated signalling is through castration. Methods for castration include, but are not limited to, chemical castration and surgical castration.

“Castration” is herein defined as the reduction or elimination of sex steroid production, action and/or distribution in the body. This effectively eventually returns the patient to a pre-pubertal status when the thymus is more fully functioning than immediately prior to castration. Surgical castration removes the patient's gonads. Methods for surgical castration are well known to routinely trained veterinarians and physicians. One non-limiting method for castrating a male animal is described in the examples below. Other non-limiting methods for castrating human patients include a hysterectomy or ovariectomy procedure (to castrate women) and surgical castration to remove the testes (to castrate men). In some clinical cases, permanent removal of the gonads via physical castration may be appropriate.

Chemical castration is a less permanent version of castration. As herein defined, “chemical castration” is the administration of a chemical for a period of time, which results in the reduction or elimination of sex steroid production, action and/or distribution in the body. A variety of chemicals are capable of functioning in this manner. Non-limiting examples of such chemicals are the sex steroid inhibitors and/or analogs described below. During the chemical delivery, and for a period of time afterwards, the patient's hormone production may be turned off or reduced. The castration may be reversed upon termination of chemical delivery or by delivery of the relevant sex hormones.

The terms “sex steroid analog,” “sex steroid ablating agent,” “sex steroid inhibitor,” “inhibitor of sex steroid signalling,” “modifier of sex steroid signalling,” and other similar terms are herein defined as any one or more pharmaceutical agents that will decrease, disrupt, prevent, or abolish sex steroid (and/or other hormone) mediated signalling. GnRH (also called LHRH or GnRH/LHRH herein), and analogs thereof, are non-limiting exemplary inhibitors of sex steroid signalling used throughout this application. However, as will be readily understood by one skilled in the art, in practicing the inventions provided herein, GnRH/LHRH, or analogs thereof, may be replaced with any one (or more) of a number of substitute sex steroid inhibitors or analogs (or other blocker(s) or physical castration) which are described herein, without undue experimentation.

Any pharmaceutical drug, or other method of castration, that ablates sex steroids or interrupts sex steroid-mediated signaling, may be used in the methods of the invention. For example, one non-limiting method of inhibiting sex steroid signalling, reactivating the thymus and/or enhancing the functionality of BM and immune cells, is by modifying the normal action of GnRH on the pituitary (i.e., the release of gonadotrophins, FSH and LH) and consequently reducing normal sex steroid production or release from the gonads. Thus, in one case, sex steroid ablation is accomplished by administering one or more sex hormone analogs, such as a GnRH analog. GnRH is a hypothalamic decapeptide that stimulates the secretion of the pituitary gonadotropins, leutinizing hormone (LH) and follicle-stimulating hormone (FSH). Thus, GnRH agonists (e.g., in the form of Synarel® or Lupron®) initially result in over stimulation of the receptor and through feedback mechanisms will suppress the pituitary production of FSH and LH by desensitization of LHRH receptors. These gonadotrophins normally act on the gonads to release sex steroids, in particular estrogens in females and testosterone in males; the release of which is significantly reduced by the absence of FSH and LH. The direct consequences of this are a drop in the plasma levels of sex steroids and, as a result, progressive release of the inhibitory signals on the thymus. A more rapid drop in circulating sex steroid levels can be achieved for example by the use of a GnRH antagonist.

In some embodiments, the sex steroid mediated signalling is disrupted by administration of a sex steroid analog, such as an analog of leutinizing hormone-releasing hormone (LHRH). Sex steroid analogs and their use in therapies and chemical castration are well known. Sex steroid analogs are commercially available and their use in therapies and chemical castration are well known. Such analogs include, but are not limited to, the following agonists of the LHRH receptor (LHRH-R): buserelin (e.g., buserelin acetate, trade names Suprefact® (e.g., 0.5-02 mg s.c./day)), Suprefact Depot®, and Suprefact® Nasal Spray (e.g., 2 μg per nostril, every 8 hrs.); Hoechst, also described in U.S. Pat. Nos. 4,003,884, 4,118,483, and 4,275,001); Cystorelin® (e.g., gonadorelin diacetate tetrahydrate, Hoechst); deslorelin (e.g., desorelin acetate, Deslorell®, Balance Pharmaceuticals); gonadorelin (e.g., gonadorelin hydrocholoride, trade name Factrel® (100 μg i.v. or s.c.), Ayerst Laboratories); goserelin (goserelin acetate, trade name Zoladex®, AstraZeneca, Auckland, NZ, also described in U.S. Pat. Nos. 4,100,274 and 4,128,638; GB 9112859 and GB 9112825); histrelin (e.g., histrelin acetate, Supprelin®, (s.c., 10 μg/kg.day), Ortho, also described in EP 217659); leuprolide (leuprolide acetate, trade name Lupron® or Lupron Depot®), Abbott/TAP, Lake Forest, Ill., also described in U.S. Pat. Nos. 4,490,291 3,972,859, 4,008,209, 4,992,421, and 4,005,063; DE 2509783); leuprorelin (e.g., leuprorelin acetate, trade name Prostap SR® (e.g., single 3.75 mg dose s.c. or i.m./month)); Prostap3® (e.g., single 11.25 mg dose s.c. every 3 months), Wyeth, USA, also described in Plosker et al., (1994) Drugs 48:930); lutrelin (Wyeth, USA, also described in U.S. Pat. No. 4,089,946); Meterelin® (e.g., Avorelina (e.g., 10-15 mg slow-release formulation), also described in EP 23904 and WO 91/18016); nafarelin (e.g., trade name Synarel® (i.n. 200-1800 μg/day), Syntex, also described in U.S. Pat. No. 4,234,571; WO 93/15722; and EP 52510); and triptorelin (e.g., triptorelin pamoate; trade names Trelstar LA® (11.25 mg over 3 months), Trelstar LA Debioclip® (pre-filled, single dose delivery), LA Trelstar Depot® (3.75 mg over one month), and Decapeptyl®, Debiopharm S. A., Switzerland, also described in U.S. Pat. Nos. 4,010,125, 4,018,726, 4,024,121, and 5,258,492; EP 364819). LHRH analogs also include, but are not limited to, the following antagonists of the LHRH-R: abarelix (trade name Plenaxis™ (e.g., 100 mg i.m. on days 1, 15 and 29, then every 4 weeks thereafter), Praecis Pharmaceuticals, Inc., Cambridge, Mass.) and cetrorelix (e.g., cetrorelix acetate, trade name Cetrotide™ (e.g., 0.25 or 3 mg s.c.), Zentaris, Frankfurt, Germany). Additional sex steroid analogs include Eulexin® (e.g., flutamide (e.g., 2 capsules 2×/day, total 750 mg/day), Schering-Plough Corp., also described in FR 7923545, WO 86/01105 and PT 100899), and dioxane derivatives (e.g., those described in EP 413209), and other LHRH analogs such as are described in EP 181236, U.S. Pat. Nos. 4,608,251, 4,656,247, 4,642,332, 4,010,149, 3,992,365, and 4,010,149. Combinations of agonists, combinations of antagonists, and combinations of agonists and antagonists are also included. One non-limiting analog of the invention is deslorelin (described in U.S. Pat. No. 4,218,439). For a more extensive list, of analogs, see Vickery et al., (1984) LHRH AND ITS ANALOGS: CONTRACEPTIVE & THERAPEUTIC APPLICATIONS (Vickery et al., eds.) MTP Press Ltd., Lancaster, Pa. Each analog may also be used in modified form, such as acetates, citrates and other salts thereof, which are well known to those in the art.

One non-limiting example of administration of a sex steroid ablating agent is a subcutaneous/intradermal injection of a “slow-release” depot of GnRH agonist (e.g., one, three, or four month Lupron® injections) or a subcutaneous/intradermal injection of a “slow-release” GnRH-containing implant (e.g., one or three month Zoladex®, e.g., 3.6 mg or 10.8 mg implant). These could also be given intramuscularly (i.m.), intravenously (i.v.) or orally, depending on the appropriate formulation. Another example is by subcutaneous injection of a “depot” or “impregnated implant” containing, for example, about 30 mg of Lupron® (e.g., Lupron Depot® (leuprolide acetate for depot suspension) TAP Pharmaceuticals Products, Inc., Lake Forest, Ill.). A 30 mg Lupron® injection is sufficient for four months of sex steroid ablation to allow the thymus to rejuvenate and export new naïve T cells into the blood stream.

Many of the mechanisms of inhibiting sex steroid signalling described herein are well known, and some of these drugs, in particular the GnRH angonists, have been used for many years in the treatment of disorders of the reproductive organs, such as some hormone sensitive cancers including breast and prostate cancer, endometriosis, reproductive disorders, hirsuitism, precocious puberty, sexual deviancy, and in the control of fertility.

In certain examples, the thymus of the patient is ultimately reactivated by sex steroid ablation and/or interruption or disruption of sex steroid-mediated signalling. In some cases, disruption reverses the hormonal status of the patient. According to the methods of the invention, the hormonal status of the recipient is reversed such that the hormones of the recipient approach pre-pubertal levels. By lowering the level of sex steroid hormones in the recipient, the signalling of these hormones to the thymus is lowered, thereby allowing the thymus to be reactivated. The patient may be pubertal or post-pubertal, or the patient has (or has had) a disease that, at least in part, atrophied the thymus. Alternatively, the patient has (or has had) a treatment of a disease, wherein the treatment of the disease, at least in part, atrophied the thymus of the patient. Such treatment may be anti-viral, immunosuppression, chemotherapy, and/or radiation treatment. In other embodiments, the patient is menopausal or has had sex steroid (or other hormonal levels) decreased by another means, e.g., trauma, drugs, etc.

Sex steroid ablation or interruption of sex steroid mediated signalling has one or more direct effects on the BM and/or cells of the immune system, wherein functionality is improved. The effects may occur prior to, or concurrently with, thymic reactivation.

In some embodiments, sex steroid ablation or inhibition of sex steroid signalling is accomplished by administering an anti-androgen such as an androgen blocker (e.g., bicalutamide, trade names Cosudex® or Casodex®, 5-500 mg, e.g., 50 mg po QID, AstraZeneca, Auckland, NZ), either alone or in combination with an LHRH analog or any other method of castration. Sex steroid ablation or interruption of sex steroid signalling may also be accomplished by administering cyproterone acetate (trade name, Androcor®, Schering AG, Germany; e.g., 10-1000 mg, 100 mg bd or tds, or 300 mg IM weekly), a 17-hydroxyprogesterone acetate, which acts as a progestin, either alone or in combination with an LHRH analog or any other method of castration. Other anti-androgens may be used (e.g., antifungal agents of the imidazole class, such as liarozole (Liazol® e.g., 150 mg/day, an aromatase inhibitor) and ketoconazole, flutamide (trade names Euflex® and Eulexin®, Schering Plough Corp, N.J.; 50-500 mg e.g., 250 or 750 mg po QID), megestrol acetate (Megace® e.g., 480-840 mg/day or nilutamide (trade names Anandron®, and Nilandron®, Roussel, France e.g., orally, 150-300 mg/day)). Antiandrogens are often important in therapy, since they are commonly utilized to address flare by GnRH analogs. Some antiandrogens act by inhibiting androgen receptor translocation, which interrupts negative feedback resulting in increased testosterone levels and minimal loss of libido/potency. Another class of anti-androgens useful in the present invention are the selective androgen receptor modulators (SARMS) (e.g., quinoline derivatives, bicalutamide (trade name Cosudex® or Casodex®, as above), and flutamide (trade name Eulexin®, e.g., orally, 250 mg/day)). Other well known anti-androgens include 5 alpha reductase inhibitors (e.g., dutasteride, (e.g., po 0.5 mg/day)) which inhibits both 5 alpha reductase isoenzymes and results in greater and more rapid DHT suppression; finasteride (trade name Proscar®; 0.5-500 mg, e.g., 5 mg po daily), which inhibits 5 alpha reductase 2 and consequent DHT production, but has little or no effect on testosterone or LH levels.

In other embodiments, sex steroid ablation or inhibition of sex steroid signalling is accomplished by administering anti-estrogens either alone or in combination with an LHRH analog or any other method of castration. Some anti-estrogens (e.g., anastrozole (trade name Arimidex®), and fulvestrant (trade name Faslodex®, 10-1000 mg, e.g., 250 mg IM monthly)) act by binding the estrogen receptor (ER) with high affinity similar to estradiol and consequently inhibiting estrogen from binding. Faslodex® binding also triggers conformational change to the receptor and down-regulation of estrogen receptors, without significant change in FSH or LH levels. Other non-limiting examples of anti-estrogens are tamoxifen (trade name Nolvadex®); Clomiphene (trade name Clomid®) e.g., 50-250 mg/day, a non-steroidal ER ligand with mixed agonist/antagonist properties, which stimulates release of gonadotrophins; diethylstilbestrol ((DES), trade name Stilphostrol®) e.g., 1-3 mg/day, which shows estrogenic activity similar to, but greater than, that of estrone, and is therefore considered an estrogen agonist, but binds both androgen and estrogen receptors to induce feedback inhibition on FSH and LH production by the pituitary, diethylstilbestrol diphosphate e.g., 50 to 200 mg/day; as well as danazol, droloxifene, and iodoxyfene, which each act as antagonists. Another class of anti-estrogens which may be used either alone or in combination with other methods of castration, are the selective estrogen receptor modulators (SERMS) (e.g., toremifene (trade name Fareston®, 5-1000 mg, e.g., 60 mg po QID), raloxofene (trade name Evista®), and tamoxifen (trade name Nolvadex®, 1-1000 mg, e.g., 20 mg po bd), which behaves as an agonist at estrogen receptors in bone and the cardiovascular system, and as an antagonist at estrogen receptors in the mammary gland). Estrogen receptor downregulators (ERDs) (e.g., tamoxifen (trade name, Nolvadex®)) may also be used in the present invention.

Other non-limiting examples of methods of inhibiting sex steroid signalling which may be used either alone or in combination with other methods of castration include aromatase inhibitors and other adrenal gland blockers (e.g., Aminoglutethimide, formestane, vorazole, exemestane, anastrozole (trade name Arimidex®, 0.1-100 mg, e.g., 1 mg po QID), which lowers estradiol and increases LH and testosterone), letrozole (trade name Femara®, 0.2-500 mg, e.g., 2.5 mg po QID), and exemestane (trade name Aromasin®) 1-2000 mg, e.g., 25 mg/day); aldosterone antagonists (e.g., spironolactone (trade name, Aldactone®) e.g., 100 to 400 mg/day), which blocks the androgen cytochrome P-450 receptor; and eplerenone (a selective aldosterone-receptor antagonist), antiprogestogens (e.g., medroxypregesterone acetate, e.g., 5 mg/day, which inhibits testosterone syntheses and LH synthesis); and progestins and anti-progestins such as the selective progesterone response modulators (SPRM) (e.g., megestrol acetate e.g., 160 mg/day, mifepristone (RU 486, Mifeprex®, e.g., 200 mg/day)); and other compounds with estrogen/antiestrogenic activity, (e.g., phytoestrogens, flavones, isoflavones and coumestan derivatives, lignans, and industrial compounds with phenolic ring (e.g., DDT)). Also, anti-GnRH vaccines (see, e.g., Hsu et al., (2000) Cancer Res. 60:3701; Talwar, (1999) Immunol. Rev. 171:173-92), or any other pharmaceutical which mimics the effects produced by the aforementioned drugs, may also be used. In addition, steroid receptor based modulators, which may be targeted to be thymic and/or BM specific, may also be developed and used. Many of these mechanisms of inhibiting sex steroid signalling are well known. Each drug may also be used in modified form, such as acetates, citrates and other salts thereof, which are well known to those skilled in the art.

Because of the complex and interwoven feedback mechanisms of the hormonal system, administration of sex steroids may result in inhibition of sex steroid signalling. For example, estradiol decreases gonadotropin production and sensitivity to GNRH action. However, higher levels of estradiol result in gonadotropin surge. Likewise, progesterone influences frequency and amount of LH release. In men, testosterone inhibits gonadotropin production. Estrogen administered to men decreases LH and testosterone, and anti-estrogen increases LH.

In other embodiments, prolactin is inhibited in the patient. Another means of inhibiting sex steroid mediated signalling may be by means of direct or indirect modulation of prolactin levels. Prolactin is a single-chain protein hormone synthesized as a prohormone. The normal values for prolactin in males and nonpregnant females typically range from about 0 to 20 ng/ml, but in pregnancy the range is typically about 10 to 300 ng/ml. Overall, several hundred different actions have been reported for prolactin. Prolactin stimulates breast development and milk production in females. Abnormal prolactin is known to be involved in pituitary tumors, menstrual irregularities, infertility, impotence, and galactorrhea (breast milk production). A considerable amount of research is in progress to delineate the role of prolactin in normal and pathologic immune responses. It appears that prolactin has a modulatory role in several aspects of immune function, yet there is evidence to suggest that hyperprolactinemia is immunosuppressive (Matera L, (1997) Neuroimmunomodulation 4(4):171-80). Administration of prolactin in pharmacological doses is associated with a decreased survival and an inhibition of cellular immune functions in septic mice. (Oberbeck R, (2003) J Surg Res. 113(2):248-56). There are also a large number of drugs which impair dopaminergic inhibition of prolactin and give rise to hyperprolactinemia. Antidopaminergic agents include haloperidol, fluphenazine, sulpiride, metoclopramide and gastrointestinal prokinetics (e.g., bromopride, clebopride, domperidone and levosulpiride) which have been exploited clinically for the management of motor disorders of the upper gastrointestinal tract.

Inhibin A and B peptides made in the gonads in response to gonadotropins, down-regulates the pituitary and suppress FSH. Activin normally up-regulates GnRH receptors and stimulate FSH synthesis; however, over production may shut down sex steroid production. Thus these hormones may also be the target of inhibition of sex steroid-mediated signalling.

In certain embodiments, an LHRH-R antagonist is delivered to the patient, followed by an LHRH-R agonist. For example, the antagonist can be administered as a single injection of sufficient dose to cause castration within 5-8 days (this is normal for, e.g., Abarelix). When the sex steroids have reached this castrate level, the agonist is given. This protocol abolishes or limits any spike of sex steroid production, before the decrease in sex steroid production, that might be produced by the administration of the agonist. In an alternate embodiment, an LHRH-R agonist that creates little or no sex steroid production spike is used, with or without the prior administration of an LHRH-R antagonist.

### Inhibition of Sex Steroid Signalling

Sex steroids comprise a large number of the androgen, estrogen and progestin family of hormone molecules. Non-limiting members of the progestin family of C21 steroids include progesterone, 17α-hydroxy progesterone, 20α-hydroxy progesterone, pregnanedione, pregnanediol and pregnenolone. Non-limiting members of the androgen family of C19 steroids include testosterone, androstenedione, dihydrotesterone (DHT), androstanedione, androstandiol, dehydroepiandrosterone and 17α-hydroxy androstenedione. Non-limiting members of the estrogen family of C17 steroids include estrone, estradiol-17α, and estradiol-17β.

Signalling by sex steroids is the net result of complex outcomes of the components of the pathway that includes biosynthesis, secretion, metabolism, compartmentalization and action. Parts of this pathway are not fully understood; nevertheless, there are numerous existing and potential mechanisms for achieving inhibition of sex steroid signalling. In one aspect of the present invention, inhibition of sex steroid signalling is achieved by modifying the bioavailable sex steroid hormone levels at the cellular level, the so called “free” levels, by altering biosynthesis or metabolism, the binding to sex steroid receptors on or in target cells, and/or intracellular signalling of sex steroids.

It is possible to influence the signalling pathways either directly or indirectly. The direct methods include methods of influencing sex steroid biosynthesis and metabolism, binding to the respective receptor and intracellular modification of the signal. The indirect methods include those methods known to influence sex steroid hormone production and action, such as the peptide hormone and growth factors present in the pituitary gland and the gonad. The latter include, but are not limited to, follicle stimulating hormone (FSH), luteinizing hormone (LH) and activin made by the pituitary gland, and inhibin, activin and insulin-like growth factor-1 (IGF-1) made by the gonad.

The person skilled in the art will appreciate that inhibition of sex steroid signalling may take place by making the aforementioned modifications at the level of the relevant hormone, enzyme, receptor, binding molecule and/or ligand, either by direct action upon that molecule or by action upon a precursor of that molecule, including a nucleic acid that encodes or regulates it, or a molecule that can modify the action of sex steroid.

### Direct Methods of Inhibiting Signalling

Biosynthesis

The rate of biosynthesis is the major rate determining step in the production of steroid hormones and hence the bioavailability of “free” hormone in serum. Inhibition of a key enzyme such as P450 cholesterol side chain cleavage (P450scc), early in the pathway, will reduce production of all the major sex steroids. On the other hand, inhibition of enzymes later in the pathway, such as P450 aromatase (P450arom) that converts androgens to estrogens, or 5α-reductase that converts testosterone to DHT, will only effect the production of estrogens or DHT, respectively. Another important facet of sex steroid hormone biosynthesis is the family of oxidoreductase enzymes that catalyze the interconversion of inactive to bioactive steroids, for example, androstenedione to testosterone or estrone to estradiol-17 [symbol] by 17-hydroxysteroid dehydrogenase (17-HSD). These enzymes are tissue and cell specific and generally catalyze either the reduction or oxidation reaction, e.g., 17β HSD type 3 is found exclusively in the Leydig cells of the testes, whereas 17β HSD type 1 is found in the ovary. They therefore offer the possibility of specifically reducing production of the active forms of androgens or estrogens.

There are many known inhibitors of the enzymes in the steroid biosynthesis pathway that are either already in clinical use or are under development. Some examples of these, together with their treatment modalities, are listed above. It is important that the action of these enzyme inhibitors does not unduly influence production of other steroids such as glucocorticoids and mineralocorticoids from the adrenal gland that are essential for metabolic stability. When using such inhibitors, it may be necessary to provide the patient with replacement glucocorticoids and sometimes mineralocorticoids.

Sex steroid biosynthesis occurs in varied sites and utilizing multiple pathways, predominantly produced in the ovaries and testes, but there is some production in the adrenals, as well as synthesis of derivatives in other tissues, such as fat. Thus, multiple mechanisms of inhibiting sex steroid signalling may be required to ensure adequate inhibition to achieve the present invention.

Metabolism and Compartmentalization

Sex steroid hormones have a short half-life in blood, generally only several minutes, due to the rapid metabolism, particularly by the liver, and clearance by the kidney and fat. Metabolism includes conjugation by glycosylation and sulphation, as well as reduction. Some of these metabolites retain biological activity either as prohormones, for example, estrone sulphate, or through intrinsic bioactivity, such as the reduced androgens. Any interference in the rate of metabolism can influence the “free” levels of sex steroid hormones. However methods of achieving this are not currently available as are methods of influencing biosynthesis.

Another method of reducing the level of “free” sex steroid hormone is by compartmentalization by binding of the sex steroid hormone to proteins present in the serum such as sex hormone binding globulin, corticosteroid-binding globulin, albumin and testosterone-estradiol binding globulin. Binding to sex steroid ligands, such as carrier molecules may make sex steroids unavailable for receptor binding. Increased binding may result from increased levels of carriers, such as SHBG, or introduction of other ligands which bind the sex steroids, such as soluble receptors. Alternatively, decreased levels of carrier molecules may make sex steroids more susceptible to degradation. cActive or passive immunization against a particular sex steroid hormone is a form of compartmentalization. There are examples in the literature of this approach successfully increasing ovulation rates in animals after immunization against estrogen or androgen. Sex steroids are secreted from cells in secretory vesicles. Inhibition or modification of the secretory mechanism is another method of inhibiting sex steroid signaling.

Receptors and Intracellular Signalling

The sex steroids act on cells via specific receptors that can be either intracellular or, as shown more recently, on the target cell membrane.

The intracellular receptors are members of the nuclear receptor superfamily. They are located in the cytoplasm of the cell and are transported to the nucleus after binding with the sex steroid hormone where they alter the transcription of specific genes. Receptors for the sex steroid hormones exist in several forms. Well known in the literature are two forms of the progesterone receptor, PRA and PRB, and three forms of the estrogen receptor, ERα, ERβ1 and ERβ2. Transcription of genes in response to the binding of the sex steroid hormone receptor to the steroid response element in the promoter region of the gene can be modified in a number of ways. Co-activators and co-repressors exist within the nucleus of the target cell that can modify binding of the steroid-receptor complex to the DNA and thereby effect transcription. The identity of many of these co-activators and co-repressors are known, and methods of modifying their actions on steroid receptors are the topic of current research. Examples of the transcription factors involved in sex steroid hormone action are NF-1, SP1, Oct-1 and THIID. These co-regulators are required for the full action of the steroids. Methods of modifying the actions of these nuclear regulators could involve the balance between activator and repressor by the use of antagonists or through control of expression of the genes encoding the regulators.

More recently, specific receptors for estrogens and progesterone have been identified on the membranes of cells whose structures are different from the intracellular PR. Unlike the classical steroid receptors that act on the genome, these receptors deliver a rapid, non-genomic action via intracellular pathways that are not yet fully understood. One report suggests that estrogens interacting with membrane receptors activate the sphingosine pathway that is related to cell proliferation.

There are methods available or in development to alter the action of steroids via their cytoplasmic receptors. In this case, antiandrogens, antiestrogens, and antiprogestins that interact with the specific steroid receptors are well known in the literature and are in clinical use, as described above. Their action may be to compete for, or block the receptor, to modify receptor levels, sensitivity, conformation, associations or signalling. These drugs come in a variety of forms, steroidal and non-steroidal, competitive and non-competitive. Of particular interest are the selective receptor modulators (SARMS, SERMS and SPRM) which are targeted to particular tissues and are exemplified above.

Down regulation of receptors can be achieved in 2 ways; first, by excess agonist (steroid ligand), and second, by inhibiting transcription of the respective gene that encodes the receptor. The first method can be achieved through the use of selective agonists, such as tamoxifen. The second method is not yet in clinical use.

### Indirect Methods of Inhibiting Signalling

Biosynthesis

One of the indirect methods of inhibiting sex steroid signalling involves down-regulation of the biosynthesis of the respective steroid by a modification to the availability or action of the pituitary gonadotrophins, FSH and LH, that are responsible for driving the biosynthesis of the sex steroid hormones in the gonad. One established inhibitor of FSH secretion is inhibin, a hormone produced by the gonads in response to FSH. Administration of inhibin to animals has been shown to reduce FSH levels in serum due to a decrease in the pituitary secretion of FSH. The best known way of accomplishing a reduction in both gonadotrophins is via the hypothalamic hormone, GnRH/LHRH, which drives the pituitary synthesis and secretion of FSH and LH. Agonists and antagonists of GnRH that reduce the secretion of FSH and LH, and hence gonadal sex steroid production, are now available for clinical use, as described herein.

Another indirect method of reducing the biosynthesis of sex steroid hormones is to modify the action of FSH and LH at the level of the gonad. This could be achieved by using antibodies directed against FSH and LH, or molecules designed to compete with FSH and LH for their respective receptors on gonadal cells that produce the sex steroid hormones. Another method of modifying the action of FSH and LH on gonadal cells is by a co-regulator of gonadotrophin action. For example, activin can reduce the capacity of the theca cells of the ovary and the Leydig cells of the testes to produce androgen in response to LH.

Modification may take place at the level of hormone precursors, such as inhibition of cleavage of a signal peptide, for example the signal peptide of GnRH.

Receptors and Intracellular Signalling

Indirect methods of altering the signalling action of the sex steroid hormones include down-regulation of the receptor pathways leading to the genomic or non-genomic actions of the steroids. An example of this is the capacity of progesterone to down-regulate the level of ER in target tissues. Future methods include treatment with molecules known to influence the co-regulators of the receptors in the cell nucleus leading to a decrease in the capacity of the cell to respond to the steroid.

Additional Factors

While the stimulus for the direct and indirect effects on BM functionality, BM lymphopoiesis, and immune cell functionality is fundamentally based on the inhibition of the effects of sex steroids and/or the direct effects of the LHRH analogs, it may be useful to include additional substances which can act in concert to enhance or increase (additive, synergistic, or complementary) the thymic, BM, and/or immune cell effects and functionality. Additional substances may or may not be used. Such compounds include, but are not limited to, cytokines and growth factors, such as interleukin-2 (IL-2; 100,000 to 1,000,000 IU, e.g., 600,000 IU/Kg every 8 hours by IV repeat doses), interleukin-7 (IL-7; 10 ng/kg/day to 100 mcg/kg/day subject to therapeutic discretion), interleukin-15 (IL-15; 0.1-20 mug/kg IL-15 per day), interleukin-II (IL-11; 1-1000 μg/kg) members of the epithelial and fibroblast growth factor families, stem cell factor (SCF; also known as steel factor or c-kit ligand; 0.25-12.5 mg/ml), granulocyte colony stimulating factor (G-CSF; 1 and 15 μg/kg/day IV or SC), granulocyte macrophage stimulating factor (GM-CSF; 50-1000 μg/sq meter/day SC or IV), insulin dependent growth factor (IGF-1), and keratinocyte growth factor (KGF; 1 μg/kg to 100 mg/kg/day) (see, e.g., Sempowski et al., (2000) J. Immunol. 164:2180; Andrew and Aspinall, (2001) J. Immunol. 166:1524-1530; Rossi et al., (2002) Blood 100:682); erythropoietin (EPO; 10-500 units/kg IV or SC). A non-exclusive list of other appropriate hematopoietins, CSFs, cytokines, lymphokines, hematopoietic growth factors and interleukins for simultaneous or serial co-administration with the present invention includes, Meg-CSF (Megakaryocyte-Colony Stimulating Factor, more recently referred to as c-mpl ligand), MIF (Macrophage Inhibitory Factor), LIF (Leukemia Inhibitory Factor), TNF (Tumor Necrosis Factor), IGF, platelet derived growth factor (PDGF), M-CSF, IL-1, IL-4, IL-5, IL-6, IL-8, IL-9, IL-10, IL-12, IL-13, LIF, flt3/flk2, human growth hormone, B-cell growth factor, B-cell differentiation factor and eosinophil differentiation factor, or combinations thereof.

One or more of these additional compound(s) may be given once at the initial LHRH analog (or other castration method) application. Each treatment may be given in combination with the agonist, antagonist or any other form of sex steroid disruption. Since the growth factors have a relatively rapid half-life (e.g., in the hours), they may need to be given each day (e.g., every day for 7 days or longer). The growth factors/cytokines may be given in the optimal form to preserve their biological activities, as prescribed by the manufacturer, e.g., in the form of purified proteins. However, additional doses of any one or combination of these substances may be given at any time to further stimulate the functionality of the BM and other immune cells. In certain cases, sex steroid ablation or interruption of sex steroid signalling is done concurrently with the administration of additional cytokines, growth factors, or combinations thereof. In other cases, sex steroid ablation or interruption of sex steroid signalling is done sequentially with the administration of additional cytokines, growth factors, or combinations thereof.

The term “mobilizing agent” is herein defined as agents such as SDF-1 (e.g., AMD3100), growth hormone, GM-CSF, G-CSF and chemotherapeutics (e.g., cyclophosphamide), which enhance mobilization of stem cells from the BM.

G-CSF and GM-CSF are known to mobilize the production of granulocytes (primarily neutrophils) and macrophages, respectively, and also result in increased production of DC from the BM, which help provide a non-specific immune response in the patient to antigenic challenge (Janeway et al., (2001) Immunobiology 5^{th} Ed., p. 325). Clinically, G-CSF and GM-CSF are used, for example, to decrease the incidence of infection (as manifested by febrile neutropenia) in patients with non-myeloid malignancies receiving myelosuppressive anti-cancer drugs, which are typically associated with a significant incidence of severe neutropenia and fever. Additionally, both of these drugs are approved clinically to prevent infections in patients receiving HSCT. Both G-CSF and GM-CSF are currently used in patients undergoing peripheral blood progenitor cell collection or therapy. Colony stimulating factors (CSFs), which stimulate the differentiation and/or proliferation of BM stem cells, have generated much interest because of their therapeutic potential for restoring depressed levels of hematopoietic stem cell-derived cells. CSFs, in both human and murine systems, have been identified and distinguished according to their activities. For example, granulocyte-CSF (G-CSF) and macrophage-CSF (M-CSF) stimulate the in vitro formation of neutrophilic granulocyte and macrophage colonies, respectively, while GM-CSF and interleukin-3 (IL-3) have broader activities and stimulate the formation of both macrophage, neutrophilic and eosinophilic granulocyte colonies. IL-3 also stimulates the formation of mast, megakaryocyte and pure and mixed erythroid colonies (when erythropoietin is added). GM-CSF accelerates recovery of neutrophils and maintains functional capacity, yet has little demonstrable effect on platelet recovery. In contrast, IL-3 promotes a slower increase in the recovery of neutrophils and monocytes while accelerating the recovery of platelets.

Thus, G-CSF and/or GM-CSF are used in some of the methods of the invention. Sex steroid ablation, together (sequentially or concurrently) with G-CSF and/or GM-CSF therapy results in an increase in the output from the BM of both lymphoid and myeloid cells which, in turn, significantly improves both the short and long term outcomes for patients suffering, or likely to suffer from, infections. In another method, the CSFs are administered 3-4 days after chemotherapy or radiation therapy. Clinical outcomes already associated with the use of the CSFs are also greatly enhanced by an interruption to sex steroid signalling. In particular, using the methods of the instant invention together with CSF's, allows for much greater infection control in patients receiving, e.g., cancer radiation or chemotherapy. Additionally, if the immune system can be effectively and promptly “rebooted,” increased dosages and/or frequency of chemotherapy drugs or radiation therapy may be used. This may occur with or without the introduction of allogeneic or autogenic HSC, which would further enhance the timely return of immune system functionality. Castration will also result in a lower number of HSC that have to be transplanted, which will be useful when only a limited number of HSC can be obtained from a donor or when cord blood stem cells are used for transplant.

For instance, the concurrent use of two separate classes of drugs (e.g., a GnRH analog, such as Lupron®, and an androgen blocker, such as Cosudex®) may allow for the same immune system regeneration but may require a reduced dosage of the G-CSF or GM-CSF. Similarly, the concurrent use of these two separate classes of drugs may allow for a greater, or more prolonged, rejuvenation of immune system cells, while utilizing the same dosage of G-CSF or GM-CSF. Additionally, the concurrent use of two separate classes of drugs may allow for the same rejuvenation of immune system cells, while utilizing a reduced dosage (i.e., a reduction compared to the “normally” used dosages used for the treatment of prostate cancer, endometriosis, or breast cancer) of the drug, or combination of drugs, used to ablate or interrupt sex steroid signalling. Further, the concurrent use of these two separate classes of drugs allows for a greater, or prolonged, rejuvenation of immune system cells, while utilizing a reduced dosage of the drug, or combination of drugs, used to ablate or interrupt sex steroid signalling.

Indications

The use of drugs known to cause sex steroid ablation, or which interrupt sex steroid signalling, either alone or in combination, with or without the aforementioned growth factors and cytokines, may be used for the following: reduction of infections associated with a number of treatment regimens; rejuvenation of the BM following ablative therapy (see, e.g., Example 19); as an adjunct in enabling HSC engraftment (see, e.g., Example 22); as an adjunct in the effective management of allogeneic or autologous organ or cell transplants (see, e.g., Examples 21 and 22, and co-pending, co-owned U.S. Ser. Nos. 10/419,039 and 10/749,119); vaccination protocols (see, e.g., Examples 10-13, 29, and co-owned, co-pending U.S. Ser. Nos. 10/418,747 and 10/748,450); management of various autoimmune diseases (see, e.g., Examples 32-35 and co-owned, co-pending U.S. Ser. Nos. 10/419,066 and 10/749,118); treatment or management of the consequences of various infectious diseases (see, e.g., Examples 3 and 14); and improvement in the prevention and treatment of various cancers (see, e.g., Example 13, and co-owned, co-pending U.S. Ser. Nos. 10/418,727 and 10/749,122), and various gene therapy protocols (see Example 14 and co-owned, co-pending U.S. Ser. No. 10/419,068 and 10/748,851).

The use of these drugs in these diseases will either result in more effective treatment outcomes or will result in the overall treatment protocols being more efficient. Additionally, as described in, e.g., Examples 25 and 26, the doses or administration of the various chemotherapy drugs (or doses of radiation therapy) may be altered such that they now produce less side effects and/or result in better quality of life outcomes for the patients. Moreover, the co-administration of the various cytokines and growth factors may allow for a reduced number of HSC that need to be transplanted. For example, using the method of the invention, it may now be possible to use human cord blood for adult HSCT, since a reduced number of cells is required to obtain engraftment.

Pharmaceutical Compositions

The compounds used in this invention may be supplied in any pharmaceutically acceptable carrier or may be supplied without a carrier. Formulations of pharmaceutical compositions can be prepared according to standard methods (see, e.g., Remington, The Science and Practice of Pharmacy, Gennaro A. R., ed., 20^{th} edition, Williams & Wilkins PA, USA (2000)). Non-limiting examples of pharmaceutically acceptable carriers include physiologically compatible coatings, solvents and diluents. For parenteral, subcutaneous, intravenous, and intramuscular administration; the compositions may be protected such as by encapsulation. Alternatively, the compositions may be provided with carriers that protect the active ingredient(s), while allowing a slow release of those ingredients. Numerous polymers and co-polymers are known in the art for preparing time-release preparations, such as various versions of lactic acid/glycolic acid co-polymers. See, for example, U.S. Pat. No. 5,410,016, which uses modified polymers of polyethylene glycol (PEG) as a biodegradable coating.

Formulations intended to be delivered orally can be prepared as liquids, capsules, tablets, and the like. These compositions can include, for example, excipients, diluents, and/or coverings that protect the active ingredient(s) from decomposition. Such formulations are well known (see, e.g., Remington, The Science and Practice of Pharmacy, Gennaro A. R., ed., 20^{th} edition, Williams & Wilkins PA, USA (2000)).

In any of the formulations of the invention, other compounds that do not negatively affect the activity of the LHRH analogs (i.e., compounds that do not block the ability of an LHRH analog to disrupt sex steroid hormone signalling) may be included. Examples are various growth factors and other cytokines as described herein.

Dose

Doses of a sex steroid analog or inhibitor used, in accordance with the invention, to disrupt sex steroid hormone signalling, can be readily determined by a routinely trained physician or veterinarian, and may also be determined by consulting medical literature (e.g., THE PHYSICIAN'S DESK REFERENCE, 52ND EDITION, Medical Economics Company, 1998).

The dosage regimen involved in a method for treating the above-described conditions will be determined by the attending physician considering various factors which modify the action of drugs, e.g., the condition, body weight, sex and diet of the patient, the severity of any illness, time of administration and other clinical factors. Progress of the treated patient can be monitored by periodic assessment of the hematological profile, e.g., differential cell count and the like.

The dosing recited above is adjusted to compensate for additional components in the therapeutic composition. These include co-administration with other CSF, cytokine, lymphokine, interleukin, hematopoietic growth factor; co-administration with chemotherapeutic drugs and/or radiation; and various patient-related issues as identified by the attending physician, such as factors which modify the action of drugs, e.g., the condition, body weight, sex and diet of the patient, the severity of any illness, time of administration and other clinical factors.

In addition to dosing described above, for example, LHRH analogs and other sex steroid analogs can be administered in a one-time dose that will last for a period of time (e.g., 3 to 6 months). In certain cases, the formulation will be effective for one to two months. The standard dose varies with type of analog used, but is readily determinable by those skilled in the art without undue experimentation. In general, the dose is between about 0.01 mg/kg and about 10 mg/kg, or between about 0.01 mg/kg and about 5 mg/kg.

The length of time of sex steroid inhibition or LHRH/GnRH analog treatment varies with the degree of thymic atrophy and damage and is readily determinable by those skilled in the art without undue experimentation. For example, the older the patient, or the more the patient has been exposed to T cell depleting reagents, such as chemotherapy or radiotherapy, the longer it is likely that they will require treatment, for example, with GnRH. Four months is generally considered long enough to detect new T cells in the blood. Methods of detecting new T cells in the blood are known in the art. For instance, one method of T cell detection is by determining the existence of T cell receptor excision circles (TREC's), which are formed when the TCR is being formed and are lost in the cell after it divides. Hence, TREC's are only found in new (naïve) T cells. TREC levels are an indicator of thymic function in humans. These and other methods are described in detail in WO/00 230,256.

Dose varies with the sex steroid inhibitor or, e.g., anti-sex steroid vaccine or other blocker used. In certain cases, a dose may be prepared to last as long as a periodic epidemic lasts. For example, “flu season” occurs usually during the winter months. A formulation of an LHRH analog can be made and delivered as described herein to protect a patient for a period of two or more months starting at the beginning of the flu season, with additional doses delivered every two or more months until the risk of infection decreases or disappears.

The formulation can be made to enhance the immune system. Alternatively, the formulation can be prepared to specifically deter infection by, e.g., influenza (flu) viruses while also enhancing the immune system. This latter formulation may include genetically modified (GM) cells that have been engineered to create resistance to flu viruses (see below). The GM cells can be administered with the sex steroid analog or LHRH analog formulation or separately, both spatially and/or in time. As with the non-GM cells, multiple doses over time can be administered to a patient to create protection and prevent infection with the flu virus over the length of the flu season.

As will be understood by persons skilled in the art, at least some of the means for disrupting sex steroid signalling will only be effective as long as the appropriate compound is administered. As a result, an advantage of certain embodiments of the present invention is that once the desired immunological affects of the present invention have been achieved (2-3 months), the treatment can be stopped and the subject's reproductive system will return to normal.

Delivery of Agents for Chemical Castration

Administration of sex steroid ablating agents may be by any method which delivers the agent into the body. Thus, the sex steroid ablating agent may be administered, in accordance with the invention, by any route including, without limitation, intravenous, subdermal, subcutaneous, intramuscular, topical, and oral routes of administration.

In addition to the methods described above, delivery of the compounds for use in the methods of this invention may be accomplished via a number of methods known to persons skilled in the art. One standard procedure for administering chemical inhibitors to inhibit sex steroid mediated signalling utilizes a single dose of an LHRH agonist that is effective for three months. For this, a simple, one-time i.v. or i.m. injection would not be sufficient as the agonist would be cleared from the patient's body well before the three months are over. Instead, a depot injection or an implant may be used, or any other means of delivery of the inhibitor that will allow slow release of the inhibitor. Likewise, a method for increasing the half-life of the inhibitor within the body, such as by modification of the chemical, while retaining the function required herein, may be used.

Useful delivery mechanisms include, but are not limited to, laser irradiation of the skin. This embodiment is described in more detail in co-owned, co-pending U.S. Ser. No. 10/418,727 and also in U.S. Pat. Nos. 4,775,361, 5,643,252, 5,839,446, 6,056,738, 6,315,772, and 6,251,099. Another useful delivery mechanism includes the creation of high pressure impulse transients (also called stress waves or impulse transients) on the skin. This embodiment is described in more detail in co-owned, co-pending U.S. Ser. No. 10/418,727 and also U.S. Pat. Nos. 5,614,502 and 5,658,822. Each method may be accompanied or followed by placement of the compound(s) with or without carrier at the same locus. One method of this placement is in a patch placed and maintained on the skin for the duration of the treatment.

Timing

In one case, the administration of agents (or other methods of castration) that ablate sex steroids or interrupt sex steroid signalling occurs prior to, e.g., chemotherapy or radiation regimen that is likely to cause some BM marrow cell ablation and/or damage to circulating immune cells.

Cells

Injection of hematopoietic progenitor cells, e.g., broadly defined as CD34⁺ hematopoietic cells (ideally autologous), can enhance the degree and kinetics of thymic regrowth and/or increases in immune cell and BM functionality and engraftment without, prior to or concurrently with, thymic regeneration. HSC may also be further defined as Thy-1 low and CD38−; CD34+CD38−. Thy-1 low cells which also lack markers of other cell lineages (lin-ve) are the more primitive HSC being longer lasting or having longer-term repopulating capacity.

The methods of the various inventions described herein can be supplemented by the addition of, e.g., CD34⁺ HSC and/or epithelial stem cells. In one instance, these cells are autologous or syngeneic and have been obtained from the patient or twin prior to thymus reactivation. The HSC can be obtained by sorting CD34⁺ or CD34^{lo} cells from the patient's blood and/or BM. The number of HSC can be enhanced in several ways including, but not limited to, by administering G-CSF (Neupogen, Amgen) to the patient prior to collecting cells, culturing the collected cells in SCGF, and/or administering G-CSF to the patient after CD34⁺ cell supplementation. Alternatively, the CD34⁺ cells need not be sorted from the blood or BM if their population is enhanced by prior injection of G-CSF into the patient.

HSC may be used for genetic modification. These may be derived from BM, peripheral blood, or umbilical cord, or any other source of HSC, and may be either autologous or nonautologous. Also useful are lymphoid and myeloid progenitor cells, mesenchymal stem cells also found in the bone marrow and epithelial stem cells, also either autologous or nonautologous. The stem cells may also include umbilical cord blood. They may also include stem cells which have the potential to form into many different cell types, e.g., embryonic stem cells and adult stem cells, now found in may tissues, e.g., BM, pancreas, brain, and the olfactory system.

In the event that nonautologous (donor) cells are used, tolerance to these cells is created during or after thymus reactivation. During or after the initiation of blockage of sex steroid mediated signalling, the relevant (genetically modified (GM) or non-genetically modified) donor cells are transplanted into the recipient. These cells, ideally stem or progenitor cells, are incorporated into and accepted by the thymus wherein they create tolerance to the donor by eliminating any newly-produced T cells which, by chance, could be reactive against them. They are then “belonging to the recipient” and may become part of the production of new T cells and DC by the thymus. The resulting population of T cells recognize both the recipient and donor as self, thereby creating tolerance for a graft from the donor (see co-owned, co-pending U.S. Ser. No. 10/419,039 and PCT/IB01/02740).

In another embodiment, the administration of stem or precursor donor cells (genetically modified or not genetically modified) comprises cells from more than one individual, so that the recipient develops tolerance to a range of MHC types, enabling the recipient to be considered a suitable candidate for a cell, tissue or organ transplant more easily or quickly since they are an MHC match to a wider range of donors.

The present invention also provides methods for incorporation of foreign DC into a patient's thymus. This may be accomplished by the administration of donor cells to a recipient to create tolerance in the recipient. The donor cells may be HSC, epithelial stem cells, adult or embryonic stem cells, or hematopoietic progenitor cells. The donor cells may be CD34⁺ HSC, lymphoid progenitor cells, or myeloid progenitor cells. In some cases, the donor cells are CD34+ or CD34lo HSC. The donor HSC may develop into DC in the recipient. The donor cells may be administered to the recipient and migrate through the peripheral blood system to the reactivating thymus either directly or via the BM. To enhance thymic incorporation for tolerance induction, the stem cells may also be injected intrathymically in combination with activation of thymic regrowth through use of sex steroid inhibitors, e.g., LHRH/GnRH analogues. Even non-HSC are likely to be induced to form into DC within the thymic microenvironment and its content of appropriate growth factors for such cells.

The uptake into the thymus of the hematopoietic precursor cells is substantially increased in the inhibition or absence of sex steroids. These cells become integrated into the thymus and produce DC, NK, NKT, and T cells in the same manner as do the recipient's cells. The result is a chimera of T cells, DC and the other cells. The incorporation of donor DC in the recipient's thymus means that T cells produced by this thymus will be selected such that they are tolerant to donor cells. Such tolerance allows for a further transplant from the donor (or closely matched to the donor) of cells, tissues and organs with a reduced need for immonusuppressive drugs since the transplanted material will be recognized by the recipient's immune system as self.

Optional Genetic Modification of Stem or Progenitor Cells

The present disclosure also comprises methods for optionally altering the immune system of an individual and methods of gene therapy. This is accomplished by the administration of GM cells to a recipient and through disruption of sex steroid mediated signalling. The invention further comprises methods of gene therapy through enhancing the functionality of BM and/or immune cells in conjunction with a regenerating thymus, or alternatively, prior to, or without reactivation of the thymus.

The genetically modified cells may be HSC, epithelial stem cells, embryonic or adult stem cells, or myeloid or lymphoid progenitor cells. In one embodiment, the genetically modified cells are CD34⁺ or CD34^{lo} HSC, lymphoid progenitor cells, or myeloid progenitor cells. In another embodiment, the genetically modified cells are CD34⁺ or CD34^{lo} HSC. The genetically modified cells are administered to the patient and migrate through the peripheral blood system to the thymus. The uptake into the thymus of these hematopoietic precursor cells is substantially increased in the absence of sex steroids. These cells become integrated into the thymus and produce dendritic cells and T cells carrying the genetic modification from the altered cells. The results are a population of T cells with the desired genetic change that circulate in the peripheral blood of the recipient, and the accompanying increase in the population of cells, tissues and organs caused by reactivation of the patient's thymus.

Within 3-4 weeks of the start of blockage of sex steroid mediated signalling (approximately 2-3 weeks after the initiation of LHRH treatment), the first new T cells are present in the blood stream. Full development of the T cell pool, however, may take 3-4 (or more) months.

The present disclosure also comprises methods for gene therapy using genetically modified hematopoietic stem cells, lymphoid progenitor cells, myeloid progenitor cells, epithelial stem cells, or combinations thereof (GM cells). Previous attempts by others to deliver such cells as gene therapy have been unsuccessful, resulting in negligible levels of the modified cells. The present disclosure provides a new method for delivery of these cells which promotes uptake and differentiation of the cells into the desired T cells. The modified cells are injected into a patient. The modified stem and progenitor cells are taken up by the thymus and converted into T cells, dendritic cells, and other cells produced in the thymus. Each of these new cells contain the genetic modification of the parent stem/progenitor cell.

In one embodiment, the methods of the invention use genetically modified HSC, lymphoid progenitor cells, myeloid progenitor cells, epithelial stem cells or combinations thereof (collectively referred to as GM cells) to produce an immune system resistant to attack by particular antigens.

An appropriate gene or polynucleotide (i.e., the nucleic acid sequence defining a specific protein) that will create or induce resistance to one or more infectious or other agents is engineered into the stem and/or progenitor cells. By introducing the specific gene into the HSC, the cell differentiates into, e.g., an APC, it expresses the protein as a peptide expressed in the context of MHC class I or II. This expression will greatly increase the number of APC “presenting” the desired antigen than would normally occur, thereby increasing the chance of the appropriate T cell recognizing the specific antigen and responding.

Many antineoplastic or cytotoxic anticancer drugs used in the clinic today cause moderate to severe bone marrow toxicity (e.g., vinblastine, cisplatin, methotrexate, alkylating agents, anti-folate, a vinca alkaloid and anthracyclines). In another embodiment of the present invention, drug resistance genes can be introduced into HSC to confer resistance to anticancer drugs. Such genes include, for example, dihydrofolate reductase. The use of the present invention to provide HSC which are resistant to the cytotoxic effects of these chemotherapeutics may allow for the greater use of these drugs and/or less side effects by reducing the incidence and severity of myelosupporession (Podda et al., (1992) Proc. Natl. Acad. Sci. USA 89:9676; Banerjee et al., (1994) Stem Cells 12:378).

The modified stem and progenitor cells are taken up by the thymus and converted into T cells, DC, and other cells produced in the thymus. Each of these new cells contains the genetic modification of the parent stem/progenitor cell, and is thereby completely or partially resistant to infection or damage by the agent or agents. B cells are also increased in number in the bone marrow, blood and peripheral lymphoid organs, such as the spleen and lymph nodes, within e.g., two weeks of castration. In one embodiment, a patient has already been in contact with an agent, or is at a high risk of doing so.

The person may be given a sex steroid analog to activate their thymus, and/or to improve their bone marrow function, which includes the increased ability to take up and produce HSC. The person may be injected with their own HSC, or may be injected with HSC from an appropriate donor, which has, e.g., treatment with G-CSF for 3 days (2 injections, subcutaneously per day) followed by collection of HSC from the blood on days 4 and 5. The HSC may be transfected or transduced with a gene (e.g., encoding the protein, peptide, or antigen from the agent) to produce the required protein or antigen. Following injection into the patient, the HSC enter the bone marrow and eventually some evolve into antigen presenting cells (APC) throughout the body. The antigen is expressed in the context of MHC class I and/or MHC class II molecules on the surface of these APC. By expressing the desired antigen, the APC improve the activation of T and B lymphocytes. The transplanted HSC may also enter the thymus, develop into DC, and present the antigen in question to developing T lymphocytes. If present in low numbers (e.g., <0.1% of thymus cells) the DC can bias the selection of new T cells to those reactive to the antigen. If the particular DC are present in high numbers, the same principle can be used to delete the new T cells which are potentially reactive to the antigen, which may be used in the prevention or treatment of autoimmune diseases.

In one embodiment, a patient is infected with HIV. In a specific embodiment, the method for treating this patient includes the following steps, which are provided in more detail below: (1) treatment with Highly Active Anti-Retrovirus Therapy (HAART) to lower the viral titer, which treatment continues throughout the procedure to prevent or reduce infection of new T cells; (2) ablation of T cells (immunosuppression); (3) blockage of sex steroid mediated signalling, for example, by administering a sex steroid analog, such as an LHRH analog; (4) at the time the thymus begins reactivating, administration of GM cells that have been modified to contain a gene that expresses a protein that will prevent HIV infection, prevent HIV replication, disable the HIV virus, or other action that will stop the infection of T cells by HIV; (5) if the GM cells are not autologous, administration of the donor cells before or concurrently with thymus reactivation will prime the immune system to recognize the donor cells as self; and (6) when the thymic chimera is established and the new cohort of mature T cells have begun exiting the thymus, reduction and eventual elimination of immunosuppression.

During or after the castration step, hematopoietic stem or progenitor cells or epithelial stem cells from the donor may be transplanted into the recipient patient. These cells are accepted by the thymus as belonging to the recipient and become part of the production of new T cells and DC by the thymus. The resulting population of T cells recognize both the recipient (and donor, in the case of nonautologus transplants) as self. Tolerance for a graft from the donor may also be created in the recipient. The graft may be cells, tissues or organs of the donor, or combinations thereof.

In one embodiment, thymic grafts can be used in the methods of the invention to improve engraftment of the donor cells or tolerance to the donor graft. In some embodiments, thymic grafts are used when the patient is athymic, when the patient's thymus is resistant to regeneration, or to hasten regeneration. In certain embodiments, a thymic xenograft to induce tolerance is used (e.g., U.S. Pat. No. 5,658,564). In other embodiments, an allogenic thymic graft is used.

As herein defined, the phrase “creating tolerance” or “inducing tolerance” in a patient, and other similar phrases, refers to complete, as well as partial tolerance induction (e.g., a patient may become either more tolerant, or completely tolerant, to the graft, as compared to a patient that has not been treated according to the methods of the invention). Tolerance induction can be tested, e.g., by an MLR reaction, using methods known in the art.

Thus, in one method, a patient receives a HSCT during or after castration. In one case, the patient is injected with their own HSC. In another case, the patient is injected with HSC from an appropriate donor. The patient or donor may or may not be pretreated with G-CSF (e.g., 2 s.c. injections per day for three days, followed by collection of HSC from the blood on days 4 and 5). In one case, hematopoietic cells are supplied to the patient before or concurrently with thymic reactivation, which increases the immune capabilities of the patient's body. The transplanted cells may or may not be genetically modified. The transplanted cells may be HSC, epithelial stem cells, or hematopoietic progenitor cells. The transplanted cells may be CD34⁺ HSC, lymphoid progenitor cells, or myeloid progenitor cells. In certain cases, the transplanted cells are CD34⁺ or CD34^{lo} HSC. The HSC may or may not be genetically modified.

In certain methods, the HSC are transfected or transduced with a gene (e.g., encoding the protein, peptide, or antigen from the agent or other gene of interest) to produce a protein or antigen of interest. In one example, the methods of the invention use genetically modified HSC, lymphoid progenitor cells, myeloid progenitor cells, epithelial stem cells or combinations thereof (collectively referred to as “GM cells”) to produce an immune system resistant to attack by particular antigens (see, e.g., Example 14). This method is described in more detail in co-owned U.S. Ser. Nos. 09/758,910, 10/419,068, and 10/399,213.

The uptake into the thymus of HSC is substantially increased in the absence of sex steroids. These cells become integrated into the thymus and produce DC and T cells carrying the genetic modification from the altered cells. The results are a population of T cells with the desired genetic change that circulate in the peripheral blood of the recipient, and the accompanying increase in the population of cells, tissues and organs caused by a reactivating thymus, which are capable of rapid, specific responses to antigen. Within three to four weeks of the start of blockage of sex steroid mediated signalling (approximately two to three weeks after the initiation of LHRH treatment), the first new T cells may be present in the blood stream. Full development of the T cell pool may take three to four months.

Following injection into the patient, the HSC enter the bone and bone marrow from the blood and then some exit back to the blood to be eventually converted into T cells, DC, APC throughout the body. Antigens are expressed in the context of MHC class I and/or MHC class II molecules on the surface of these APC. In the case of GM cells, by expressing the desired antigen, the APC improve the activation of T and B lymphocytes. The transplanted HSC may also enter the thymus, develop into DC, and present the antigen in question to developing T lymphocytes.

If present in low numbers (e.g., <0.1% of thymus cells) the DC can bias the selection of new T cells to those reactive to the antigen. If the particular DC are present in high numbers, the same principle can be used to delete the new T cells which are potentially reactive to the antigen, which may be used in the prevention of autoimmune diseases. B cells are also increased in number in the BM, blood and peripheral lymphoid organs, such as the spleen and lymph nodes, within e.g., two weeks of castration.

In the case of the HSC being GM for a specific trait, each of the new cells contain the genetic modification of the parent stem/progenitor cell, and is thereby completely or partially resistant to infection or damage by the agent or agents.

Methods for isolating and transducing stems cells and progenitor cells are well known to those skilled in the art. Examples of these types of processes are described, for example, in PCT Publication Nos. WO 95/08105, WO 96/33281, WO 96/33282, U.S. Pat. Nos. 5,681,559, 5,199,942, 5,559,703, 5,399,493, 5,061,620.

Antisense Polynucleotides

The term “antisense” is herein defined as a polynucleotide sequence which is complementary to a polynucleotide of the present invention. The polynucleotide may be DNA or RNA. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated.

Catalytic Nucleic Acids

The term “catalytic nucleic acid” is herein defined as a DNA molecule or DNA containing molecule (also known in the art as a “deoxyribozyme” or “DNAzyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T and U, as well as derivatives thereof. Derivatives of these bases are well known in the art.

Typically, the catalytic nucleic acid contains an antisense sequence for specific recognition of a target nucleic acid, and a nucleic acid cleaving enzymatic activity. The catalytic strand cleaves a specific site in a target nucleic acid. The types of ribozymes that are particularly useful in this invention are the hammerhead ribozyme (Haseloff and Gerlach (1988) Nature 334:585), Perriman et al., (1992) Gene 113:157, and the hairpin ribozyme (Shippy et al., (1999) Mol. Biotechnol. 12:117).

dSRNA

Double stranded RNA (dsRNA) is particularly useful for specifically inhibiting the production of a particular protein. Although not wishing to be limited by theory, one group has provided a model for the mechanism by which dsRNA can be used to reduce protein production (Dougherty and Parks, (1995), Curr. Opin. Cell Biol. 7:399). This model has more recently been modified and expanded (Waterhouse et al., (1998) Proc. Natl. Acad. Sci. USA 95:13959). This technology relies on the presence of dsRNA molecules that contain a sequence that is essentially identical to the mRNA of the gene of interest, in this case an mRNA encoding a polypeptide according to the first aspect of the invention. Conveniently, the dsRNA can be produced in a single open reading frame in a recombinant vector or host cell, where the sense and antisense sequences are flanked by an unrelated sequence which enables the sense and anti-sense sequences to hybridize to form the dsRNA molecule with the unrelated sequence forming a loop structure. The design and production of suitable dsRNA molecules for the present invention are well within the capacity of a person skilled in the art, particularly considering Dougherty and Parks, (1995), Curr. Opin. Cell Biol. 7:399; Waterhouse et al., (1998) Proc. Natl. Acad. Sci. USA 95:13959; and PCT Publication Nos. WO 99/32619, WO 99/53050, WO 99/49029, and WO 01/34815.

Anti-HIV Constructs

Those skilled in the art would be able to develop suitable anti-HIV constructs for use in the present invention. Indeed, a number of anti-HIV antisense constructs and ribozymes have already been developed and are described, for example, in U.S. Pat. Nos. 5,811,275, 5,741,706, and 5,144,019, and PCT Publication No. WO 94/26877 and Australian Patent Application No. 56394/94.

Genes

Useful genes and gene fragments (polynucleotides) for use in the methods of the invention involving GM HSCTs include those that code for resistance to infection of T cells by a particular infectious agent or agents. Such infectious agents include, but are not limited to, HIV, T cell leukemia virus, and other viruses that cause lymphoproliferative diseases.

With respect to HIV/AIDS, a number of genes and/or gene fragments may be used, including, but not limited to, the nef transcription factor; a gene that codes for a ribozyme that specifically cuts HIV genes, such as tat and rev (Bauer et al., (1997) Blood 89:2259); the trans-dominant mutant form of HIV-1 rev gene, RevM10, which has been shown to inhibit HIV replication (Bonyhadi et al., (1997) J. Virol. 71:4707); an overexpression construct of the HIV-1 rev-responsive element (RRE) (Kohn et al., (1999) Blood 94:368); any gene that codes for an RNA or protein whose expression is inhibitory to HIV infection of the cell or replication; and fragments and combinations thereof.

These genes or gene fragments may be used in a stably expressible form. The term “stably expressible” is herein defined to mean that the product (RNA and/or protein) of the gene or gene fragment (“functional fragment”) is capable of being expressed on at least a semi-permanent basis in a host cell after transfer of the gene or gene fragment to that cell, as well as in that cell's progeny after division and/or differentiation. This requires that the gene or gene fragment, whether or not contained in a vector, has appropriate signalling sequences for transcription of the DNA to RNA. Additionally, when a protein coded for by the gene or gene fragment is the active molecule that affects the patient's condition, the DNA also codes for translation signals.

In most cases, the genes or gene fragments are contained in vectors. Those of ordinary skill in the art are aware of expression vectors that may be used to express the desired RNA or protein.

Expression vectors are vectors that are capable of directing transcription of DNA sequences contained therein and translation of the resulting RNA. Expression vectors are capable of replication in the cells to be genetically modified, and include plasmids, bacteriophage, viruses, and minichromosomes. Alternatively, the gene or gene fragment may become an integral part of the cell's chromosomal DNA. Recombinant vectors and methodology are well-known to those skilled in the art.

Expression vectors useful for expressing the proteins of the present invention may comprise an origin of replication. Suitably constructed expression vectors comprise an origin of replication for autonomous replication in the cells, or are capable of integrating into the host cell chromosomes. Such vectors may also contain selective markers, a limited number of useful restriction enzyme sites, a high copy number, and strong promoters. Promoters are DNA sequences that direct RNA polymerase to bind to DNA and initiate RNA synthesis; strong promoters cause such initiation at high frequency.

In one example, the DNA vector construct comprises a promoter, enhancer, and a polyadenylation signal. The promoter may be selected from the non-limiting group consisting of HIV, such as the Long Terminal Repeat (LTR), Simian Virus 40 (SV40), Epstein Barr virus (EBV), cytomegalovirus (CMV), Rous sarcoma virus (RSV), Moloney virus, mouse mammary tumor virus (MMTV), human actin, human myosin, human hemoglobin, human muscle creatine, human metalothionein. In another example, an inducible promoter is used so that the amount and timing of expression of the inserted gene or polynucleotide can be controlled.

The enhancer may be selected from the group including, but not limited to, human actin, human myosin, human hemoglobin, human muscle creatine and viral enhancers such as those from CMV, RSV and EBV. The promoter and enhancer may be from the same or different gene.

The polyadenylation signal may be selected, for example, from the group consisting of: LTR polyadenylation signal and SV40 polyadenylation signal, particularly the SV40 minor polyadenylation signal among others.

The expression vectors of the present invention may be operably linked to DNA coding for an RNA or protein to be used in this invention, i.e., the vectors are capable of directing both replication of the attached DNA molecule and expression of the RNA or protein encoded by the DNA molecule. Thus, for proteins, the expression vector must have an appropriate transcription start signal upstream of the attached DNA molecule, maintaining the correct reading frame to permit expression of the DNA molecule under the control of the control sequences and production of the desired protein encoded by the DNA molecule. Expression vectors may include, but are not limited to, cloning vectors, modified cloning vectors and specifically designed plasmids or viruses. An inducible promoter may be used so that the amount and timing of expression of the inserted gene or polynucleotide can be controlled.

One having ordinary skill in the art can produce DNA constructs which are functional in cells. In order to test expression, genetic constructs can be tested for expression levels in vitro using tissue culture of cells of the same type of those to be genetically modified.

Methods of Genetic Modification

Standard recombinant methods can be used to introduce genetic modifications into the cells being used for gene therapy. For example, retroviral vector transduction of cultured HSC is one successful method known in the art (Belmont and Jurecic (1997) “Methods for Efficient Retrovirus-Mediated Gene Transfer to Mouse Hematopoietic Stem Cells,” in Gene Therapy Protocols (P. D. Robbins, ed.), Humana Press, pp. 223-240; Bahnson et al., (1997) “Method for Retrovirus-Mediated Gene Transfer to CD34⁺-Enriched Cells,” in Gene Therapy Protocols (P. D. Robbins, ed.), Humana Press, pp. 249-263). Additional vectors include, but are not limited to, those that are adenovirus derived or lentivirus derived, and Moloney murine leukemia virus-derived vectors.

Also useful for genetic modification of HSC are the following methods: particle-mediated gene transfer such as with the gene gun (Yang, N.-S, and P. Ziegelhoffer, (1994) “The Particle Bombardment System for Mammalian Gene Transfer,” In PARTICLE BOMBARDMENT TECHNOLOGY FOR GENE TRANSFER (Yang, N.-S, and Christou, P., eds.), Oxford University Press, New York, pp. 117-141), liposome-mediated gene transfer (Nabel et al., (1992) Hum. Gene Ther. 3:649), coprecipitation of genetically modified vectors with calcium phosphate (Graham and Van Der Eb, (1973) Virol. 52:456), electroporation (Potter et al., (1984) Proc. Natl. Acad. Sci. USA 81:7161), and microinjection (Capecchi, (1980) Cell 22:479), as well as any other method that can stably transfer a gene or oligonucleotide, which may be in a vector, into the HSC and other cells to be genetically modified such that the gene will be expressed at least part of the time.

Skewing of Developing TCR Repertoire Towards, or Away from, Specific Antigens: Allergies and Autoimmune Diseases

The ability to enhance the uptake into the thymus of hematopoietic stem cells means that the nature and type of dendritic cells can be manipulated. For example, the stem cells can be transfected with specific gene(s) which eventually become expressed in the dendritic cells in the thymus (and elsewhere in the body). Such genes can include those which encode specific antigens for which an immune response would be detrimental, as in autoimmune diseases and allergies.

This aspect of the invention stems from the discovery that direct effects of sex steroid inhibition on the BM functionality and immune cell functionality, and on the eventual reactivation of the thymus of an autoimmune patient, will facilitate in overcoming an autoimmune disease suffered by that patient. This same principle also applies to patients suffering from allergies. As the thymus is reactivating, a new or modified immune system is created, one that no longer recognizes and/or responds to a self antigen.

In accordance with the invention, the following protocol may be applied. A patient diagnosed with an autoimmune disease (e.g., type I diabetes) is first immunosuppressed to stop disease progression. This may be done by administering an immunosuppressant (e.g., cyclosporine or rapamycin) alone or together with anti-T and B cell antibodies, such as anti-CD3 or anti-T cell gamma globulin to get rid of T cells and anti-CD19, CD20, or CD21 to get rid of B cells. At the same time that the patient is being immunosuppressed, sex steroid analogs (or other methods of castration) may be administered. His own T cells may then be mobilized with GCSF. If his autoimmunity arose as a result of a cross-reaction of his T cells with a pathogen he had previously encountered, the ablation of the T cells will remove the auto-reactive T cells, and the newly developed T cells will not continue to recognize his cells (e.g., h is β-islet cells) as foreign. In this manner, his autoimmune disease is alleviated. Moreover, once his autoimmune disease has been alleviated, the sex steroid ablation therapy can be stopped, thereby restoring the patient's fertility.

In another non-limiting example of the invention, the autoimmune patient is reconstituted with allogeneic stem cells. In some embodiments, these allogeneic stem cells are umbilical cord blood cells, which do not include mature T cells.

In some embodiments, the transplanted HSC may follow full myeloablation or myelodepletion, and thus result in a full HSC transplant (e.g., 5×10⁶ cells/kg body weight per transplant). In some embodiments, only minor myeloablation need be achieved, for example, 2-3 Gy irradiation (or 300 rads) followed by administration of about 3-4×10⁵ cells/kg body weight. In some embodiments, T cell depletion (TCD), and/or another method of immune cell depletion, is used (see, e.g., Example 2). It may be that as little as 10% chimerism may be sufficient to alleviate the symptoms of the patient's allergy or autoimmune disease. In some embodiments, the donor HSC are from umbilical cord blood (e.g., 1.5×10⁷ cells/kg for recipient engraftment).

In other embodiments, patients begin to receive Lupron up to 45 days before myelo-ablative chemotherapy and continue on the Lupron concurrently with the BMT such that the total length of exposure to the drug is around 9 months (equivalent to 3 injections as each Lupron injection delivers drug over a 3 month period). At various intervals over the course of study, blood samples are collected for analysis of T cell numbers (particularly of new thymic emigrants) and functions (specifically, response to T cell stimuli in vitro). This embodiment is also generally applicable to HSCT for other purposes described herein (e.g., HSCT following cancer radiation or chemotherapy).

In other embodiments, the transplanted HSC may follow lymphoablation. In some embodiments, T cells and/or B cells may be selectively ablated, to remove cells, as needed (e.g., those cells involved in autoimmunity or allergy). The selection can involve deletion of cells that are activated, or of a cell type involved in the autoimmune or allergic response. The cells may be selected based upon cell surface markers, such as CD4, CD8, B220, thy 1, TCR, CD3, CD5, CD7, CD25, CD26, CD23, CD30, CD38, CD49b, CD69, CD70, CD71, CD95, CD96, antibody specificity or Ig chain, or upregulated cytokine receptors, e.g., IL2-R β chain, TGFβ. One well known method for depletion is the use of antilymphocyte globulin. Other methods of selecting and sorting cells are well known and include magnetic and fluorescent cell separation, centrifugation, and more specifically, hemapheresis, leukopheresis, and lymphopheresis.

In some embodiments, HSCT is performed without myeloablation, myelodepletion, lymphodepletion, T cell ablation, and/or other selective immune cell ablation.

In other embodiments, the methods of the invention further comprise immunosuppressing the patient by e.g., administration of an immunosuppressing agent (e.g., cyclosporin, prednisone, ozothioprine, FK506, Imunran, and/or methotrexate) (see, e.g., U.S. Pat. No. 5,876,708). In one embodiment, immunosuppression is performed in the absence of HSCT. In one embodiment, immunosuppression is performed in conjunction with (e.g., prior to, concurrently with, or after) HSCT. In another embodiment, immunosuppresion is performed in the absence of myeloablation, lymphoablation, T cell ablation and/or other selective immune cell ablation, deletion, or depletion. In yet another embodiment, immunosuppression is performed in conjunction with (e.g., prior to, concurrently with, or after) myeloablation, lymphoablation, T cell ablation, and/or other selective immune cell ablation, deletion, or depletion.

As described above, myeloablation, myelodepletion, lymphoablation, immunosuppression, T cell ablation, and/or other selective immune cell ablation, are nonlimiting exemplary types of immune cell ablation, which are used throughout this application. The general term “immune cell depletion” is defined herein as encompassing each of these methods, i.e., myeloablation, myelodepletion, lymphoablation, T cell ablation, and/or other selective immune cell ablation (e.g., B cell or NK cell depletion). As will be readily understood by one skilled in the art, in practicing the inventions provided herein, any one of these “depletion” methods may be replaced with any one (or more) of the other “depletion” methods.

In one embodiment, NK cells are depleted. NK antibodies usefule for depleting the NK populations are known in the art. For example, one source of anti-NK antibody is anti-human thymocyte polyclonal anti-serum. U.S. Pat. No. 6,296,846 describes NK and T cell depletion methods, as well as non-myeloablative therapy and formation of a chimeric lymphohematopoietic population, all of which may be used in the methods of the invention.

In some embodiments, the methods of the invention further comprise, e.g., prior to HSCT, absorbing natural antibodies from the blood of the recipient by hemoperfusing an organ (e.g., the liver or kidney) obtained from the donor.

In another embodiment the present invention further includes a T cell help-reducing treatment, such as increasing the level of the activity of a cytokine which directly or indirectly (e.g., by the stimulation or inhibition of the level of activity of a second cytokine) promotes tolerance to a graft (e.g., IL-10, IL-4, or TGF-β), or which decreased the level of the activity of a cytokine which promotes rejection of a graft (i.e., a cytokine which is antagonistic to or inhibits tolerance (e.g., IFN β, IL-1, IL-2, or IL-12)). In some embodiments, a cytokine is administered to promote tolerance. The cytokine may be derived from the donor species or from the recipient species (see, e.g., U.S. Pat. No. 5,624,823, which describes DNA encoding porcine interleukin-10 for such use). The duration of the help-reducing treatment may be approximately equal to, or is less than, the period required for mature T cells of the recipient species to initiate rejection of an antigen after first being stimulated by the antigen (in humans this is usually 8-12 days). In other embodiments, the duration is approximately equal to or is less than two-, three-, four-, five-, or ten times the period required for mature T cells of the recipient to initiate rejection of an antigen after first being stimulated by the antigen. The short course of help-reducing treatment may be administered in the presence or absence of a treatment which may stimulate the release of a cytokine by mature T cells in the recipient, e.g., in the absence of Prednisone (17,21-dihydroxypregna-1,4-diene-3,11,20-trione). The help-reducing treatment may be begun before or about the time the graft is introduced. The short course of help-reducing treatment may be pre-operative or post-operative. In some embodiments, the donor and recipient are class I matched.

In yet further embodiments, where the antigen is not an auto-antigen but, rather, an external antigen (e.g., pollen or seafood), similar strategies can be employed. If the allergy arose from some chance activation of an aberrant T or B cell clone, immunosuppression to remove T cells and B cells, followed by (or concurrent with) thymus regeneration will remove the cells causing the allergic response. Since the allergy arose from the chance activation of an aberrant T or B cell clone, it is unlikely to arise again and, the newly regenerated thymus may also create regulatory T cells. While there may be auto-reactive IgE still circulating in the patient, these will eventually disappear, since the cells secreting them are effectively depleted. Once the immune system has been re-established, the sex steroid ablation therapy can be stopped, and the patient's fertility restored.

The present invention provides methods for treating autoimmune disease without a BMT, with BMT, or with GM cells as described herein. The methods of the invention may further comprise an organ or cell transplant to repair or replace damaged cells, tissues or organs. For example, in IDDM, a patient may require an islet cell transplant to replace damaged islet cells. Prevention of clinical symptoms of autoimmune disease may be achieved using the methods of the present invention, where a patient has pre-clinical symptoms or familial predisposition.

In further embodiments of the invention, genetic modification of the HSC may be employed if the antigen involved in the autoimmune disease or allergy is known. For example, in multiple sclerosis, the antigen may be myelin glycoprotein (MOG), myelin oligodendroglial protein, myelin basic protein or proteolipid protein. In pernicious anemia, the antigen may be the gastric proton pump. In type I diabetes, the antigen may be pro-insulin (J. Clin. Invest. (2003) 111:1365), GAD, or an islet cell antigen. T cell epitopes of type II collagen have been described with rheumatoid arthritis in (Ohnishi et al., (2003) Int. J. Mol. Med. 1:331). For Hashimoto's thyroiditis, an antigen is thyroid peroxidase, and for Graves disease an antigen is the thyroid-stimulating hormone receptor (Dawe et al., (1993) Springer Semin. Immunopathol. 14:285). Systemic lupus erythematosus antigens include DNA, histones, ribosomes, snRNP, scRNP, e.g., H1 histone protein. In particular Ro (SS-A) and La (SS-B) ribonucleo-protein antigens (e.g., Ro60 and Ro52) are associated with a patient's systemic lupus erythematosus (SLE) and rheumatoid arthritis. Myasthenia gravis antigens include acetylcholine receptor alpha chain, and some T cell epitopes are described in Atassi et al., (2001) Crit. Rev. Immunol. 21:1. Likewise, certain allergic reactions are in response to known antigens (e.g., allergy to feline saliva antigen in cat allergies). In these situations, the donor HSC may first be genetically modified to express the antigen prior to being administered to the recipient. HSC may be isolated based on their expression of CD34. These cells can then be administered to the patient together with inhibitors of sex steroid mediated signalling, such as GnRH analogs, which enhances the functionality of the BM. Accordingly, the genetically-modified HSC not only develop into DC, and so tolerize the newly formed T cells, but they also enter the BM as DC and delete new, autoreactive or allergic B cells. Thus, central tolerance to the auto-antigen or allergen is achieved in both the thymus and the bone marrow, thereby alleviating the patient's autoimmune disease or allergic symptoms. In some embodiments, immune cell depletion or suppression is also used.

In another example for the depletion of hyperreactive T cells, for which the target antigen is known, thymic epithelial stem cells (e.g., autologous epithelial stem cells) can be transfected with the gene encoding the specific antigen for which tolerance is desired. Thymic epithelial progenitor cells can be isolated from the thymus itself (especially in the embryo) by their labeling with the Ab MTS 24 or its human counterpart (see Gill et al., (2002) Nat. Immunol. 3:635).

Thus, in accordance with the invention, the basic principle is stop ongoing autoimmune disease or prevent one developing in highly predictive cases (e.g., in familial predisposition) with T cell and/or B cell (as appropriate) depletion, followed by rebuilding a new tolerant immune system. First, the autoimmune disease is diagnosed, and a determination is made as to whether or not there is a familial (genetic) predisposition. Next, a determination is made as to whether or not there had been a recent prolonged infection in the patient which may have lead to the autoimmune disease through antigen mimicry or inadvertent clonal activation. In practice, it may not be possible to determine the cause of the disease. Next, T cell depletion is performed and, as appropriate, B cell depletion is performed (or other immune cell depletion), combined with chemotherapy, radiation therapy and/or anti-B cell reagents (e.g., CD19, CD20, and CD21) or antibodies to specific Ig subclasses (anti IgE). The bone marrow and immune cell functionality is improved by administering GnRH to the patient. Simultaneous with this is the injection of HSC which have been in vitro transfected with a gene encoding the autoantigen to enter the rejuvenating thymus and convert to DC for presentation of the autoantigen to developing T cells, thereby inducing tolerance. The transfected HSC will also produce the antigen in the bone marrow, and present the antigen to developing immature B cells, thereby causing their deletion, similar to that occurring to T cells in the thymus. Use of the immunosuppressive regimes (anti-T, -B therapy) would overcome any untoward activation of pre-existing potentially autoreactive T and B cells. Moreover, in the case of non-obvious genetic predisposition, GnRH may be combined with G-CSF injection to increase blood levels of autologous HSC to enhance the thymic regrowth.

In some embodiments, hematopoietic or lymphoid stem and/or progenitor cells from a donor (e.g., an MHC-matched donor) are transplanted into the recipient to increase the speed of ultimate regeneration of the thymus. In another embodiment, these cells are transplanted from a healthy donor, without autoimmune disease or allergies, to replace aberrant stem and/or progenitor cells in the patient.

In one embodiment, a patient's autoimmune disease is eliminated at least in part by clearance of the patient's T cell population. Sex steroid mediated signalling is disrupted. Upon repopulation of the peripheral blood with new T cells, the aberrant T cells that failed tolerance induction to self remain eliminated from the T cell population.

In another embodiment, a patient's immune system cells causing allergies are eliminated by the same lymphocyte ablation treatements accompanied by disruption of sex steroid mediated signalling to enhance thymic T cell development, to allow repopulation of the peripheral blood stream with a “clean” population of T cells. In other cases, allergies and autoimmune diseases are alleviated following sex steroid signalling disruption due to increased functionality of the BM and other immune cells.

Prevention

The invention further provides methods for preventing, increasing resistance to, or treating an infection of a patient through enhancing the functionality of BM and/or immune cells in conjunction with a regenerating thymus, or alternatively, prior to, or without reactivation of the thymus. At this stage, the patient's immune system is enhanced, rejuvenated and reactivated, thereby increasing its response to foreign antigens such as viruses and bacteria. This is shown, for example, in FIGS. 13-17, which show the effects of thymic reactivation on the mouse immune system, as demonstrated with viral (HSV) challenge. The mice having prior reactivation of the thymus demonstrate resistance to HSV infection, while those not having thymic reactivation (aged thymus) have higher levels of HSV infection. It is well known that the mouse immune system is very similar to the human immune system and is used as a model for human disease. Thus, results in mice can be projected to show human responses. This is reinforced by the data showing the effects of thymic reactivation in humans. The ability to increase the functionality of immune cells is exemplified in Example 23, in which TCR responsiveness and proliferation is increased in castrated mice as early as 3-7 days following castration, and before thymus regeneration.

Improvement of Vaccine Response

The present disclosure is in the field of “active vaccinations,” where an antigen is administered to a patient whose immune system then responds to the antigen by forming an immune response against the antigen. Vaccination may include both prophylactic and therapeutic vaccines. As will be appreciated by those in the art, the methods of the invention may be used with virtually any method of vaccination in combination with sex steroid inhibition without undue experimentation. In some embodiments, the vaccination is given prior to or concurrently with, thymic reactivation. Multiple doses (e.g., booster immunizations) may also be given as needed.

In one embodiment, the vaccine is a killed or inactivated vaccine (e.g., by heat or other chemicals). In another embodiment, the vaccine is an attenuated vaccine (e.g., poliovirus and smallpox vaccines). In another embodiment, the vaccine is a subunit vaccine (e.g., hepatitis B vaccine, in which hepatitis B surface antigen (HBsAg) is the agent-specific protein).

In another embodiment, the vaccine is a recombinant vaccine. One type of recombinant vaccine is an attenuated vaccine in which the agent (e.g., a virus) has specific virulence-causing genes deleted, which renders the virus non-virulent. Another type of recombinant vaccine employs the use of infective, but non-virulent, vectors, which are genetically modified to insert a gene encoding target antigen. Examples of recombinant vaccines are vaccinia virus vaccines.

In yet another embodiment, the vaccine is a DNA vaccine. DNA-based vaccines generally use bacterial plasmids to express protein immunogens in vaccinated hosts. Recombinant DNA technology is used to clone cDNAs encoding immunogens of interest into eukaryotic expression vectors. Vaccine plasmids are then amplified in bacteria, purified, and directly inoculated into the hosts being vaccinated. DNA can be inoculated by a needle injection of DNA in saline, or by a gene gun device which delivers DNA-coated gold beads into the skin. Methods for preparation and use of such vaccines will be well-known to, or may be readily ascertained by, those of ordinary skill in the art.

There are several parameters that can influence the nature and extent of immune responses: the level and type of antigen, the site of vaccination, the availability of appropriate APC, the general health of the individual, and the status of the T and B cell pools. Of these, T cells are the most vulnerable because of the marked sex steroid-induced shutdown in thymic export that becomes profound from the onset of puberty and the global suppression of T cell responses by sex steroids. Any vaccination program should therefore only be logically undertaken when the level of potential responder T cells is optimal with respect to both the existence of naïve T cells representing a broad repertoire of specificity, and the presence of normal ratios of Th1 to Th2 cells and Th to Tc cells. The type of T cell help that supports an immune response determines whether the raised antibody will be C′-dependent and phagocyte-mediated defenses will be mobilized (a type 1 response), or whether the raised antibody will be C′-independent and phagocyte-independent defenses will be mobilized (a type 2 response) (for reviews, see Fearon and Locksley (1996) Science 272:50; Seder and Paul (1994) Annu. Rev. Immunol. 12:635). Historically, type 1 responses have been associated with the raising of cytotoxic T cells and type 2 responses with the raising of antibody. Thus, the level and type of cytokines generated may also be manipulated to be appropriate for the desired response (e.g., some diseases require Th1 responses, and some require Th2 responses, for protective immunity). This includes codelivery of Th1- or Th2-type cytokines (e.g., delivery of recombinant cytokines or DNA encoding cytokines) to shift the immune response patterns in the patient. Immunostimulatory CpG oligonucletides have also been utilized to shift immune response to various vaccine formulations to a more Th1-type response.

By the methods described herein, sex steroid inhibition results in increased BM and immune cell functionality without, prior to, or concurrently with thymic regrowth, which allows for improved immune responsiveness to vaccines.

This procedure can be combined with any other form of immune system stimulation, including adjuvant, accessory molecules, and cytokine therapies. For example, useful cytokines include, but are not limited to, interleukin 2 (IL-2) and IL-15 as a general immune growth factor, IL-4 to skew the response to Th2 (humoral immunity), and IFNγ to skew the response to Th1 (cell mediated, inflammatory responses), IL 12 to promote Th1 and IL10 to promote Th2 cells. Accessory molecules include, but are not limited to, inhibitors of CTLA4, which enhance the general immune response by facilitating the CD28/B7.1, B7.2 stimulation pathway, which is normally inhibited by CTLA4.

Recombinant gene expression vectors may be used for the vaccination methods of the invention. The recombinant vectors may be plasmids or cosmids, which include the antigen coding polynucleotides of the invention, but may also be viruses or retroviruses. The vectors used in the polynucleotide vaccines may be “naked” (i.e., not associated with a delivery vehicle such as liposomes, colloidal particles, etc.). For convenience, the term “plasmid” as used in this disclosure will refer to plasmids or cosmids, depending on which is appropriate to use for expression of the peptide of interest (where the choice between the two is dictated by the size of the gene encoding the peptide of interest). A commonly used plasmid vector which may be used is pBR322.

Various viral vectors that can be utilized in the vaccination methods of the invention include adenovirus, herpes virus, vaccinia, or an RNA virus such as a retrovirus. Retroviral vectors may be derivatives of a murine or avian retrovirus. Examples of retroviral include, but are not limited to, Moloney murine leukemia virus (MoMuLV), Harvey murine sarcoma virus (HaMuS-V), murine mammary tumor virus (MuMTV), and Rous Sarcoma Virus (RSV). Other plasmids and viral vectors useful in the vaccination methods of the invention are well known in the vaccine art.

Effects on BM and HSC

The present invention provides methods for increasing the function of BM in a patient, including increasing production of HSC and enhancing haemopoiesis. These methods are useful in a number of applications. For example, one of the difficult side effects of chemotherapy or radiotherapy, whether given for cancer or for another purpose, can be its negative impact on the patient's BM. Depending on the dose of chemotherapy, the BM may be damaged or ablated and production of blood cells may be impeded. Administration of a dose of a sex steroid analog (such as an LHRH analog) according to this invention after chemotherapy treatment aids in recovery from the damage done by the chemotherapy to the BM and blood cells. Alternatively, administration of the LHRH analog in the weeks prior to delivery of chemotherapy increases the population of HSC and other blood cells so that some of the deleterious effects of chemotherapy will be decreased.

The improvement in BM function may be applicable to, for example, patients with blood disorders. The term “blood disorders” is herein defined as any disorder or malady that involves the cells of the blood system in a patient, either directly or indirectly, including, but not limited to, disorders associated with hematopoiesis, e.g., leukemia. Thus, for example, the methods of the present invention are useful to replace the cancerous blood system cells with new cells from a donor (matched or unmatched) in an allogenic HSCT, or following autologous HSCT with the patient's own cells.

Increased HSC production by the BM causes consequential increase in red blood cells, which are, in turn, useful for management of RBC production. This can be easily determined by looking for, e.g., increased hematocrit.

In some examples, the increased HSC are CD34+ or CD34lo HSC. Mobilized HSCs (e.g., using G-CSF) can assist in the “repair” or rejuvenation of tissues, such as with heart tissue and lung tissue. HSC have the potential to generate non-hematopoietic tissue. While much of the work has been carried out in vitro, a study at the Mayo Clinic, Rochester has shown that after BMT a small number of cardiomyocytes are donor derived. Similarly, the Beaumont Hospital, Michigan has used HSC to repair damaged heart muscle, although it is unclear whether the HSC become myocytes or vasculature. Mice experiments have also shown the potential of HSC to become insulin producing β-cells. Other work has shown HSC are capable of becoming skeletal muscle (myocytes), liver (hepatocytes), bone, connective tissue, epithelial tissue (e.g. of lung, gut and skin), vasculature, neurons, and islet cells.

The methods described herein are useful to repair damage to the BM and/or assist in the replacement of blood cells that may have been injured or destroyed by various therapies (e.g., cancer chemotherapy drugs, radiation therapy) or diseases (e.g., HIV, chronic renal failure).

In some chemotherapy regimens, such as high dose chemotherapy to treat any of the blood cancers, ablation of the BM is a desired effect. The methods of the invention may be used immediately after ablation occurs to stimulate the BM and increase the production of HSC and their progeny blood cells, so as to decrease the patient's recovery time. Following administration of the chemotherapy, usually allowing one or more days for the chemotherapy to clear from the patient's body, a dose of LHRH analog according to the methods described herein is administered to the patient. This can be in conjunction with the administration of autologous or heterologous BM or hematopoietic stem or progenitor cells, as well as other factors such as colony stimulating factors (CSFs) and stem cell factor (SCF).

Alternatively, a patient may have suboptimal (or “tired) BM function and may not be producing sufficient or normal numbers of HSC and other blood cells. This can be caused by a variety of conditions, including normal aging, prolonged infection, post-chemotherapy, post-radiation therapy, chronic disease states including cancer, genetic abnormalities, and immunosuppression induced in transplantation. Further, radiation, such as whole-body radiation, can have a major impact on the BM productivity. These conditions can also be either pre-treated to minimize the negative effects (such as for chemotherapy and/or radiation therapy), or treated after occurrence to reverse the effects.

Effects on T Cells

Sex steroids in males and females can be inhibited temporarily by taking disruptors of sex steroid mediated signalling (e.g., GnRH agonists). It has been shown that loss of steroids causes a reactivation of thymus function and enhanced production of naïve T cells. Furthermore, even before those new T cells have had a chance to leave the thymus, the pre-existing T cells are much more sensitive to stimulation, which results in a much more effective immune response. This increased responsiveness is evident within days of the loss of sex steroids (see, e.g., Example 23). This may be because there are no inhibitory effects of sex steroids. This may be because of the production of “helper” or “adjuvant” factors by the reactivating or reactivated thymus, which are able to costimulate the T cells in conjunction with the foreign stimulus. Also, since many cells of the immune system have surface receptors for GnRH, the GnRH itself may provide an additional stimulation for the T cells. Since the effect of the loss of sex steroids on peripheral T cells is so rapid, GnRH can be given as a single treatment simultaneously with the delivery of, for example, a vaccine. A one month formulation is useful which has the beneficial effects of stimulating immune responses but without the side effects of longer loss of sex steroids. A subsequent “booster” injection of the antigen can also be administered.

Sex steroid inhibitors (e.g., GnRH analogs) are useful to boost all forms of immunotherapy in a cancer patient, particularly for the removal of cancer cells which have escaped chemotherapy or surgery, but also for the defense against opportunistic infections. These analogs may also be used prophylacticly to improve immune response to vaccination programs designed to prevent, e.g., infections or cancer.

The methods of the invention utilize inhibition of sex steroid signalling. Sex steroids suppress the function of the thymus, BM, and also T and B lymphocytes throughout the body, which are concentrated in the major lymphoid areas of the body including, but not limited to, the blood, lymph nodes, mucosal tissue (e.g., respiratory, gastrointestinal, genital). It has surprisingly been discovered that ablation of sex steroids and/or interruption of sex steroid-mediated signalling may be used not only to regenerate the thymus (and thus the number and “quality” of T cells), but also to improve the functionality of pre-existing and newly produced T cells (and other cells of the immune system) either without, prior to, or concurrently with, thymus regeneration.

A poor immune response can have immediate and clinically important consequences. It can mean an increased susceptibility to common infections (e.g., influenza), increased susceptibility to cancers and tumors, and/or poor responsiveness to vaccinations.

An increase in the number and/or proportion of naïve T cells in the total T cell pool has a positive, immediate therapeutic effect on a number of clinical (or potentially clinical) conditions and diseases, including, but not limited to, cancer, immunodeficiency (particularly viral infections, e.g., Acquired Immune Deficiency Syndrome (AIDS) and Severe Acute Respiratory Syndrome (SARS) or influenza), autoimmunity, transplantation, allergies, as well as improving the general efficacy of vaccination programs. Each of these applications is described in detail in co-owned and co-pending U.S. application Ser. Nos. 10/418,747, 10/419,039, 10/418,953, 10/418,727, 10/419,066, and 10/419,068.

With respect to the application of the methods of the instant invention to the prevention of viral infections, a recent example of the effects of a poorly functioning immune system is observed with the advent of the SARS virus. This virus, although related to the common cold virus, is different enough so that the mature immune system cannot recognize it. Only naïve T cells are able to deal with previously unknown infections, such as SARS. However, with few naïve T cells, and an inability to create reasonable additional quantities, the average adult is highly susceptible to this disease, whereas children seem able to cope with it. This demographic is reflected in the mortality figures—the average age for death is approximately 50 years, with no pre-pubescent deaths being recorded.

In one example, inhibition of sex steroid signalling (e.g., using LHRH/GnRH analogs) is used to boost the responsiveness of T and B lymphocytes to stimulation with antigen. This stimulation may be a microorganism (e.g., bacteria, virus, fungi, parasites, etc.) entering the body.

In another example, inhibition of sex steroid signalling (e.g., using LHRH/GnRH analogs) is used to enhance the immune response to a vaccine antigen.

In one nonlimiting example, inhibition of sex steroid signalling (e.g., using LHRH/GnRH analogs) occurs prior to immune system challenge. This allows time for the loss or sex steroids to occur. The inhibition of sex steroid signalling (e.g., using LHRH/GnRH analogs) may also be accomplished simultaneously or sequentially with the administration of the stimulant to act as an “adjuvant” for enhancing the immune response directly (which could be mediated via direct signalling at the cell surface, increase in cytokines, decrease in inhibitors, etc.). The inhibition of sex steroid signalling (e.g., using LHRH/GnRH analogs) may also be given on multiple occasions. The immediate effects are due to enhancing the functionality of pre-existing lymphoid (and non-lymphoid) cells. With time, the reduction of sex steroids increases the production and functionality of T cells, B cells and APC, which additively, synergistically, or complementarily continue to enhance the response. Increases of new T cells, B cells, and other immune cells, such as APC, together with increased sensitivity of pre-existing T cells, B cells, and APC to stimulation, may then be used for the generation of more efficacious immune responses to primary infection, secondary infection, vaccination, etc.

Thus, disruptors of sex steroid signalling are used according to the methods of the instant invention, to cause a clinically positive effect by initiating an increased activation or functionality of these immune cells even before these drugs have been able to cause significant thymic regrowth.

These drugs, according to the invention, also may be used to assist in the replacement of blood cells that may have been injured or destroyed by various other therapies or diseases including, but not limited to, cancer chemotherapy drugs and/or cancer radiation therapy, as wells as diseases, such as chronic renal failure. As described in more detail elsewhere herein, the use of sex steroid inhibition drugs in combination with G-CSF, GM-CSF, erythropoietin (EPO), SCF, or other hormones or cytokines, may also be used to further improve the enhancement of the production of blood cells by those compounds.

The phrase “modifying the T-cell population makeup” is herein defined as altering the nature and/or ratio of T cell subsets defined functionally and by expression of characteristic molecules. Examples of these characteristic molecules include, but are not limited to, the T cell receptor, CD4, CD8, CD3, CD25, CD28, CD44, CD45, CD62L and CD69.

“Increasing the number of cells” e.g., T-cells, is herein defined as an absolute increase in the number of T cells in a subject in the thymus and/or in circulation and/or in the spleen and/or in the BM and/or in peripheral tissues such as lymph nodes, gastrointestinal, urogenital and respiratory tracts. This phrase also refers to a relative increase in T cells, for instance when compared to B cells.

A “subject having a depressed or abnormal T-cell population or function” includes an individual infected with the human immunodeficiency virus, especially one who has AIDS, or any other virus or infection which attacks T cells or any T cell disease for which a defective gene has been identified. Furthermore, this phrase includes any post-pubertal individual, especially an aged person who has decreased immune responsiveness and increased incidence of disease as a consequence of post-pubertal thymic atrophy.

In cases where the subject is infected with HIV, it is useful that the HSC are genetically modified such that they and their progeny, in particular T cells, macrophages and DC, are resistant to infection and/or destruction with the HIV virus. The genetic modification may involve introduction into HSC one or more nucleic acid molecules which prevent viral replication, assembly and/or infection. The nucleic acid molecule may be a gene which encodes an antiviral protein, an antisense construct, a ribozyme, a dsRNA and a catalytic nucleic acid molecule.

In cases where the subject has defective T cells, the HSC may be genetically modified to normalize the defect. For diseases such as T cell leukaemias, the modification may include the introduction of nucleic acid constructs or genes, which normalize the HSC and inhibit or reduce its likelihood of becoming a cancer cell.

It will be appreciated by those skilled in the art that the present methods may be useful in treating any T cell disorder which has a defined genetic basis.

The methods of the present invention are also useful for the treatment of AIDS, where the treatment involves reduction of viral load, increases in T cell number and functionality, reactivation of thymic function through inhibition of sex steroids signalling. The patients may receive HSC which have been genetically modified such that all progeny (e.g., T cells and DC) are resistant to further HIV infection. This means that not only will the patient be depleted of HIV virus and no longer susceptible to general infections because the T cells have returned to normal levels, but the new T cells being resistant to HIV will be able to remove any remnant viral infected cells. In principle, a similar strategy could be applied to gene therapy in HSC for any T cell defect or any viral infection which targets T cells.

Effect on B Cells

As with other cells of the immune system, B cell function is also diminished with age, which is in part due to the decline in T cell production and consequent lack of T cell help. However, there are also significant age-associated changes in B cell function (Hu et al., (1993) Int., Immunol. 5:1035-1039). Despite B cell numbers remaining relatively constant throughout life due to tightly regulated homeostatic mechanisms, there is a decrease in export from the BM, clonal expansion of peripheral B cells, and a narrowing of the antibody response (LeMaoult et al., (1999) J. Immunol. 162:6384-6391). Decreased antibody response to foreign antigens in the aged are thought to be primarily due to a decline in T cell help (Hu et al., (1993) Int. Immunol. 5:1035-1039; LeMaoult et al., (1999) J. Immunol. 162:6384-6391). However, defective class switching (Weksler et al., (2000) Vaccine 18:1624-1628) and a preferential loss of high affinity antibodies may play a role (Nicoletti et al., (1993) J. Immunol. 150:543-549).

Castration of aged mice results in an increase in IL-7 responsive B cell progenitors, including late pro-B cells, pre-B cells, and immature B cells (Ellis et al., (2001) Int. Immunol. 13:553-558). The absolute number of B cells in the periphery is also increased (Ellis et al., (2001) Int. Immunol. 13:553-558). This increase in circulating B cells is largely due to an increase in the number of recent BM emigrants (CD45R^{lo}CD24^{hi}) and these cells remain at an elevated level for up to 54 days after castration (Ellis et al., (2001) Int. Immunol. 13:553-558).

Thus, the methods of the invention may be used to increase the number and functionality of B cells without, prior to, or concurrently with thymic regeneration. This may be useful for, e.g., increased control (by prevention or treatment) of bacterial infections in normal patients, in patients with compromised immune systems, such as those patients receiving a disease treatment regimen. Increased B cell number and functionality may also be useful following surgery and/or in burn victims, and in other instances wherein the patient's immune system is compromised.

Effects on DC

The present invention also provides methods for increasing DC functionality and/or DC number. Following sex steroid ablation (e.g., following delivery of an LHRH analog) DC are increased in the thymus, and in the periphery, which may also assist the T cell stimulation. DC are important antigen presenting cells and increased numbers and/or function may be useful in improving responsiveness to agents, e.g., cancers. Enhanced DC functionality may also be useful in achieving resistance to agents such as allergens or self-antigens (in the case of autoimmune disorders).

Effects on Platelets

The present invention also provides methods for increasing platelet cell number and/or functionality. Thrombocytopenia is common and has a variety of causes including, but not limited to, poor BM and splenomegaly. The condition generally results in bleeding disorders that are very difficult to treat. The most common diseases associated with thrombocytopenia include leukemia, aplastic anemia, cirrhosis, and Gauchers disease. Massive blood replacement is often needed because platelets have a short half life in stored blood which is used for transfusions.

Effect on NK, NKT, and Treg Cells

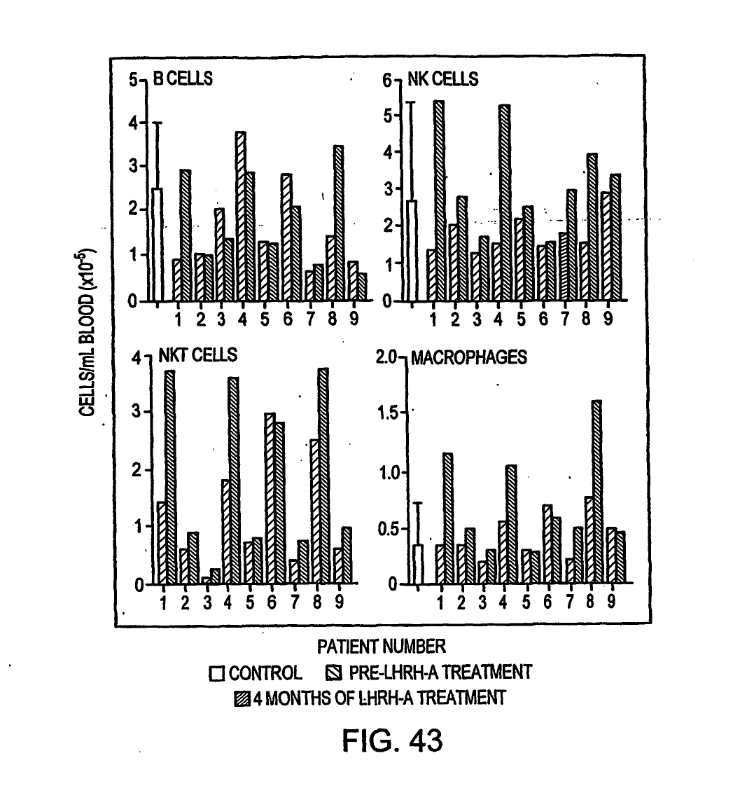

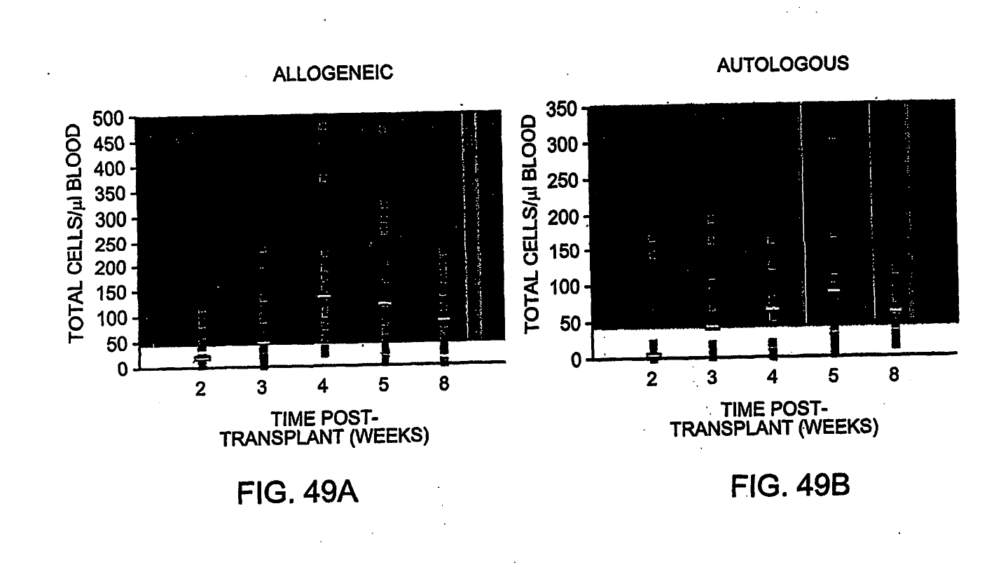

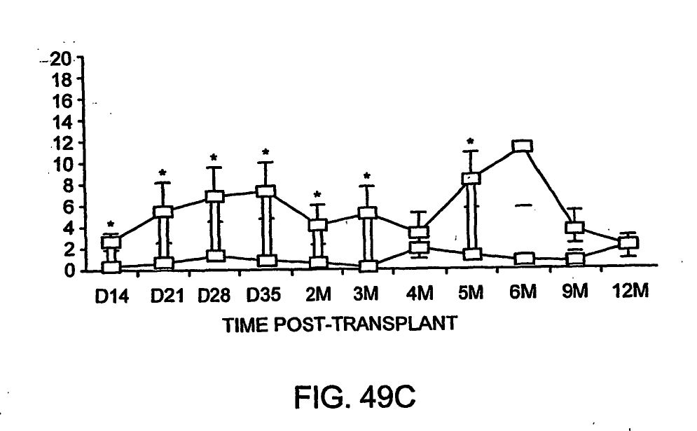

The present invention also provides methods for increasing NK and NKT cell number and/or functionality (see, e.g., Example 17 and FIGS. 43 and 49). This may be useful in the treatment of diseases displaying NK deficiencies, e.g., Crohn's disease, Chediak-Highashi syndrome. Impaired NK cell function has been reported in patients with connective tissue diseases including lupus and rheumatoid arthritis. NK cells are important in defending against cancer and infectious agents. Other non-limiting diseases associated with NK cell deficiency (numerical and/or functional), which may benefit from the methods of the invention include herpes virus infections (e.g., varicella-zoster virus (VZV, chicken pox, shingles), HSV, CMV, and EBV), other viral infections (e.g., measles, mumps, influenza and HIV, which are now thought to be controlled, at least in part, by NK cells), mycobacterial infections (e.g., M. tuberculosis, M. avium, which are apparently controlled by both NK and macrophage), other malignancies (e.g., solid tumors, including melanoma, breast cancer and rectal cancer), and BMT for acute myelogenous leukemia with haploidentical/mismatch donors. NKT cells are important regulators of the immune response because they are very high producers of cytokines without the need for prior activation. They have a role in preventing autoimmune disease but also promote anti-cancer effects. Tregs (commonly defined as being CD4⁺CD25⁺) are also major regulators of the immune response primarily through their cytokine production.

Macrophage

The present invention also provides methods for increasing macrophage number and functionality (see, e.g., Example 17 and FIG. 43), which would have a primary role in helping remove infectious agents particularly bacteria.

### EXAMPLES

The following Examples provide specific examples of methods of the invention, and are not to be construed as limiting the invention to their content.

### Example 1

### Reversal of Aged-Induced Thymic Atrophy

Materials and Methods

Animals. CBA/CAH and C57B16/J male mice were obtained from Central Animal Services, Monash University and were housed under conventional conditions. C57B16/J Ly5.1⁺ were obtained from the Central Animal Services Monash University, the Walter and Eliza Hall Institute for Medical Research (Parkville, Victoria) and the A.R.C. (Perth, Western Australia) and were housed under conventional conditions. Ages ranged from 4-6 weeks to 26 months of age and are indicated where relevant.

Surgical castration. Animals were anesthetized by intraperitoneal injection of 0.3 ml of 0.3 mg xylazine (Rompun®; Bayer Australia Ltd., Botany NSW, Australia) and 1.5 mg ketamine hydrochloride (Ketalar®; Parke-Davis, Caringbah, NSW, Australia) in saline. Surgical castration was performed by a scrotal incision, revealing the testes, which were tied with suture and then removed along with surrounding fatty tissue. The wound was closed using surgical staples. Sham-castration followed the above procedure without removal of the testes and was used as controls for all studies.

Bromodeoxyuridine (BrdU) incorporation. Mice received two intraperitoneal injections of BrdU (Sigma Chemical Co., St. Louis, Mo.) at a dose of 100 mg/kg body weight in 100 μl of PBS, 4-hours apart (i.e., at 4 hour intervals). Control mice received vehicle alone injections. One hour after the second injection, thymuses were dissected and either a cell suspension made for FACS analysis, or immediately embedded in Tissue Tek (O.C.T. compound, Miles Inc., Indiana), snap frozen in liquid nitrogen, and stored at −70° C. until use.

Flow Cytometric analysis. Mice were killed by CO₂ asphyxiation and thymus, spleen, and mesenteric lymph nodes were removed. Organs were pushed gently through a 200 μm sieve in cold PBS/1% FCS/0.02% Azide, centrifuged (650 g, 5 min, 4° C.), and resuspended in either PBS/FCS/Az. Spleen cells were incubated in red cell lysis buffer (8.9 g/liter ammonium chloride) for 10 min at 4° C., washed and resuspended in PBS/FCS/Az. Cell concentration and viability were determined in duplicate using a hemocytometer and ethidium bromide/acridine orange and viewed under a fluorescence microscope (Axioskop; Carl Zeiss, Oberkochen, Germany).

For 3-color immunofluorescence, cells were labeled with anti-αβTCR-FITC, anti-CD4⁻PE and anti-CD8⁻APC (all obtained from Pharmingen, San Diego, Calif.) followed by flow cytometry analysis. Spleen and lymph node suspensions were labeled with either αβTCR-FITC/CD4⁻PE/CD8⁻APC or B220-B (Sigma) with CD4⁻PE and CD8⁻APC. B220-B was revealed with streptavidin-Tri-color conjugate purchased from Caltag Laboratories, Inc., Burlingame, Calif.

For BrdU detection of cells, cells were surface labeled with CD4⁻PE and CD8⁻APC, followed by fixation and permeabilization as previously described (Carayon and Bord, (1989) J. Imm. Meth. 147:225). Briefly, stained cells were fixed overnight at 4° C. in 1% paraformaldehyde (PFA)/0.01% Tween-20. Washed cells were incubated in 5001 μl DNase (100 Kunitz units, Roche, USA) for 30 mins at 37° C. in order to denature the DNA. Finally, cells were incubated with anti-BrdU-FITC (Becton-Dickinson) for 30 mins. at room temperature, washed and resuspended for FACS analysis.

For BrdU analysis of TN subsets, cells were collectively gated out on Lin-cells in APC, followed by detection for CD44-biotin and CD25-PE prior to BrdU detection. All antibodies were obtained from Pharmingen (San Diego, Calif.).

For 4-color Immunofluorescence, thymocytes were labeled for CD3, CD4, CD8, B220 and Mac-1, collectively detected by anti-rat Ig-Cy5 (Amersham, U.K.), and the negative cells (TN) gated for analysis. They were further stained for CD25PE (Pharmingen, San Diego, Calif.) and CD44-B (Pharmingen, San Diego, Calif.) followed by Streptavidin-Tri-color (Caltag, Calf.) as previously described (Godfrey and Zlotnik, (1993) Immunol. Today 14:547). BrdU detection was then performed as described above.

Samples were analyzed on a FACSCalibur™(Becton-Dickinson). Viable lymphocytes were gated according to 0° and 90° light scatter profiles and data was analyzed using CellQuest™ software (Becton-Dickinson).

Immunohistology. Frozen thymus sections (4 μm) were cut using a cryostat (Leica) and immediately fixed in 100% acetone.

For two-color immunofluorescence, sections were double-labeled with a panel of monoclonal antibodies: MTS6, 10, 12, 15, 16, 20, 24, 32, 33, 35 and 44 (Godfrey et al., (1990) Immunol. 70:66; Table 1) produced in this laboratory and the co-expression of epithelial cell determinants was assessed with a polyvalent rabbit anti-cytokeratin Ab (Dako, Carpinteria, Calif.). Bound mAb was revealed with FITC-conjugated sheep anti-rat Ig (Silenus Laboratories, Victoria, Australia) and anti-cytokeratin was revealed with TRITC-conjugated goat anti-rabbit Ig (Silenus Laboratories, Victoria, Australia).

For BrdU detection of sections, sections were stained with either anti-cytokeratin followed by anti-rabbit-TRITC or a specific mAb, which was then revealed with anti-rat Ig-Cγ3 (Amersham, Uppsala, Sweden). BrdU detection was then performed as previously described (Penit et al., (1996) Proc. Natl. Acad. Sci, USA 86:5547). Briefly, sections were fixed in 70% Ethanol for 30 mins. Semi-dried sections were incubated in 4M HCl, neutralized by washing in Borate Buffer (Sigma), followed by two washes in PBS. BrdU was detected using anti-BrdU-FITC (Becton-Dickinson).

For three-color immunofluorescence, sections were labeled for a specific MTS mAb together with anti-cytokeratin. BrdU detection was then performed as described above.

Sections were analyzed using a Leica fluorescent and Nikon confocal microscopes.

Migration studies (i.e., Analysis of recent thymic emigrants (RTE)). Animals were anesthetized by intraperitoneal injection of 0.3 ml of 0.3 mg xylazine (Rompun®; Bayer Australia Ltd., Botany NSW, Australia) and 1.5 mg ketamine hydrochloride (Ketalar®; Parke-Davis, Caringbah, NSW, Australia) in saline.

Details of the FITC labeling of thymocytes technique are similar to those described elsewhere (Scollay et al., (1980) Proc. Natl. Acad. Sci, USA 86:5547; Berzins et al., (1998) J. Exp. Med. 187:1839). Briefly, thymic lobes were exposed and each lobe was injected with approximately 10 μm of 350 μg/ml FITC (in PBS). The wound was closed with a surgical staple, and the mouse was warmed until fully recovered from anesthesia. Mice were killed by CO₂ asphyxiation approximately 24 hours after injection and lymphoid organs were removed for analysis.

After cell counts, samples were stained with anti-CD4-PE and anti-CD8⁻APC, then analyzed by flow cytometry. Migrant cells were identified as live-gated FITC⁺ cells expressing either CD4 or CD8 (to omit autofluorescing cells and doublets). The percentages of FITC⁺ CD4 and CD8 cells were added to provide the total migrant percentage for lymph nodes and spleen, respectively. Calculation of daily export rates was performed as described by Berzins et al., ((1998) J. Exp. Med. 187:1839).

Data analyzed using the unpaired student ‘t’ test or nonparametrical Mann-Whitney U-test was used to determine the statistical significance between control and test results for experiments performed at least in triplicate. Experimental values significantly differing from control values are indicated as follows: *p≦0.05, **p≦0.01 and ***p≦0.001.

Results

### I. The Effect of Age on Thymocyte Populations.

### (i) Thymic Weight and Thymocyte Number

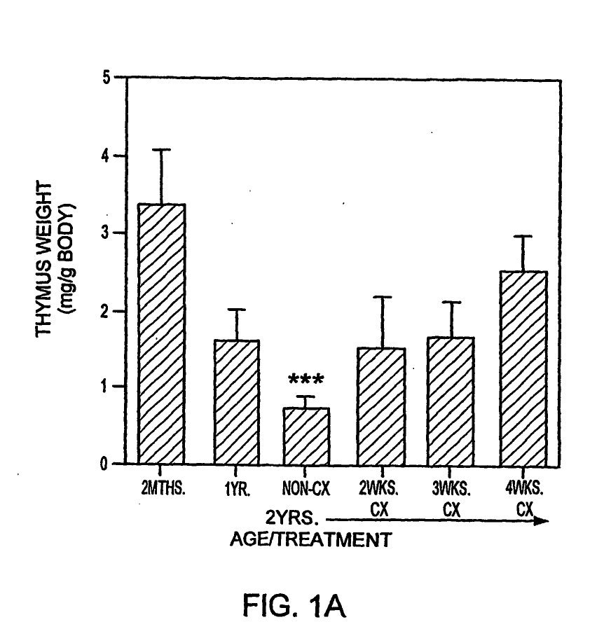

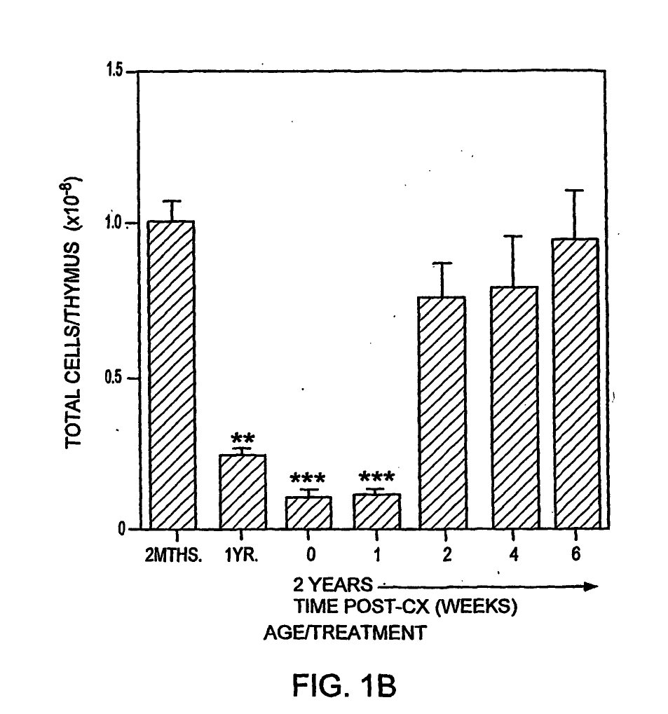

With increasing age there is a highly significant (p≦0.0001) decrease in both thymic weight (FIG. 1A) and total thymocyte number (FIG. 1B) in mice. Relative thymic weight (mg thymus/g body) in the young adult has a mean value of 3.34 which decreases to 0.66 at 18-24 months of age (adipose deposition limits accurate calculation). The decrease in thymic weight can be attributed to a decrease in total thymocyte numbers: the 1-2 month (i.e., young adult) thymus contains about 6.7×10⁷ thymocytes, decreasing to about 4.5×10⁶ cells by 24 months. By removing the effects of sex steroids on the thymus by castration, thymocyte cell numbers are regenerated and by 4 weeks post-castration, the thymus is equivalent to that of the young adult in both weight (FIG. 1A) and cellularity (FIG. 1B). Interestingly, there was a significant (p≦0.001) increase in thymocyte numbers at 2 weeks post-castration (1.2×10⁸), which is restored to normal young levels by 4 weeks post-castration.

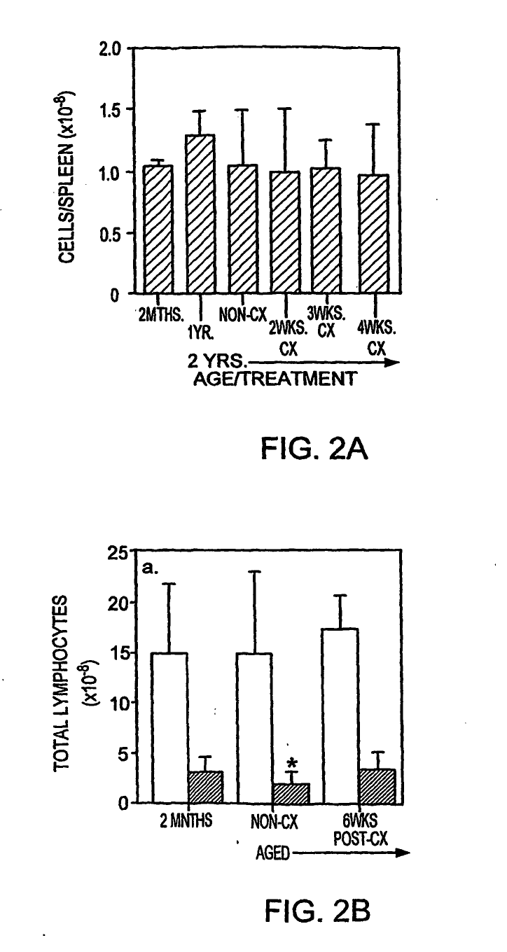

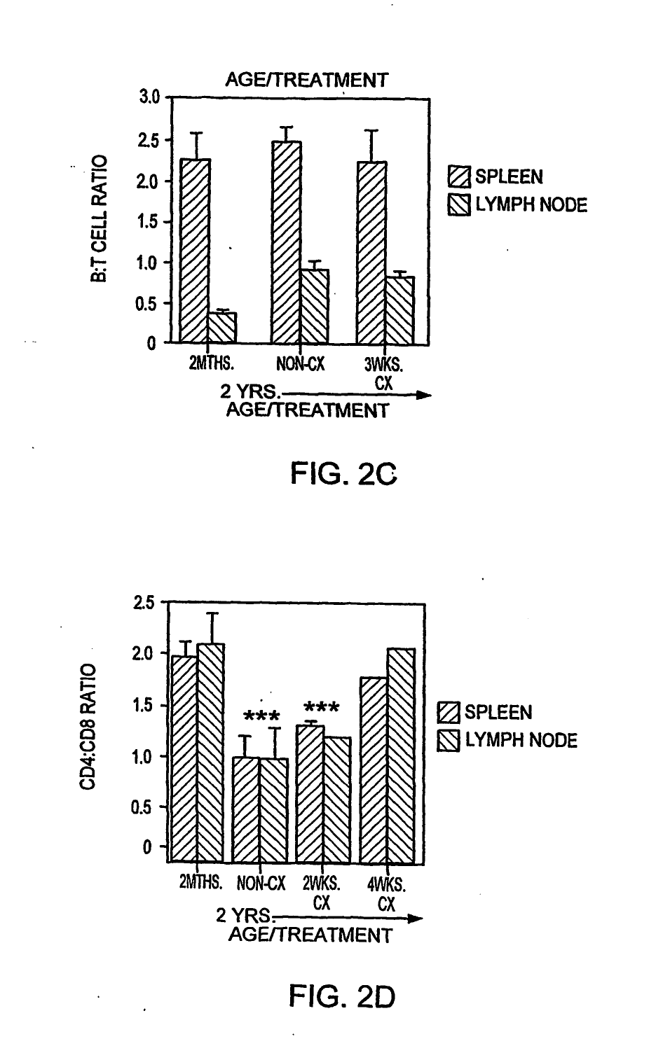

The decrease in T cell numbers produced by the thymus is not reflected in the periphery, with spleen cell numbers remaining constant with age (FIGS. 2A and 2B). Homeostatic mechanisms in the periphery were evident since the B cell to T cell ratio in spleen and lymph nodes was not affected with age and the subsequent decrease in T cell numbers reaching the periphery (FIG. 2C). However, the ratio of CD4⁺ to CD8⁺ T cell significantly decreased (p≦0.001) with age from 2:1 at 2 months of age, to a ratio of 1:1 at 2 years of age (FIG. 2D). Following castration and the subsequent rise in T cell numbers reaching the periphery, no change in peripheral T cell numbers was observed: splenic T cell numbers and the ratio of B:T cells in both spleen and lymph nodes was not altered following castration (FIGS. 2A-C). The reduced CD4:CD8 ratio in the periphery with age was still evident at 2 weeks post-castration but was completely reversed by 4 weeks post-castration (FIG. 2D)

(ii) Thymocyte Subpopulations with Age and Post-Castration.

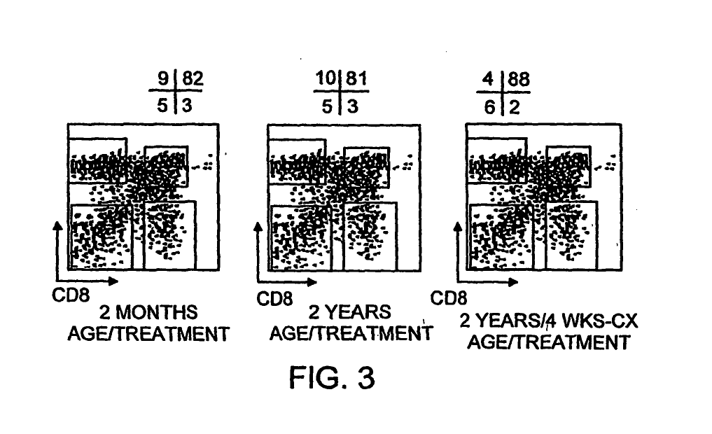

To determine if the decrease in thymocyte numbers seen with age was the result of the depletion of specific cell populations, thymocytes were labeled with defining markers in order to analyze the separate subpopulations. In addition, this allowed analysis of the kinetics of thymus repopulation post-castration. The proportion of the main thymocyte subpopulations was compared with those of the young adult (2-4 months) thymus (FIG. 3) and found to remain uniform with age. In addition, further subdivision of thymocytes by the expression of αβTCR revealed no change in the proportions of these populations with age. At 2 and 4 weeks post-castration, thymocyte subpopulations remained in the same proportions and, since thymocyte numbers increase by up to 100-fold post-castration, this indicates a synchronous expansion of all thymocyte subsets rather than a developmental progression of expansion.

The decrease in cell numbers seen in the thymus of aged (2 year old) animals thus appears to be the result of a balanced reduction in all cell phenotypes, with no significant changes in T cell populations being detected. Thymus regeneration occurs in a synchronous fashion, replenishing all T cell subpopulations simultaneously rather than sequentially.

### II. Proliferation of Thymocytes

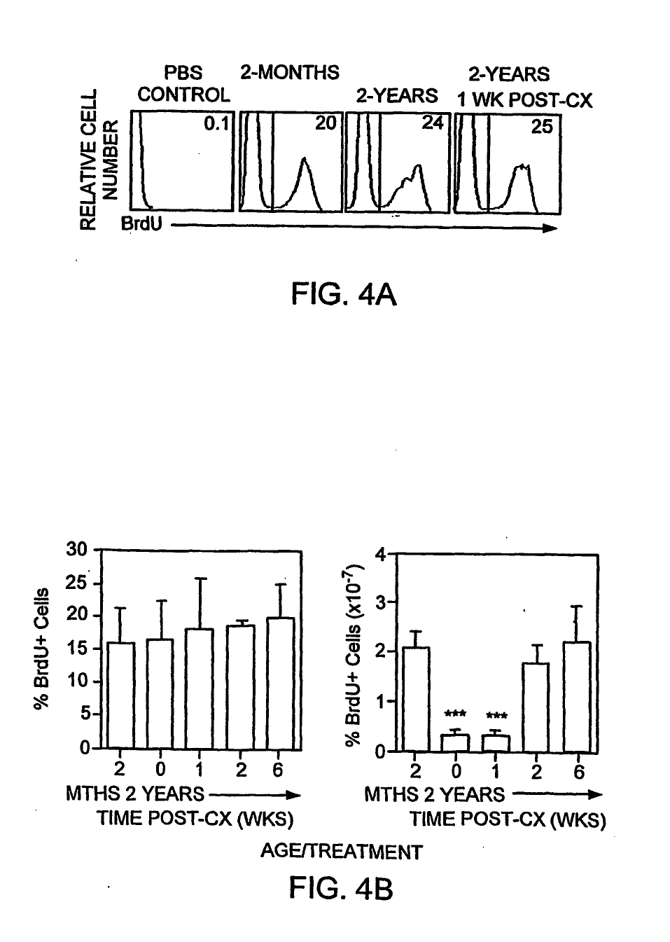

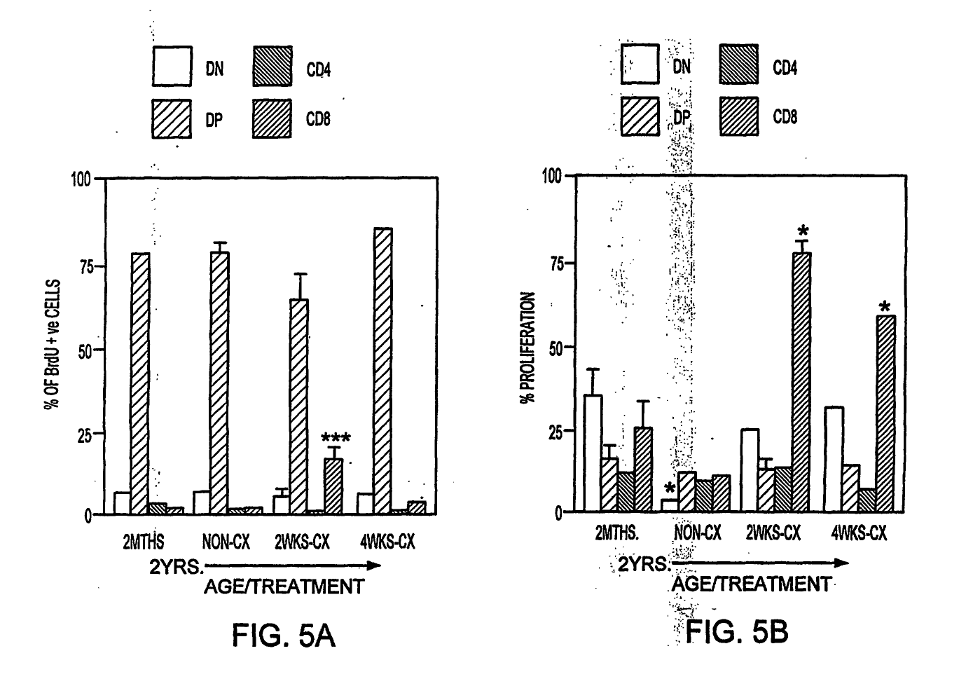

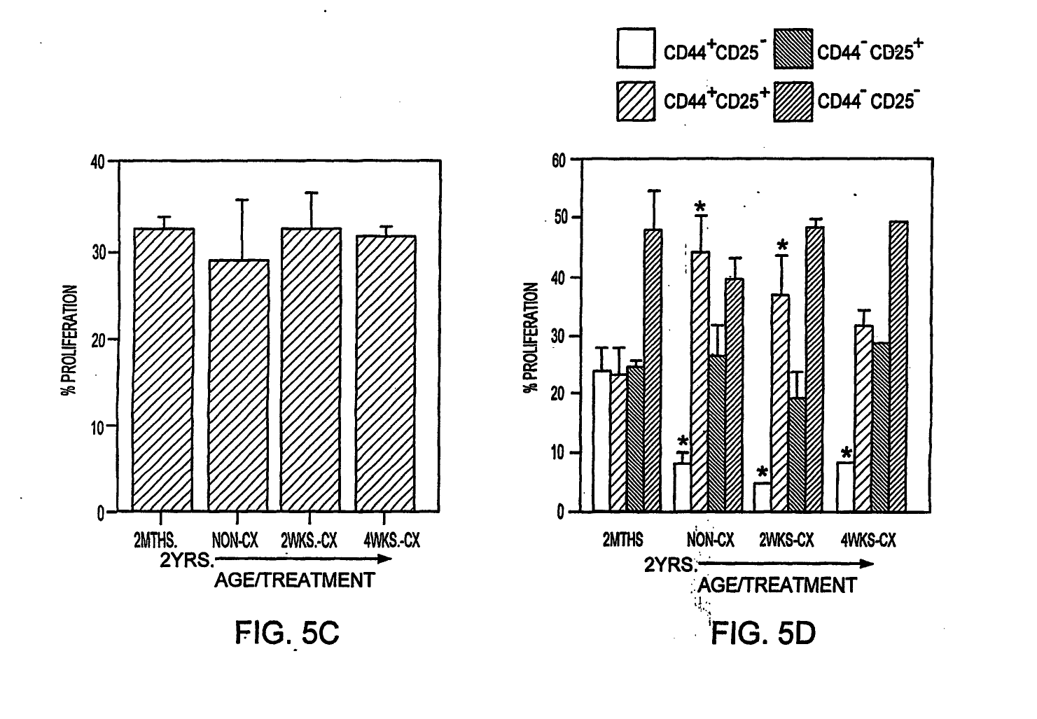

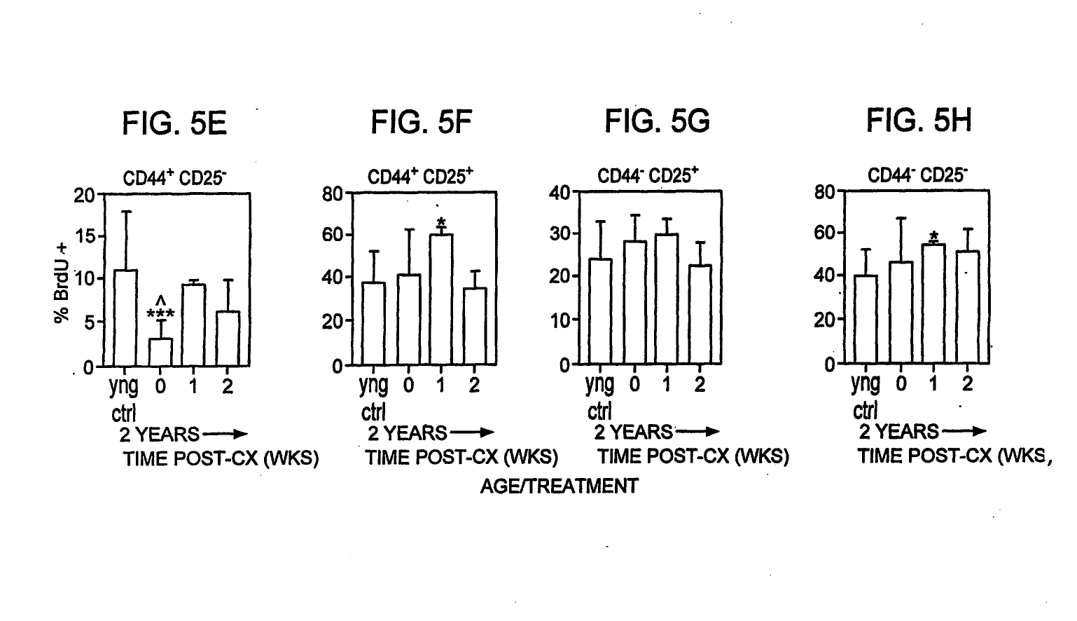

As shown in FIGS. 4A-4B, 15-20% of thymocytes were proliferating at 2-4 months of age. The majority (about 80%) of these are double positive (DP) (i.e., CD4⁺, CD8⁺) with the triple negative (TN) (i.e., CD3⁻CD4⁻CD8⁻) subset making up the second largest population at about 6% (FIG. 5A). These TN cells are the most immature cells in the thymus and encompass the intrathymic precursor cells. Accordingly, most division is seen in the subcapsule and cortex by immunohistology. Some division is seen in the medullary regions aligning with FACS analysis which revealed a proportion of single positive (i.e., CD4⁺CD8- or CD4⁻CD8⁺) cells (9% of CD4⁺ T cells and 25% of CD8⁺ T cells) in the young (2 months) thymus, dividing (FIG. 5B).

Although cell numbers were significantly decreased in the aged mouse thymus (2 years old), the total proportion of proliferating thymocytes remained constant (FIGS. 4B and 5C), but there was a decrease in the proportion of dividing cells in the CD4⁻CD8⁻ and proliferation of CD4⁻CD8⁺ T cells was also significantly (p≦0.001) decreased (data not shown). Immunohistology revealed the distribution of dividing cells at 1 year of age to reflect that seen in the young adult (2-4 months); however, at 2 years, proliferation is mainly seen in the outer cortex and surrounding the vasculature with very little division in the medulla.

As early as one week post-castration there was a marked increase in the proportion of proliferating CD4⁻CD8⁻ cells and the CD4⁻CD8⁺ cells (data not shown). Castration clearly overcomes the block in proliferation of these cells with age. There was a corresponding proportional decrease in proliferating CD4⁺CD8⁻ cells post-castration (data not shown). At 2 weeks post-castration, although thymocyte numbers significantly increase, there was no change in the overall proportion of thymocytes that were proliferating, again indicating a synchronous expansion of cells (FIGS. 4A, 4B, and 5C). Immunohistology revealed the localization of thymocyte proliferation and the extent of dividing cells to resemble the situation in the 2-month-old thymus by 2 weeks post-castration.

The DN subpopulation, in addition to the thymocyte precursors, contains αβTC [symbol] ³⁰ CD4⁻CD8⁻ thymocytes, which are thought to have down-regulated both co-receptors at the transition to SP cells (Godfrey and Zlotnik, (1993) Immunol. Today 14:547). By gating on these mature cells, it was possible to analyze the true TN compartment (CD3⁻CD4⁻CD8⁻) and their subpopulations expressing CD44 and CD25. FIGS. 5E-H illustrate the extent of proliferation within each subset of TN cells in young, old and castrated mice. This showed a significant (p<0.001) decrease in proliferation of the TN1 subset (CD44⁺CD25⁻CD3⁻CD4⁻ CD8⁻), from about 10% in the normal young to around 2% at 18 months of age (FIG. 5E) which was restored by 1 week post-castration.

### III. Thymocyte Emigration

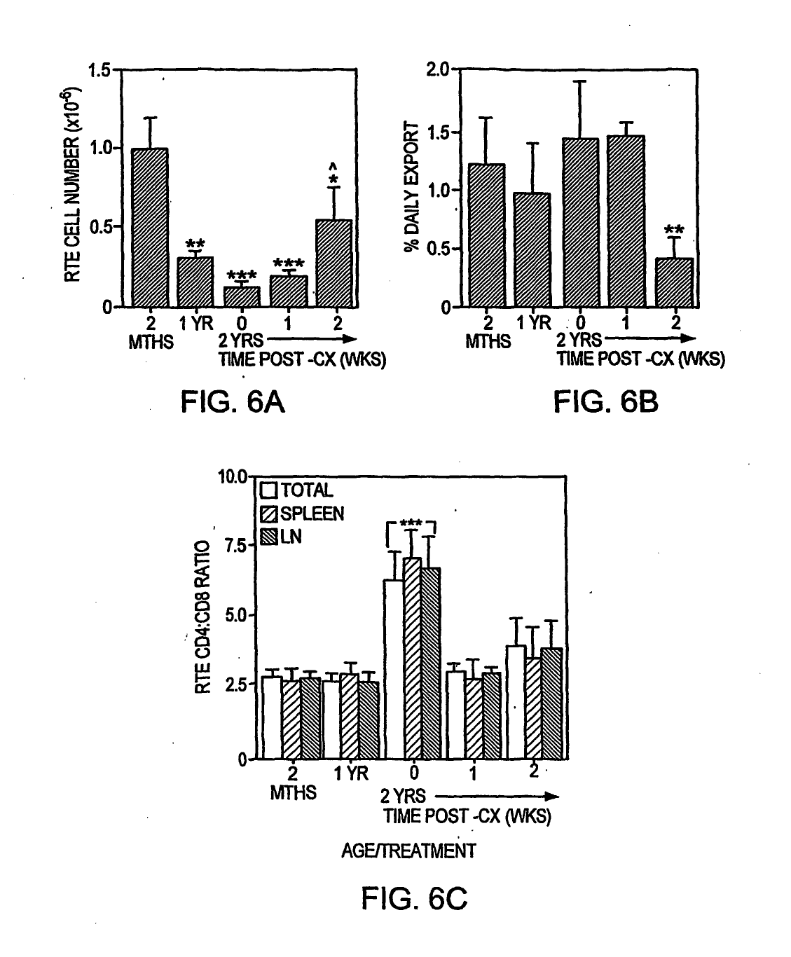

Approximately 1% of T cells migrate from the thymus daily in the young mouse (Scollay et al., (1980) Proc. Natl. Acad. Sci, USA 86:5547). Migration in castrated mice was found to occur at a proportional rate equivalent to the normal young mouse at 14 months and even 2 years of age, although significantly (p≦0.0001) reduced in number (FIGS. 6A and 6B). There was an increase in the CD4:CD8 ratio of the recent thymic emigrants from about 3:1 at 2 months to about 7:1 at 26 months (FIG. 6C). By 1 week post-castration, this ratio had normalized (FIG. 6C). By 2 weeks post-castration, cell number migrating to the periphery had substantially increased with the overall rate of migration reduced to 0.4% reflecting the expansion of the thymus (FIG. 6B).

Discussion

It has been shown that an aged thymus, although severely atrophic, maintains its functional capacity with age, with T cell proliferation, differentiation and migration occurring at levels equivalent to the young adult mouse. Although thymic function is regulated by several complex interactions between the neuro-endocrine-immune axes, the atrophy induced by sex steroid production exerts the most significant and prolonged effects illustrated by the extent of thymus regeneration post-castration.

Thymus weight is significantly reduced with age as shown previously (Hirokawa and Makinodan (1975) J. Immunol. 114:1659; Aspinall (1997) J. Immunol. 158:3037)) and correlates with a significant decrease in thymocyte numbers. The stress induced by the castration technique, which may result in further thymus atrophy due to the actions of corticosteroids, is overridden by the removal of sex steroid influences with the 2-week castrate thymus increasing in cellularity by 20-30 fold from the pre-castrate thymus. By 3 weeks post-castration, the aged thymus shows a significant increase in both thymic size and cell number, surpassing that of the young adult thymus presumably due to the actions of sex steroids already exerting themselves in the 2 month old mouse.

The data confirms previous findings that emphasize the continued ability of thymocytes to differentiate and maintain constant subset proportions with age (Aspinall (1997) J. Immunol. 158:3037). In addition, thymocyte differentiation has been shown to occur simultaneously post-castration, indicative of a synchronous expansion in thymocyte subsets. Since thymocyte numbers are decreased significantly with age, proliferation of thymocytes was analyzed to determine if this was a contributing factor in thymus atrophy.

Proliferation of thymocytes was not affected by age-induced thymic atrophy or by removal of sex-steroid influences post-castration with about 14% of all thymocytes proliferating. However, the localization of this division differed with age: the 2 month mouse thymus shows abundant division throughout the subcapsular and cortical areas (TN and DP T cells) with some division also occurring in the medulla. Due to thymic epithelial disorganization with age, localization of proliferation was difficult to distinguish but appeared to be less uniform in pattern than the young and relegated to the outer cortex. By 2 weeks post-castration, dividing thymocytes were detected throughout the cortex and were evident in the medulla with similar distribution to the 2 month thymus.

The phenotype of the proliferating population as determined by CD4 and CD8 analysis, was not altered with age or following castration. However, analysis of proliferation within thymocyte subpopulations revealed a significant decrease in proliferation of both the TN and CD8⁺ cells with age. Further analysis within the TN subset on the basis of the markers CD44 and CD25, revealed a significant decrease in proliferation of the TN1 (CD44⁺CD25⁻) population which was compensated for by an increase in the TN2 (CD44⁻CD25⁺) population. These abnormalities within the TN population reflect the findings by Aspinall (1997) J. Immunol. 158:3037). Surprisingly, the TN subset was proliferating at normal levels by 2 weeks post-castration, indicative of the immediate response of this population to the inhibition of sex-steroid action. Additionally, at both 2 weeks and 4 weeks post-castration, the proportion of CD8⁺ T cells that were proliferating was markedly increased from the control thymus, possibly indicating a role in the re-establishment of the peripheral T cell pool.

Thymocyte migration was shown to occur at a constant proportion of thymocytes with age conflicting with previous data by Scollay et al., ((1980) Proc. Natl. Acad. Sci., USA 86:5547), who showed a ten-fold reduction in the rate of thymocyte migration to the periphery. The difference in these results may be due to the difficulties in intrathymic FITC labelling of 2 year old thymuses or the effects of adipose deposition on FITC uptake. However, the absolute numbers of T cells migrating was decreased significantly as found by Scollay resulting in a significant reduction in ratio of RTEs to the peripheral T cell pool. This will result in changes in the periphery predominantly affecting the T cell repertoire (Mackall et al., (1995) N. Eng. J. Med. 332:143). Previous papers (e.g., Mackall et al., (1995) N. Eng. J. Med. 332:143) have shown a skewing of the T cell repertoire to a memory rather than naïve T cell phenotype with age. The diminished T cell repertoire, however, may not cope if the individual encounters new pathogens, possibly accounting for the rise in immunodeficiency in the aged. Obviously, there is a need to re-establish the T cell pool in immunocompromised individuals. Castration allows the thymus to repopulate the periphery through significantly increasing the production of naïve T cells.

In the periphery, T cell numbers remained at a constant level as evidenced in the B:T cell ratios of spleen and lymph nodes, presumably due to peripheral homeostasis (Mackall et al., (1995) N. Eng. J. Med. 332:143; Berzins et al., (1998) J. Exp. Med. 187:1839). However, disruption of cellular composition in the periphery was evident with the aged thymus showing a significant decrease in CD4:CD8 ratios from 2:1 in the young adult to 1:1 in the 2 year mouse, possibly indicative of the more susceptible nature of CD4⁺ T cells to age or an increase in production of CD8⁺ T cells from extrathymic sources. By 2 weeks post-castration, this ratio has been normalized, again reflecting the immediate response of the immune system to surgical castration.

The above findings have shown firstly that the aged thymus is capable of functioning in a nature equivalent to the pre-pubertal thymus. In this respect, T cell numbers are significantly decreased but the ability of thymocytes to differentiate is not disturbed. Their overall ability to proliferate and eventually migrate to the periphery is again not influenced by the age-associated atrophy of the thymus. However, two important findings were noted. Firstly, there appears to be an adverse affect on the TN cells in their ability to proliferate, correlating with findings by Aspinall ((1997) J. Immunol. 158:3037). This defect could be attributed to an inherent defect in the thymocytes themselves. Yet, the data shown here and in previous work, has shown thymocyte differentiation, although diminished, still occurs and stem cell entry from the BM is also not affected with age (Hirokawa (1998), “Immunity and Aging,” in PRINCIPLES AND PRACTICE OF GERIATRIC MEDICINE, (M. Pathy, ed.) John Wiley and Sons Ltd; Mackall and Gress (1997) Immunol. Rev. 160:91). Secondly, the CD8⁺ T cells were significantly diminished in their proliferative capacity with age and, following castration, a significantly increased proportion of CD8⁺ T cells proliferated as compared to the 2 month mouse. The proliferation of mature T cells is thought to be a final step before migration (Suda and Zlotnik (1991) J. Immunol. 146:3068), such that a significant decrease in CD8⁺ proliferation would indicate a decrease in their migrational potential. This hypothesis is supported by the finding that the ratio of CD4:CD8 T cells in RTEs increased with age, indicative of a decrease in CD8 T cells migrating. Alternatively, if the thymic epithelium is providing the key factor for the CD8 T cell maintenance, whether a lymphostromal molecule or cytokine influence, this factor may be disturbed with increased sex-steroid production. By removing the influence of sex-steroids, the CD8 T cell population can again proliferate optimally.

The defect in proliferation of the TN1 subset which was observed indicates that loss of cortical epithelium affects thymocyte development at the crucial stage of TCR gene rearrangement whereby the cortical epithelium provides factors such as IL-7 and SCF necessary for thymopoiesis (Godfrey and Zlotnik (1993) Immunol. Today 14:547; Aspinall (1997) J. Immunol. 158:3037). Indeed, IL-7^{-/-} and IL-7R^{-/-} mice show similar thymic morphology to that seen in aged mice (Wiles et al., (1992) Eur. J. Immunol. 22:1037; Zlotnik and Moore (1995) Curr. Opin. Immunol. 7:206; von Freeden-Jeffry (1995) J. Exp. Med. 181:1519).

In conclusion, the aged thymus still maintains its functional capacity, however, the thymocytes that develop in the aged mouse are not under the stringent control by thymic epithelial cells as seen in the normal young mouse due to the lack of structural integrity of the thymic microenvironment. Thus the proliferation, differentiation and migration of these cells will not be under optimal regulation and may result in the increased release of autoreactive/immunodysfunctional T cells in the periphery. The defects within both the TN and, particularly, CD8⁺ populations, may result in the changes seen within the peripheral T cell pool with age. Restoration of thymus function by castration will provide an essential means for regenerating the peripheral T cell pool and, thus, in re-establishing immunity in immunosuppressed, immunodeficient, or immunocompromised individuals.

### Example 2

### Reversal of Chemotherapy- or Radiation-Induced Thymic Atrophy

Materials and Methods

Materials and methods were as described in Example 1. In addition, the following methods were used.

BM Reconstitution. Recipient mice (34 month-old C57BL6/J) were subjected to 5.5 Gy irradiation twice over a 3-hour interval. One hour following the second irradiation dose, mice were injected intravenously with 5×10⁶ donor BM cells. BM cells were obtained by passing RPMI-1640 media through the tibias and femurs of donor (2-month old congenic C57BL6/J Ly5.1⁺) mice, and then harvesting the cells collected in the media.

Irradiation. 3-4 month old mice were subjected to 625 rads of whole body v-irradiation.

T cell Depletion Using Cyclophosphamide. Old mice (e.g., 2 years old) were injected with cyclophosphamide (200 mg/kg body wt over two days) and castrated.

Results

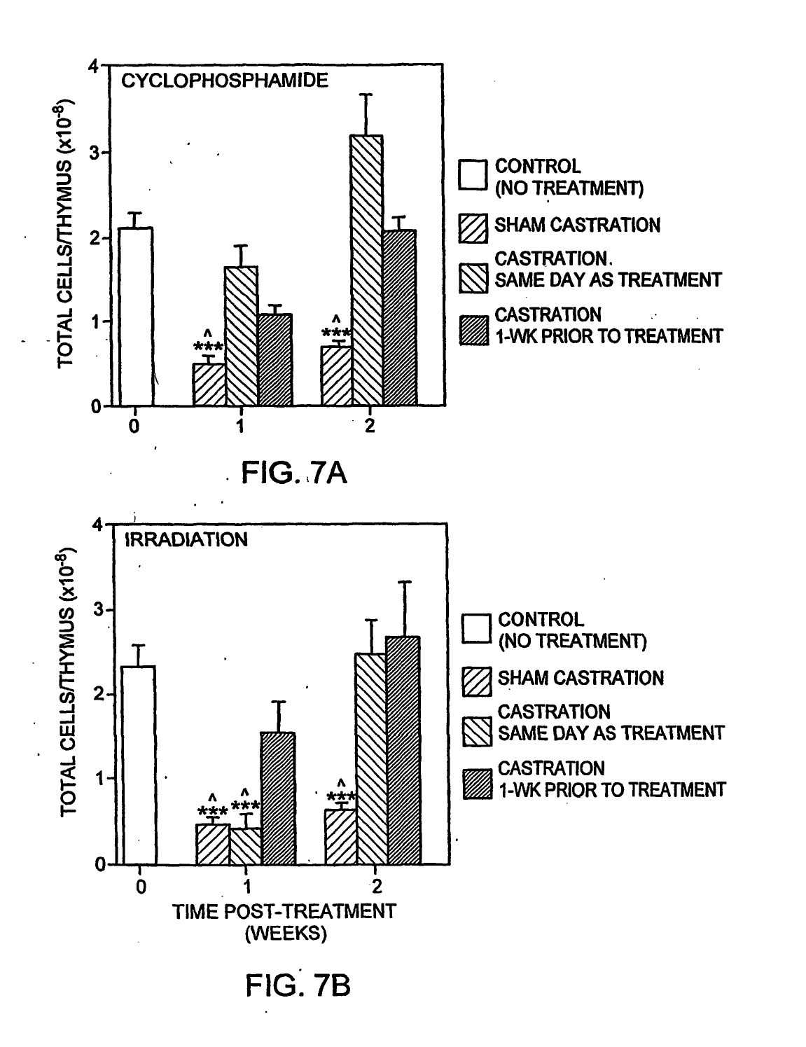

Castration-enhanced regeneration following severe T cell depletion (TCD). For both models of T cell depletion studied (chemotherapy using cyclophosphamide or sublethal irradiation using 625 rads), castrated (Cx) mice showed a significant increase in the rate of thymus regeneration compared to their sham-castrated (ShCx) counterparts (FIGS. 7A and 7B). By 1 week post-treatment, castrated mice showed significant thymic regeneration even at this early stage (FIGS. 7 and 9-11). In comparison, non-castrated animals showed severe loss of DN and DP thymocytes (rapidly-dividing cells) and subsequent increase in proportion of CD4 and CD8 cells (radio-resistant). This is best illustrated by the differences in thymocyte numbers with castrated animals showing at least a 4-fold increase in thymus size even at 1 week post-treatment. By 2 weeks, the non-castrated animals showed relative thymocyte normality with regeneration of both DN and DP thymocytes. However, proportions of thymocytes are not yet equivalent to the young adult control thymus. Indeed, at 2 weeks, the vast difference in regulation rates between castrated and non-castrated mice was maximal (by 4 weeks thymocyte numbers were equivalent between treatment groups).

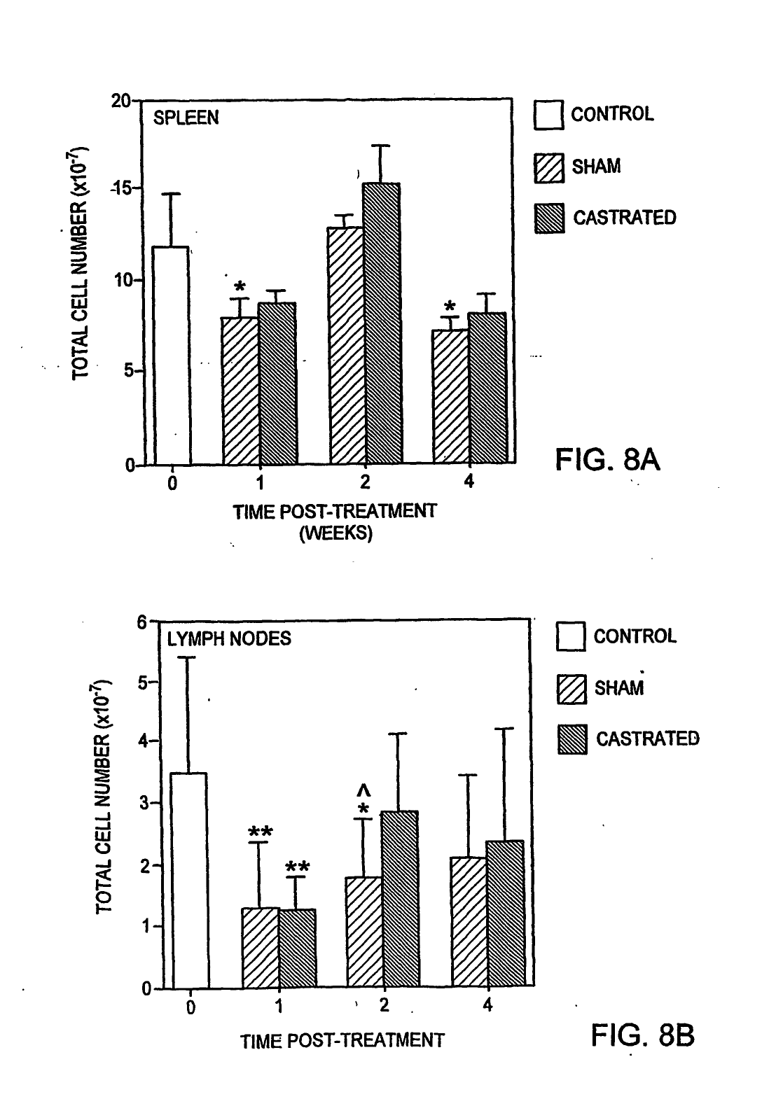

Thymus cellularity was significantly reduced in ShCx mice 1-week post-cyclophosphamide treatment compared to both control (untreated, aged-matched; p≦0.001) and Cx mice (p≦0.05) (FIG. 7A). No difference in thymus regeneration rates was observed at this time-point between mice castrated 1-week earlier or on the same day as treatment, with both groups displaying at least a doubling in the numbers of cells compared to ShCx mice (FIGS. 7A and 8A). Similarly, at 2-weeks post-cyclophosphamide treatment, both groups of Cx mice had significantly (5-6 fold) greater thymocyte numbers (p≦0.001) than the ShCx mice (FIG. 7A). In control mice there was a gradual recovery of thymocyte number over 4 weeks but this was markedly enhanced by castration, even within one week (data not shown). Similarly, spleen and lymph node numbers were increased in the castrate mice after one week (data not shown).

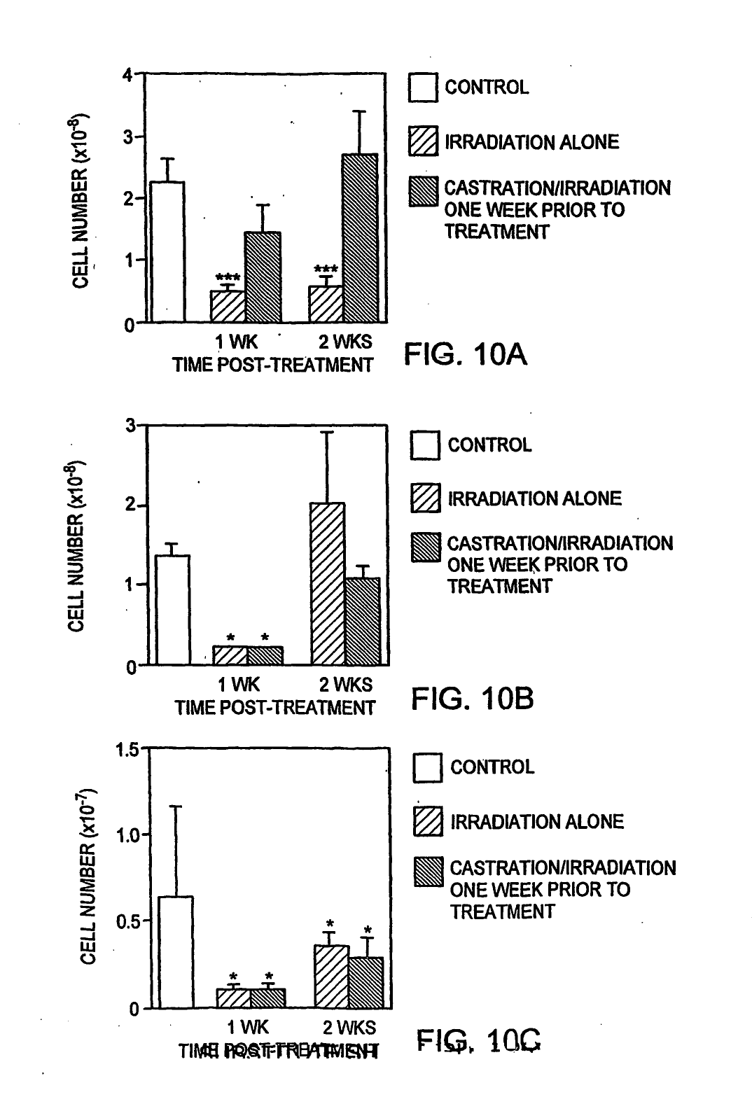

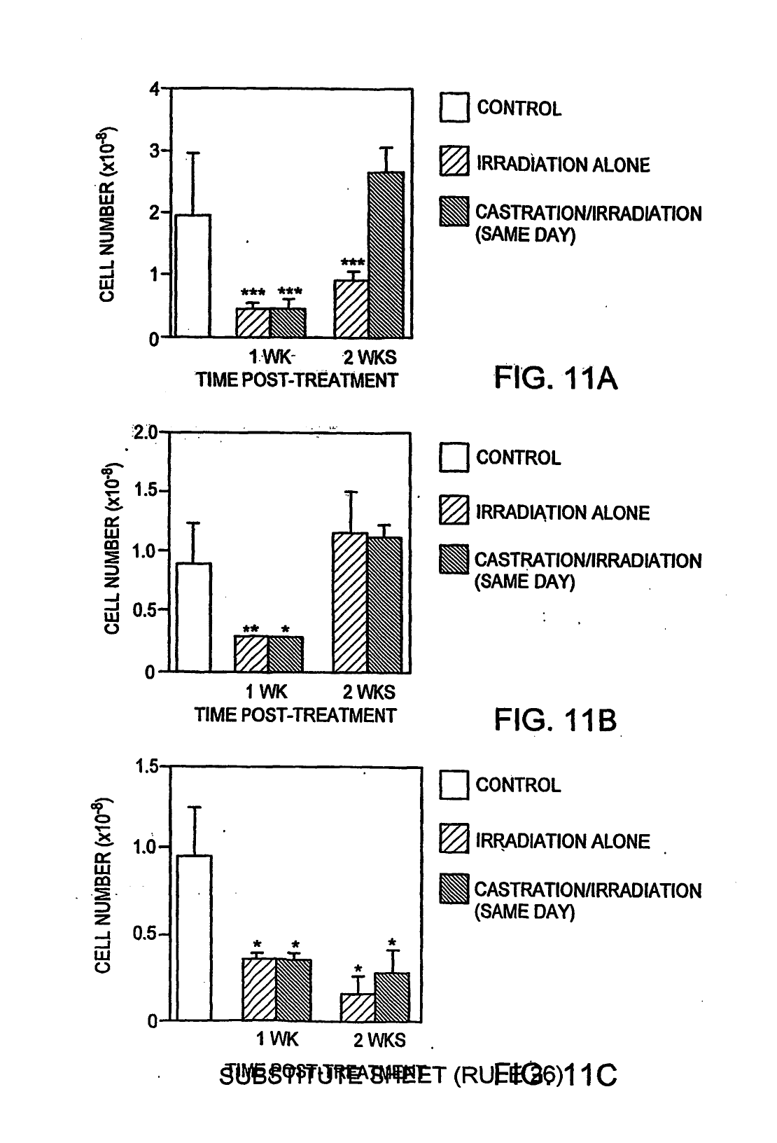

The effect of the timing of castration on thymic recovery was examined by castration one week prior to either irradiation (FIG. 10) or on the same day as irradiation (FIG. 11). When performed one week prior, castration had a more rapid impact on thymic recovery (FIG. 10A compared to FIG. 11A). By two weeks, the same-day castration had “caught up” with the thymic regeneration in mice castrated one week prior to treatment. In both cases, there were no major effects on spleen or lymph nodes (FIGS. 10B and 10C, and FIGS. 11B and 11C), respectively.

Following irradiation treatment, both ShCx and mice castrated on the same day as treatment (SDCx) showed a significant reduction in thymus cellularity compared to control mice (p≦0.001) (FIGS. 7B and 11A) and mice castrated 1-week prior to treatment (p≦0.01) (FIG. 7B). At 2 weeks post-treatment, the castration regime played no part in the restoration of thymus cell numbers with both groups of castrated mice displaying a significant enhancement of thymus cellularity post-irradiation (PIrr) compared to ShCx mice (p≦0.001) (FIGS. 7B, 10A, and 11A). Therefore, castration significantly enhances thymus regeneration post-severe T cell depletion, and it can be performed at least 1-week prior to immune system insult.

Interestingly, thymus size appears to “overshoot” the baseline of the control thymus. It takes about 3-4 weeks for thymocytes to migrate through and out into the periphery. Therefore, although proportions within each subpopulation are equal, numbers of thymocytes are building before being released into the periphery.

Following cyclophosphamide treatment of young mice (about 2-3 months), total lymphocyte numbers within the spleen of Cx mice, although reduced, were not significantly different from control mice throughout the time-course of analysis (data not shown). However, ShCx mice showed a significant decrease in total splenocyte numbers at 1- and 4-weeks post-treatment (p≦0.05) (FIG. 8A). Within the lymph nodes, a significant decrease in cellularity was observed at 1-week post-treatment for both sham-castrated and castrated mice (p≦0.01) (FIG. 8B), possibly reflecting the influence of stress steroids. By 2-weeks post-treatment, lymph node cellularity of castrated mice was comparable to control mice, however, sham-castrated mice did not restore their lymph node cell numbers until 4-weeks post-treatment, with a significant (p≦0.05) reduction in cellularity compared to both control and Cx mice at 2-weeks post-treatment (FIG. 8B). These results indicate that castration may enhance the rate of recovery of total lymphocyte numbers following cyclophosphamide treatment.

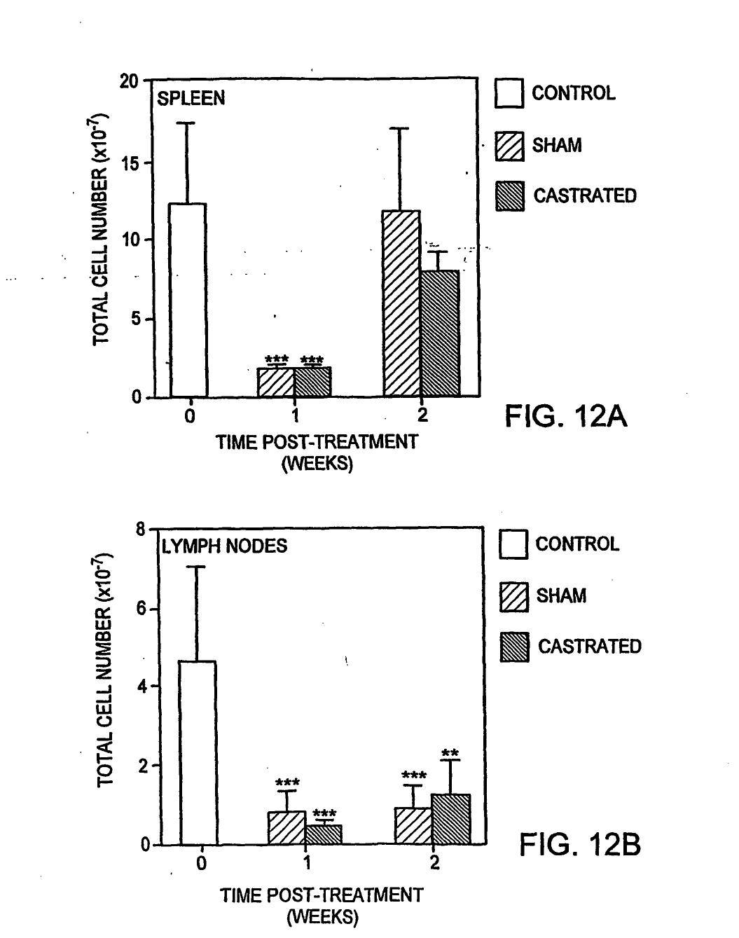

Sublethal irradiation (625 rads) induced a profound lymphopenia such that at 1-week post-treatment, both treatment groups (Cx and ShCx) showed a significant reduction in the cellularity of both spleen and lymph nodes (p≦0.001) compared to control mice (FIGS. 12A and 12B). By 2 weeks post-irradiation, spleen cell numbers were similar to control values for both castrated and sham-castrated mice (FIG. 12A), while lymph node cell numbers were still significantly lower than control values (p≦0.001 for sham-castrated mice; p≦0.01 for castrated mice) (FIG. 12B). No significant difference was observed between the Cx and ShCx mice.

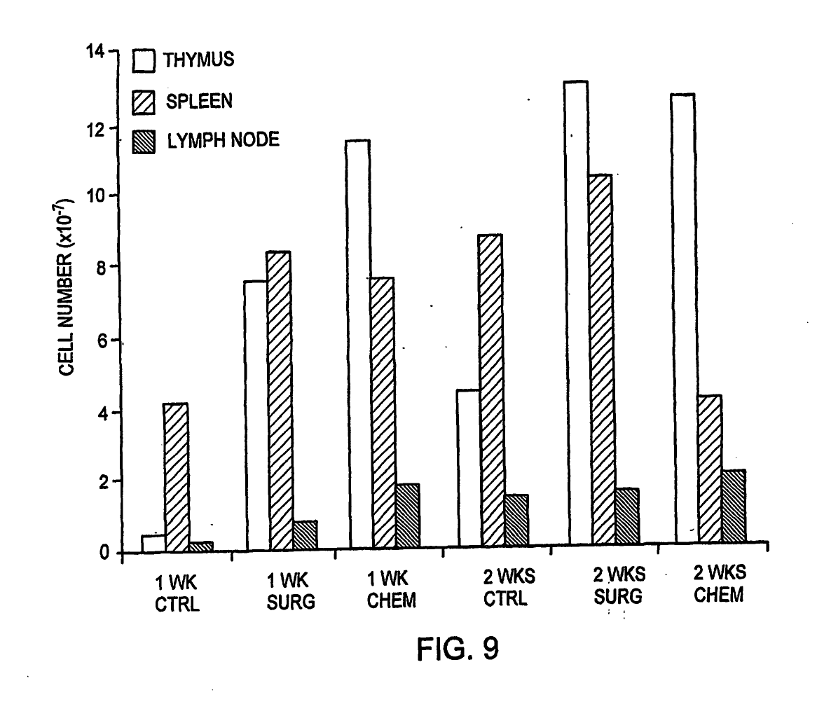

FIG. 9 illustrates the use of chemical castration compared to surgical castration in enhancement of T cell regeneration. The chemical used in this example, Deslorelin (an LHRH-A), was injected for four weeks, and showed a comparable rate of regeneration post-cyclophosphamide treatment compared to surgical castration. The enhancing effects were equivalent on thymic expansion and also the recovery of spleen and lymph node. The kinetics of chemical castration are slower than surgical, that is, mice take about 3 weeks longer to decrease their circulating sex steroid levels. However, chemical castration is still as effective as surgical castration and can be considered to have an equivalent effect.

Discussion

The impact of castration on thymic structure and T cell production was investigated in animal models of immunodepletion. Specifically, Example 2 examined the effect of castration on the recovery of the immune system after sublethal irradiation and cyclophosphamide treatment. These forms of immunodepletion act to inhibit DNA synthesis and therefore target rapidly dividing cells. In the thymus these cells are predominantly immature cortical thymocytes, but all subsets are effected (Fredrickson and Basch (1994) Dev. Comp. Immunol. 18:251). In normal healthy aged animals, the qualitative and quantitative deviations in peripheral T cells seldom lead to pathological states. However, major problems arise following severe depletion of T cells because of the reduced capacity of the thymus for T cell regeneration. Such insults occur in HIV/AIDS and, particularly, following chemotherapy and radiotherapy in cancer treatment (Mackall et al, (1995) N. Eng. J. Med. 332:143).

In both sublethally irradiated and cyclophosphamide treated mice, castration markedly enhanced thymic regeneration. Castration was carried out on the same day as and seven days prior to immunodepletion in order to appraise the effect of the predominantly corticosteroid induced, stress response to surgical castration on thymic regeneration. Although increases in thymus cellularity and architecture were seen as early as one week after immunodepletion, the major differences were observed two weeks after castration. This was the case whether castration was performed on the same day or one week prior to immunodepletion.

Immunohistology demonstrated that in all instances, two weeks after castration the thymic architecture appeared phenotypically normal, while that of non-castrated mice was disorganized. Pan epithelial markers demonstrated that immunodepletion caused a collapse in cortical epithelium and a general disruption of thymic architecture in the thymii of non-castrated mice. Medullary markers supported this finding. Interestingly, one of the first features of castration-induced thymic regeneration was a marked upregulation in the extracellular matrix, identified by MTS 16.

Flow cytometry analysis data illustrated a significant increase in the number of cells in all thymocyte subsets in castrated mice. At each time-point, there was a synchronous increase in all CD4, CD8 and αβ-TCR—defined subsets following immunodepletion and castration. This is an unusual but consistent result. Since T cell development is a progressive process, it was expected that there would be an initial increase in precursor cells (contained within the CD4⁻CD8⁻ gate), and this may have occurred before the first time-point. Moreover, since precursors represent a very small proportion of total thymocytes, a shift in their number may not have been detectable. The effects of castration on other cells, including macrophages and granulocytes, were also analyzed. In general, there was little alteration in macrophage and granulocyte numbers within the thymus.

In both irradiation and cyclophosphamide models of immunodepletion, thymocyte numbers peaked at every two weeks and decreased four weeks after treatment. Almost immediately after irradiation or chemotherapy, thymus weight and cellularity decreased dramatically, and approximately 5 days later the first phase of thymic regeneration began. The first wave of reconstitution (days 5-14) was brought about by the proliferation of radioresistant thymocytes (predominantly double negatives) which gave rise to all thymocyte subsets (Penit and Ezine (1989) Proc. Natl. Acad. Sci., USA 86:5547). The second decrease, observed between days 16 and 22, was due to the limited proliferative ability of the radioresistant cells coupled with a decreased production of thymic precursors by the BM (also effected by irradiation). The second regenerative phase was due to the replenishment of the thymus with BM derived precursors (Huiskamp et al., (1983) Radiat. Res. 95:370).

In adult mice the development from a HSC to a mature T cell takes approximately 28 days (Shortman et al., (1990) Sem. Immunol. 2:3). Therefore, it is not surprising that little change was seen in peripheral T cells up to four weeks after treatment. The periphery would be supported by some thymic export, but the majority of the T cells found in the periphery up to four weeks after treatment would be expected to be proliferating cyclophosphamide or irradiation resistant clones expanding in the absence of depleted cells. Several long term changes in the periphery would be expected post-castration including, most importantly, a diversification of the TCR repertoire due to an increase in thymic export.

### Example 3

### Thymic Regeneration Following Inhibition of Sex Steroids Results in Restoration of Deficient Peripheral T Cell Function

Materials and Methods

Materials and methods were as described in Examples 1 and 2. In addition, the following methods were used.

HSV-1 Immunization. Aged (≧18 months) mice were surgically castrated 6 weeks after castration (following thymus reactivation). Following anesthetic, mice were injected in the hind leg (foot-hock) with 4×10⁵ plaque forming units (pfu) of HSV-1 (KOS strain) in sterile PBS using a 20-gauge needle. Infected mice were housed in isolated cages and humanely killed on D5 post-immunization at which time the popliteal (draining) lymph nodes were removed for analysis.

Virus was obtained from Assoc. Prof. Frank Carbone (Melbourne University). Virus stocks were grown and titrated on VERO cell monolayers in MEM supplemented with 5% FCS (Gibco-BRL, Australia).

Analysis of the draining (popliteal) lymph nodes was performed on D5 post-infection. For HSV-1 studies, popliteal lymph node cells were stained for anti-CD25-PE, anti-CD8-APC and anti-Vβ10-biotin. For detection of DC, an FcR block was used prior to staining for CD45.1-FITC, I-A^{b}-PE and CD11c-biotin. All biotinylated antibodies were detected with streptavidin-PerCP. For detection of HSC, BM cells were gated on Lin⁻ cells by collectively staining with anti-CD3, CD4, CD8, Gr-1, B220 and Mac-1 (all conjugated to FITC). HSC were detected by staining with CD117-APC and Sca-1-PE. For TN thymocyte analysis, cells were gated on the Lin⁻ population and detected by staining with CD44-biotin, CD25-PE and c-kit-APC.

Cytotoxicity Assay of Lymph Node Cells. Lymph node cells were incubated for three days at 37° C., 6.5% CO₂. Specificity was determined using a non-transfected cell line (EL4) pulsed with gB₄₉₈₋₅₀₅ peptide (gBp) and EL4 cells alone as a control. A starting effector:target ratio of 30:1 was used. The plates were incubated at 37° C., 6.5% CO₂ for four hours and then centrifuged 650_{gmax} for 5 minutes. Supernatant (100 μl) was harvested from each well and transferred into glass fermentation tubes for measurement by a Packard Cobra auto-gamma counter.

Results

To determine the functional consequences of thymus regeneration (e.g., whether castration can enhance the immune response, herpes simplex virus (HSV) immunization was examined as it allows the study of disease progression and role of CTL. Castrated mice were found to have a qualitatively and quantitatively improved responsiveness to the virus.

Mice were immunized in the footpad and the popliteal (draining) lymph node analyzed at D5 post-immunization. In addition, the footpad was removed and homogenized to determine the virus titer at particular time-points throughout the experiment. The regional (popliteal) lymph node response to HSV-1 infection (FIGS. 13-17) was examined.

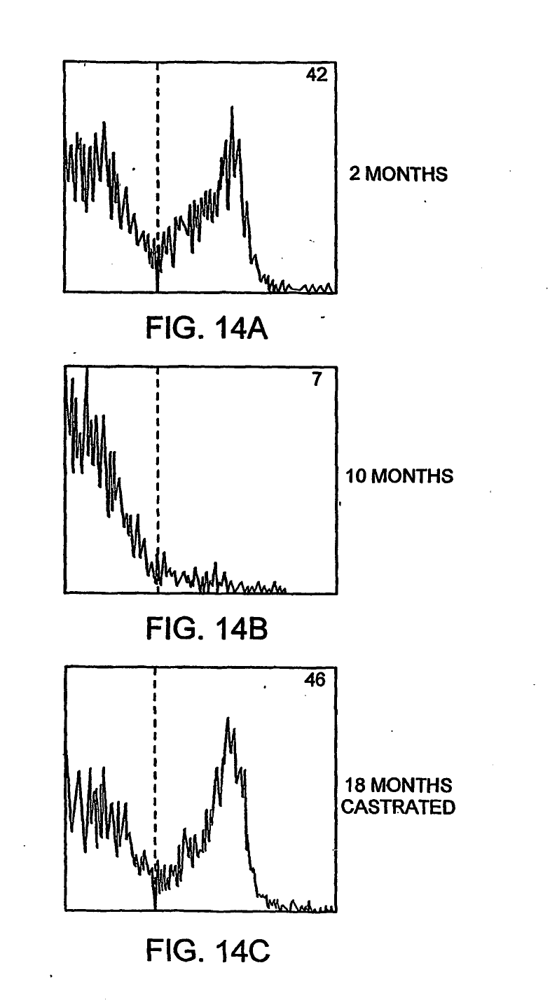

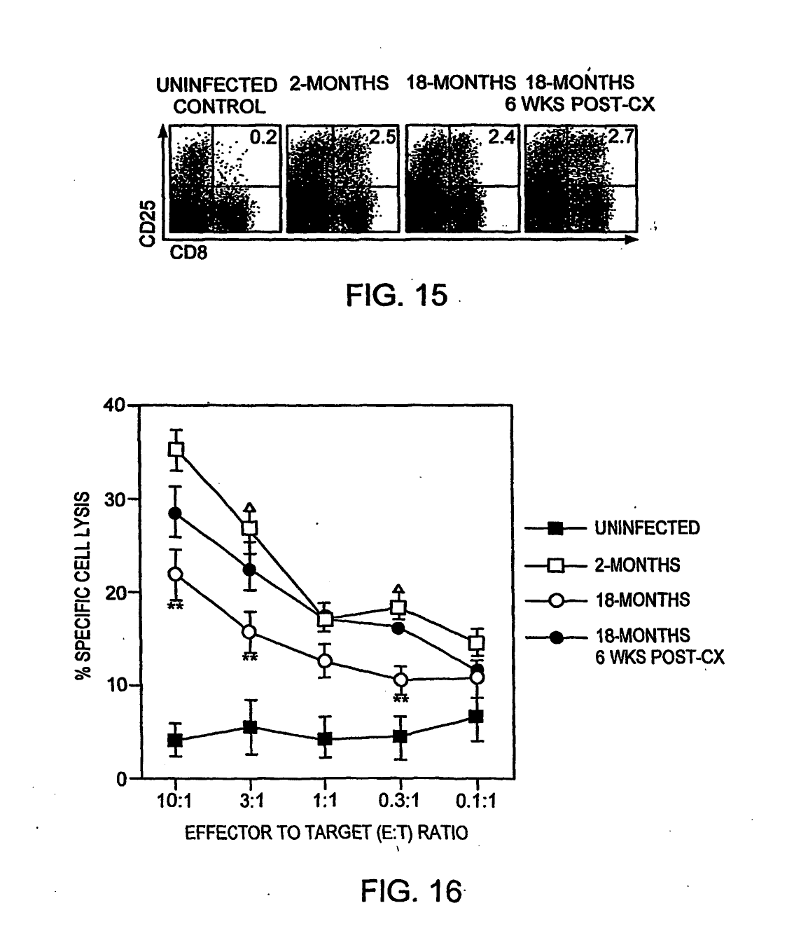

A significant decrease in lymph node cellularity was observed with age (FIGS. 13A, 13B, and 14). At D5 (i.e., 5 days) post-immunization, the castrated mice have a significantly larger lymph node cellularity than the aged mice (data not shown). Although no difference in the proportion of activated (CD8⁺CD25⁺) cells was seen with age or post-castration (FIG. 15), activated cell numbers within the lymph nodes were significantly increased with castration when compared to the aged controls (data not shown). Further, activated cell numbers correlated with that found for the young adult (data not shown), indicating that CTLs were being activated to a greater extent in the castrated mice, but the young adult may have an enlarged lymph node due to B cell activation. This was confirmed with a CTL assay detecting the proportion of specific lysis occurring with age and post-castration (FIG. 16). Aged mice showed a significantly reduced target cell lysis at effector:target ratios of 10:1 and 3:1 compared to young adult (2-month) mice (FIG. 16). Castration restored the ability of mice to generate specific CTL responses post-HSV infection (FIG. 16).

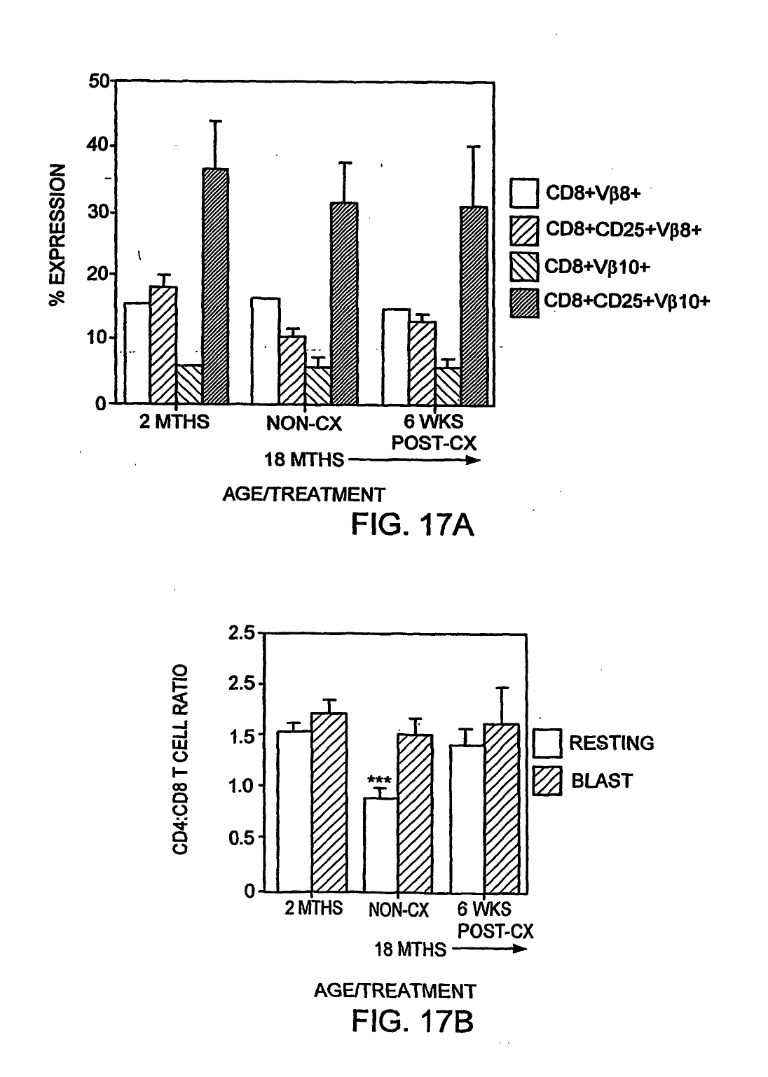

In addition, while overall expression of Vβ10 by the activated cells remained constant with age (data not shown), a subgroup of aged (18-month) mice showed a diminution of this clonal response (FIGS. 14A-C). By six weeks post-castration, the total number of infiltrating lymph node cells and the number of activated CD25⁺CD8⁺ cells had increased to young adult levels (data not shown). More importantly, however, castration significantly enhanced the CTL responsiveness to HSV-infected target cells, which was greatly reduced in the aged mice (FIG. 16) and restored the CD4:CD8 ratio in the lymph nodes (FIG. 17B). Indeed, a decrease in CD4⁺ T cells in the draining lymph nodes was seen with age compared to both young adult and castrated mice (FIG. 17B), thus illustrating the vital need for increased production of T cells from the thymus throughout life in order to get maximal immune responsiveness.

### Example 4

### Inhibition of Sex Steroids Enhances Uptake of New Hematopoietic Precursor Cells into the Thymus which Enables Chimeric Mixtures of Host and Donor Lymphoid Cells (T, B, and DC)

Materials and methods were as described in Examples 1-3. In addition, the following techniques were used:

Previous experiments have shown that microchimera formation plays an important role in organ transplant acceptance. DC have also been shown to play an integral role in tolerance to graft antigens. Therefore, the effects of castration on thymic chimera formation and dendritic cell number was studied.

In order to assess the role of stem cell uptake in thymus regeneration, BM reconstitution was performed as described in Example 2.

For the syngeneic experiments, three month old mice (n=4) were used per treatment group. All controls were age matched and untreated.

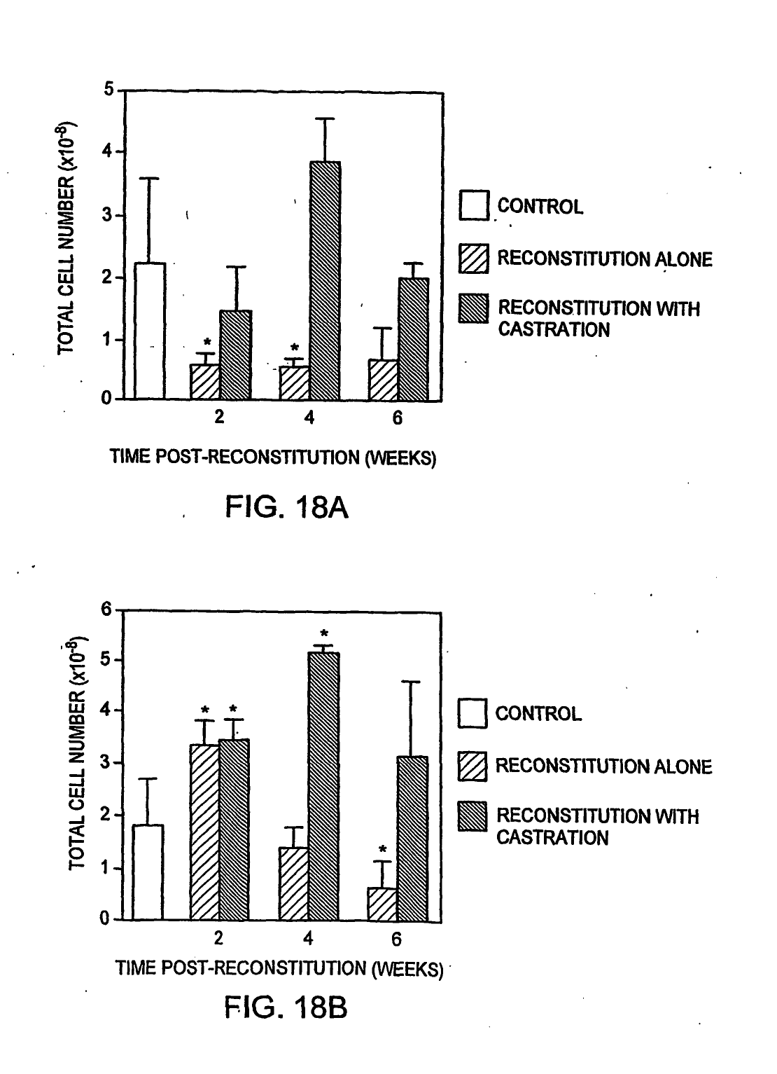

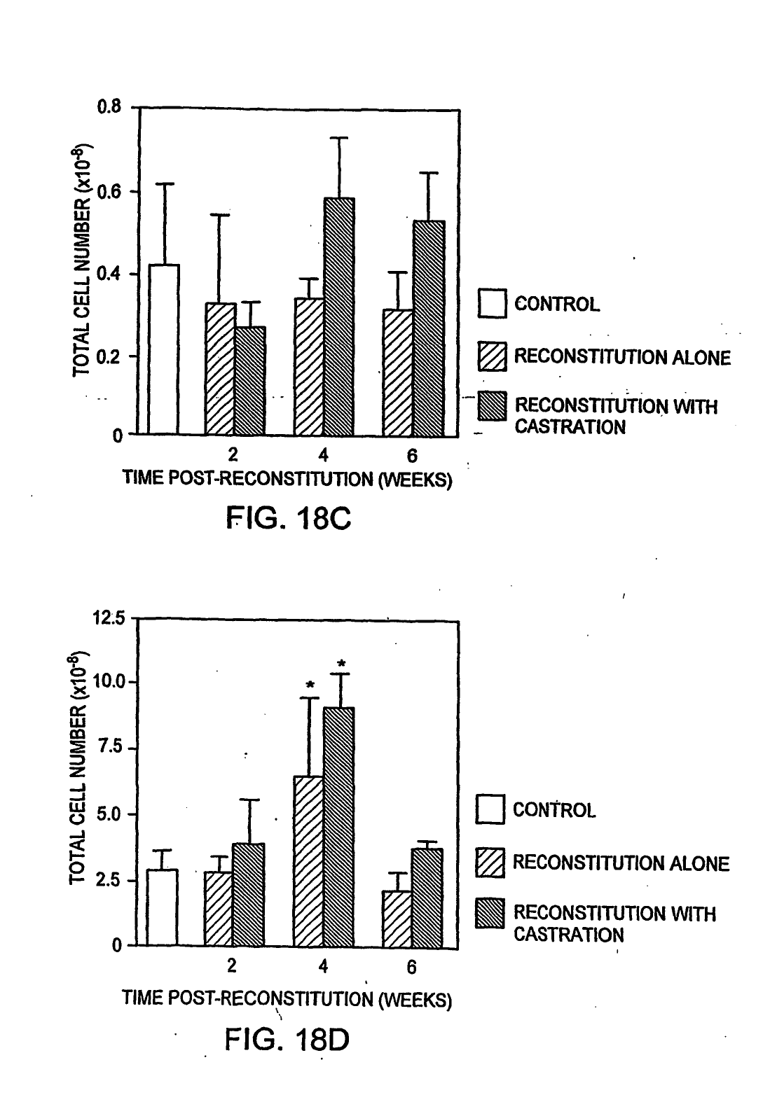

The total thymus cell numbers of castrated and noncastrated reconstituted mice were compared to untreated age matched controls and are summarized in FIG. 18A. In mice castrated 1 day prior to reconstitution, there was a significant increase (p≦0.01) in the rate of thymus regeneration compared to sham-castrated (ShCx) control mice. Thymus cellularity in the sham-castrated mice was below untreated control levels (7.6×10⁷±5.2×10⁶) 2 and 4 weeks after congenic BMT, while thymus cellularity of castrated mice had increased above control levels at 4-weeks post-BMT (FIG. 18A). At 6 weeks, cell numbers remained below control levels. However, those of castrated mice were three-fold higher than the non-castrated mice (p≦0.05) (FIG. 18A).

There were also significantly more cells (p≦0.05) in the BM of castrated mice 4 weeks after BMT (FIG. 18D). BM cellularity reached untreated control levels (1.5×10⁷±1.5×10⁶) in the sham-castrates by 2 weeks, whereas BM cellularity was increased above control levels in castrated mice at both 2 and 4 weeks after congenic BMT (FIG. 18D). Mesenteric lymph node cell numbers were decreased 2-weeks after irradiation and reconstitution in both castrated and noncastrated mice; however, by the 4 week time-point, cell numbers had reached control levels. There was no statistically significant difference in lymph node cell number between castrated and non-castrated treatment groups (FIG. 18C). Spleen cellularity reached untreated control levels (1.5×10⁷±1.5×10⁶) in the sham-castrates and castrates by 2 weeks but dropped off in the sham group over 4-6 weeks, whereas the castrated mice still had high levels of spleen cells (FIG. 18B). Again, castrated mice showed increased lymphocyte numbers at these time-points (i.e., 4 and 6 weeks post-reconstitution) compared to non-castrated mice (p≦0.05) although no difference in total spleen cell number between castrated and non-castrated treatment groups was seen at 2-weeks (FIG. 18B).

Thus, in mice castrated 1 day prior to reconstitution, there was a significant increase (p≦0.01) in the rate of thymus regeneration compared to sham-castrated (ShCx) control mice (FIG. 18A). Thymus cellularity in the sham-castrated mice was below untreated control levels (7.6×10⁷±5.2×10⁶) 2 and 4 weeks after congenic BMT, while thymus cellularity of castrated mice had increased above control levels at 4-weeks post-BMT (FIG. 18A). Castrated mice had significantly increased congenic (Ly5.2) cells compared to non-castrated animals.

In non-castrated mice, there was a profound decrease in thymocyte number over the 4 week time period, with little or no evidence of regeneration (data not shown). In the castrated group, however, by two weeks there was already extensive thymopoiesis which by four weeks had returned to control levels, being 10-fold higher than in non-castrated mice. Flow cytometric analysis of the thymuses with respect to CD45.2 (donor-derived antigen) demonstrated that no donor derived cells were detectable in the non-castrated group at 4 weeks, but remarkably, virtually all the thymocytes in the castrated mice were donor-derived at this time-point (data not shown). Given this extensive enhancement of thymopoiesis from donor-derived hemopoietic precursors, it was important to determine whether T cell differentiation had proceeded normally. CD4, CD8 and TCR defined subsets were analyzed by flow cytometry. There were no proportional differences in thymocytes subset proportions 2 weeks after reconstitution (data not shown). This observation was not possible at 4 weeks, because the non-castrated mice were not reconstituted with donor-derived cells. However, at this time-point the thymocyte proportions in castrated mice appear normal.

In a parallel set of experiments, 3 month old, young adults, C57/BL6 mice were castrated or sham-castrated 1 day prior to BMT. For congenic BMT, the mice were subjected to 800 rads TBI and IV injected with 5×10⁶ Ly5.1⁺ BM cells. Mice were killed 2 and 4 weeks later and the BM, thymus and spleen were analyzed for immune reconstitution. Donor/Host origin was determined with anti-CD45.1 antibody, which only reacts with leukocytes of donor origin.

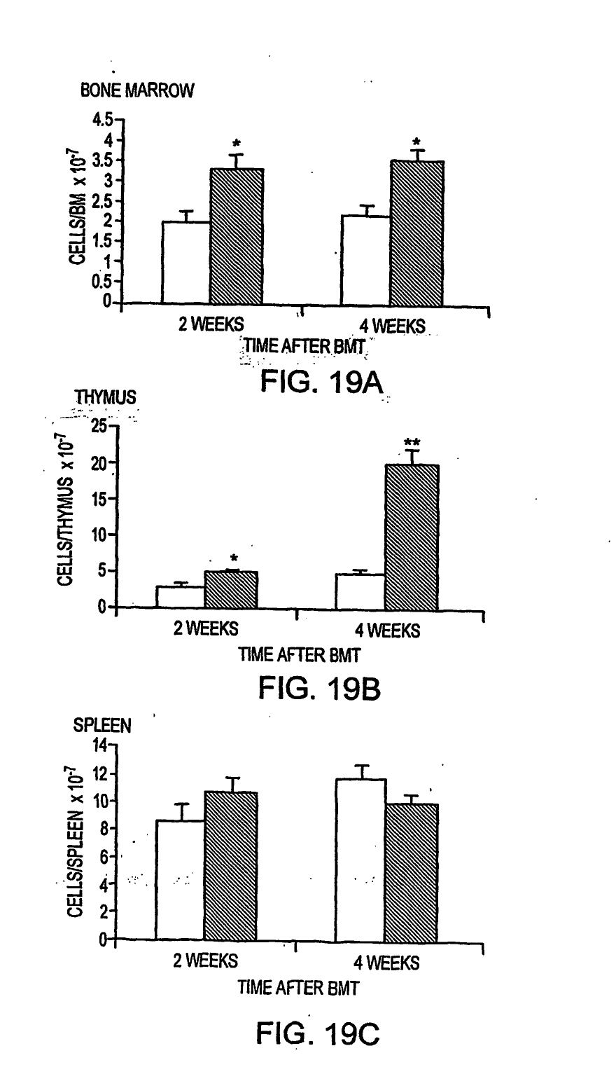

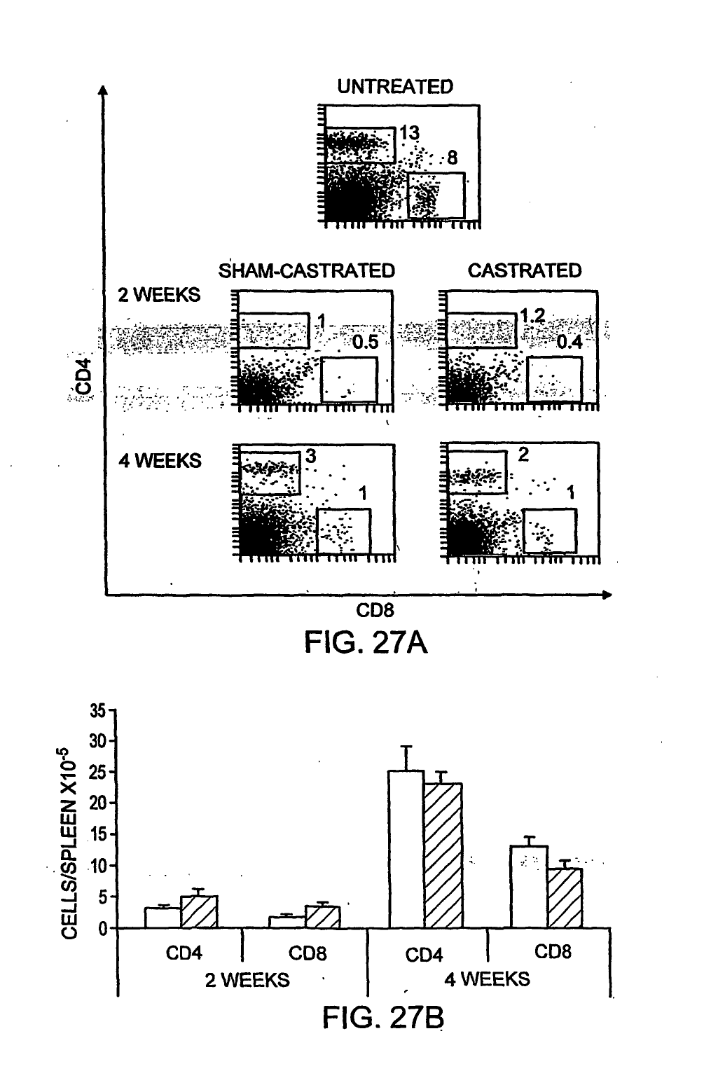

The results from this parallel set of experiments are shown in FIGS. 19-28.

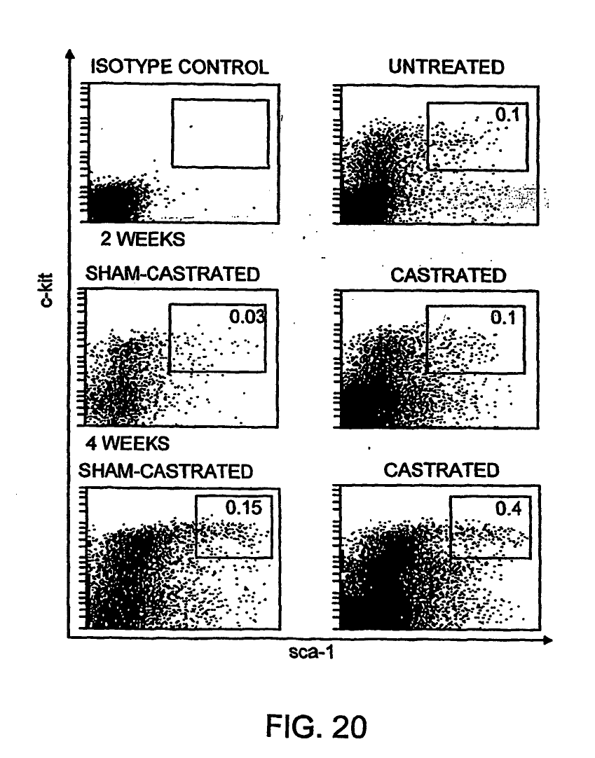

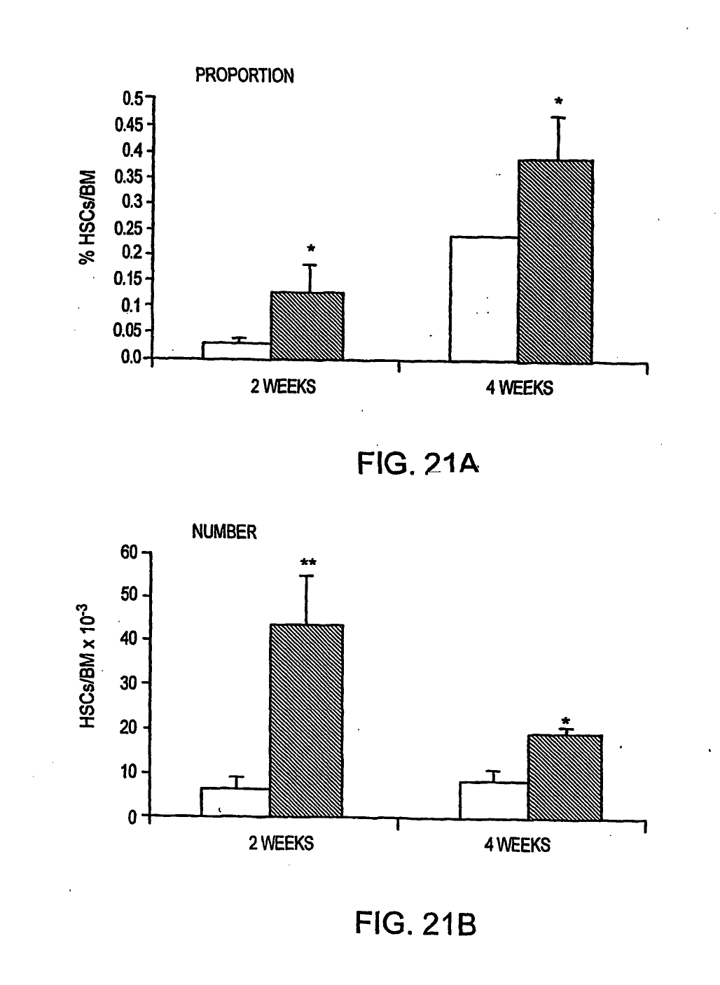

FIGS. 20 and 21 show an increase in the number and proportion of donor-derived HSC in the BM of castrated animals. This indicates improved engraftment and suggests faster recovery from BMT.

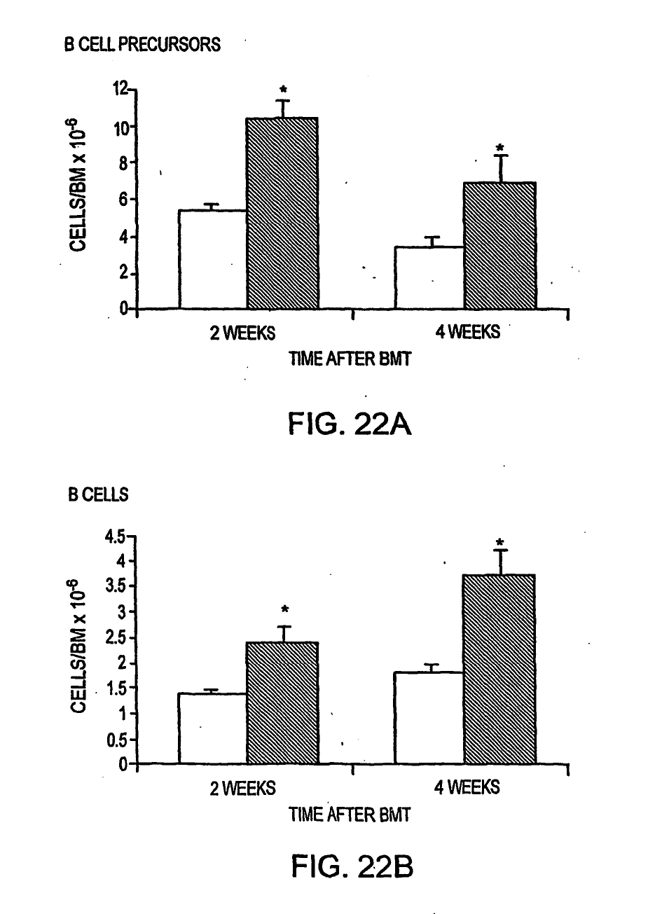

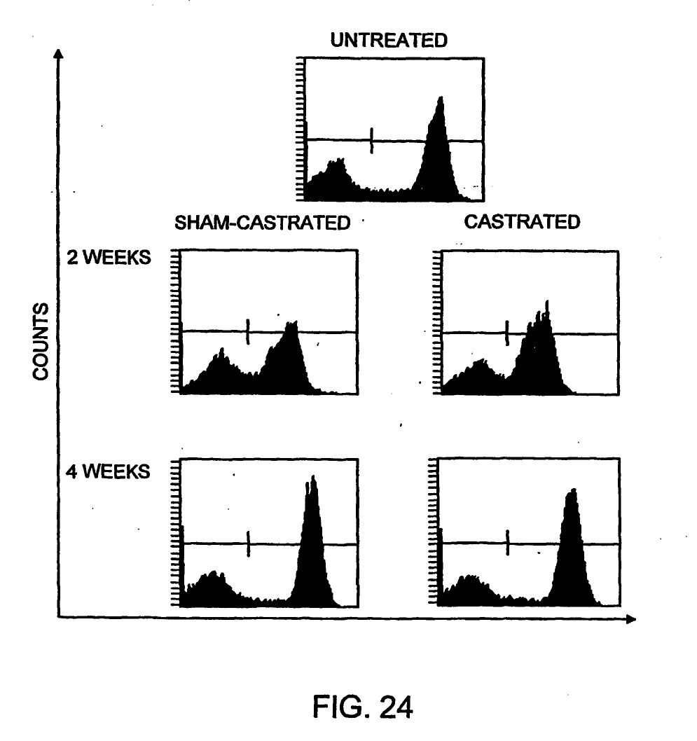

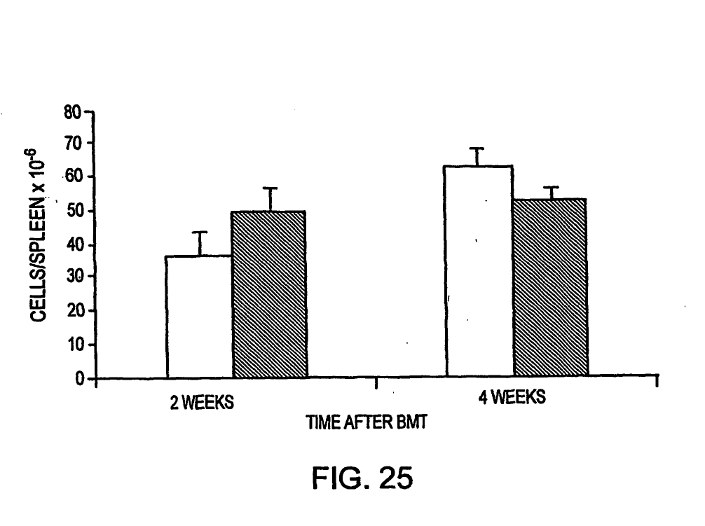

FIG. 22 shows an increase in donor-derived B cell precursors and B cells in the BM of castrated mice. However, FIGS. 24 and 25 show castration does not alter the number or proportion of B cells in the periphery at 2 and 4 weeks post castration.

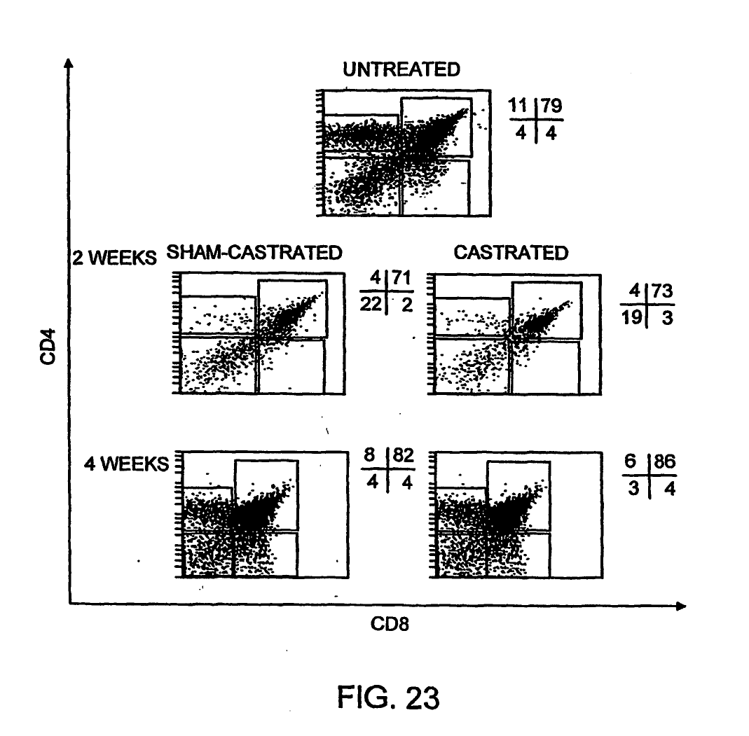

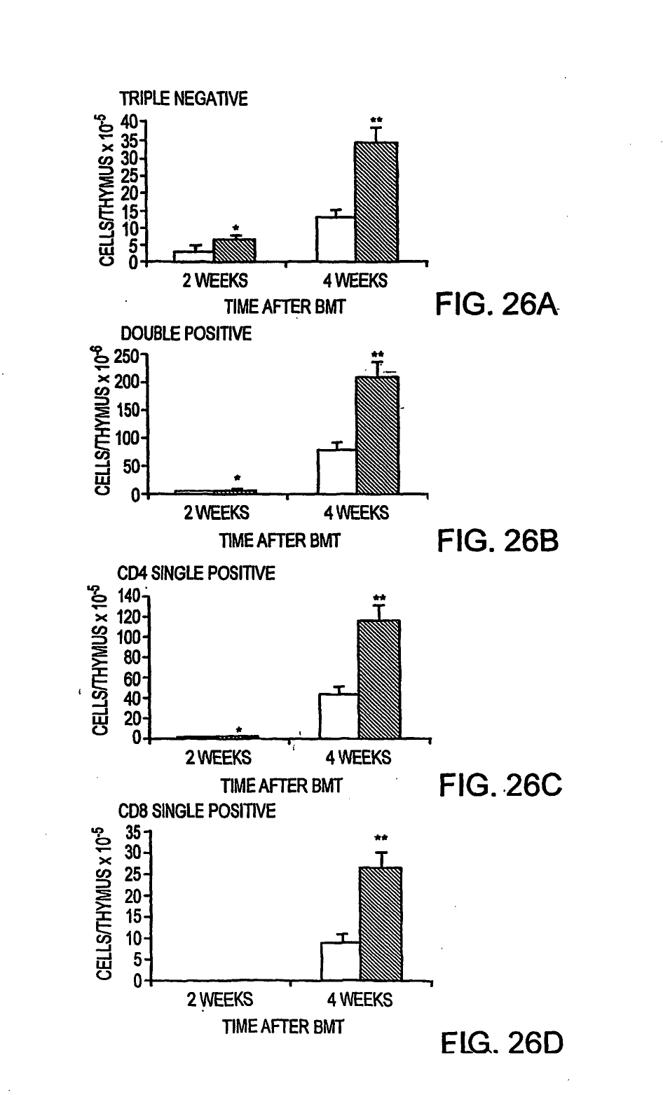

FIG. 26 shows castration increased numbers of donor-derived TN, DP, CD4 and CD8 cells in the thymus. However, FIG. 23 shows castration does not alter the donor thymocyte proportions of CD4 and CD8 cells. In the periphery, there are very few CD4 or CD8 cells and, at the time-points considered, there was no increase in these cells with castration.

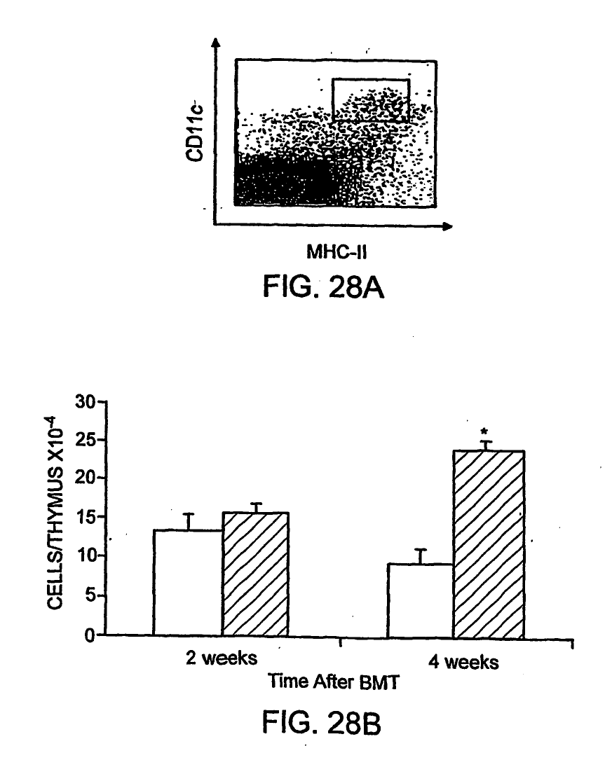

Importantly, FIG. 28 shows an increased number of donor DC in the thymus by 4 weeks post castration.

Discussion

Example 4 shows the influence of castration on syngeneic and congenic BM transplantation. Starzl et al., (1992) Lancet 339:1579 reported that microchimeras evident in lymphoid and nonlymphoid tissue were a good prognostic indicator for allograft transplantation. That is, it was postulated that they were necessary for the induction of tolerance to the graft (Starzl et al., (1992) Lancet 339:1579). Donor-derived DC were present in these chimeras and were thought to play an integral role in the avoidance of graft rejection (Thomson and Lu (1999) Immunol. Today 20:20). DC are known to be key players in the negative selection processes of thymus and if donor-derived DC were present in the recipient thymus, graft reactive T cells may be deleted.

In order to determine if castration would enable increased chimera formation, a study was performed using syngeneic fetal liver transplantation. The results showed an enhanced regeneration of thymii of castrated mice. These trends were again seen when the experiments were repeated using congenic (Ly5) mice. Due to the presence of congenic markers, it was possible to assess the chimeric status of the mice. As early as two weeks after fetal liver reconstitution there were donor-derived dendritic cells detectable in the thymus, the number in castrated mice being four-fold higher than that in noncastrated mice. Four weeks after reconstitution, the non-castrated mice did not appear to be reconstituted with donor-derived cells, suggesting that castration may, in fact, increase the probability of chimera formation. Given that castration not only increases thymic regeneration after lethal irradiation and fetal liver reconstitution and that it also increases the number of donor-derived dendritic cells in the thymus, along-side stem cell transplantation, this approach increases the probability of graft acceptance.

### Example 5

### Immune Cell Depletion

In order to prevent interference with the graft by the existing T cells in the potential graft recipient patient, the patient underwent T cell depletion (ablation). One standard procedure for this step is as follows. The human patient received anti-T cell antibodies in the form of a daily injection of 15 mg/kg of Atgam (xeno anti-T cell globulin, Pharmacia Upjohn) for a period of 10 days in combination with an inhibitor of T cell activation, cyclosporin A, 3 mg/kg, as a continuous infusion for 3-4 weeks followed by daily tablets at 9 mg/kg, as needed. This treatment did not affect early T cell development in the patient's thymus, as the amount of antibody necessary to have such an affect cannot be delivered due to the size and configuration of the human thymus. The treatment was maintained for approximately 4-6 weeks to allow the loss of sex steroids followed by the reconstitution of the thymus.

The prevention of T cell reactivity may also be combined with inhibitors of second level signals such as interleukins, accessory molecules (e.g., antibodies blocking, e.g., CD28), signal transduction molecules or cell adhesion molecules to enhance the T cell ablation or other immune cell depletion. The thymic reconstitution phase would be linked to injection of donor HSC (obtained at the same time as the organ or tissue in question either from blood, pre-mobilized from the blood with G-CSF (2 intradermal injections/day for 3 days) or collected directly from the BM of the donor). The enhanced levels of circulating HSC would promote uptake by the thymus (activated by the absence of sex steroids and/or the elevated levels of GnRH). These donor HSC would develop into intrathymic DC and cause deletion of any newly formed T cells which by chance would be “donor-reactive.” This would establish central tolerance to the donor cells and tissues and thereby prevent or greatly minimize any rejection by the host. The development of a new repertoire of T cells would also overcome the immunodeficiency caused by the T cell-depletion regime.

The depletion of peripheral T cells minimizes the risk of graft rejection because it depletes non-specifically all T cells including those potentially reactive against a foreign donor. Simultaneously, however, because of the lack of T cells, the procedure induces a state of generalized immunodeficiency which means that the patient is highly susceptible to infection, particularly viral infection.

### Example 6

### Sex Steroid Ablation Therapy

The patient was given sex steroid ablation therapy in the form of delivery of an LHRH agonist. This was given in the form of either Leucrin (depot injection; 22.5 mg) or Zoladex® (implant; 10.8 mg), either one as a single dose effective for 3 months. This was effective in reducing sex steroid levels sufficiently to reactivate the thymus. In some cases it is also necessary to deliver a suppresser of adrenal gland production of sex steroids. Cosudex® (5 mg/day or 50 mg/day) may also be given as one tablet per day for the duration of the sex steroid ablation therapy. Alternatively, the patient is given a GnRH antagonist, e.g., Cetrorelix or Abarelix, as a subcutaneous injection

Reduction of sex steroids in the blood to minimal values takes about 1-3 weeks post surgical castration, and about 3-4 weeks following chemical castration. In some cases it is necessary to extend the treatment to a second 3 month injection/implant. The thymic expansion may be increased by simultaneous enhancement of blood HSC, either as an allogeneic donor (in the case of grafts of foreign tissue) or autologous HSC (by injecting the host with G-CSF to mobilize these HSC from the BM to the thymus).

### Example 7

### Alternative Delivery Method

In place of the 3-month depot or implant administration of the LHRH agonist, alternative methods can be used. In one example, the patient's skin may be irradiated by a laser such as an Er:YAG laser, to ablate or alter the skin so as to reduce the impeding effect of the stratum corneum.

Laser ablation or alteration is described in U.S. Pat. Nos. 6,251,100, 6,419,642 and 4,775,361.

In another example, delivery is by means of laser generated pressure waves. A dose of LHRH agonist is placed on the skin in a suitable container, such as a plastic flexible washer (about 1 inch in diameter and about 1/16 inch thick), at the site where the pressure wave is to be created. The site is then covered with target material such as a black polystyrene sheet about 1 mm thick. A Q-switched solid state ruby laser (20 ns pulse duration, capable of generating up to 2 joules per pulse) is used to generate a single impulse transient, which hits the target material. The black polystyrene target completely absorbs the laser radiation so that the skin is exposed only to the impulse transient, and not laser radiation. The procedure can be repeated daily, or as often as required, to maintain the circulating blood levels of the agonist.

### Example 8

### Administration of Donor Cells

Where practical, the level of hematopoietic stem cells (HSC) in the donor blood is enhanced by injecting into the donor granulocyte-colony stimulating factor (G-CSF) at 10 μg/kg for 2-5 days prior to cell collection (e.g., one or two injections of 10 μg/kg per day for each of 2-5 days). The donor may also be injected with LHRH agonist and/or a cytokine, such as G-CSF or GM-CSF, prior to (e.g., 7-14 days before) collection to enhance the level or quality of stem cells in the blood. CD34⁺ donor cells are purified from the donor blood or BM, such as by using a flow cytometer or immunomagnetic beading. Antibodies that specifically bind to human CD34 are commercially available (from, e.g., Research Diagnostics Inc., Flanders, N.J.; Miltenyi-Biotec, Germany). Donor-derived HSC are identified by flow cytometry as being CD34⁺. These CD34⁺ HSC may also be expanded by in vitro culture using feeder cells (e.g., fibroblasts), growth factors such as stem cell factor (SCF), and LIF to prevent differentiation into specific cell types. At approximately 3-4 weeks post LHRH agonist delivery (i.e., just before or at the time the thymus begins to regenerate) the patient is injected with the donor HSC, optimally at a dose of about 24×10⁶ cells/kg. G-CSF may also be injected into the recipient to assist in expansion of the donor HSC. If this timing schedule is not possible because of the critical nature of clinical condition, the HSC could be administered at the same time as the GnRH. It may be necessary to give a second dose of HSC approximately 2-3 weeks later to assist in the thymic regrowth and the development of donor DC (particularly in the thymus). Once the HSC have engrafted (i.e., incorporated into) and/or migrated to the BM and thymus, the effects should be permanent since HSC are self-renewing.

The reactivating or reactivated thymus takes up the donor HSC and converts them into donor-type T cells and DC, while converting the recipient's HSC into recipient-type T cells and DC. By inducing deletion by cell death, or by inducing tolerance through immunoregulatory cells, the donor and host DC tolerize any new T or NK cells that are potentially reactive with donor or recipient cells.

### Example 9

### Transplantation of Graft HSC

While the recipient is still undergoing continuous T cell depletion and/or other immune cell depletion and/or immunosuppressive therapy, the HSC are transplanted from the donor to the recipient patient. The recipient thymus has been activated by GnRH treatment and infiltrated by exogenous HSC.

Within about 3-4 weeks of LHRH therapy, the first new T cells are present in the blood stream of the recipient. However, in order to allow production of a stable chimera of host and donor hematopoietic cells, immunosuppressive therapy may be maintained for about 3-4 months. The new T cells are purged of potentially donor reactive and host reactive cells, due to the presence of both donor and host DC in the reactivating thymus. Having been positively selected by the host thymic epithelium, the T cells retain the ability to respond to normal infections by recognizing peptides presented by host APC in the peripheral blood of the recipient. The incorporation of donor DC into the recipient's lymphoid organs establishes an immune system situation virtually identical to that of the host alone, other than the tolerance of donor cells, tissue and organs. Hence, normal immunoregulatory mechanisms are present. These may also include the development of regulatory T cells which switch on or off immune responses using cytokines such as IL4, 5, 10, TGF-beta, TNF-alpha.

### Example 10

### Immunization and Prevention of Viral Infection (Influenza)

Influenza viruses are segmented RNA viruses that cause highly contagious acute respiratory infections. The major problem associated with vaccine development against influenza is that these viruses have the ability to escape immune surveillance and remain in a host population by altering antigenic sites on the hemagglutinin (HA) and neuraminidase (N) envelope glycoproteins by phenomena termed antigenic drift and antigenic shift. The primary correlate for protection against influenza virus is neutralizing antibody against HA protein that undergoes strong selection for antigenic drift and shift. However, much more conserved antigenic cross-reactivities for different strains of influenza virus occur between internal proteins, such as the nucleoprotein (NP) (Shu et al., (1993) J. Virol. 67:2723). CTL and protection from influenza challenge following immunization with a polynucleotide encoding NP has previously been shown (Ulmer et al., (1993) Science 259:1745).

Materials and Methods

Surgical Castration. BALB/c mice are anesthetized by intraperitoneal injection of 30-40 μl of a mixture of 5 ml of 100 mg/ml ketamine hydrochloride (Ketalar®; Parke-Davis, Caringbah, NSW, Australia) plus 1 ml of 20 mg/ml xylazine (Rompun®; Bayer Australia Ltd., Botany NSW, Australia) in saline. Surgical castration is performed as described elsewhere herein by a scrotal incision, revealing the testes, which are tied with suture and then removed along with surrounding fatty tissue. The wound is closed using surgical staples. Sham-castrated mice prepared following the above procedure without removal of the testes are used as controls.

Chemical Castration. Mice are injected i.m. with 10 mg/kg Lupron® (a GnRH agonist) as a 1 month slow release formulation. Alternatively, mice are injected with a GnRH antagonist (e.g., Cetrorelix or Abarelix). Confirmation of loss of sex steroids is performed by standard radioimmunoassay of plasma samples following manufacturer's instructions. Castrate levels (<0.5 ng testosterone or estrogen/ml) should normally be achieved by 3-4 weeks post injection.

Preparation of Influenza A/PR/8/34 Subunit Vaccine. Purified influenza A/PR/8/34 (H1N1) subunit vaccine preparation is prepared following methods known in the art. Briefly, the surface hemagglutinin (HA) and neuraminidase (NA) antigens from influenza A/PR/8/34 particles are extracted using a non-ionic detergent (7.5% N-octyl-β-o-thioglucopyranoside). After centrifugation, the HA/NA-rich supernatant (55% HA) is used as the subunit vaccine.

Influenza A/PR/8/34 Subunit Immunization. Approximately 6 weeks following surgical castration or about 8 weeks following chemical castration, mice are immunized with 100 μl of formalin-inactivated influenza A/PR/8/34 virus (about 7000 HAU) injected subcutaneously.

Booster immunizations can optionally be performed at about 4 weeks (or later) following the primary immunization. Freund's complete adjuvant (CFA) is used for the primary immunization and Freund's incomplete adjuvant is used for the optional booster immunizations.

Alternatively, the influenza A/PR/8/34 subunit vaccine preparation (see above) may be intramuscularly injected directly into, e.g., the quadriceps muscle, at a dose of about 1 μg to about 10 μg dilute in a volume of 40 μl sterile 0.9% saline.

Plasmid DNA. Preparation of plasmid DNA expression vectors are readily known in the art (see, e.g., Current Protocols In Immunology, Unit 2.14, John E. Coligan et al., (eds), Wiley and Sons, New York, N.Y. (1994), and yearly updates including 2002). Briefly, the complete influenza A/PR/8/34 nucleoprotein (NP) gene or hemagglutinin (HA) coding sequence is cloned into an expression vector, such as, pCMV, which is under the transcriptional control of the cytomegalovirus (CMV) immediate early promoter.

Empty plasmid (e.g., pCMV with no insert) is used as a negative control. Plasmids are grown in Escherichia coli DH5α or HB101 cells using standard techniques and purified using Qiagen® Ultra-Pure®-100 columns (Chatsworth, Calif.) according to manufacturer's instructions. All plasmids are verified by appropriate restriction enzyme digestion and agarose gel electrophoresis. Purity of DNA preparations is determined by optical density readings at 260 and 280 nm. All plasmids are resuspended in TE buffer and stored at −20° C. until use.

DNA Immunization. Methods of DNA immunization are well known in the art. For instance, methods of intradermal, intramuscular, and particle-mediated (“gene gun”) DNA immunizations are described in detail in, e.g., Current Protocols In Immunology, Unit 2.14, John E. Coligan et al., (eds), Wiley and Sons, New York, N.Y. (1994), and yearly updates including 2002).

Cytokine-encoding DNAs are optionally administered to shift the immune response to a desired Th1- or a Th2-type immune response. Th1-inducing genetic adjuvants include, e.g., IFN-γ and IL-12. Th2-inducing genetic adjuvants include, e.g., IL-4, IL-5, and IL-10. For review of the preparation and use of Th1- and Th2-inducing genetic adjuvants in the induction of immune response (see, e.g., Robinson, et al., (2000) Adv. Virus Res. 55: 1).

Influenza A/PR/8/34 Virus Challenge. In an effort to determine if castrated mice are better protected from influenza virus challenge (with and without vaccination) as compared to their sham-castrated counterparts, metofane-anesthetized mice are challenged by intranasal inoculation of 50 μl of influenza A/PR/8/34 (H1N1) influenza virus containing allantoic fluid diluted 10⁻⁴ in PBS/2% BSA (50-100 LD₅₀; 0.25 HAU). Mice are weighed daily and sacrificed following >20% loss of pre-challenge weight. At this dose of challenge virus, 100% of naïve mice should succumb to influenza infection by 4-6 days.

Sublethal infections are optionally done to activate memory T cells, but use a 10⁻⁷ dilution of virus. Sublethal infections may also be optionally done to determine if non-immunized, castrated mice have better immune responses than the sham-castrated controls, as determined by ELISA, cytokine assays (Th), CTL assays, etc. outlined below. Viral titers for lethal and sublethal infections may be optimized prior to use in these experiments.

Enzyme-Linked Immunosorbant Assays. At various time periods pre- and post-immunization (or pre- and post-infection), mice from each group are bled, and individual mouse serum is tested using standard quantitative enzyme-linked immunosorbant assays (ELISA) to assess anti-HA or —NP specific IgG levels in the serum. IgG1 and IgG2a levels may optionally be tested, which are known to correlate with Th2 and Th1-type antibody responses, respectively.

Preparation and Stimulation of Splenocytes for Cytokine Production. Spleens are aseptically harvested from the various groups of mice (n=2-3) and pooled in p60 petri dishes containing about 4 ml RPMI-10 media (RPMI-1640, 10% fetal bovine serum, 50 μg/ml gentamycin). Spleens are prepared and RBC lysed using standard procedures. Cells are then counted, and resuspended in RPMI-10 containing 80 U/ml rat IL-2 (Sigma, St. Louis, Mo.) to a final cell concentration of 2×10⁷ cells/ml. One hundred microliters of cells are dispensed into wells of a 96-well tissue culture plate for a final concentration of 2×10⁶ cells/well. Stimulations are conducted by adding 100 μl of the appropriate peptide or inactivated influenza virus diluted in RPMI-10. CD8⁺ T cells are stimulated with either the K^{d}-restricted HA₅₃₃₋₅₄₁ peptide (IYSTVASSL; SEQ ID NO: 1) (Winter, Fields, and Brownlee (1981) Nature 292:72) or the K^{d}-restricted NP₁₄₇₋₁₅₅ peptide (TYQRTRALV; SEQ ID NO:2) (Rotzschke et al., (1990) Nature 348:252). CD4⁺ T cells are stimulated with inactivated influenza virus (13,000 HAU per well of boiled influenza virus plus 13,000 HAU per well of formalin-inactivated influenza virus) plus anti-CD28 (1 μg/ml) and anti-CD49d (1 μg/ml) (Waldrop et al., (1998) J. Immunol. 161:5284). Negative control stimulations are done with media alone. Cells are then incubated as described below to detect extracellular cytokines by ELISA or intracellular cytokines by FACS staining.

Chromium Release Assay for CTL. CTL responses to influenza HA and NP are measured using procedures well known to those in the art (see, e.g., Current Protocols In Immunology, John E. Coligan et al., (eds), Unit 3, Wiley and Sons, New York, N.Y. (1994), and yearly updates including 2002). The synthetic peptide HA₅₃₃₋₅₄₁ IYSTVASSL (SEQ ID NO:1) (Winter, Fields, and Brownlee (1981) Nature 292:72) or NP₁₄₇₋₁₅₅ TYQRTRALV (SEQ ID NO:2) (Rotzschke et al., (1990) Nature 348:252) are used as the peptide in the target preparation step. Responder splenocytes from each animal are washed with RPMI-10 and resuspended to a final concentration of 6.3×10⁶ cells/ml in RPMI-10 containing 10 U/ml rat IL-2 (Sigma, St. Louis, Mo.). Stimulator splenocytes are prepared from naïve, syngeneic mice and suspended in RPMI-10 at a concentration of 1×10⁷ cells/ml. Mitomycin C is added to a final concentration of 25 μg/ml. Cells are incubated at 37° C./5% CO₂ for 30 minutes and then washed 3 times with RPMI-10. The stimulator cells are then resuspended to a concentration of 2.4×10⁶ cells/ml and pulsed with HA peptide at a final concentration of 9×10⁶M or with NP peptide at a final concentration of 2×10⁶M in RPMI-10 and 10 U/ml IL-2 for 2 hours at 37° C./5% CO₂. The peptide-pulsed stimulator cells (2.4×10⁶) and responder cells (6.3×10⁶) are then co-incubated in 24-well plates in a volume of 2 ml SM media (RPMI-10, 1 mM non-essential amino acids, 1 mM sodium pyruvate) for 5 days at 37° C./5% CO₂. A chromium-release assay is used to measure the ability of the in vitro stimulated responders (now called effectors) to lyse peptide-pulsed mouse mastocytoma P815 cells (MHC matched, H-2d). P815 cells are labeled with ⁵¹Cr by taking 0.1 ml aliquots of p815 in RPMI-10 and adding 25 μl FBS and 0.1 mCi radiolabeled sodium chromate (NEN, Boston, Mass.) in 0.2 ml normal saline. Target cells are incubated for 2 hours at 37° C./5% CO₂, washed 3 times with RPMI-10 and resuspended in 15 ml polypropylene tubes containing RPMI-10 plus HA (9×10⁻⁶M) or NP (1×10⁻⁶) peptide. Targets are incubated for 2 hours at 37° C./5% CO₂. The radiolabeled, peptide-pulsed targets are added to individual wells of a 96-well plate at 5×10⁴ cells per well in RPMI-10. Stimulated responder cells from individual immunization groups (now effector cells) are collected, washed 3 times with RPMI-10, and added to individual wells of the 96-well plate containing the target cells for a final volume of 0.2 ml/well. Effector to target ratios are 50:1, 25:1, 12.5:1 and 6.25:1. Cells are incubated for 5 hours at 37° C./5% CO₂ and cell lysis is measured by liquid scintillation counting of 25 μl aliquots of supernatants. Percent specific lysis of labeled target cells for a given effector cell sample is [100×(Cr release in sample-spontaneous release sample)/(maximum Cr release-spontaneous release sample)]. Spontaneous chromium release is the amount of radioactive released from targets without the addition of effector cells. Maximum chromium release is the amount of radioactivity released following lysis of target cells after the addition of TritonX-100 to a final concentration of 1%. Spontaneous release should not exceed 15%.

Detection of IFNγ or IL-5 in Bulk Culture Supernatants by ELISA. Bulk culture supernatants may be tested for IFNγ and IL-5 cytokine levels, which are known to correlate with Th1 and Th2-type response, respectively. Pooled splenocytes are incubated for 2 days at 37° C./5% CO₂, and then supernatants are harvested and pooled. All ELISA antibodies and purified cytokines are purchased from Pharmingen (San Diego, Calif.). Fifty microliters of purified anti-cytokine monoclonal antibody diluted to 5 μg/ml (rat anti-mouse IFNγ) or 3 μg/ml (rat anti-mouse IL-5) in coating buffer (0.1 M NaHCO₃, pH 8.2) is distributed per well of a 96-well ELISA plate (Corning, Corning, N.Y.) and incubated overnight at 4° C. Plates are washed, blocked, and rewashed with PBS-T. Standards (recombinant mouse cytokine) and samples are added to wells at various dilutions in RPMI-10 and incubated overnight at 4° C. for maximum sensitivity. Plates are washed 6 times with PBS-T. Biotinylated rat anti-mouse cytokine detecting antibody is diluted in PBS-T to a final concentration of 2 μg/ml and 100 μl was distributed per well. Plates are incubated for 1 hour at 37° C. and then washed 6 times with PBS-T. Streptavidin-AP (Gibco BRL, Grand Island, N.Y.) is diluted 1:2000 according to manufacturer's instructions, and 1001 is distributed per well. Plates are incubated for 30 mins. and washed an additional 6 times with PBS-T. Plates are developed by adding 100 μl/well of AP developing solution (BioRad, Hercules, Calif.) and incubating at room temperature for 50 minutes. Reactions are stopped by addition of 100 μl 0.4 M NaOH and read at OD₄₀₅. Data are analyzed using Softmax Pro Version 2.21 computer software (Molecular Devices, Sunnyvale, Calif.).

Intracellular Cytokine Staining and FACS Analysis. Splenocytes may be tested for intracellular IFNγ and IL-5 cytokine levels, which are known to correlate with Th1 and Th2-type response, respectively. Pooled splenocytes are incubated for 5-6 hours at 37° C. in a humidified atmosphere containing 5% CO₂. A Golgi transport inhibitor, Monensin (Phammingen, San Diego, Calif.), is added at 0.14 μl/well according to the manufacturer's instructions, and the cells are incubated for an additional 5-6 hours (Waldrop et al., (1998) J. Immunol. 161:5284). Cells are thoroughly resuspended and transferred to a 96-well U-bottom plate. All reagents (GolgiStop kit and antibodies) are purchased from Pharmingen (San Diego, Calif.) unless otherwise noted, and all FACS staining steps are done on ice with ice-cold reagents. Plates are washed 2 times with FACS buffer (1×PBS, 2% BSA, 0.1% w/v sodium azide). Cells are surface stained with 50 μl of a solution of 1:100 dilutions of rat anti-mouse CD80-APC, —CD69-PE, and —CD16/CD32 (FcγIII/RII; ‘Fc Block’) in FACS buffer. For tetramer staining (see below), cells were similarly stained with CD8β-TriColor, CD69-PE, CD16/CD32, and HA- or NP-tetramer-APC in FACS buffer. Cells are incubated in the dark for 30 mins. and washed 3 times with FACS buffer. Cells are permeabilized by thoroughly resuspending in 100 μl of Cytofix/Cytoperm solution per well and incubating in the dark for 20 mins. Cells are washed 3 times with Permwash solution. Intracellular staining is completed by incubating 50 μl per well of a 1:100 dilution of rat anti-mouse IFNγ-FITC in Permwash solution in the dark for 30 min. Cells are washed 2 times with Permwash solution and 1 time with FACS buffer. Cells are fixed in 2001 of 1% paraformaldehyde solution and transferred to microtubes arranged in a 96-well format. Tubes are wrapped in foil and stored at 4° C. until analysis (less than 2 days). Samples are analyzed on a FACScan® flow cytometer (Becton Dickenson, San Jose, Calif.). Compensations are done using single-stained control cells stained with rat anti-mouse CD8⁻FlTC, —PE, -TriColor, or -APC. Results are analyzed using FlowJo Version 2.7 software (Tree Star, San Carlos, Calif.).

Tetramers. HA and NP tetramers may be used to quantitate HA- and NP-specific CD8⁺ T cell responses following HA or NP immunization. Tetramers are prepared essentially as described previously (Flynn et al., (1998) Immunity 8:683). The present example utilizes the H-2 K^{d} MHC class I glycoprotein complexed with the synthetic influenza A/PR/8/34 virus peptide HA₅₃₃₋₅₄₁ (IYSTVASSL; SEQ ID NO: 1) (Winter, Fields, and Brownlee (1981) Nature 292:72) or NP₁₄₇₋₁₅₅ (TYQRTRALV; SEQ ID NO:2) (Rotzschke et al., (1990) Nature 348:252).

It is noted that the methods described in this example are applicable to a wide array agents, with only minor variations, which would be readily determinable by those skilled in the art.

### Example 11

### Immunization and Prevention of Parasitic Infection (Malaria)

The circumsporozoite protein (CSP) is a target of pre-erythocytic immunity (Hoffman et al., (1991) Science 252:520). In the Plasmodium yoelii (P. yoelii) rodent model system, passive transfer P. yoelii CSP-specific monoclonal antibodies (Charoenvit et al., (1991) J. Immunol. 146:1020), as well as adoptive transfer of P. yoelii CSP-specific CD8⁺ T cells (Rodrigues et al., (1991) Int. Immunol. 3:579, Weiss et al., (1992) J. Immunol. 149:2103) and CD4⁺ T cells (Renia et al., (1993) J. Immunol. 150:1471) are protective. Numerous vaccines designed to protect mice against sporozoites by inducing immune responses against the P. yoelii CSP have been evaluated.

Any Plasmodium sporozoite proteins known in the art capable of inducing protection against malaria usable in this invention may be used, such as P. falciparum, P. vivax, P. malariae, and P. ovale CSP; SSP2 (TRAP); Pfs16 (Sheba); LSA-1; LSA-2; LSA-3; MSA-1 (PMMSA, PSA, p185, p190); MSA-2 (Gymmnsa, gp56, 38-45 kDa antigen); RESA (Pf155); EBA-175; AMA-1 (Pf83); SERA (p113, p126, SERP, Pf140); RAP-1; RAP-2; RhopH3; PfHRP-II; Pf55; Pf35; GBP (96-R); ABRA (p110); Exp-1 (CRA, Ag5.1); Aldolase; Duffy binding protein of P. vivax; Reticulocyte binding proteins; HSP70-1 (p75); Pfg25; Pfg28; Pfg48/45; and Pfg230.

Materials and Methods

Castration. Surgical and/or chemical castration is performed as above.

Parasites. The 17XNL (nonlethal) strain of P. yoelii is used as described previously (U.S. Pat. No. 5,814,617).

Preparation of Irradiated P. yoelii Sporozoites. Preparation of irradiated P. yoelii sporozoites for immunization has been described previously (see, e.g., Franke et al., (2000) Infect. Immun. 68:3403). Briefly, sporozoites are isolated by the discontinuous gradient technique (Pacheco et al., (1979) J. Parisitol. 65:414) from infected Anopheles stephens mosquitoes that have been irradiated at 10,000 rads (¹³⁷Ce).

Immunization with Irradiated P. yoelii Sporozoites. Mice are intravenously immunized with 50,000 sporozoites at approximately 6 weeks following surgical castration or about 8 weeks following chemical castration via the tail vein. Booster immunizations of 20,000 to 30,000 sporozoites are optionally given at 4 weeks and 6 weeks following the primary immunization (see, e.g., Franke et al., (2000) Infect. Immun. 68:3403).

Plasmid DNA and DNA Immunization. Plasmid DNA encoding the full length P. yoelli CSP are known in the art. For instance, the pyCSP vector described in detail in Sedegah et al., ((1998) Proc. Natl. Acad. Sci. USA 95:7648) may be used.

Methods of DNA immunization are also well known in the art. For instance, methods of intradermal, intramuscular, and particle-mediated (“gene gun”) DNA immunizations are described in detail in, e.g., Current Protocols In Immunology, Unit 2.14, John E. Coligan et al., (eds), Wiley and Sons, New York, N.Y. (1994), and yearly updates including 2002.

Peptide Immunization. Methods of P. yoelii CSP peptide preparation are known in the art (see, e.g., Franke et al., (2000) Infect Immun. 68:3403).

Chromium Release Assay for CTL. Since CD8⁺ CTL against the P. yoelii CSP have been shown to adoptively transfer protection (Weiss et al., (1992) J. Immunol. 149:2103), and CD8⁺ T cells are required for the protection against P. yoelii induced by immunization with irradiated sporozoites (Weiss et al., (1988) Proc. Natl. Acad. Sci. USA 85:573), it must be determined if P. yoelii CSP vaccination (e.g., irradiated sporozoite, CSP peptide, or CSP DNA immunizations) elicits a CSP-specific CTL.

CTL responses are measured using procedures well known to those in the art (see, e.g., Current Protocols In Immunology, John E. Coligan et al., (eds), Unit 3, Wiley and Sons, New York, N.Y. (1994, and yearly updates including 2002). The general procedure described elsewhere herein for influenza HA and NP is used except that the cells are pulsed with the synthetic P. yoelli CSP peptide (281-296; SYVPSAEQILEFVKQI; SEQ ID NO:3).

Inhibition of Liver Stage Development Assay. The liver stage development assay and acquisition of mouse hepatocytes from mouse livers by in situ collagenase perfusion have been described previously (Franke et al., (1999) Vaccine 17:1201; Franke et al., (2000) Infect. Immun. 68:3403). Hepatocyte cultures are seeded onto eight-chamber Lab-Tek plastic slides at 1×10⁵ cells/chamber and incubated with 7.5×10⁴ P. yoelli sporozoites for 3 hours. The cultures are then washed and cultured for an additional 24 hours at 37° C./5% CO₂. Effector cells are obtained as described above for the chromium release assay for CTL and are added and cultured with the infected hepatocytes for about 24-48 hours. The cultures are then washed, and the chamber slides are fixed for 10 mins. in ice-cold absolute methanol. The chamber slides are then incubated with a monoclonal antibody (NYLS1 or NYLS3, both described previously in U.S. Pat. No. 5,814,617) directed against liver stage parasites of P. yoelii before incubating with FITC-labeled goat anti-mouse Ig. The number of liver-stage schizonts in triplicate cultures is then counted using an epifluorescence microscope. Percent inhibition is calculated using the formula [(control-test)/control)×100].

Infection and Challenge. For a lethal challenge dose, the ID₅₀ of P. yoelli sporozoites must be determined prior to experimental challenge. However, it is also initially possible to inject mice intravenously in the tail vein with a dose of about 50 to 100 P. yoelii sporozoites (non-lethal, strain 17XNL). Forty-two hours after intravenous inoculation, mice are sacrificed and livers are removed. Single cell suspensions of hepatocytes in medium are prepared, and 2×10⁵ hepatocytes are placed into each of 10 wells of a multi-chamber slide. Slides may be dried and frozen at −70° C. until analysis. To count the number of schizonts, slides are dried and incubated with NYLS1 before incubating with FITC-labeled goat anti-mouse Ig, and the numbers of liver-stage schizonts in each chamber are counted using fluorescence microscopy.

Once it is demonstrated that castration and/or immunization reduces the numbers of infected hepatocytes, blood smears are obtained to determine if immunization protects against blood stage infection. Mice are considered protected if no parasites are found in the blood smears at 5-14 days post-challenge.

To test the preventative efficacy of castration alone (no vaccination) from a P. yoelli sporozoite primary infection, castrated mice are infected and analyzed as described above. Sham-castrated mice are used as controls.

Human Studies. After establishing the efficacy in mice, large numbers of humans are immunized in a double blind placebo controlled field trial.

### Example 12

### Immunization and Prevention of Bacterial Infection (TB Ag85)

Tuberculosis (TB) is a chronic infectious disease of the lung caused by the pathogen Mycobacterium tuberculosis, and is one of the most clinically significant infections worldwide. (see, e.g., U.S. Pat. No. 5,736,524; for review see Bloom and Murray, (1993), Science 257, 1055.

M. tuberculosis is an intracellular pathogen that infects macrophages. Immunity to TB involves several types of effector cells. Activation of macrophages by cytokines, such as IFNγ, is an effective means of minimizing intracellular mycobacterial multiplication. Acquisition of protection against TB requires both CD8⁺ and CD4⁺ T cells (see, e.g., Orme et al., (1993) sJ. Infect. Dis. 167:1481). These cells are known to secrete Th1-type cytokines, such as IFNγ, in response to infection, and possess antigen-specific cytotoxic activity. In fact, it is known in the art that CTL responses are useful for protection against M. tuberculosis (see, e.g., Flynn et al., (1992) Proc. Natl. Acad. Sci. USA 89:12013).

Predominant T cell antigens of TB are those proteins that are secreted by mycobacteria during their residence in macrophages. These T cell antigens include, but are not limited to, the antigen 85 complex of proteins (85A, 85B, 85C) (Wiker and Harboe, ((1992) Microbiol. Rev. 56: 648) and ESAT-6 (Andersen (1994) Infect. Immunity, 62:2536). Other T cell antigens have also been described in the art (see, e.g., Young and Garbe (1991) Res. Microbiol. 142:55; Andersen (1992) J. Infect. Dis. 166:874; Siva and Lowrie (1994) Immunol. 82:244; Romain et al., (1993) Proc. Natl. Acad. Sci. USA 90:5322; and Faith et al., (1991) Immunol. 74:1).

The genes for each of the three antigen 85 proteins (A, B, and C) have been cloned and sequenced (see, e.g., Borremans et al., (1989) Infect. Immunity 57:3123); DeWit et al., (1994) DNA Seq. 4:267), and have been shown to elicit strong T cell responses following both infection and vaccination.

Materials and Methods

Castration of Mice. Surgical and/or chemical castration of BALB/c or C57BL/6 mice is performed as above.

Protein Immunization. General methods for Mycobacterium tuberculosis (TB) bacilli purification and immunization are known in the art (see, e.g., Current Protocols In Immunology, Unit 2.4, John E. Coligan et al., (eds), Wiley and Sons, New York, N.Y. (1994), and yearly updates including 2002). The purified TB may be prepared using preparative SDS-PAGE. Approximately 2 mg of the TB protein is loaded across the wells of a standard 1.5 mm slab gel using a large-tooth comb. An edge of the gel may be removed and stained following electrophoresis to identify the TB protein band on the gel. The gel region that contains the TB protein band is then sliced out of the gel, placed in PBS at a final concentration 0.5 mg purified TB protein per ml, and stored at 4° C. until use. The purified TB protein may then be emulsified with an equal volume of complete Freund's adjuvant (CFA) for immunization.

Approximately 6 weeks following surgical castration or about 8 weeks following chemical castration, 2 ml of the purified TB (0.5 mg/ml in PBS) is emulsified 2 ml CFA and stored at 4° C. The TB/CFA mixture is slowly drawn into and expelled through a 3 ml glass syringe attached to a 19-gauge needle, being certain to avoid excessive air bubbles. Once the emulsion is at a homogenous concentration, the needle is replaced by a 22-gauge needle, and all air bubbles are removed. The castrated and sham-castrated mice are injected intramuscularly with a 50 μl volume of the TB/CFA emulsion (immunization may also be done via the intradermal or subcutaneous routes). M. bovis BCG may also be used in a vaccine preparation.

A booster immunization can optionally be performed 4-8 weeks (or later) following the primary immunization. The TB adjuvant emulsion is prepared in the same manner described above, except that incomplete Freund's adjuvant (IFA) is used in place of CFA for all booster immunizations. Further booster immunizations can be performed at 2-4 week (or later intervals) thereafter.

Plasmid DNA. Suitable Ag85-encoding DNA sequences and vectors have been described previously (see, e.g., U.S. Pat. No. 5,736,524). Other suitable expression vectors would be readily ascertainable by those skilled in the art.

Antigen 85 DNA Immunization. Methods of DNA immunization are well known in the art. For instance, methods of intradermal, intramuscular, and particle-mediated (“gene gun”) DNA immunizations are described in detail in, e.g., Current Protocols In Immunology, Unit 2.14, John E. Coligan et al., (eds), Wiley and Sons, New York, N.Y. (1994), and yearly updates including 2002).

Cytokine-encoding DNAs are optionally administered to shift the immune response to a desired Th1- or a Th2-type immune response. Th1-inducing genetic adjuvants include, e.g., IFN-γ and IL-12. Th2-inducing genetic adjuvants include, e.g., IL-4, IL-5, and IL-10. For review of the preparation and use of Th1- and Th2-inducing genetic adjuvants in the induction of immune response, see, e.g., Robinson, et al., (2000) Adv. Virus Res. 55:1-74.

Approximately 6 weeks following surgical castration or about 8 weeks following chemical castration, mice are intramuscularly injected with 200 μg of DNA diluted in 100 μl saline.

Booster DNA immunizations are optionally administered at 4 weeks post-prime and 2 weeks post-boost.

Enzyme-Linked Immunosorbant Assays. At various time periods pre- and post-immunization, mice from each group are bled, and individual mouse serum is tested using standard quantitative ELISA to assess anti-Ag85 specific IgG levels in the serum. IgG1 and IgG2a levels may optionally be tested, which are known to correlate with Th2 and Th-type antibody responses, respectively.

Serum is collected at various time-points pre- and post-prime and post-boost, and analyzed for the presence of anti-Ag85 specific antibodies in serum. Basic ELISA methods are described elsewhere herein, except purified Ag85 protein is used.

Cytokine Assays. Spleen cells from vaccinated mice are analyzed for cytokine secretion in response to specific Ag85 restimulation, as described, e.g., in Huygen et al., (1992) Infect. Immunity 60:2880, and in U.S. Pat. No. 5,736,524. Briefly, spleen cells are incubated with culture filtrate (CF) proteins from M. bovis BCG purified Ag85A or the C57BL/6 T cell epitope peptide (amino acids 241-260).

Four weeks post-prime and 2 weeks post-boost (or later), cytokines are assayed using standard bio-assays for IL-2, IFNγ and IL-6, and by ELISA for IL-4 and IL-10 using methods well known to those in the art. See, e.g., Current Protocols In Immunology, Unit 6, John E. Coligan et al., (eds), Wiley and Sons, New York, N.Y. (1994), and yearly updates including 2002.

Mycobacterial Infection and Challenge. To test the efficacy of the vaccinations, mice are challenged by intravenous injection of live M. bovis BCG (0.5 mg). At various time-points post-challenge, BCG multiplication is analyzed in both mouse spleens and lungs. Positive controls are naïve mice (castrated and/or sham-castrated as appropriate) receiving a challenge dose.

To test the efficacy of sex steroid ablation to prevent primary infection, live M. bovis BCG are injected similarly to that described in the challenge experiment above. Sham-castrated mice are used as controls.

The number of colony-forming units (CFU) in the spleen and lungs of the challenged, vaccinated mice, as well as in the lungs of the castrated, primary infected mice is expected to be substantially lower than in negative control animals, which is indicative with protection in the live M. bovis challenge model.

### Example 13

### Immunization and Prevention of Cancer

To determine if sex steroid ablation is effective in preventing cancer and/or in eliciting a protective immune response following vaccination with a cancer antigen, the following studies are performed.

Materials and Methods

Castration. C57BL/6 mice surgical and/or chemical castration is performed as above.

CEA Immunization. Approximately 6 weeks following surgical castration or about 8 weeks following chemical castration, mice are inoculated with an adenovirus vector encoding the human carcinoembryonic antigen (CEA) gene (MC38-CEA-2) (Conry et al., (1995) Cancer Gene Ther. 2:33), such as AdCMV-hcea described in U.S. Pat. No. 6,348,450. Alternatively, a plasmid DNA encoding the human CEA gene is injected into the mouse (e.g., intramuscularly into the quadriceps muscle) utilizing one of the various methods of DNA vaccination described elsewhere herein.

Tumor Challenge. To assess the efficacy of sex steroid ablation on anti-tumor activity of mice immunized with CEA, mice are subjected to a tumor challenge. At various time-points post-immunization, syngeneic tumor cells expressing the human CEA gene (MC38-CEA-2) (Conry et al., (1995) Cancer Gene Ther. 2:33) are inoculated into the mice. Mice are observed every other day for development of palpable tumor nodules. Mice are sacrificed when the tumor nodules exceed 1 cm in diameter. The time between inoculation and sacrifice is the survival time.

To test the efficacy of sex steroid ablation preventing tumors, tumor cells expressing the human CEA gene are inoculated into castrated, non-vaccinated mice as outlined above. Sham-castrated mice are used as controls.

### Example 14

### Transplantation of Genetically Modified HSC (Gene Therapy)

### I. SCID-hu Mouse Model

Materials and Methods

Mice. SCID-hu mice are prepared essentially as described previously (see, e.g., Namikawa et al., (1990) J. Exp. Med. 172:1055 and Bonyhadi et al., (1997) J. Virol. 71:4707) by surgical transplantation of human fetal liver and thymus fragments into CB-17 scid/scid mice. Methods for the construction of SCID-hu Thy/Liv mice can also be found, e.g., in Current Protocols In Immunology, Unit 4.8, John E. Coligan et al., (eds), Wiley and Sons, New York, N.Y. (1994), and yearly updates including 2002.

Surgical Castration of Mice. The SCID-hu mice are anesthetized by intraperitoneal injection of 3040 μl of a mixture of 5 ml of 100 mg/ml ketamine hydrochloride (Ketalar®; Parke-Davis, Caringbah, NSW, Australia) plus 1 ml of 20 mg/ml xylazine (Rompun®; Bayer Australia Ltd., Botany NSW, Australia) in saline. Surgical castration is performed as described above by a scrotal incision, revealing the testes, which are tied with suture and then removed along with surrounding fatty tissue. The wound is closed using surgical staples. Sham-castrated mice prepared following the above procedure without removal of the testes are used as controls.

Chemical Castration. Chemical castration is performed as above.

Isolation of Human CD34⁺ HSC. Human cord blood (CB) HSC is collected and processed using techniques well known to those skilled in the art (see, e.g., DiGusto et al., (1997) Blood, 87:1261 (1997), Bonyhadi et al., (1997) J. Virol. 71:4707). A portion of each CB sample is HLA phonotyped for the MA2.1 surface molecule. CD34⁺ cells are enriched using immunomagnetic beads using the method described in Bonyhadi et al., ((1997) J. Virol. 71:4707). Briefly, CB cells are incubated with anti-CD34 antibody (QBEND-10, Immunotech) and then washed and resuspended at a final concentration of 2×10⁷ cells/ml. CD34⁺ cells are then enriched using goat-anti-mouse IgG1 magnetic beads (Dynal) following manufacturer's instructions. The CD34⁺ cells are then incubated with 50 μl of glycoprotease (O-sialoglycoprotein endopeptidase), which causes release of the CD34⁺ cells from the immunomagnetic beads. The beads are removed using a magnet, and the cells are then subjected to flow cytometry using conjugated anti-CD34-PE to determine the total level of CD34⁺ cells present in the population. Alternatively, the cells are magnetically labeled with anti-CD34 and sorted on an autoMACS™. The autoMACS™ may be used for magnetic pre-sorting of cells before further flow cytometric sorting. For example, anti-FITC- or anti-PE MACS® MicroBeads, may be added to the FITC or PE stained cells. Then the cells are sorted on the autoMACS™ according to their magnetic labeling. The positive and negative fractions may then be collected for sorting by flow cytometry.

Optionally, HSC are expanded ex vivo with IL-3, IL-6, and either SCF or LIF (10 ng/ml each).

RevM10 Vectors and Preparation of Genetically Modified (GM) HSC. RevM10 is known in the art, and has been described extensively in studies of GM HSC for the survival of T cells in HIV-infected patients (see, e.g., Woffendin et al., (1996) Proc. Natl. Acad. Sci. USA, 93:2889; for review, see Amado et al., (1999) Front. Biosci. 4:d468). The HIV Rev protein is known to affect viral latency in HIV-infected cells and is essential for HIV replication. RevM10 is a derivative of Rev because of mutations within the leucine-rich domain of Rev that interacts with cell factors. RevM10 has a substitution of aspartic acid for leucine at position 78 and of Leucine for glutamic acid at position 79. The result of these mutations is that RevM10 is able to compete effectively with the wild-type HIV Rev for binding to the Rev-responsive element (RRE).

Any of the RevM10 gene transfer vectors known and described in the art may be used. For example, the retroviral RevM10 vector, pLJ-RevM10, is used to transduce the HSC. The pLJ-RevM10 vector has been shown to enhance T cell engraftment after delivery into HIV-infected individuals (Ranga et al., (1998) Proc. Natl. Acad. Sci. USA 95:1201). Other methods of construction and retroviral vectors suitable for the preparation of GM HSC are well known in the art (see, e.g., Bonyhadi et al., (1997) J. Virol. 71:4707).

In another example, the pRSV/TAR RevM10 plasmid is used for non-viral vector delivery using particle-mediated gene transfer into the isolated target HSC essentially as described in Woffendin et al., (1994) Proc. Natl. Acad. Sci. USA, 91:11581. The pRSV/TAR RevM10 plasmid contains the Rous sarcoma virus (RSV) promoter and tat-activation response element (TAR) from −18 to +72 of HIV is used to express the RevM10 open reading frame may also be used (Woffendin et al., (1994) Proc. Natl. Acad. Sci. USA, 91:11581; Liu et al., (1997) Gene Ther. 1:32). In vitro transfection of this plasmid into human PBL has previously been shown to provide resistance to HIV infection (Woffendin et al., (1994) Proc. Natl. Acad. Sci. USA, 91:11581).

A marker gene, such as the Lyt-2α (murine CD8α) gene, may also be incorporated into the RevM10 vector for ease of purification and analysis of GM HSC by FACS analysis in subsequent steps (see, e.g., Bonyhadi et al., (1997) J. Virol. 71:4707).

A ΔRev10, which contains a deletion of the methionine (Met) initiation codon (ATG), as well as a linker comprising a series of stop codons inserted in-frame into the BglII site of the RevM10 gene, is constructed and used as a negative control (see, e.g., Bonyhadi et al., (1997) J. Virol. 71:4707).

Injection of GM HSC Into Mice. SCID-hu mice are analyzed, and the mice determined to be HLA mismatched (MA2.1). With respect to the human donor, HSC are given approximately 400 rads of total body irradiation (TBI) about four months following the thymic and liver grafts in an effort to eliminate the cell population. After TBI, mice are reconstituted with the RevM10 GM HSC (see above) as described previously (see, e.g., DiGusto et al., (1997) Blood, 87:1261, Bonyhadi et al., (1997) J. Virol. 71:4707). Control mice are injected with unmodified HSC or with HSC that have been modified with the ΔRevM10 gene or an irrelevant gene.

Analysis of GM HSC by Flow Cytometry. Approximately 8 to 12 weeks after GM HSC reconstitution, the Thy/Liv grafts are removed, and the thymocytes are obtained and analyzed for the HLA pheonotype (MA2.1), and the distribution of CD4⁺, CD8⁺, and Lyt2 (the “marker” murine homolog of CD8α) surface expression using methods of flow cytometry and FACS analysis readily known to those skilled in the art (see, e.g., Bonyhadi et al., (1997) J. Virol. 71:4707; see also Current Protocols In Immunology, Units 4.8 and 5, John E. Coligan et al., (eds), Wiley and Sons, New York, N.Y. (1994), and yearly updates including 2002). Thymocytes are also tested for transgenic DNA with primers specific for the RevM10 gene using standard PCR methods.

Analysis of GM HSC Resistance to HIV Infection. Approximately 8 to 12 weeks (or later) after GM HSC reconstitution, the Thy/Liv grafts are removed and the thymocytes are obtained from the GM HSC reconstituted SCID-hu mice. The thymocytes are stimulated in vitro and infected with the JR-CSF molecular isolate of HIV-1 as described previously (Bonyhadi et al., (1997) J. Virol. 71:4707). Briefly, the thymocytes are stimulated in vitro in the presence of irradiated allogeneic feeder cells (10⁶ peripheral blood mononuclear cells/ml and 10⁵ JY cells/ml) in RPMI medium containing 10% FCS, 50 μg/ml streptomycin, 50 U/G penicillin G, 1× MEM vitamin solution, 1× insulin transferring-sodium selenite medium supplement (Sigma), 40 U human rIL-2/ml, and 2 μg/ml phytohemagglutinin (PHA) (Sigma). About every 10 days, cells are restimulated with feeder cells and PHA as described previously in Vandekerckhove et al., (1992) J. Exp. Med. 1:1033. Approximately 5 days after stimulation, cells were sorted on the basis of donor HLA phenotype (MA2.1) and Lyt2 (the “marker” murine homolog of CD8α). Sorted cells are restimulated and may be expanded to increase the cell composition to greater than about 90% purity. CD4⁺/Lyt2⁺ cells are then sorted out and an aliquot of approximately 5×10⁴ of the sorted cells are placed in multiple wells of a 96-well U bottom tissue culture plate. About 200 TCID₅₀ of EW, an HIV-1 primary isolate, or 1000 TCID₅₀ of JR-CSF, an HIV-1 molecular isolate, are added to each well. Methods of virus stock preparation have been described previously (Bonyhadi et al., (1993) Nature, 363:728). Medium is changed every day from days 3 to 12. Aliquots of supernatant are collected every other day and stored at −80° C. until use. Tissue culture supernatants are then analyzed using a p24 ELISA following manufacturer's instructions (Coulter).

### II. Therapy of HIV Infected Individual

Materials and Methods.

Isolation of Human CD34⁺ HSC. As most HIV infected patients have very low titers of HSC, it is possible to use a donor to supply cells. Where practical, the level of HSC in the donor blood is enhanced by injecting into the donor granulocyte-colony stimulating factor (G-CSF) at 10 μg/kg for 2-5 days prior to cell collection.

In this example, human cord blood (CB) HSC are collected and processed using techniques well known to those skilled in the art (see, e.g., DiGusto et al., (1997) Blood, 87:1261; Bonyhadi et al., (1997) J. Virol. 71:4707). A portion of each CB sample is HLA phenotyped, and the CD34⁺ donor cells are purified from the donor blood (or BM), such as by using a flow cytometer or immunomagnetic beading, essentially as described above. Donor-derived HSC are identified by flow cytometry as being CD34⁺.

Optionally, HSC are expanded ex vivo with IL-3, IL-6, and either SCF or LIF (10 ng/ml each).

RevM10 Vectors and Preparation of Genetically Modified (GM) HSC. Any of the RevM10 gene transfer vectors known and described in the art, including those described in the mouse studies above, may be used. Methods of gene transduction using GM retroviral vectors or gene transfection using particle-mediated delivery are also well known in the art, and are described elsewhere herein.

As described above, a retroviral vector may be constructed to contain the trans-dominant mutant form of HIV-1 rev gene, RevM10, which has been shown to inhibit HIV replication (Bonyhadi et al., (1997) J. Virol. 71:4707). Amphotropic vector-containing supernatants are generated by infection with filtered supernatants from ecotropic producer cells that were transfected with the vector.

The collected CD34⁺ cells are optionally pre-stimulated for 24 hours in LCTM media supplemented with IL-3, IL-6 and SCF or LIF (10 ng/ml each) to induce entry of the cells into the cell cycle.

In this example, CD34⁺-enriched HSC undergo transfection by a linearized RevM10 plasmid utilizing particle-mediated (“gene gun”) transfer essentially as described in Woffendin et al., (1996) Proc. Natl. Acad. Sci. USA, 93:2889. However, if retroviral transduction is done, supernatants containing the vectors are repeatedly added to the cells for 2-3 days to allow transduction of the vectors into the cells.

HAART Treatment of HIV-Infected Patients. HAART therapy is begun before T cell depletion and sex steroid ablation, and therapy is maintained throughout the procedure to reduce the viral titer.

T Cell Depletion. T cell depletion is performed as given in Example 5 to remove as many HIV infected cells as possible.

Sex Steroid Ablation Therapy. The HIV-infected patient is given sex steroid ablation therapy as described in Example 6.

Injection of GM HSC Into Patients. Prior to injection, the GM HSC are expanded in culture for approximately 10 days in X-Vivo 15 medium comprising II-2 (Chiron, 300 IU/ml). At approximately 1-3 weeks post LHRH agonist delivery, just before or at the time the thymus begins to reactivate, the patient is injected with the genetically modified HSC, optimally at a dose of about 24×10⁶ cells/kg. Optionally, G-CSF may also be injected into the recipient to assist in expansion of the GM HSC.

Immediately prior to patient infusion, the GM HSC are washed four times with Dulbecco's PBS. Cells are resuspended in 100 ml of saline comprising 1.25% human albumin and 4500 U/ml IL-2, and infused into the patient over a course of 30 mins.

Following sex steroid ablation and injection of the GM HSC in the HIV-infected patient, all new T cells (as well as DC, macrophages, etc.) are resistant to subsequent infection by this virus. Injection of allogeneic HSC into a patient undergoing thymic reactivation means that the HSC will enter the thymus. The reactivating or reactivated thymus takes up the genetically modified HSC and converts them into donor-type T cells and DC, while converting the HSC of the recipient into recipient-type T cells and DC. By inducing deletion by cell death, or by inducing tolerance through immunoregulatory cells, the donor DC will tolerize any T cells that are potentially reactive with recipient.

When the thymic chimera is established, and the new cohort of mature T cells have begun exiting the thymus, reduction and eventual elimination of immunosuppression occurs.

Post-Infusion Studies. Following infusion, the persistence and half life of GM HSC in the HIV-infected patient is tested periodically using limiting dilution PCR of PBL samples obtained from the patient essentially as described in Woffendin et al., (1996) Proc. Natl. Acad. Sci. USA, 93:2889. The relative level of GM HSC in the infected patient is compared to the negative control patient that received the ΔRevM10 vector.

Various standard hematologic (e.g., CD4⁺ T cell counts), immunologic (e.g., neutralizing antibody titers), and virologic (e.g., viral titer) studies are also performed using methods well known to those skilled in the art.

Termination of Immunosuppression. Termination of immunosuppression is performed as given in Example 16.

### Example 15

### Alternative Protocols

In the event of a shortened time available for transplantation of donor cells, tissue or organs, the timeline as used in Examples 1-14 is modified. T cell ablation or other immune cell depletion and sex steroid ablation are begun at the same time. T cell ablation or other immune cell depletion is maintained for about 10 days, while sex steroid ablation is maintained for around 3 months. In one embodiment, HSC transplantation is performed when the thymus starts to reactivate, at around 10-12 days after start of the combined treatment.

In an even more shortened time table, the two types of ablation and the HSC transplant are started at the same time. In this event, T cell ablation or other immune cell depletion is maintained 3-12 months, for example, for 3-4 months.

### Example 16

### Termination of Immunosuppression

When the thymic chimera is established and the new cohort of mature T cells have begun exiting the thymus, blood is taken from the patient and the T cells examined in vitro for their lack of responsiveness to donor cells in a standard mixed lymphocyte reaction (see, e.g., Current Protocols In Immunology, John E. Coligan et al., (eds), Wiley and Sons, New York, N.Y. (1994), and yearly updates including 2002). If there is no response, the immunosuppressive therapy is gradually reduced to allow defense against infection. If there is no sign of rejection, as indicated in part by the presence of activated T cells in the blood, the immunosuppressive therapy is eventually stopped completely. Because the HSC have a strong self-renewal capacity, the hematopoietic chimera so formed will be stable theoretically for the life of the patient (that is, a normal, non-tolerized and non-grafted person).

### Example 17

### Use of LHRH Agonist to Reactivate the Thymus in Humans

Materials and Methods:

In order to show that a human thymus can be reactivated by the methods of this invention, these methods were used on patients who had been treated with chemotherapy for prostate cancer.

Patients. Sixteen patients with Stage I-III prostate cancer (assessed by their prostate specific antigen (PSA) score) were chosen for analysis. All subjects were males aged between 60 and 77 who underwent standard combined androgen blockade (CAB) based on monthly injections of GnRH agonist 3.6 mg goserelin acetate (Zoladex®) or 7.5 mg leuprolide (Lupron®) treatment per month for 4-6 months prior to localized radiation therapy for prostate cancer, as necessary.

FACS Analysis. The appropriate antibody cocktail (20 μl) was added to 200 μl whole blood and incubated in the dark at room temperature (RT) for 30 mins. RBC were lysed and remaining cells washed and resuspended in 1% PFA for FACS analysis. Samples were stained with antibodies to CD19⁻FITC, CD4⁻FITC, CD8⁻APC, CD27⁻FITC, CD45RA-PE, CD45RO-CyChrome, CD62L-FITC and CD56-PE (all from Pharmingen, San Diego, Calif.).

Statistical Analysis. Each patient acted as an internal control by comparing pre- and post-treatment results and were analyzed using paired student t-tests or Wilcoxon signed rank tests.

Results:

Prostate cancer patients were evaluated before and 4 months after sex steroid ablation therapy. The results are summarized in FIGS. 19-23. Collectively, the data demonstrates qualitative and quantitative improvement of the status of T cells in many patients.

### I. The Effect of LHRH Therapy on Total Numbers of Lymphocytes and T Cells Subsets Thereof:

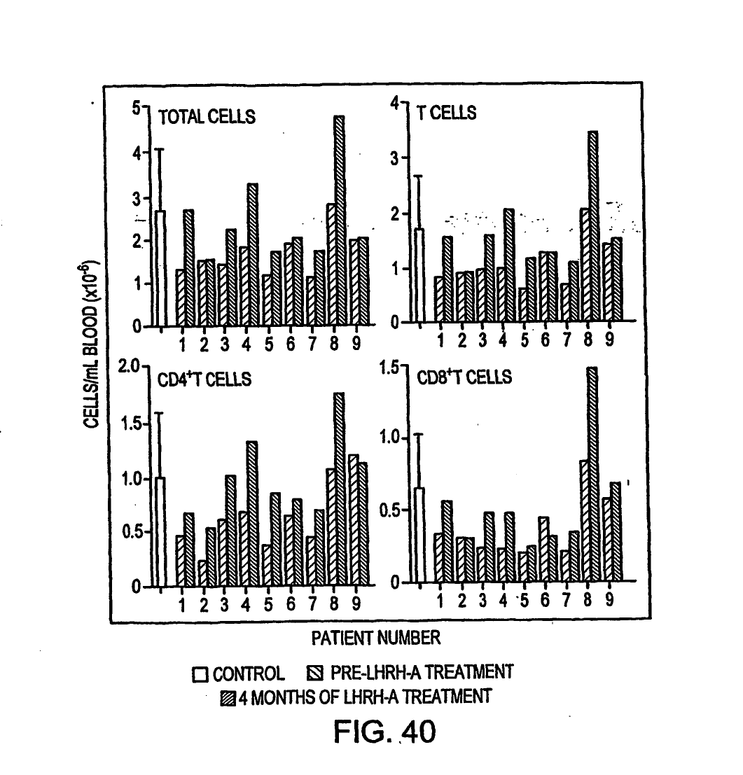

The phenotypic composition of peripheral blood lymphocytes was analyzed in patients (all >60 years) undergoing LHRH agonist treatment for prostate cancer (FIG. 40). Patient samples were analyzed before treatment and 4 months after beginning LHRH agonist treatment. Total lymphocyte cell numbers per ml of blood were at the lower end of control values before treatment in all patients.

Following treatment, six out of nine patients showed substantial increases in total lymphocyte counts (in some cases a doubling of total cells was observed). Correlating with this was an increase in total T cell numbers in six out of nine patients. Within the CD4⁺ subset, this increase was even more pronounced with eight out of nine patients demonstrating increased levels of CD4⁺ T cells. A less distinctive trend was seen within the CD8⁺ subset with four out of nine patients showing increased levels, albeit generally to a smaller extent than CD4⁺ T cells.

### II. The Effect Of LHRH Therapy on the Proportion of T Cell Subsets:

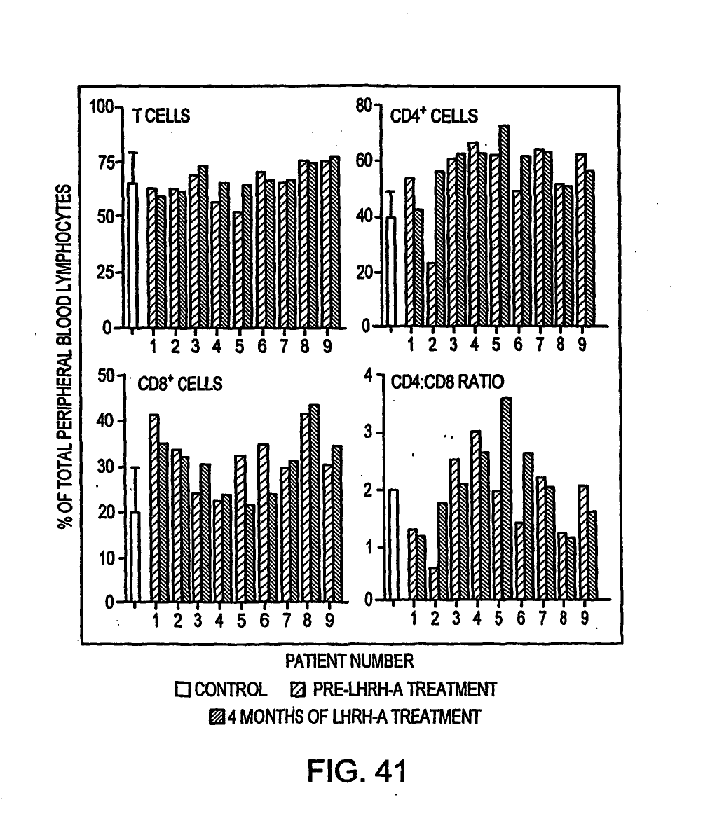

Analysis of patient blood before and after LHRH agonist treatment demonstrated no substantial changes in the overall proportion of T cells, CD4⁺ or CD8⁺ T cells and a variable change in the CD4⁺:CD8⁺ ratio following treatment (FIG. 41). This indicates that there was little effect of treatment on the homeostatic maintenance of T cell subsets despite the substantial increase in overall T cell numbers following treatment. All values were comparative to control values.

### III. The Effect of LHRH Therapy on the Proportion of B Cells and Myeloid Cells:

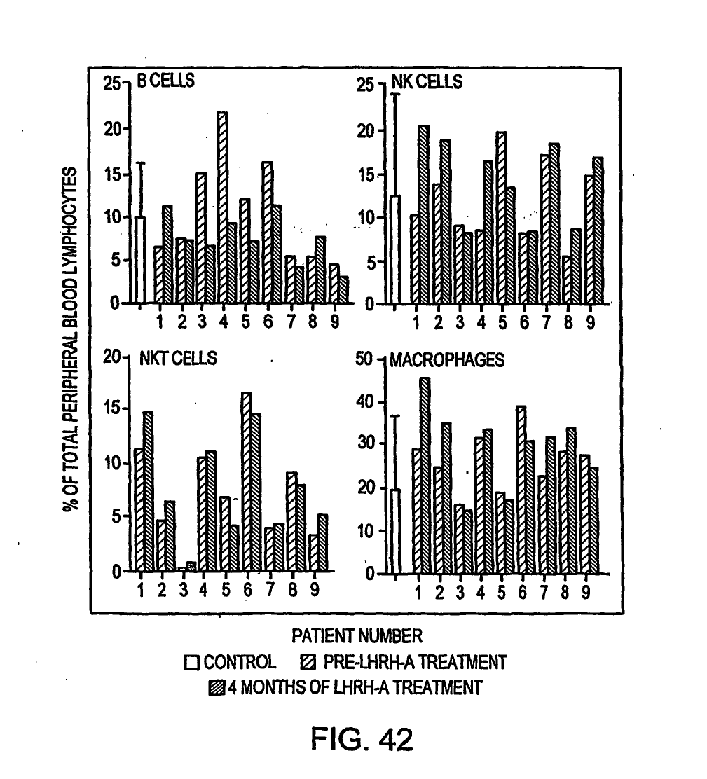

Analysis of the proportions of B cells and myeloid cells (NK, NKT and macrophages) within the peripheral blood of patients undergoing LHRH agonist treatment demonstrated a varying degree of change within subsets (FIG. 42). While NK, NKT and macrophage proportions remained relatively constant following treatment, the proportion of B cells was decreased in four out of nine.

### IV. The Effect of LHRH Agonist Therapy on the Total Number of B Cells and Myeloid Cells:

Analysis of the total cell numbers of B and myeloid cells within the peripheral blood post-treatment showed clearly increased levels of NK (five out of nine patients), NKT (four out of nine patients) and macrophage (three out of nine patients) cell numbers post-treatment (FIG. 43). B cell numbers showed no distinct trend with two out of nine patients showing increased levels; four out of nine patients showing no change, and three out of nine patients showing decreased levels.

V. The Effect of LHRH Therapy on the Level of Naïve Cells Relative to Memory Cells:

The major changes seen post-LHRH agonist treatment were within the T cell population of the peripheral blood. In particular, there was a selective increase in the proportion of naïve (CD45RA⁺) CD4⁺ cells, with the ratio of naïve (CD45RA⁺) to memory (CD45RO⁺) in the CD4⁺ T cell subset increasing in six out of nine patients (data not shown).

### VI. Conclusion

Thus, it can be concluded that LHRH agonist treatment of an animal such as a human having an atrophied thymus can induce regeneration of the thymus. A general improvement has been shown in the status of blood T lymphocytes in these prostate cancer patients who have received sex-steroid ablation therapy. It is likely that such cells are derived from the thymus as no other source of mainstream (TCRαβ+CD8 αβ chain) T cells has been described. Gastrointestinal tract T cells are predominantly TCR γδ or CD8 α chain.

### Example 18

### Regeneration of the Peripheral Immune Cell Pool Following Hematopoietic Stem Cell Transplantation in Humans

I. Allogeneic and Autologous HSCT

This example relates to clinical trials undertaken with HSCT patients. To assess the clinical potential for restoring thymus and bone marrow function in humans, prostate cancer patients (>60 years) who routinely undergo sex-steroid ablation therapy based on LHRH-agonist (chemical castration) treatment have been analyzed. Patients were examined at the time of presentation and after 4-months of treatment, by which time serum testosterone concentration was at castrate levels for all patients.

Materials and Methods:

Patients. Eighty-two patients were all due to undergo high-dose therapy (HDT) with PBSCT for malignant disease or bone marrow failure (n=22 for allogeneic control patients, n=20 for allo LHRH-A treated patients, n=20 for autologous controls, and n=20 for autologous LHRH-A treated patients). Test patients were given 3.6 mg (effective for 4 weeks) Zoladex (LHRH-A) 3-weeks prior to autologous or allogeneic stem cell transplantation and then monthly injections for 4 months. All patients were analyzed pre-treatment, weekly for 5 weeks after transplantation and then monthly up to 12 months. Ethics approval was obtained from The Alfred Committee for Ethical Research on Humans (Trial Number 01/006).

FACS Analysis of Whole Peripheral Blood. The appropriate antibody cocktail (20 μl) was added to 200 μl whole blood and incubated in the dark at room temperature (RT) for 30 mins. RBC were lysed and remaining cells washed and resuspended in 1% PFA for FACS analysis. Samples were stained with antibodies to CD19-FITC, CD4-FITC, CD8⁻APC, CD27-FITC, CD45RA-PE, CD45RO-CyChrome, CD62L-FITC and CD56-PE (all from Pharmingen, San Diego, Calif.).

Ki67 Analysis. For detection of proliferating cells, samples were surface stained with CD27-FITC, CD45RO-CyChrome, and CD4- or CD8⁻APC (Pharmingen, San Diego, Calif.). Following red cell lysis, samples were incubated for 20 min., RT, in the dark in 500 μl of 1× FACS permeabilizing solution (Becton-Dickinson, USA 1× solution was made from 10× stock in R.O.H₂O). Washed samples (2 ml FACS buffer, 5 mins., 600_{gmax}, RT) were incubated with either anti-Ki67-PE or anti-Ki67-FITC (or the appropriate isotype controls) for 30 mins. at RT, in the dark. Samples were then washed and resuspended in 1% PFA for analysis.

Preparation of PBMC. Purified Lymphocytes were Prepared for T-Cell Stimulation assays and TREC analysis, by ficoll-hypaque separation and following centrifugation, the plasma layer was removed and stored at −20° C. prior to analysis of sex steroid levels. Cells not used for T lymphocyte stimulation assays were resuspended in freezing media and stored in liquid nitrogen prior to TREC analysis.

T Lymphocyte Stimulation Assay. For mitogen stimulation, purified lymphocytes were plated out in 96-well round-bottom plates at a concentration of 1×10⁵ cells/well in 100 μl of RPMI-FCS. Cells were incubated at 37° C., 5% CO₂ with PHA in doses from 1-10 μg/ml. For TCR-specific stimulation, cells were incubated for 48 hours on plates previously coated with purified anti-CD3 (1-10 μg/ml) and anti-CD28 (10 μtg/ml). Following plaque formation (48-72 hours), 1 μCi of ³H-thymidine was added to each well and plates incubated for a further 16-24 hours. Plates were harvested onto filter mats and incorporation of ³H-Thymidine was determined using liquid scintillation on a β-counter (Packard-Coulter, USA).

TREC Analysis:

Cell Sorting. Frozen samples were rapidly thawed and stained with anti-CD4-FITC and anti-CD8-APC for 30 mins. on ice, washed (2 ml FACS buffer) and fixed with 3% formalin in PBS (with agitation). Samples were incubated for a further 30 mins., washed and resuspended in 500 μl FACS buffer for sorting. CD4⁺ and CD8⁺ cell populations were sorted on a MoFlo® cell sorter (Cytomation Inc.).

DNA Isolation. Cells were sorted and resuspended in Proteinase K (PK) digestion buffer (2×10⁵ cells/20 μl of a 0.8 mg/mL solution). Samples were incubated for 1 hour at 56° C. followed by 10 mins. at 95° C. to inactivate the proteinase.

Real-Time PCR Using Molecular Beacons. Real-time PCR for analysis of TREC content in sorted cells was performed as described previously (Zhang et al., (1999) J. Exp. Med. 190:725). The primers were sense, 5′-GGATGGAAAACACAGTGTGACATGG-3′ (SEQ ID NO:4) and antisense, 5′-CTGTCAACAAAGGTGATGCCACATCC-3′ (SEQ ID NO:5). One cycle of denaturation (95° C. for 10 mins.) was performed, followed by 45 cycles of amplification (94° C. for 30 s, 60° C. for 30 s, and 72° C. for 30s). To normalize for cell equivalents in the input DNA, a separate real-time PCR assay was used to quantify the CCR5 coding sequence, which contains no pseudogenes.

Statistical Analysis. Statistical analysis was performed using Instat II software. For prostate cancer HSCT studies, a Mann-Whitney U-test was performed. For human studies, each patient acted as an internal control by comparing pre- and post-treatment results and were analyzed using paired student t-tests or Wilcox on signed rank tests.

Results:

FIG. 49 depicts analysis of natural killer (NK) cell recovery at various time-points (2-8 weeks) following HSCT in control patients. As shown in FIGS. 49A-B, respectively, a similar trend was observed for both control allogeneic and autologous transplant recipients. In contrast, allogeneic patients who were given LHRH-A treatment 3 weeks prior to HSCT showed a significantly higher number of NKT (Vβ24+Vβ11+) cells from D14-5M post-transplant (FIG. 49C; data is expressed as mean ±1 SEM of 6-20 patients; *=p≦0.05). NKT cells were analyzed based on their Vβ24⁺Vβ11⁺ phenotype.

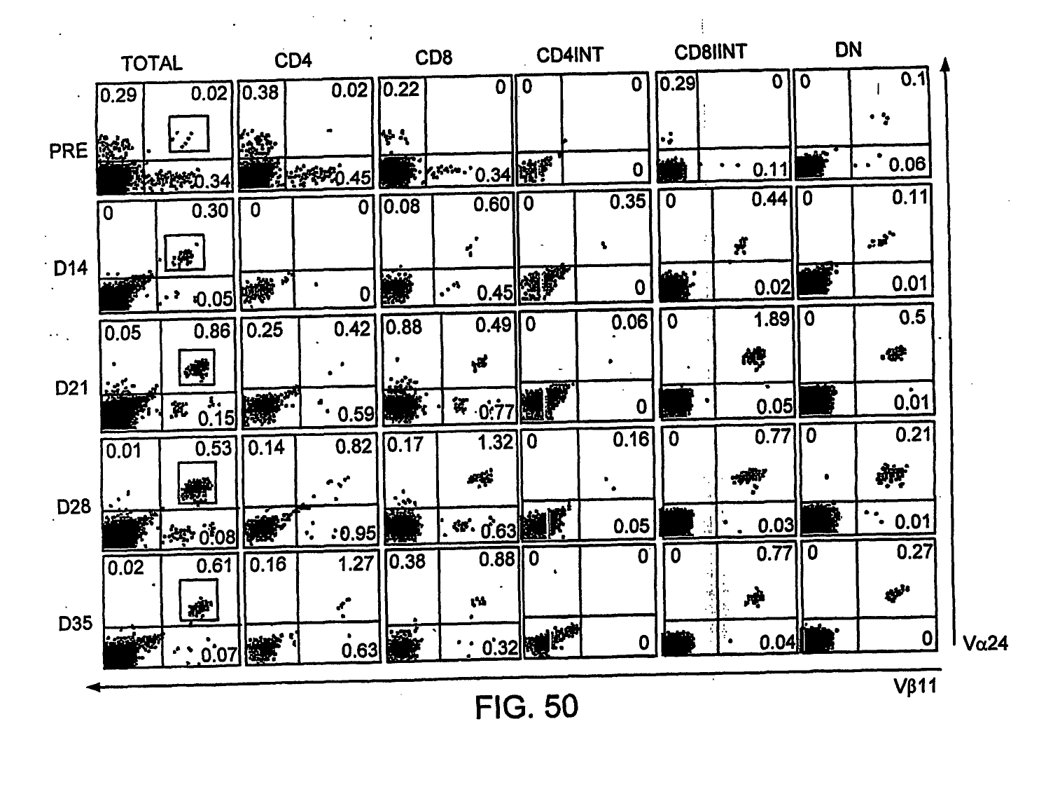

FIG. 50 depicts FACS analysis of NKT cell reconstitution at various time-points (day 14, 21, 28 and 35) following HSCT in control patients. An early recovery was observed in allogeneic patients and was seen predominantly within the CD8⁺ population early post-transplant, which indicated extrathymic routes of regeneration. Also, CD4⁺ NKT cells were evident from 1 month post-transplant.

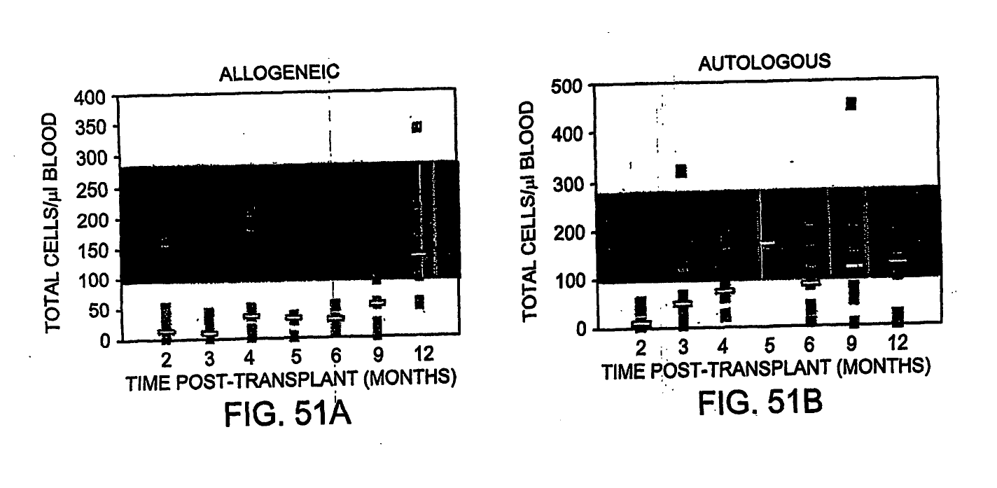

FIG. 51 depicts B cell reconstitution following HSCT at various time-points (2-12 months) following HSCT in control patients. As shown in FIG. 51B, B cell regeneration occurs relatively faster in autologous transplant patients as compared to that of allogeneic patients (FIG. 51A). However, a return to control values (shaded) was not evident until at least 6 months post-transplant in both groups.

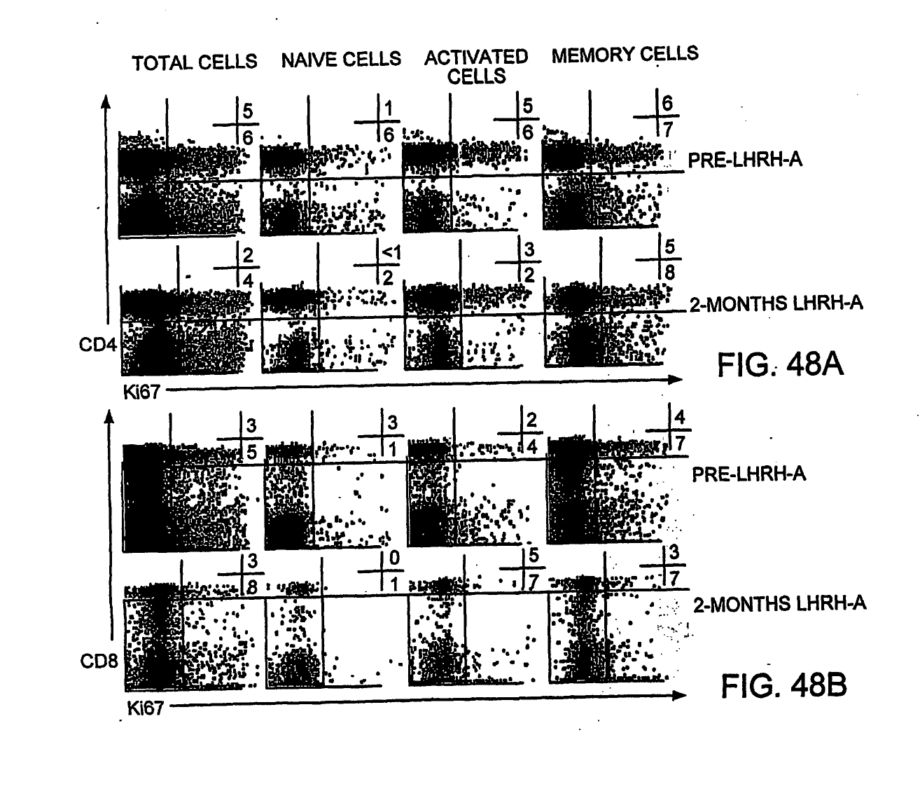

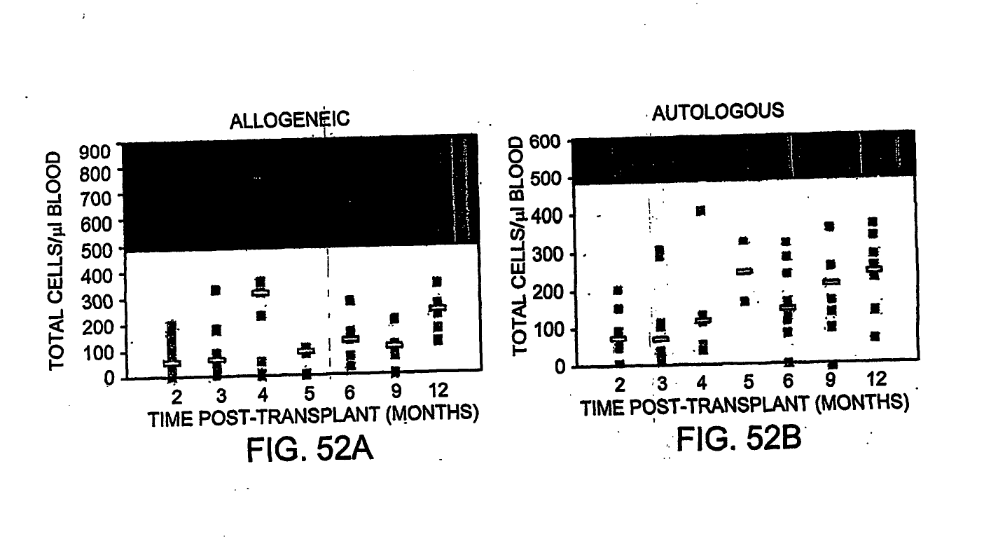

FIG. 52 depicts CD4⁺ reconstitution following HSCT at various time-points (2-12 months) following HSCT in control patients. While B cell numbers were returning to control values by 6 months post-transplant (see FIGS. 48A-B), CD4⁺ T cell numbers were severely reduced, even at 12 months post-transplant, in both autologous (FIG. 52B) and allogeneic (FIG. 52A) recipients.

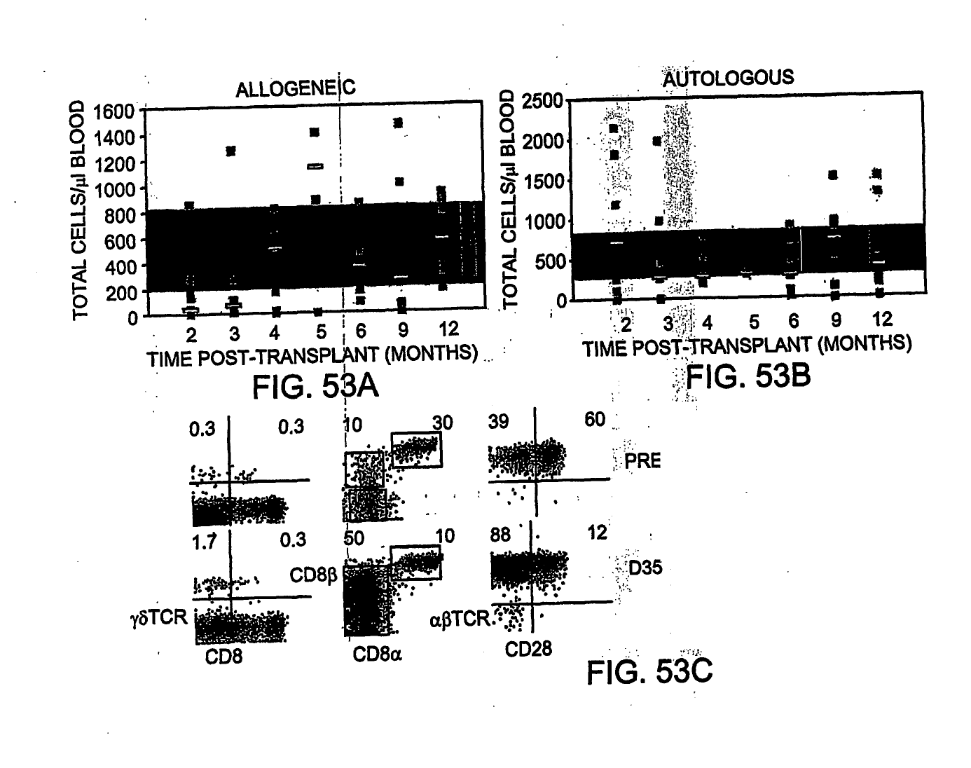

FIG. 53 depicts CD8⁺ regeneration following HSCT at various time-points (2-12 months) following HSCT in control patients. As shown in FIGS. 53A-B, CD8⁺ T cell numbers regenerated quite rapidly post-transplant in both allogeneic and autologous recipients, respectively. However, as shown in FIG. 53C, the CD8⁺ T cells are mainly of extrathymic origin as indicated by the increase in TCRγδ+ T CD8⁺ T cells, CD8αα T cells, and CD28⁻CD8⁺ T cells.

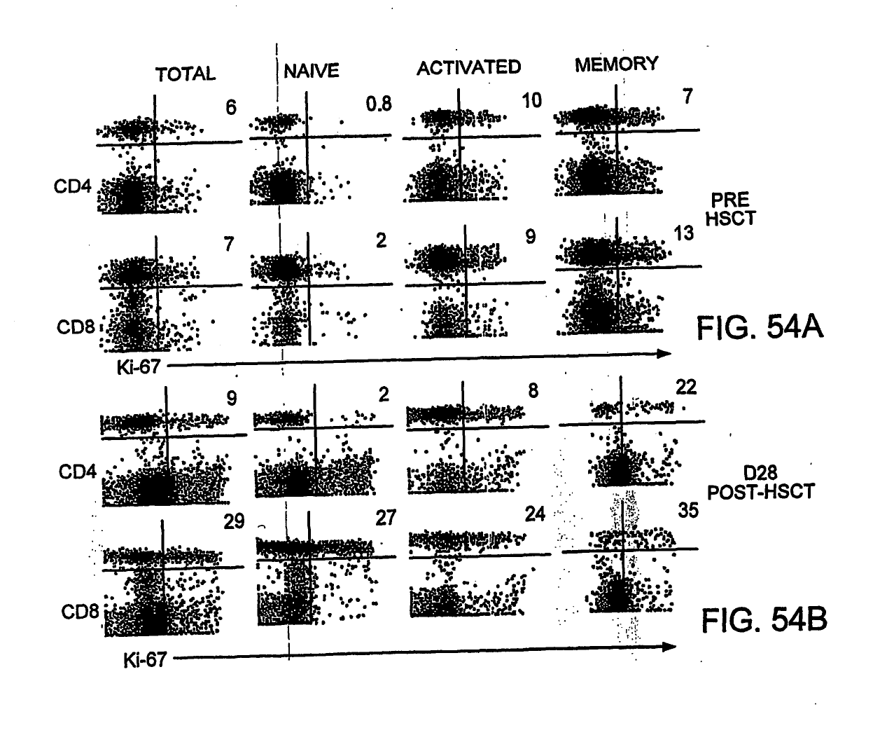

FIG. 54 depicts FACS analysis of proliferation in various populations of CD4⁺ and CD8⁺ T cells before (FIG. 54A) and 28 days after (FIG. 54B) HSCT in control patients using the marker Ki-67. Cells were analyzed on the basis of naïve, memory and activated phenotypes using the markers CD45RO and CD27. The majority of proliferation occurred in CD8⁺ T cell subset, which further indicated that these cells were extrathymically derived and that the predominance of proliferation occurred within peripheral T cell subsets.

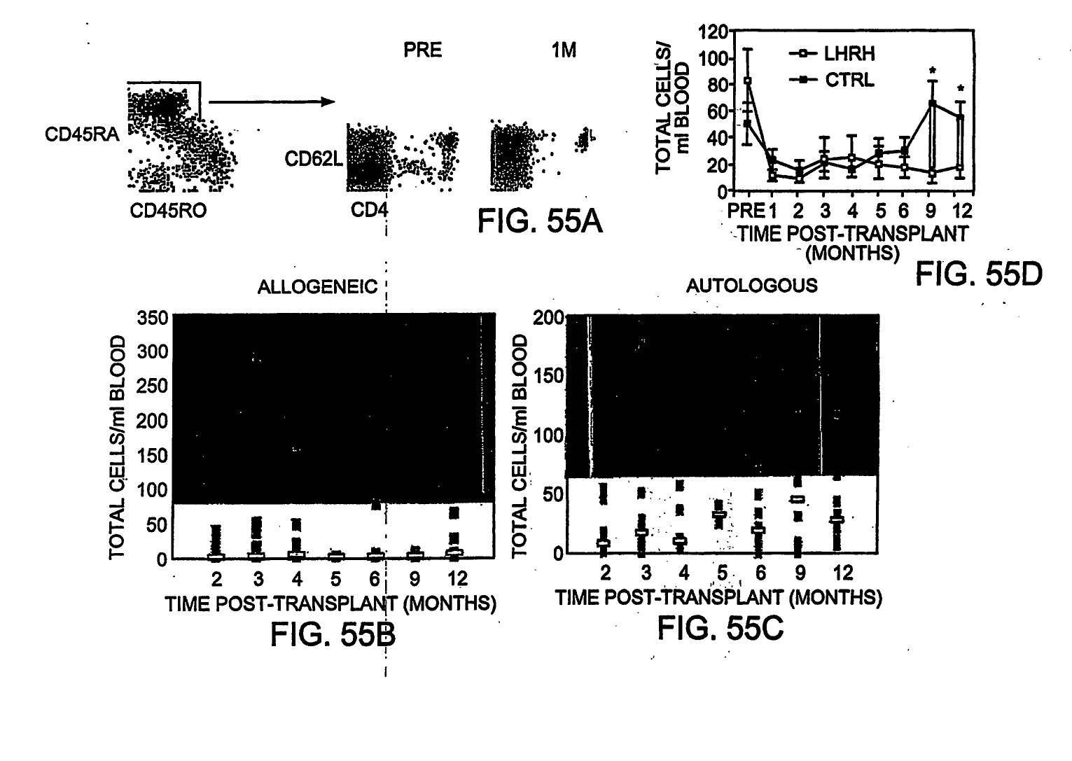

FIG. 55 depicts naïve CD4⁺ T cell regeneration at various time-points (2-12 months) following HSCT in control patients and LHRH-A treated patients. FIG. 55A depicts FACS analysis of naïve CD4⁺ T cells (CD45RA+CD45RO-CD62L+), and shows a severe loss of these cells throughout the study. As shown in FIGS. 55B-C, naïve CD4⁺ T cell began to regenerate by 12 months post-HSCT in autologous transplant patients (FIG. 55C) but were still considerably lower than the control values in allogeneic patients (FIG. 55B). These results indicated that the thymus was unable to restore adequate numbers of naïve T cells post-transplant due to the age of the patients. In contrast, in patients that were given LHRH-A 3-weeks prior to allogeneic HSCT, showed a significantly higher number of naïve CD4⁺ T cells at both 9 and 12 months post-transplant compared to controls (p≦0.05 both 9 and 12 months post-transplant compared to control (non-LHRH-A treated) (FIG. 55D)). This indicates enhanced regeneration of the thymic-dependent T cell pathway with sex steroid ablation therapy.

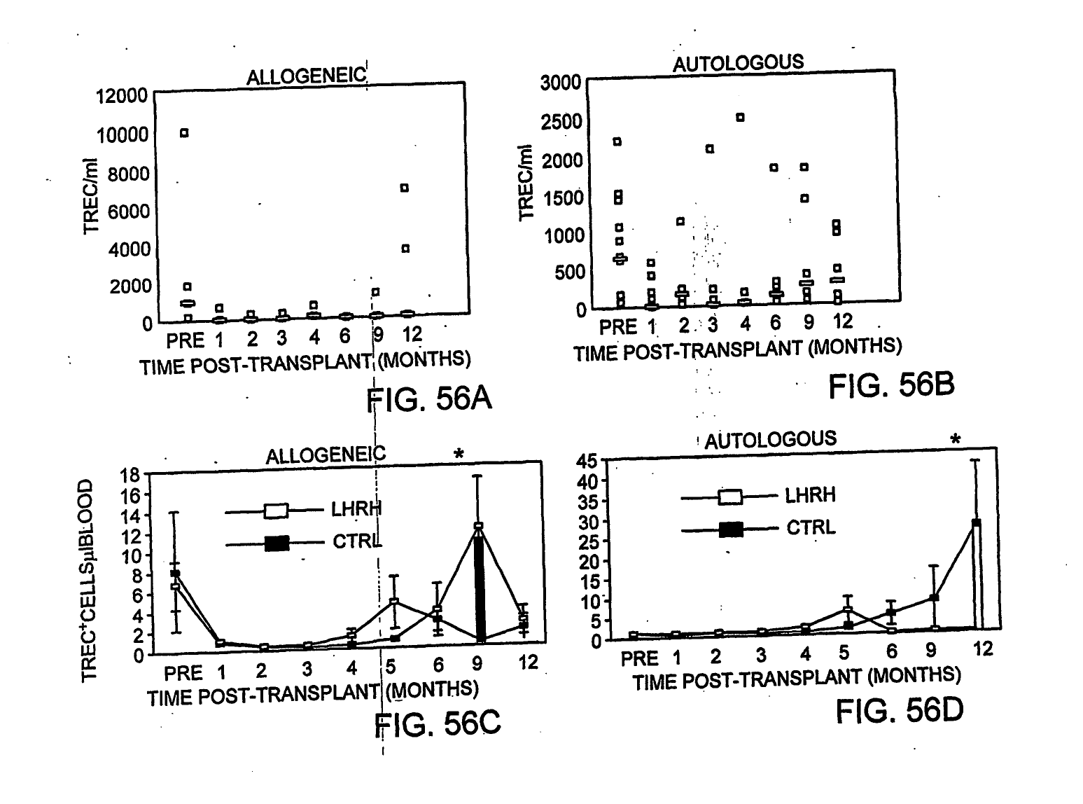

FIG. 56 depicts TREC levels at various time-points (1-12 months) following HSCT in control patients. Analysis of TREC levels, which are only seen in recent thymic emigrants (RTE), emphasized the inability of the thymus to restore levels following transplant in both allogeneic (FIG. 52A) and autologous (FIG. 52B) patients. Again, this was due to the age of the patients, as well as the lack of thymic function due to thymic atrophy, which has considerable implications in the morbidity and mortality of these patients. In contrast, patients undergoing allogeneic peripheral blood stem cell transplantation demonstrated a significant increase in CD4⁺ THEC⁺ cells/ml blood when treated with an LHRH-A prior to allogeneic transplantation (p≦0.01 at 9 months post-transplant compared to control (non-LHRH-A treated). Allogeneic patients who were given LHRH-A treatment showed a significantly higher number of CD4⁺TREC⁺ cells/ml blood at 9 months post-transplant (FIG. 56C) compared to controls. Autologous LHRH-A treated patients also showed significantly higher levels at 12 months post-transplant (FIG. 56D). This indicates enhanced regeneration of the thymus with sex steroid ablation therapy. Data is expressed as mean ±1 SEM of 5-18 patients. *=p≦0.01.

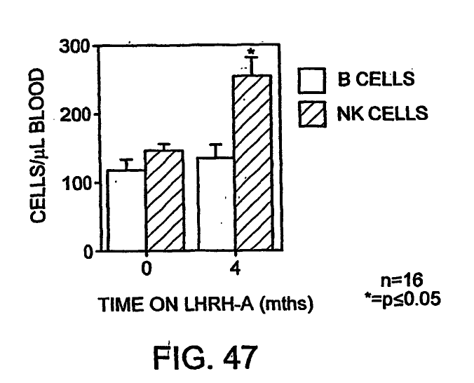

LHRH-A administration significantly increases NK but not B cell numbers in the peripheral blood. Overall, no significant change in B cell numbers was observed with LHRH-A treatment (FIG. 47). However, a significant increase in NK cell numbers was observed with treatment (p≦0.01) (FIG. 47). Therefore, removal of sex steroid results in significantly increased numbers of T cells and NK cells.

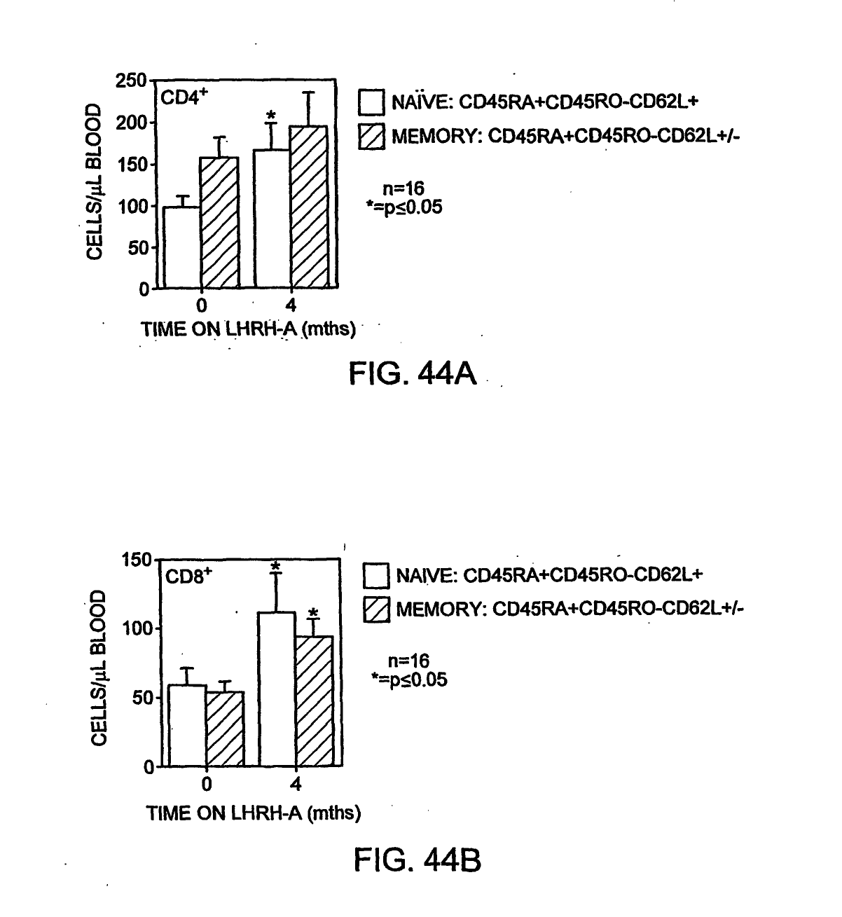

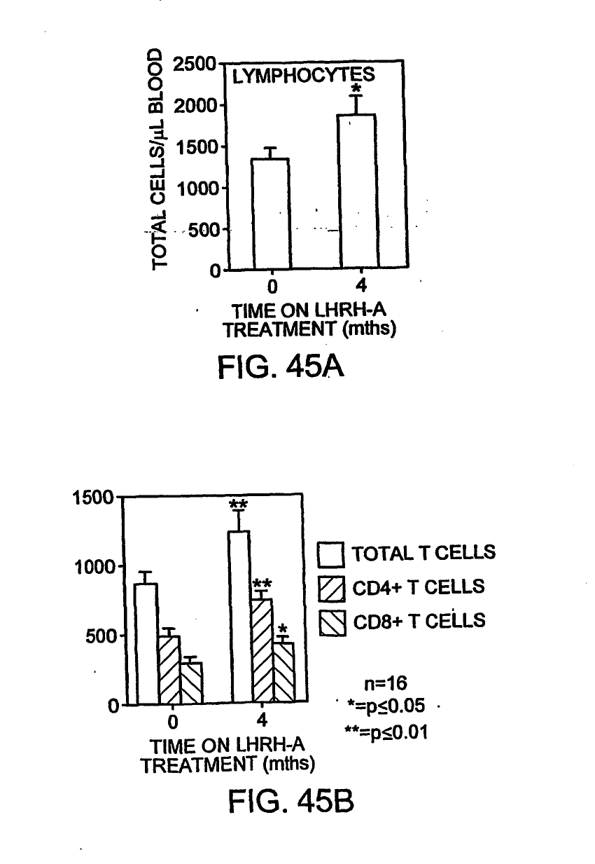

A significant increase in the total lymphocyte, T cell (predominantly CD4⁺) and NK cells was observed (FIGS. 45 and 47) consistent with previous studies of patients treated with LHRH-agonists (Garzetti et al., (1996) Obstet. Gynecol. 88: 234-40; Oliver et al., (1995) Urol. Int., 54:226-229; Umesaki et al., (1999) Gynecol. Obstet. Invest. 48:66-8). More detailed analysis of the T cell compartment revealed a significant increase in the numbers of naïve CD4⁺ T cells and both naïve and memory CD8⁺ T cells following LHRH-A treatment (FIG. 44).

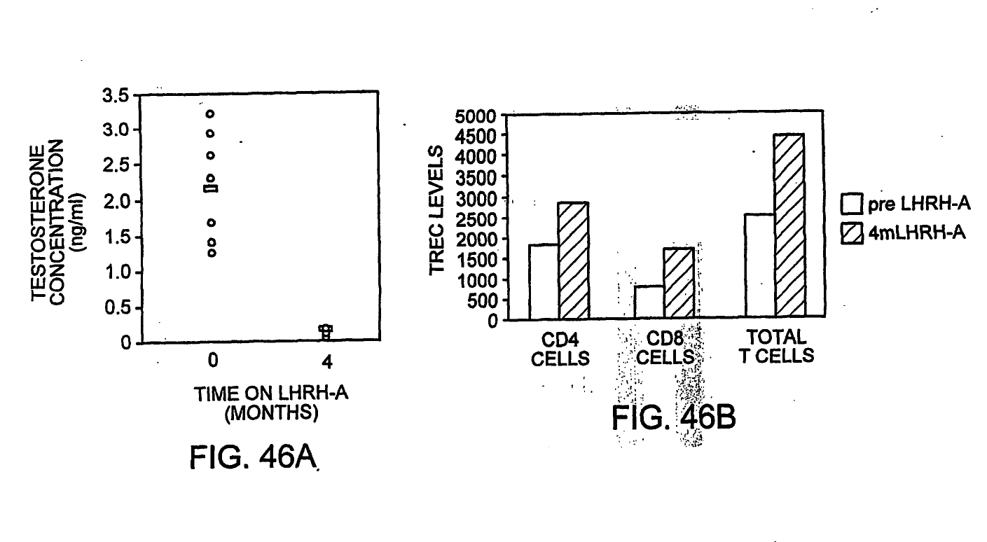

To determine if the increase in naïve T cells was through peripheral expansion (as seen, for example, with IL-7 administration (Soares et al., (1998) J. Immunol. 161:5909-5917) or as a direct result of thymic reactivation, analysis of cellular proliferation (Ki-67 antigen⁺), together with TREC levels was performed (Hazenberg et al., (2001) J. Mol. Med. 79:63140). No change in the level of proliferation was seen with agonist treatment in naïve, activated or memory populations of both CD4⁺ (FIG. 48A) and CD8⁺ T cells (FIG. 48B) (remaining at a low 2-4%). This indicates that the treatment does not directly induce proliferation of T cells, and that the levels of TRECs would not be influenced by excessive proliferation in the periphery. This does not rule out the possibility of peripheral expansion at earlier time-points. However this would presumably only account for increased activated/memory cell levels. Direct evidence for an increase in thymic function and T cell export was found following analysis of TREC levels in 10 patients (FIG. 46B). Within both the CD4⁺ and CD8⁺ T cell population, five out of ten patients showed an increase (>25% above initial presentation values) in absolute TREC levels (per ml of blood) by 4 months of LHRH-A treatment. This was also reflected in a proportional increase (per 1×10⁵ cells). This correlated with six out of ten patients showing an overall increase in total TREC levels. Only 1 patient showed a decrease in total TRECs (about 30% decrease). Since TRECs are diluted out with mitosis (Zhang et al., (1999) J. Exp. Med. 190:725-732), which could occur intrathymically as part of normal T cell development or following export (Hazenberg et al., (2001) J. Mol. Med. 79:631-40), the absolute TREC levels would represent very much an underestimate of T cell export. The marked increase in total TREC⁺ cells in the periphery following treatment with the agonist is, thus, fully consistent with regeneration of the thymus-dependent T cell pathway (Douek et al., (1998) Nature 396:690-695 (1998); Douek et al., (2001) J. Immunol. 167:6663-8; Hochberg et al., (2001) Blood 98:1116-21). Together, these data demonstrate the ability of sex steroid inhibition to improve thymic output in adult humans and provides a basis for restoring naïve T cell numbers following severe T-cell depletion in many clinical conditions.

### Example 19

### Sex Steroid Ablation Enhances Immune Reconstitution Following Hematopoietic Stem Cell Transplantation in Mice

This experiment was done to test the hypothesis that sex steroid inhibition in recipients of an allogeneic HSCT can improve their post-transplant immune reconstitution. Thus, these experiments aimed to establish whether sex steroid ablation influenced hematopoietic recovery following allogeneic HSCT. Fourteen days after HSCT, BM and thymic cell numbers were significantly increased in the castrated mice compared to sham controls. These remained elevated at day 28 at which time splenic cellularity was also increased in the castrates. In the thymus, T cell precursors and DC were significantly increased after HSCT and castration. BM precursors and developing B cells were also significantly increased after HSCT and castration. These central increases translated to a significant increase in donor-derived peripheral T and B cells after allogeneic HSCT. Every immune-enhancing strategy carries the risk of exacerbating the development of graft-versus-host disease (GVHD). Mice were castrated at the same time as GVHD induction in an allogeneic setting. There was no significant difference in GVHD incidence or severity when comparing castrated and sham-castrated mice. Furthermore, GVT activity was not diminished in the absence of sex steroid. It has been previously shown that lymphoid recovery is enhanced in allo-HSCT recipients after IL-7 treatment. The combination of IL-7 treatment and castration appeared to have an additive effect in the thymus following HSCT. These results indicate that castration and the resulting ablation of sex steroids enhance hematopoietic recovery following allogeneic HSCT without increasing GVHD and maintaining GVT.

Materials and Methods:

Reagents. Antimurine CD16/CD32 FcR block (2.4G2) and all following fluorochrome-labeled antibodies against murine antigens were obtained from Pharmingen (San Diego, Calif.): Ly-9.1(30C7), CD3(145-2C11), CD4 (RM4-5), CD8β.2(53-5.8), T-cell receptor-β (TCR-β; H57-597), CD45R/B220 (RA3-6B2), CD43 (S7), IgM-FITC (R6-60.2), CD11b (M/70), Ly-6G(Gr-1) (RB6-8C5), c-kit (2B8), Sca-1 (D7), CD11c (HL3) I-A^{k} (11-5.2), isotypic controls: rat IgG2a-k (R35-95), rat IgG2a-1 (B394), rat IgG2b-(A95-1), rat IgG1-k (R3-34), hamster IgG-group l-k (A19-3), hamster IgG-group 2-1 (Ha4/8), and 2.4G2 and Fcr (FcR blocking). Streptavidin-FITC, PercP-phycoerythrin (PE) also were obtained from Pharmingen (San Diego, Calif.). Recombinant human IL-7 was provided by Dr. Michel Morre (Cytheris, Vanves, France).

To confirm that the human recombinant IL-7 could stimulate murine cells, thymidine incorporation proliferation assays were performed with an IL-7-dependent murine pre-B cell line 2E8 and found that the human IL-7 used in the studies had a proliferative effect on murine cells that was equal to murine IL-7. Tissue culture medium consisted of RPMI 1640 supplemented with 10% heat inactivated fetal calf serum, 100 U/mL penicillin, 100 mg/mL streptomycin, and 2 mM L-glutamine (as well as 50 mM 2-mercaptoethanol for the culture of 32Dp210 cells and proliferation assays).

Mice and HSCT. Male C57BL/6J (B6, H-2b), C3FeB6F1/J([B6 3 C3H]F1; H-2b/k), B10.BR (H-2k), B6D2F1/J (H-2b/d), CBA/J (H-2k), Balb/c (H2-d), IL7−/− and KGF−/− mice were obtained from the Jackson Laboratory (Bar Harbor, Me.) and used in experiments when they were between 8 and 12 weeks of age. KGF−/− and IL7−/− were used between 4 and 7 months of age. HSCT protocols were approved by the Memorial Sloan-Kettering Cancer Center Institutional Animal Care and Use Committee. The BM cells were removed aseptically from femurs and tibias. Donor BM was depleted of T cells by incubation with anti-Thy-1.2 for 30 mins. at 4° C. followed by incubation with Low-TOX-M rabbit complement (Cedarlane Laboratories, Hornby, ON, Canada) for 1 hour at 37° C. Splenic T cells (for GVHD analysis) were obtained by purification over a nylon wool column followed by red cell removal with ammonium chloride red cell lysis buffer. Cells (5×10⁶ BM cells with or without splenic T cells and leukemia cells) were resuspended in Dulbecco modified essential medium (Life Technologies, Grand Island, N.Y.) and transplanted by tail vein infusion (0.25 mL total volume) into lethally irradiated recipients on day 0. Prior to transplantation, on day 0, recipients received 1300 cGy total body irradiation (¹³⁷Cs source) as split dose with 3 hours between doses (to reduce gastrointestinal toxicity). Mice were housed in sterilized microisolator cages and received normal chow and autoclaved hyperchlorinated drinking water (pH 3.0).

Surgical Castration. Mice were anaesthetized and a small scrotal incision was made to reveal the testes. These were sutured and removed along with surrounding fatty tissue. The wound was closed using surgical staples. Sham-castration required the same surgical procedure, except for the removal of the testes. Castration was performed one day prior to BM transplant for both immune reconstitution and GVHD studies.

Administration of IL-7. IL-7 were either given from days 0 to 13 or 21 to 27 intraperitoneally at 10 μg/day for immune reconstitution studies. PBS was injected into control mice at the same time-points.

Flow Cytometric Analysis. BM cells, splenocytes or thymocytes were washed in FACS buffer (phosphate buffered saline (PBS)/2% bovine serum albumin (BSA)/0.1% azide) and 1-3×10⁶ cells were incubated for 30 mins. at 4° C. with CD16/CD32 FcR block. Cells were then incubated for 30 mins. at 4° C. with primary antibodies and washed twice with FACS buffer. Where necessary, cells were incubated with conjugated Streptavidin for a further 30 mins. at 4° C. The stained cells were resuspended in FACS buffer and analyzed on a FACSCalibur™ flow cytometer (Becton Dickinson, San Jose, Calif.) with CellQuest™ software.

Proliferation Assays. Splenocytes (4×10⁵ cells/well) from sham-castrated (n=5) and castrated (n=5) mice were incubated for 5 days with irradiated (2000 cGy) BALB/C splenocytes as stimulators (2×10⁵ cells/well) in 96-well plates, and splenocytes (4×10⁵ cells/well) were stimulated with αCD3 (145-2c11) and αCD28 (37.51) (2.5 mg/mL as a final concentration) for 4 days. Cultures were pulsed during the final 18 hours with 1 mCi/well [3H]-thymidine, and DNA was harvested on a Harvester 96 (Packard). Stimulation indices (SI) were calculated as the ratio of stimulated cells (cpm) over unstimulated cells (cpm).

⁵¹Cr Release Assays. Target cells were labelled with 100 mCi ⁵¹Cr at 2×10⁶ cells/mL for 2 hours at 37° C. and 5% CO₂. After 3 washes, labelled targets were plated at 2.5×10³ cells/well in U-bottomed plates (Costar, Cambridge, Mass.). Splenocytes cultured with irradiated BALB/C splenocytes (1:2 ratio) for 5 days were added at various effector-to-target ratios in a final volume of 200 mL to 4 to 6 wells and incubated for 4 to 6 hours at 37° C. and 5% CO₂. Subsequently, 35 mL supernatant was removed from each well and counted in a gamma counter (Packard, Meriden, Conn.) to determine experimental release. Spontaneous release was obtained from wells receiving target cells and medium only, and total release was obtained from wells receiving 5% Triton X-100. Percent cytotoxicity was calculated by the following formula: percent toxicity=100×[(experimental release−spontaneous release)/(total release−spontaneous release)].

Detection of Alloreactive T-Cell Clones With Intracellular IFN-γ Staining Briefly, cells were incubated for 12 to 15 hours (for secondary allogeneic stimulation with T cell-depleted [TCD], irradiated stimulator cells) with Brefeldin A (10 mg/mL), harvested, washed, stained with primary (surface) fluorochrome (FITC, PerCP, and APC)-conjugated antibodies, fixed, and permeabilized with the Cytofix/Cytoperm kit (Pharmingen), and subsequently stained with αIFNγ-PE. FACS analysis was conducted by gating for the designated populations. Flow cytometer and software were used as mentioned below.

Delayed Type Hypersensitivity Assay. Sham-castrated and castrated mice were sensitized day 42 after allo-BMT by tail vein injection with 200 μl of 0.01% sheep red blood cells (Colorado Serum, Denver, Colo.) in PBS. Sensitized animals were challenged at day 46 in the right hind footpad with 50 μl of 20% sheep RBC suspension while the left hind footpad received the same volume of 50 μl of PBS solution as a control. 24 and 48 hours later, footpad swelling was measured with a dial-thickness gauge (Mitutoyo, Kanagawa, Japan). The magnitude of the response was determined by subtracting measurements of PBS-injected left footpads from the experimental right foot pads.

Assessment of GVHD. The severity of GVHD was assessed with a clinical GVHD scoring system as first described by Cooke et al. ((1996) Blood 88:3230-9). Briefly, ear-tagged animals in coded cages were individually scored every week for 5 clinical parameters (weight loss, posture, activity, fur, and skin) on a scale from 0 to 2. A clinical GVHD index was generated by summation of the 5 criteria scores (0-10). Survival was monitored daily. Animals with scores of 5 or more were considered moribund and were humanely killed.

Assessment of GVT-P815 (H-2d) Mastocytoma Induction and Assessment of Mastocytomic Death Versus Death From GVHD. B6D2F1/J recipients received 1×10³ P815 (H-2d) cells intravenously on day 0 of allogeneic HSCT (5×10⁶ T cell depleted (TCD) BM cells and 5×10⁵ T cells of C57/BL6 origin). Survival was monitored daily and the cause of death after HSCT was determined by necropsy as previously described. Briefly, death from leukemia was characterized by hepatosplenomegaly and the presence of mastocytoma cells in liver and spleen on microscopic examination; whereas, death from GVHD was defined as the absence of hepatosplenomegaly and leukemic cells in liver and spleen, and the presence of clinical symptoms of GVHD as assessed by the clinical GVHD scoring system at the time of death.

Semi-Quantitative RT-PCR. Total cellular RNA from whole BM was reverse-transcribed using Superscript II reverse transcriptase (Life Technologies, Rockville, USA). cDNA was PCR-amplified for 35 cycles (94° C. for 30 secs.; 56° C. for 30 secs.; 72° C. for 60 secs.) with PCR Master Mix (Promega, Madison, USA). HPRT: 5′CACAggACTAgAACACCT gC 3′ and 5′ gCTggTgAAAAggACCTCT 3′ TGFβ₁: 5′CTACTgCTgCAgCTC CACAg 3′ and 5′ TgCACTTgCAggAgCgCAC 3′ and KGF: 5′gCCTTgTCACg ACCTgTTTC 3′ and 5′ AgTTCACACTCgTAgCCgTTTg 3′.

Enzymatic Digestion of IL7−/− Thymii. IL7−/− mice contain a large proportion of CD45-thymic stromal cells and each thymus was subjected to enzymatic digestion in 0.125% (w/v) collagenase/dispase (Roche Applied Sciences, Indianapolis, USA) with 0.1% (w/v) DNase, releasing most of the stromal and haematopoietic cells from the thymii allowing for the accurate calculation of thymic cellularity. Anti-CD45 was used to identify CD45-stromal cells.

Statistics. All values are expressed as mean ±SEM. The Mantel-Cox log-rank test was used for survival data, and all other statistical analysis was performed with the nonparametric, unpaired Mann-Whitney U test. A P value of less than 0.05 was considered statistically significant.

Results:

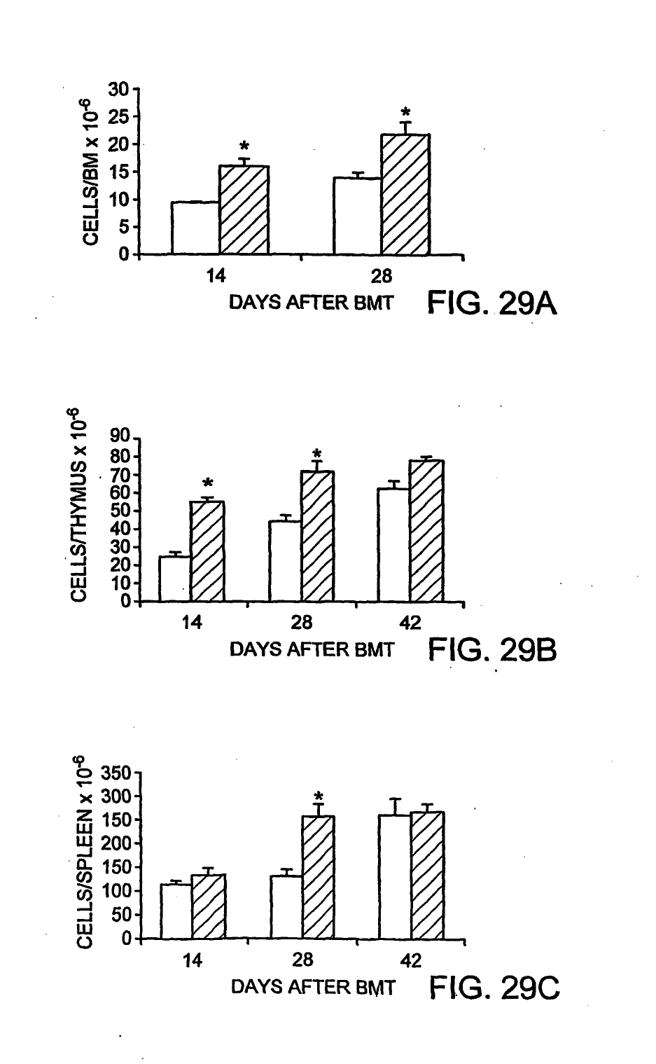

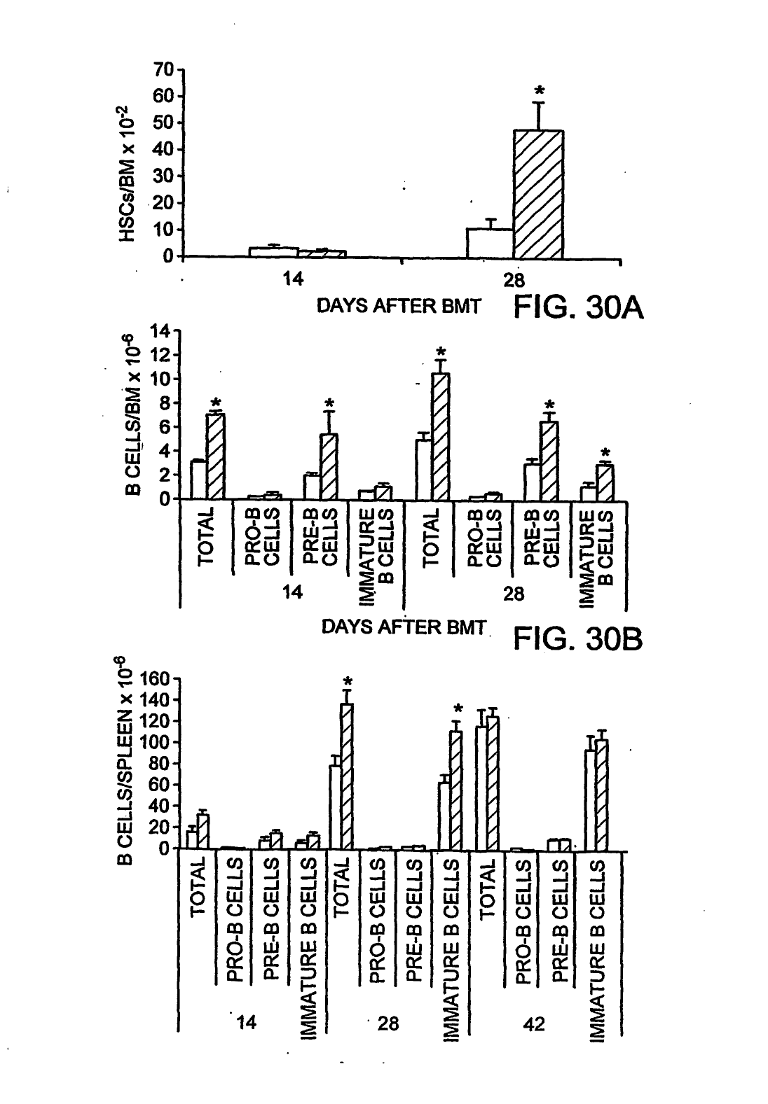

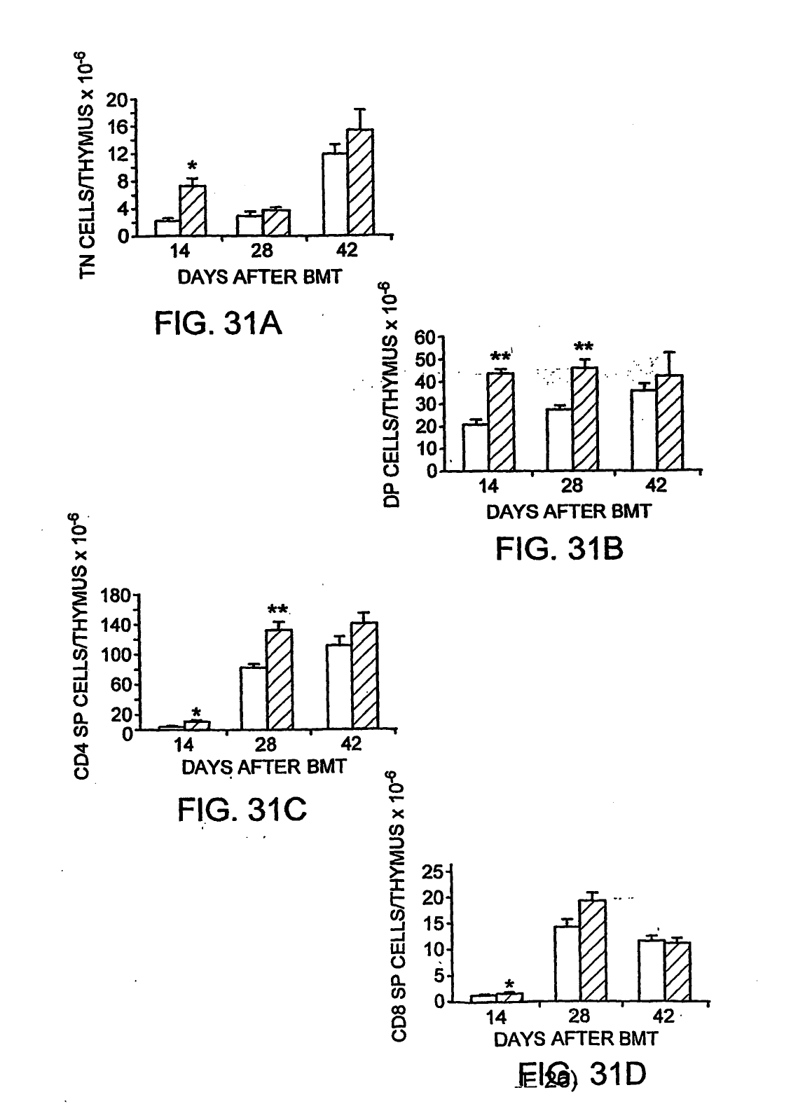

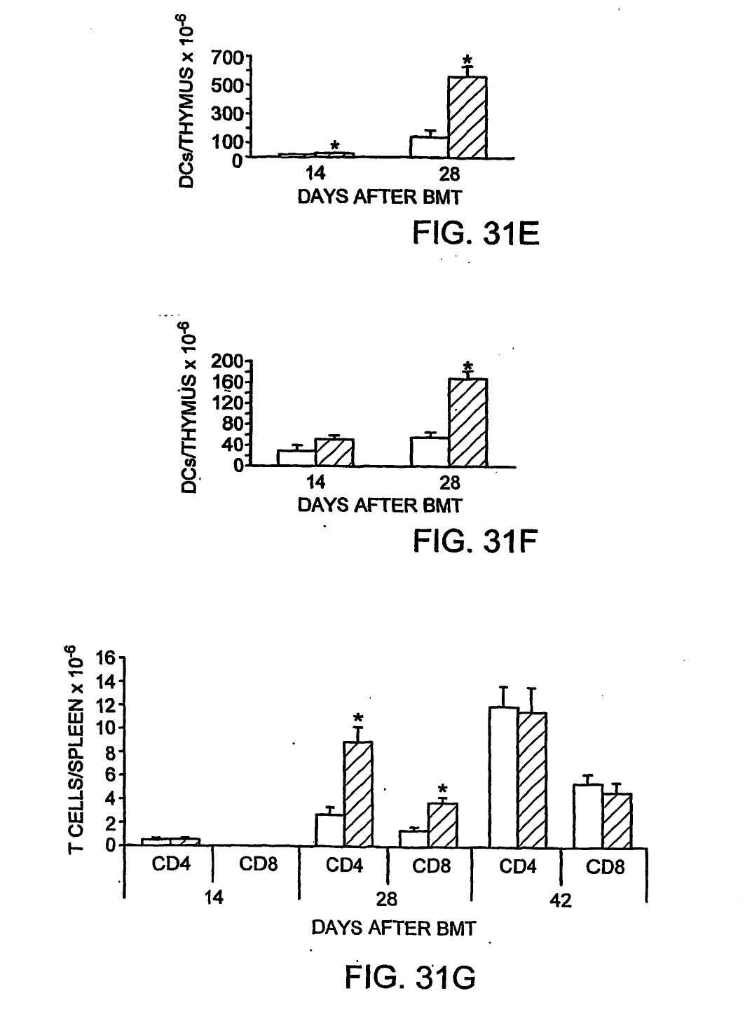

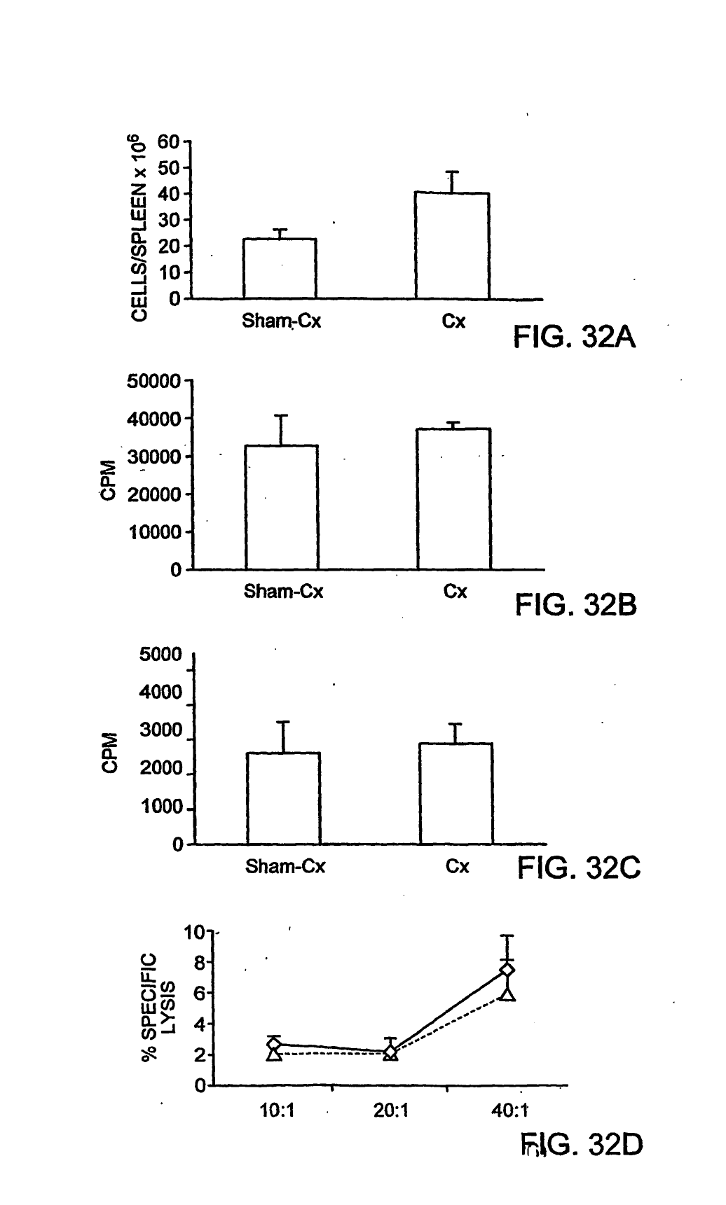

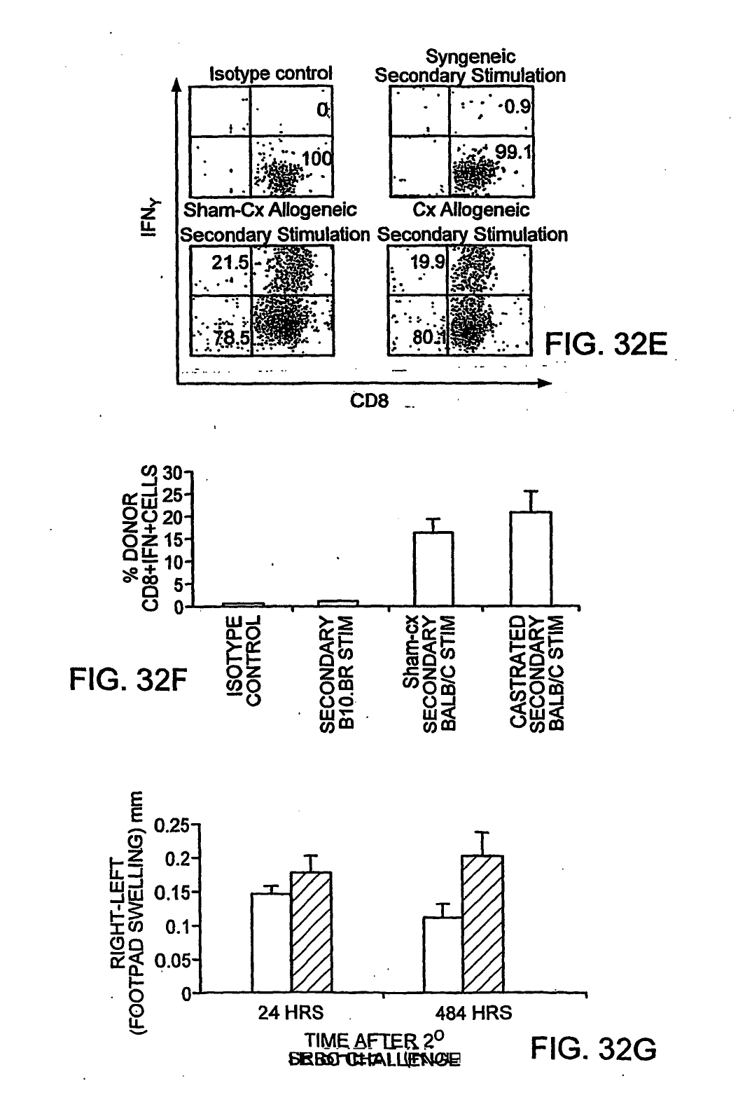

I. Castration Increases BM, Thymic and Splenic Cellularity Following Allogeneic HSCT. Male CBA mice were castrated one day prior to allogeneic HSCT. Mice were subjected to 1300 cGy total body irradiation followed by 5×10⁶ B10.BR TCD BM cells. There were significantly more cells in the BM (16×10⁶±1.4×10⁶) and thymus (55.4×10⁶±1.8×10⁶) of castrated mice, compared to the sham-castrated controls (9.5×10⁶±3.0×10⁵ and 25×10⁶±2.6×10⁶, respectively), as early as 14 days after HSCT (FIGS. 29A-B). These numbers remained significantly elevated in castrated mice 28 days after HSCT (BM: 22×10⁶±4.0×10⁶ vs. 14×10⁶±2.2×10⁶; thymus: 72×10⁶±5.9×10⁶ vs. 45×10⁶±2.9×10⁶). Splenic cellularity in the castrated mice was also significantly elevated above sham-castrated spleen cell numbers at day 28 (253×10⁶±28.4×10⁶ vs. 126×10⁶±13.9×10⁶) (FIG. 29C). The castrated mice had begun to approach pre-transplant cellularities by day 28. By 42 days after HSCT, there was no longer a significant difference between castrated and sham-castrated mice with respect to thymic and splenic cellularity. Since the sham-castrated recipients were young mice, they had active post-transplant lymphopoiesis but the time required to generate normal cellularity in the primary and secondary lymphoid tissues was markedly delayed compared to castrated recipients.
II. There are Significantly More Donor-Derived HSCs in the BM of Castrated Mice 28 Days After HSCT. Several studies have shown that sex steroids inhibit the proliferation and/or differentiation of early hematopoietic precursors (Thurmond et al., (2000) Endocrinol. 141:2309-2318; Medina et al., (2001) Nat. Immunol. 2:718; Kouro et al., (2001) Blood 97:2708). Therefore, the impact of castration on the HSC numbers in the allogeneic transplant setting has been investigated. The number of donor-derived HSC was very low in both sham-castrated and castrated mice 14 days after allogeneic HSCT (2.98×10²±1.25×10² and 2.66×10²+8.8×10¹, respectively) (FIG. 30A). However, by day +28 there are significantly more Ly9.1⁺ Lin⁻ Sca-1⁺c-kit⁺ donor-derived HSCs in the castrated mice (4.8×10³±1.1×10³), compared to the sham-castrated controls (1.1×10³±4.1×10²) (FIG. 30A).
III. Castration Prior to Allogeneic HSCT Enhances Donor-Derived B Cell Recovery. In the analysis of B cell recovery, three stages in B cell development was distinguished Pro-B cells (CD45R⁺CD43⁺IgM⁻), pre-B cells (CD45R⁺CD43⁻IgM⁻) and immature B cells (CD45R⁺CD43⁻IgM⁺). Fourteen days after allogeneic HSCT, there were significantly more pre-B cells in the BM of castrated mice (5.5×10⁶±1.7×10⁶) compared to the sham-castrated controls (2.08×10⁶±5.0×10⁴) (FIG. 30B). At 28 days, there were also significantly more pre-B cells (sham-cx: 3.1×10⁶±3.7×10⁵ c.f. cx: 6.6×10⁶±6.6×10⁵) and immature B cells (sham-cx: 1.3×10⁶±2.6×10⁵ c.f. cx: 3.0×10⁶±3.4×10⁵) in the BM of castrated mice (FIG. 30B). The increase in BM B cells and their precursors translated to a significant increase in the number of immature B cells in the spleens of castrated mice, 28 days after HSCT (sham-cx: 64.9×10⁶±6.4×10⁶ c.f. cx: 112.0×10⁶±10.0×10⁶) (FIG. 30C). These results are in agreement with previous studies that suggest that castration enhances B cell production and export from the BM.
IV. T Cell Reconstitution Following Allogeneic HSCT is Enhanced by Castration. Thymocytes and peripheral cells were divided into developmental stages on the basis of expression of CD3, CD4 and CD8: Triple Negatives (TN) (CD3⁻CD4⁻CD8⁻), double positive (DP) (CD4⁺CD8⁺), single positive CD4 (SP CD4) CD3⁺CD4⁺CD8⁻ and single positive CD8 (SP CD8) CD3⁺CD4⁻CD8⁺ (FIGS. 31A-D). As early as 14 days after allogeneic HSCT, there are significantly more TN, DP, SP CD4 and SP CD8 thymocytes in castrated mice compared to sham castrated controls. 28 days after HSCT, DP and CD4 SP cell numbers remain significantly elevated in the castrated group. By day 42, all thymocyte subsets are equivalent in sham-castrated and castrated mice. Both host and donor-derived DC are thought to play an integral role in the avoidance of graft rejection (Morelli et al., (2001) Semin. Immunol. 13:323-335). Fourteen days after allogeneic HSCT, there are significantly more host-derived CD11c^{hi}DC in the thymii of castrated mice. Both host and donor-derived DC in the thymus were significantly increased in castrated mice 28 days after allogeneic HSCT (FIGS. 31E-F). The increase in thymocyte numbers in castrated mice translated to a significant increase in the number of donor-derived mature CD4⁺ and CD8⁺ T cells in the spleens of castrated mice compared to the sham-castrated controls at day 28 (FIG. 31G).
V. On a Per Cell Basis, There is no Significant Difference Between T Cells From Sham-Castrated and Castrated Mice. In order to determine the functional potential of peripheral T cells in castrated mice after allo-HSCT, a series of in vitro assays were performed. The number of donor-derived T cells in castrated and sham-castrated controls 6 weeks after allo-HSCT are represented in FIG. 32A. The proliferative capacity of the splenic T cells was tested in 2 ways: αCD3/αCD28 cross-linking (FIG. 32B) and in a 3^{rd} party MLR (using irradiated BALB/C splenocytes as stimulators) (FIG. 32C). There is no significant difference in the proliferative capacity of peripheral T cells when comparing sham-castrated and castrated mice in either of these settings. Six weeks after allo-HSCT, splenocytes were cultured with irradiated BALB/C splenocytes (3^{rd} party) for 5 days. Following 5 days of allogeneic stimulation, the vast majority of cells in culture were CD8⁺ T cells. Half of these cells were used in a CTL (⁵¹Cr release) assay to determine the cytotoxicity of splenocytes from sham-castrated and castrated mice. Splenocytes were tested for their ability to kill ⁵ Cr loaded A20 (BALB/C B cell lymphoma tumor cell line) cells at different effector: target ratios (FIG. 32D). There was no significant difference between sham-castrated and castrated mice with respect to cytotoxicity. The other half of the cells cultured for 5 days were restimulated overnight with either 3rd party (BALB/C) or syngeneic (B10.BR) irradiated splenocytes and Brefeldin A to determine IFN-γ production. FIG. 32E shows IFNγ production by donor-derived CD8⁺ splenic T cells following BALB/C primary stimulation and either BALB/C or B10.BR secondary stimulation (control). This is represented graphically in FIG. 32F. There is no significant difference in the proportion IFN-γ producing donor-derived CD8⁺ when comparing sham-castrated and castrated mice.

In order to assess immune function in vivo, a DTH assay was used, whereby, 42 days after castration and allo-BMT, mice were sensitized with sRBCs. On day 46, they were challenged and 24 and 48 hours later footpad swelling was determined. The DTH response is significantly enhanced 48 hours after challenge when mice are castrated at the time of allo-HSCT compared to sham-castrated controls (FIG. 32G).

These functional assays demonstrate that the T cells in castrated recipients are comparable on a per cell basis with T cells from sham-castrated recipients and are capable to respond to novel antigens with intact proliferation, cytoxicity and cytokine production. However, the significantly more rapid T cell reconstitution in castrated recipients translates in an enhanced DTH response even at 6 weeks after transplant.

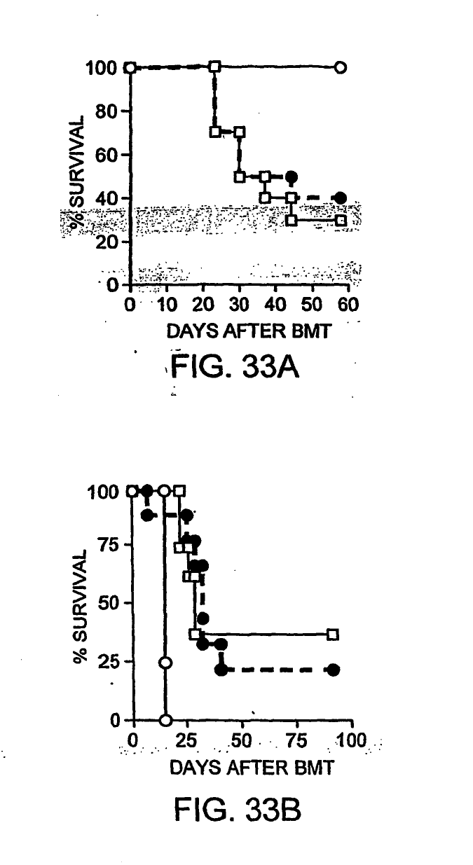

VI. Castration Prior to Allogeneic-HSCT Does Not Exacerbate GVHD and Maintains GVT Activity. Both GVHD and GVT are mediated, primarily, by alloreactive donor-derived T cells, which are transferred with the allograft. Any treatment used to enhance immune reconstitution has the potential to exacerbate GVHD or, conversely, decrease GVT activity. To establish that castration does not have a stimulatory effect on alloreactive T cells of donor origin, GVHD was induced by the addition of allogeneic donor T cells to the allograft. There was no significant difference in morbidity or mortality due to GVHD when comparing castrated and sham-castrated mice (FIG. 33A). To assess the effects of castration on GVT activity, the mastocytoma cell line P815 (H-2d) was injected into B6D2F1/J recipients at the time of transplant. Animals that died during the experiment were autopsied and the cause of mortality (tumor vs. GVHD) was determined. Mortality due to mastocytoma remained unchanged following castration (six out of nine mice) when compared to sham-castrated controls (five out of eight mice). This suggests that castration does not diminish GVT response following HSCT (FIG. 33B).
VII. IL-7 and Castration Have an Additive Effect Following Allogeneic HSCT. It has previously been shown that IL-7 treatment can increase the number of T and B cells in otherwise untreated animals and can also enhance lymphoid recovery following cyclophosphamide treatment, irradiation, syngeneic and allogeneic HSCT (Alpdogan et al., (2001) Blood 98:2256; Bolotin et al., (1996) Blood 88:1887; Faltynek et al., (1992) J. Immunol. 149:1276; Morrissey et al., (1991) J. Immunol. 146:1547). IL-7 is known to increase T cell numbers through increased thymic activity as well as peripheral expansion.

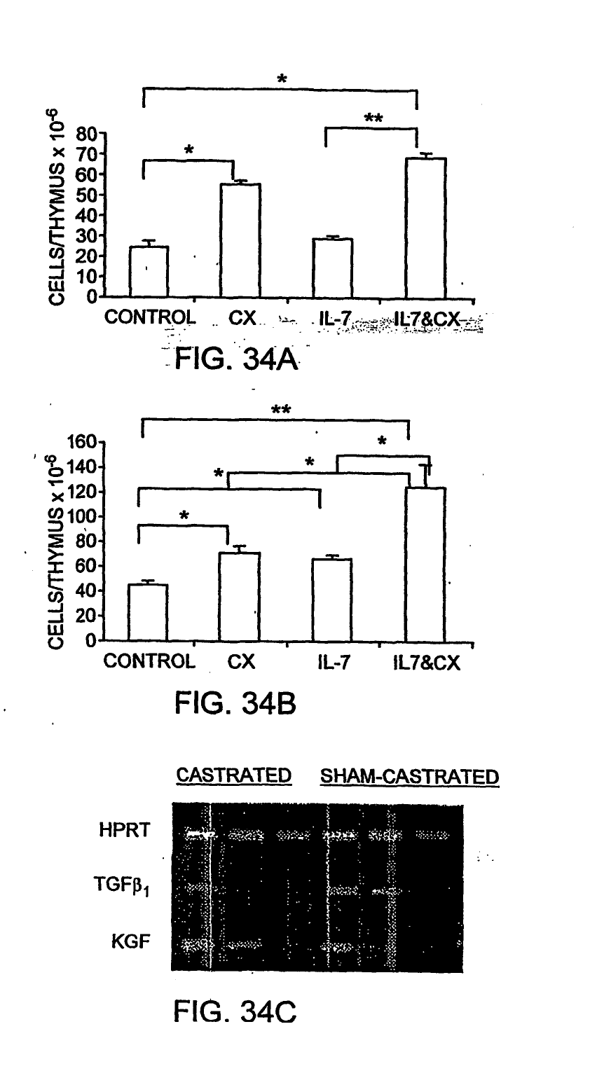

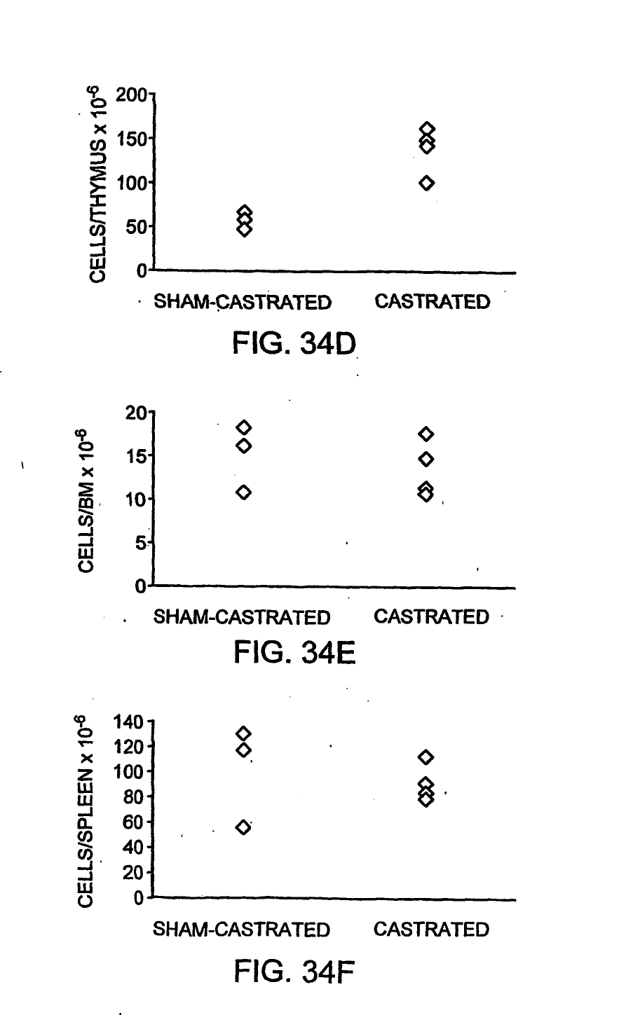

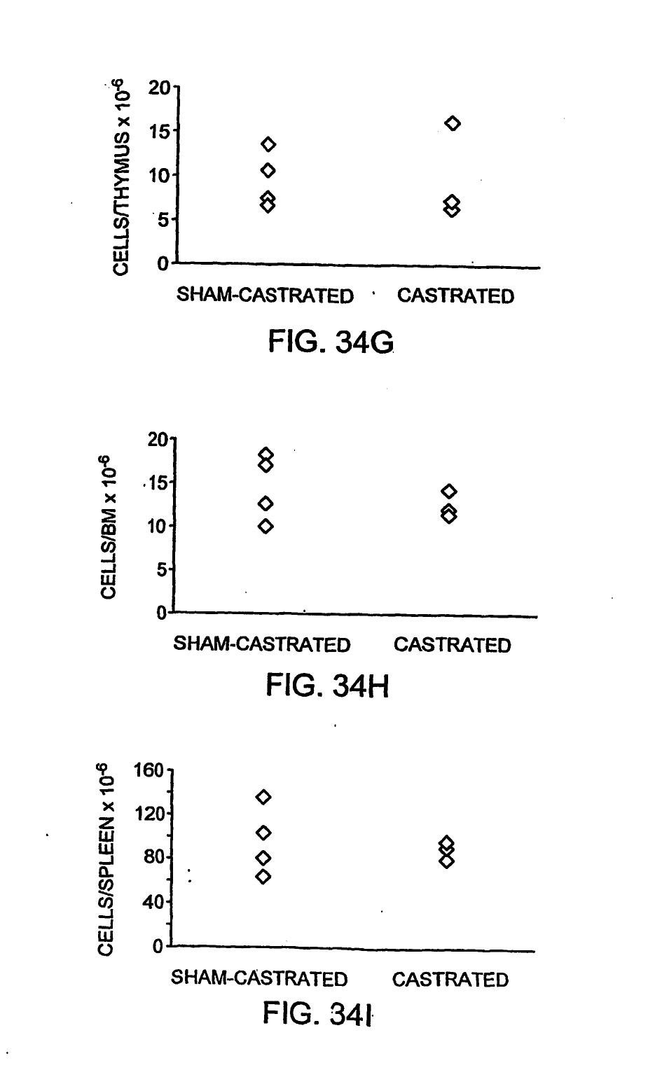

It was, therefore, decided to combine IL-7 administration with castration in recipients of allogeneic HSCT. 14 days after treatment, there are significantly more cells in the thymii of castrated mice and those given the combined treatment (castration and IL-7 administration). At this early time-point there is no difference seen between the PBS treated, sham-castrated controls and the IL-7 treated, sham-castrated mice. There is also no significant difference seen between the castrated group and those receiving the combined treatment suggesting that it is only the effects of castration acting 14 days after allo-HSCT, IL-7 treatment and castration (FIG. 34A). At a later time-point, 28 days after allo-HSCT, the cellularity of the thymii in both the castration alone group and the IL-7 alone group is significantly higher than the control group. The combination of IL-7 treatment and castration had an additive effect on thymic cellularity when analyzed 28 days after allogeneic HSCT (FIG. 34B).

VIII. Semi-Quantitative RT-PCR for IL-7, TGF-β₁ and KGF Reveals an Increase in KGF and a Decrease in TGF-β₁ Following Allo-HSCT and Castration. RT-PCR analysis of whole bone marrow cells revealed undetectable levels of IL-7 transcript in both sham-castrated and castrated mice as late as 6 weeks after allo-HSCT. When template from control, untransplanted mice was used, IL-7 was detected (data not shown). TGFβ₁ and KGF are known to be key mediators of hematopoiesis. Using HPRT equibrated template, there appears to be a decrease in TGFβ₁ and an increase in KGF 2 weeks after castration and allo-BMT (FIG. 34C).
IX. Changes That Occur Following Castration Were Seen in KGF^{-/-} Mice But Not IL7^{-/-} Mice. In order to further study the possible mechanisms behind the enhanced immune reconstitution following castration, KGF^{-/-} and IL7^{-/-} mice (4-6 months old) were castrated and 2 weeks later thymus, spleen and BM were analyzed (TGFβ₁^{-/-} mice die prepubertally, Shull et al., (1992) Nature 359:693). Thymic cellularity is significantly increased when comparing sham-castrated and castrated KGF^{-/-} mice. Although no differences were seen in the total cellularity of the BM and spleen at this early time-point, changes were seen in the B cell compartment of the BM, as seen previously in wildtype mice (Ellis et al., (2001) Int. Immunol. 13:553; data not shown). Due to the fact that a large proportion of cells in the thymi of IL7^{-/-} mice are CD45⁻ stromal cells, enzymatic digestion was used to obtain a single cell suspension when using these mice. By doing this, many more cells are released into suspension which accounts for the slightly larger thymic cellularity seen in this experiment compared to previous literature (von Freeden-Jeffry et al. (1995) J. Exp. Med. 181:1519). No differences were seen in the thymii (FIG. 34G), spleen (FIG. 34H) or BM (FIG. 34I) of IL7^{-/-} mice when comparing castrated mice and sham-castrated controls. These finding suggest an important role for IL-7 in the enhanced immune reconstitution seen following castration and allo-HSCT.

Discussion:

Recipients of an allogeneic HSCT experience a prolonged period of immune deficiency, which is associated with life-threatening infections. With increasing age of the recipient, this infection risk increases, as does the time it takes for full immunological reconstitution. The period of immunodeficiency following HSCT can be greater than one year, and recent long-term studies demonstrated a decrease in TREC+CD4⁺ T cells in older HSCT patients compared to their donors (Storek et al., (2001) Blood 98:3505); Lewin et al., (2002) Blood 100:2235). This suggests that thymic damage and the subsequent decline in T cell production may be more prolonged than once thought. The majority of post-HSCT infections are associated with a lack of CD4⁺ peripheral T cells (Storek et al., (1997) Am. J. Hematol. 54:131). Therefore, the increase in peripheral T cell number that occurs following castration may decrease the incidence of these infections leading to enhanced overall survival of transplant patients.

Many groups have focused their research on hematopoietic reconstitution following HSCT, and the most promising results have come with the use of haematopoietic growth factors and cytokines. G-CSF, for example, is used to mobilize donor stem cells (Dreger et al., (1993) Blood 81:1404). Noach et al., showed that pre-treatment with SCF and IL-11 or SCF and Flt-3 ligand resulted in enhanced donor cell engraftment ((2002) Blood 100:312). KGF appears to enhance engraftment and reconstitution in both syngeneic and allogeneic settings as well as ameliorating GVHD (Panoskaltsis-Mortari et al., (2000) Blood 96:4350; Krijanovski, et al., (1999) Blood 94:825). IL-7 enhances immune reconstitution following syngeneic HSCT (Bolotin et al., (1996) Blood 88:1887) and can enhance immune reconstitution and maintain GVT activity without exacerbation of GVHD after allogenic HSCT (Alpdogan et al., (2001) Blood 98:2256).

Several studies have shown that sex steroid ablation, be it by surgical or chemical castration, of male mice increases both BM and splenic B cell numbers (Ellis et al., (2001) Int. Immunol. 13:553; Erben et al., (2001) Horn. Metab. Res. (2001) 33:491; Wilson et al., (1995) Blood 85:1535; Masuzawa et al., (1994) J. Clin. Invest. 94:1090). The increase in peripheral B cell number is predominantly due to an increase in B220^{lo}CD24^{hi} recent BM emigrants (Ellis et al., (2001) Int. Immunol. 13:553). Olsen et al., have demonstrated that androgens enhance the production of TGF-β by stromal cells within the BM, which in turn suppresses B cell development (J. Clin. Invest. (2001) 108:697). In addition, neutralization of TGF-β in vitro reverses B cell suppression by dihydrotestosterone. TGF-β has also been shown to down-regulate stromal IL-7 production and subsequently inhibit the proliferation of B cell progenitors (Tang et al., (1997) J. Immunol. 159:117). Therefore one possible explanation for the effects of castration/androgen ablation, in this instance, following allogeneic HSCT, suppresses the production of TGF-β, in turn enhancing B cell development, explaining the increased B cell numbers in the BM and spleen of castrated mice compared to the sham-castrated controls.

The proliferation of hematopoietic stem cells is also regulated by TGF-β. Batard et al., have demonstrated that physiological concentrations of TGF-β₁ inhibit the proliferation and differentiation of HSCs in vitro ((2000) J. Cell. Sci. 113:383-90). Furthermore, disruption of TGF-β signalling in HSCs (via the transient expression of a mutant type II receptor) enhances survival and proliferation of these cells (Fan et al., (2002) J. Immunol. 168:755-62). It is, therefore, possible that the increased number of HSCs seen 28 days after allogeneic HSCT and castration may be due to a decrease in the production of TGF-β by BM stromal cells.

Both estrogen and androgen can effect the differentiation and proliferation of HSCs (Thurmond et al., (2000) Endocrinol. 141:2309-18; Medina et al., (2001) Nat. Immunol. 2:718-24; Kouro et al., (2001) Blood 97:2708-15). Estrogen directly inhibits the proliferation and differentiation of HSC, as well as some lymphoid precursor subsets (Medina et al., (2001) Nat. Immunol. 2:718; Kouro et al., (2001) Blood 97:2708). HSCs express functional estrogen receptors (ERs) and estrogen administration decreases the number of Lin⁻c-kit⁺Sca-1⁺ HSCs (Thurmond et al., (2000) Endocrinol. 141:2309; Kouro et al., (2001) Blood 97:2708). The study conducted by Thurmond et al., suggests that the transition between c-kit⁺Sca-1⁺ precursors and the more mature subsets (c-kit⁺Sca-1⁻ and c-kit⁻Sca-1⁻) is blocked when ERα is present in the hematopoietic cells of the BM (Endocrinol. (2000) 141:2309). ERs are also present on BM stromal cells (Girasole et al., (1992) J. Clin. Invest. 89:883; Smithson et al., (1995) J. Immunol. 155:3409), suggesting that estrogen may also have an effect on the production of growth factors by the stroma which, in turn, affects HSC proliferation and/or differentiation. Although most evidence suggests an indirect effect of androgens on HSCs via the BM stroma, the presence of functional androgen receptors on lymphoid components of the BM does not exclude a direct effect.

Olsen et al., have shown that it is the presence of a functional androgen receptor on the thymic epithelium but not the thymocytes that is essential for age-related thymic involution and the subsequent regeneration via sex steroid ablation (Olsen et al., (2001) Endocrinol. 142:1278).

Although the molecular mechanisms for thymic involution and/or regeneration remain unknown there are several potential candidates. Thymic IL-7 levels decline with age (Aspinall, et al., (2000) Vaccine 18:1629; Andrew et al., (2002) Exp. Gerontol. 37:455; Ortman et al., (2002) Int. Immunol. 14:813). It remains unclear as to whether this is due to a decrease in the number of cells that produce IL-7 or a decrease in the ability of the existing cells to produce the cytokine. However, IL-7 treatment of old mice can reverse age-related increases in thymic apoptosis and enhance thymopoiesis (Andrew et al., (2001) J. Immunol. 166:1524). Stem cell factor (SCF) and M-CSF mRNA expression is also decreased in the mouse thymus with age (Andrew et al., (2002) Exp. Gerontol. 37:455). At the intracellular signalling level, E2A transcription factor essential for the development of DN thymocytes is decreased, as is Foxn1 (whn) transcription factor present in and involved in the proliferation and differentiation of thymic epithelial cells (Ortman et al., (2002) Int. Immunol. 14:813). Sempowski et al. have monitored mRNA steady-state levels in aging humans and have shown a significant increase in Leukemia Inhibitory Factor (LIF), Oncostatin M, IL-6 and SCF mRNA (J. Immunol. (2000) 164: 2180).

The above studies suggest that the response to castration is multifactorial. The experiments with castration of IL7−/− mice suggest that increased production of IL-7 is an important component of the castration effect. However, an additive effect on thymic cellularity was observed when recipients were treated with both high dose IL-7 and castration, which would suggest that castration provides more thymopoietic effects than increased IL-7 levels alone.

DC are the key mediators of negative selection in the thymus (Jenkinson et al., (1985) Transplantat. 39:331; Matzinger et al., (1989) Nature 338:74-60) and in a transient setting have been implicated in inducing graft acceptance by presenting alloantigens in the thymus after transplantation, removing donor-specific T cells. Tomita et al., have demonstrated that donor-derived cells in the thymii of MHC class I mis-matched recipients mediate deletion of donor reactive cells (Tomita et al., (1994) J. Immunol. 153:1087-1098). They have also shown that thymus-derived DC injected intravenously traffic to the host thymus. Furthermore, it has been shown that intrathymic injection of both host cells pulsed with allo-antigen, donor cells, or donor soluble peptides increases graft acceptance (Garrovillo et al., (1999) Transplantation 68:1827; Ali et al., (2000)Transplantation 69:221; Garrovillo et al., (2001) Am. J. Transplant. 1:129; Oluwole et al., (1993) Transplantation 56:1523-1527). These studies show that castration significantly increased the number of host and donor-derived DC in the thymus following allogeneic HSCT. That is, sex steroid ablation enhances the differentiation and/or proliferation of thymic DC. Thus, castration, used in conjunction with hematopoietic stem cells and solid organ transplantation, increases graft acceptance.

Conclusion:

The current study has revealed that blocking sex steroids has a profound positive effect on immune reconstitution following myeloablation and HSCT. HSCs, and B and T progenitors are markedly enhanced. This provides an important platform for increasing the efficiency of engraftment and post-transplant strategies that depend on an intact hematopoietic system, such as vaccination against tumor or microbial antigens or gene therapy targeting donor HSCT. The increase in DC in the thymus have importance in inducing and sustaining tolerance to allogeneic grafts. In addition, GVHD is not exacerbated and GVT activity is not diminished in castrated recipients. These results demonstrate that transient sex steroid ablation (using, e.g., LHRH analogs) are useful as a prophylactic therapy to enhance immune reconstitution.

### Example 20

### Sex Steroid Ablation Enhances TCR-Specific Stimulation Following Hematopoietic Stem Cell Transplantation

In order to assess the functional nature of regenerated T cells, patients were analyzed for responsiveness to TCR specific stimulation.

Materials and Methods:

Patients. Test patients were given Zoladex (LHRH-A) 3 weeks prior to stem cell transplantation and then monthly injections for 4 months. All patients were analyzed pre-treatment, weekly for 5 weeks after transplantation and monthly up to 12 months. Ethics approval was obtained from The Alfred Committee for Ethical Research on Humans (Trial Number 01/006).

Preparation of PBMC. Purified lymphocytes were used for T-cell Stimulation assays and TREC analysis and were prepared as above.

T Lymphocyte Stimulation Assay. Analysis of TCR specific stimulation was performed using anti-CD3 and anti-CD28 cross-linking from 1-12 months post-transplant, unless otherwise indicated. For TCR-specific stimulation, cells were incubated for 48 hours on plates were previously coated with purified anti-CD3 (1-10 μg/ml) and anti-CD28 (10 μg/ml). Following plaque formation (48-72 hours), 1 μCi of ³H-Thymidine was added to each well and plates incubated for a further 16-24 hours. Plates were harvested onto filter mats and incorporation of ³H-thymidine was determined using liquid scintillation on a counter (Packard-Coulter, USA).

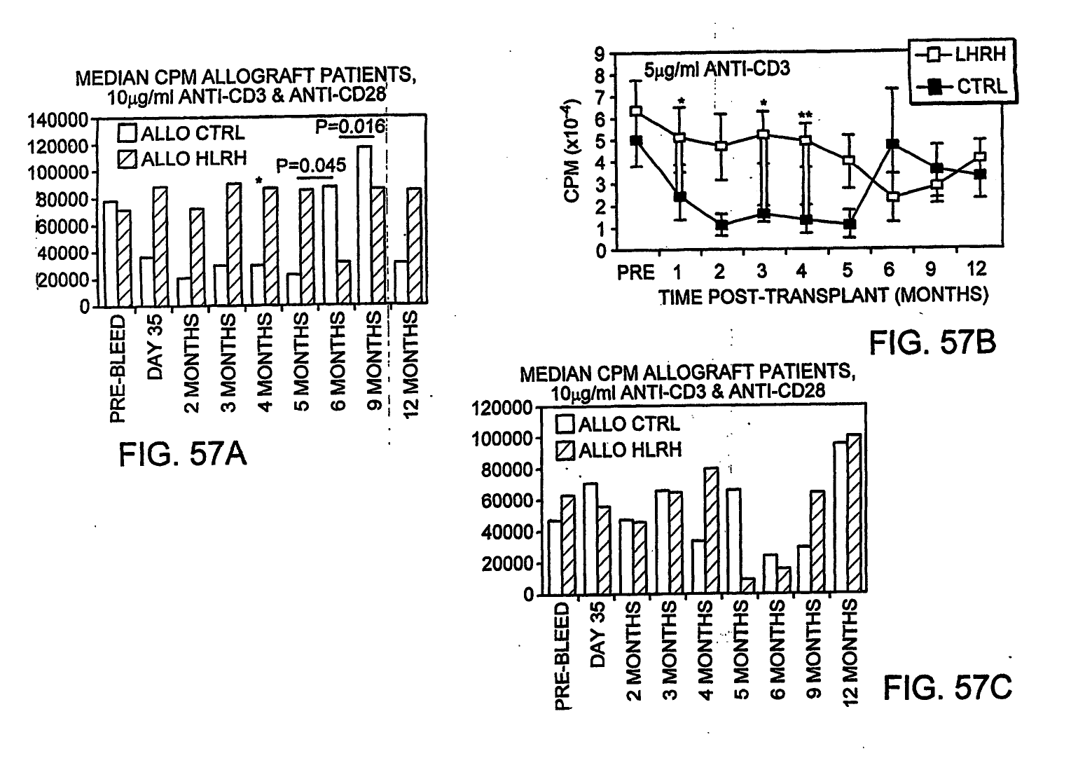

I. LHRH-A Administration Enhances Responsiveness to TCR Specific Stimulation Following Allogeneic Stem Cell Transplantation. LHRH-A treated patients showed enhanced proliferative responses (assessed by ³H-thymidine incorporation) compared to control patients at all time-points except 6 and 9 months due to low patient numbers analyzed at this time (FIGS. 57A-B). In allogeneic transplant patients treated with a LHRH-A, a significant increase in responsiveness to anti-CD3/CD28 stimulation was observed at 4 and 5 months post-transplant compared to control patients. While control patients showed an enhanced response at both 6 and 9 months post-transplant, LHRH-A treated patients showed a greater responsiveness at 12 months post-transplant. At 6 and 9 months post-transplant, control patients had similar responsiveness to pre-treatment values. However, at all other time-points, they were considerably lower. In contrast, LHRH-A treated patients had equivalent responsiveness at all time-points except 6 months compared to pre-treatment. LHRH-A treated patients showed enhanced proliferative responses (assessed by ³H-Thymidine incorporation) compared to control patients at 1, 3 and 4 months post-transplant. This indicates a contribution of direct peripheral T cell effects, as new CD4⁺ T cells are not evident until at least 1-2 months post-transplant (FIG. 57A-B).
II. LHRH-A Administration Enhances Responsiveness to TCR Specific Stimulation Following Autologous Stem Cell Transplantation. A similar response as that seen in allograft recipients was also observed with autograft recipients (FIG. 57C). Those patients treated with LHRH-A demonstrated an enhanced proliferative response to TCR stimulation at both 4 and 9 months post-transplant. LHRH-A treated patients showed enhanced proliferative responses (assessed by ³H-thymidine incorporation) compared to control patients at all time-points except 5 months. Restoration to pre-treatment values was observed by 12 months post-transplant in both control and LHRH-A treated patients.

### III. LHRH-A Administration Enhances Responsiveness to TCR Specific Stimulation Following Treatment For Chronic Cancer Sufferers.

Patients with chronic haematological malignancies who were immunosuppressed patients as determined by documented serious infection associated with: CD4<0.4×10⁹/L, or lymphoproliferative disorder (e.g., CLL, myeloma, lymphoma) and receiving regular prophylactic intravenous gammaglobulin (documented hypogammaglobulinemia), or previous treatment with fludarabine, deoxycorfomycin and 2-CdA within 4 years, or prior allogeneic or autologous stem cell transplant within 2 years, were enrolled in the study. Ethics approval was obtained from The Alfred Committee for Ethical Research on Humans (Trial number 01/006). Patents were given LHRH-A as follows, and results are presented up to 6 weeks post-LHRH-A administration.

| | |
|---|---|
| d − 1 (or before): | consent signed and pre-treatment investigations |
| | performed |
| d0: | Zoladex administered (males 10.8 mg; females |
| | 3.6 mg) |
| d + 28: | females-Zoladex 3.6 mg administered |
| d + 56: | females-Zoladex 3.6 mg administered |
| d + 84: | females and males-Zoladex 3.6 mg administered |

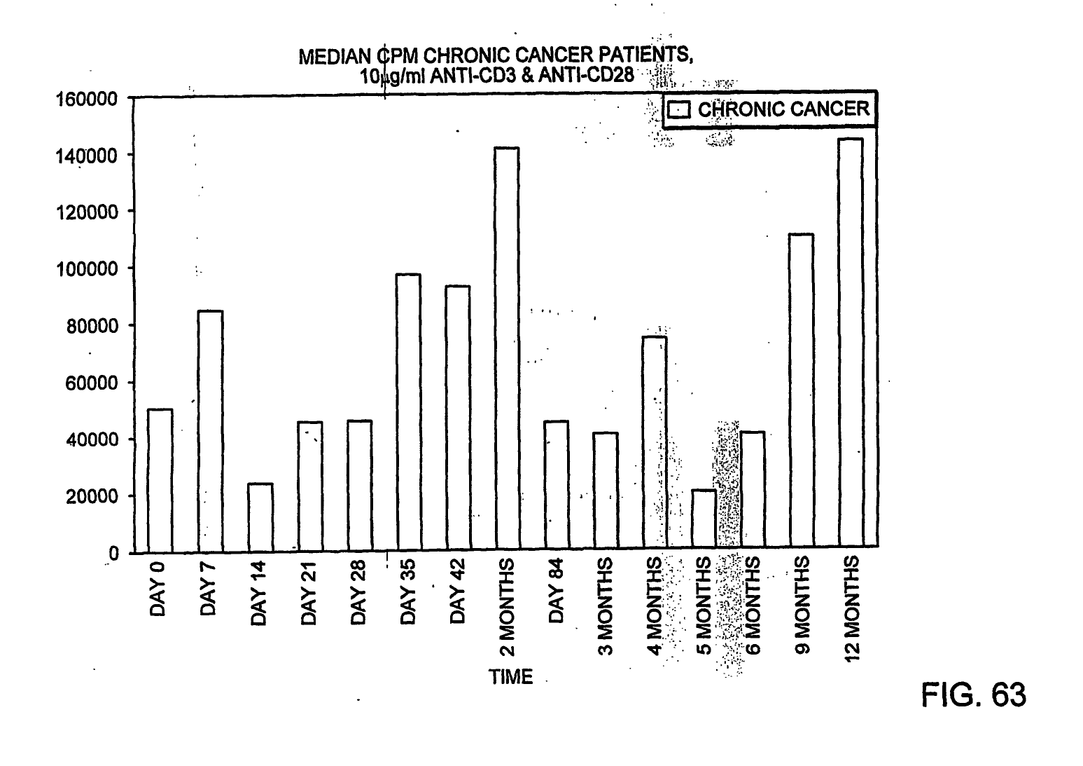

Analysis of TCR specific stimulation was performed using anti-CD3 and anti-CD28 cross-linking from D7 post-administration. LHRH-A treated patients showed enhanced proliferative responses (assessed by ³H-Thymidine incorporation) compared to pre-treatment levels in a “cyclical” fashion (FIG. 63). That is, on the day of injection, an increase in T cell proliferation was observed, and this appeared to decrease slightly prior to the subsequent injection. This also indicates the probability of a direct influence of LHRH-A on existing peripheral T cells. This reflected the administration of the agonist with monthly depot injections. These results indicate an influence directly on peripheral T cells. However, the enhanced response seen at 12 months post-treatment reflect changes in thymic-derived T cells as well, since agonist administration was ceased from 4 months for all patients.

Conclusion:

Since an increased responsiveness to TCR-specific stimulation was observed as early as D35 post-transplantation, this is due predominantly to pre-existing T cells, since newly-derived T cells only just begin to exit the thymus at this stage. The influence of newly-derived thymic-generated T cells is only observed after this time-point, so it can be concluded that the increased T cell function is due to an improvement in the pre-existing T cells and not T cells derived from the regenerated thymus.

### Example 21

### Sex Steroid Ablation Enhances Mitogenic Stimulation Following Hematopoietic Stem Cell Transplantation

In order to assess the functional nature of regenerated T cells, patients were analyzed for responsiveness to mitogenic stimulation.

Materials and Methods:

Patients. Patients were those enrolled in Clinical Trial Protocol No. 01/006, as above. Prior to stem cell transplant, patients were given LHRH-A (3 weeks prior). Patients who did not receive the agonist were used as control patients.

Preparation of PBMC. Purified lymphocytes were used for T cell stimulation assays and TREC analysis and were prepared as above.

Mitogen Stimulation Assay. Analysis of mitogenic responsiveness was performed using pokeweed mitogen (PWM) and tetanus toxoid (TT) from 1-12 months post-transplant. For mitogen stimulation, PBMC were plated out in 96-well round-bottom plates at a concentration of 1×10⁵/well in 100 μl of RPMI-FCS. Cells were incubated at 37° C., 5% CO₂ with TT (2LFAU/ml) or PWM (10 ug/ml). Following plaque formation (48-72 hours), 1 μCi of ³H-thymidine was added to each well and plates were incubated for a further 16-24 hours. Plates were harvested onto filter mats and incorporation of ³H-thymidine was determined using liquid scintillation on a β-counter (Packard-Coulter, USA).

### I. LHRH-A Administration Enhances Responsiveness to Mitogenic Stimulation Following Allogeneic Stem Cell Transplantation.

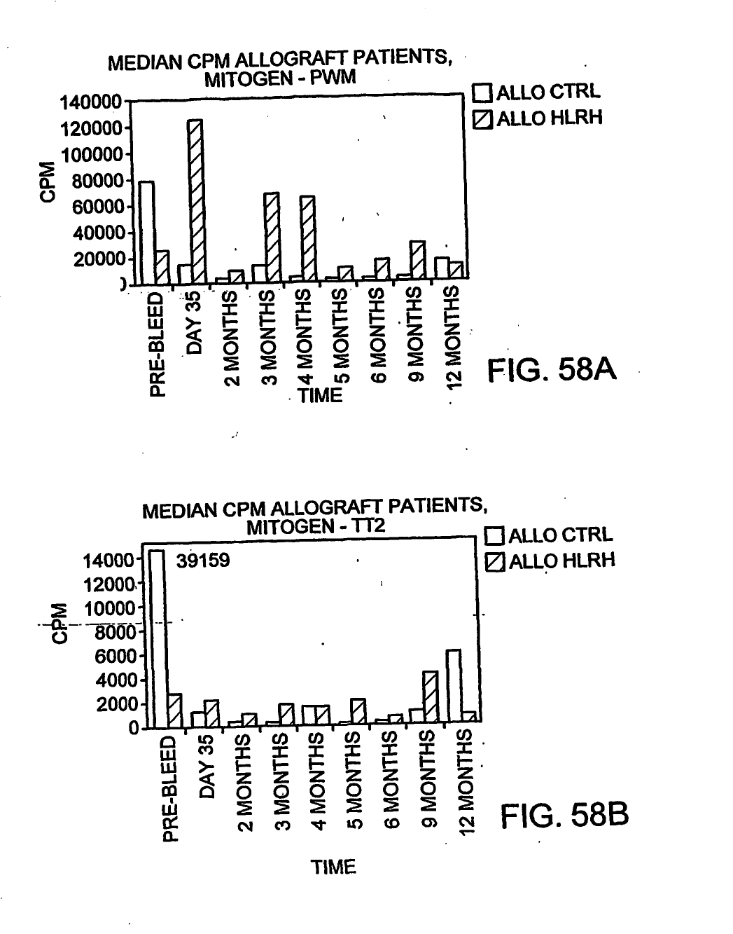

Analysis of mitogen responsiveness showed that allogeneic patients undergoing LHRH-A treatment had an increased responsiveness to PWM at all time-points post-transplant compared to controls (FIG. 58A). That is, patients treated with LHRH-A prior to stem cell transplantation showed an enhanced responsiveness to PWM stimulation at all time-points studied compared to control patients.

Similar results were evident following analysis of response to TT (FIG. 58B). LHRH-A treated patients had enhanced responses at all time-points compared to control patients except at 12 months post-transplantation.

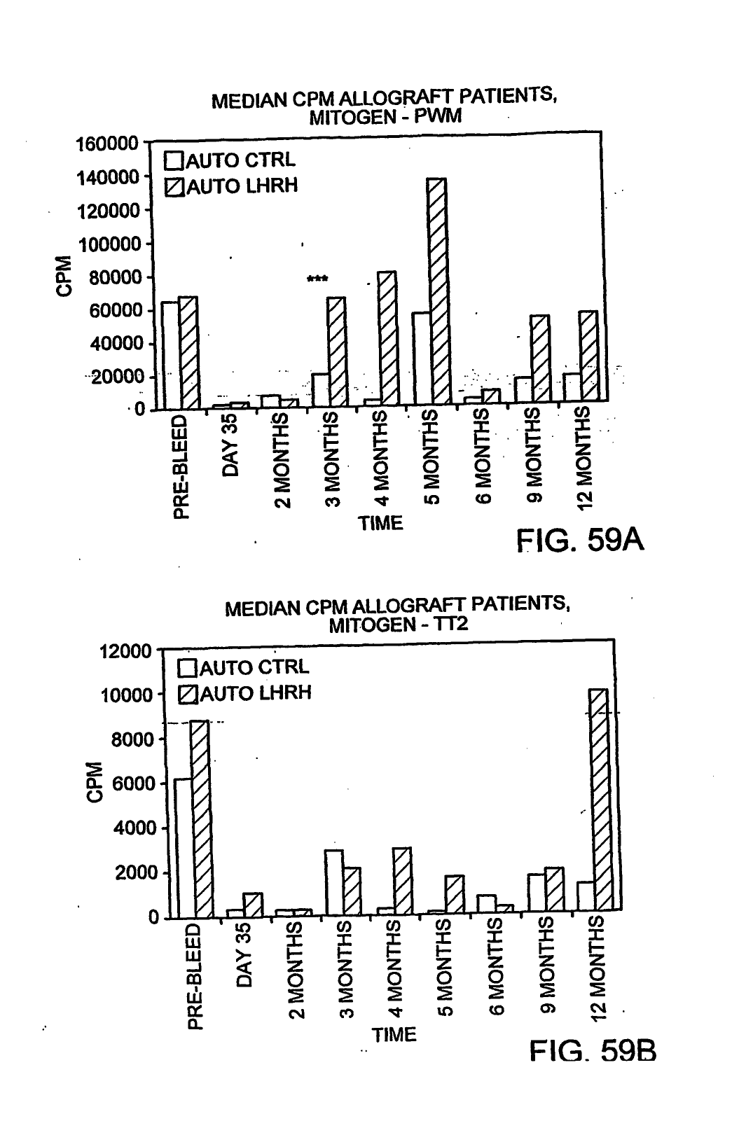

II. LHRH-A Administration Enhances Responsiveness to Mitogenic Stimulation Following Autologous Stem Cell Transplantation. Patients treated with LHRH-A prior to stem cell transplantation showed an enhanced responsiveness to PWM stimulation at the majority of time-points studied compared to control patients (p≦0.001 at 3 months) (FIG. 59A). By 12 months post-transplantation, LHRH-A treated patients had restored responsiveness to pre-treatment levels, while control patients were still considerably reduced.

Also, similar to allograft patients above (FIG. 58B), autograft patients also showed an increased response to TT when given LHRH-A prior to treatment. Patients treated with LHRH-A prior to stem cell transplantation showed an enhanced responsiveness to TT stimulation at the majority of time-points studied compared to control patients (FIG. 59B). By 12 months post-transplantation, LHRH-A treated patients had restored responsiveness to pre-treatment levels, while control patients were still considerably reduced.

Conclusion:

Since an increased responsiveness to mitogenic stimulation was observed as early as D35 post-transplantation, this is due predominantly to pre-existing T cells since newly-derived T cells are only just beginning to exit the thymus at this stage. The influence of newly-derived thymic-generated T cells is observed only after this time-point.

### Example 22

### Sex Steroid Ablation Enhances Rate of Engraftment in Hematopoietic Stem Cell Transplant Patients

Materials and Methods:

Materials and methods used for these experiments are described above. Additional materials and methods are as follows:

Allogeneic and autologous patients (control or LHRH-A treated) were analyzed for total WBC and total granulocyte or neutrophil numbers following HSCT. Three weeks prior to HSCT, patients were treated with LHRH-A. Patients who did not receive the agonist were used as control patients. Total white blood cell (WBC) counts, granulocyte (G) and neutrophil counts per μl of blood were determined up to 35 days post-transplant. A sample of whole peripheral blood was analyzed either using a Cell-Dyn 1200 automated cell counter (Abbott) or hemocytometer counts done in duplicate. This allows calculation of total white blood cells, lymphocytes and granulocyte numbers following transplant. Analysis of engraftment was performed from D14-D35 post-transplant.

Results:

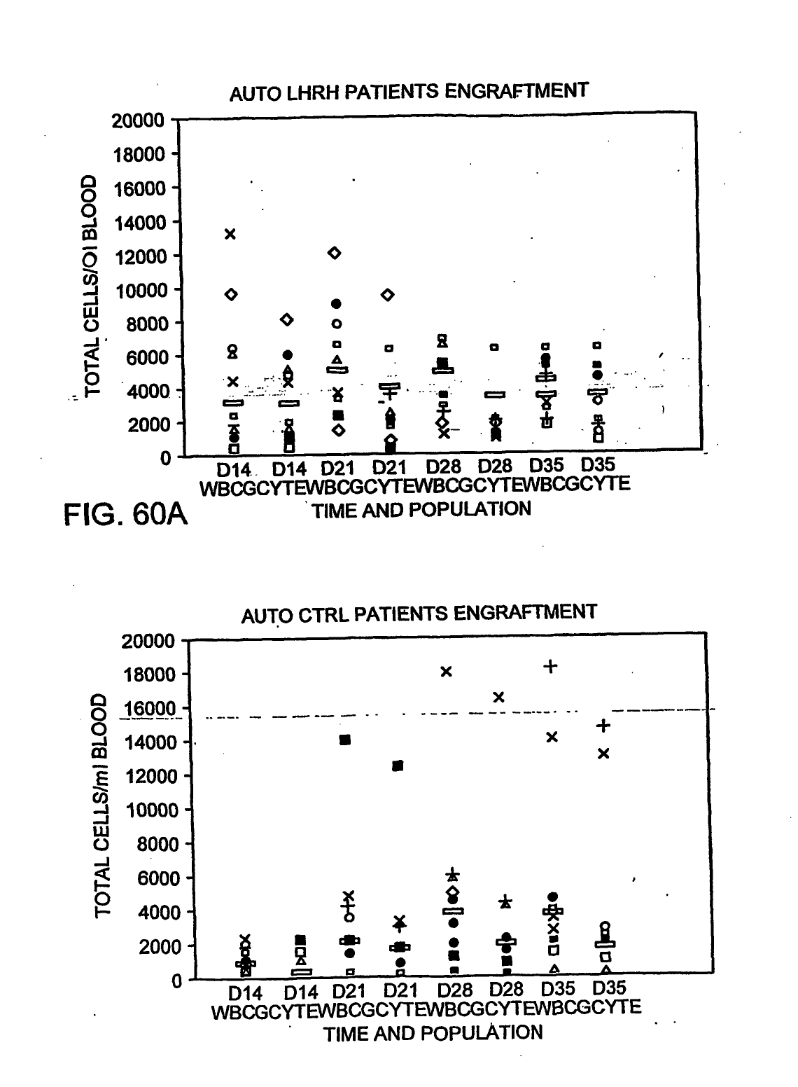

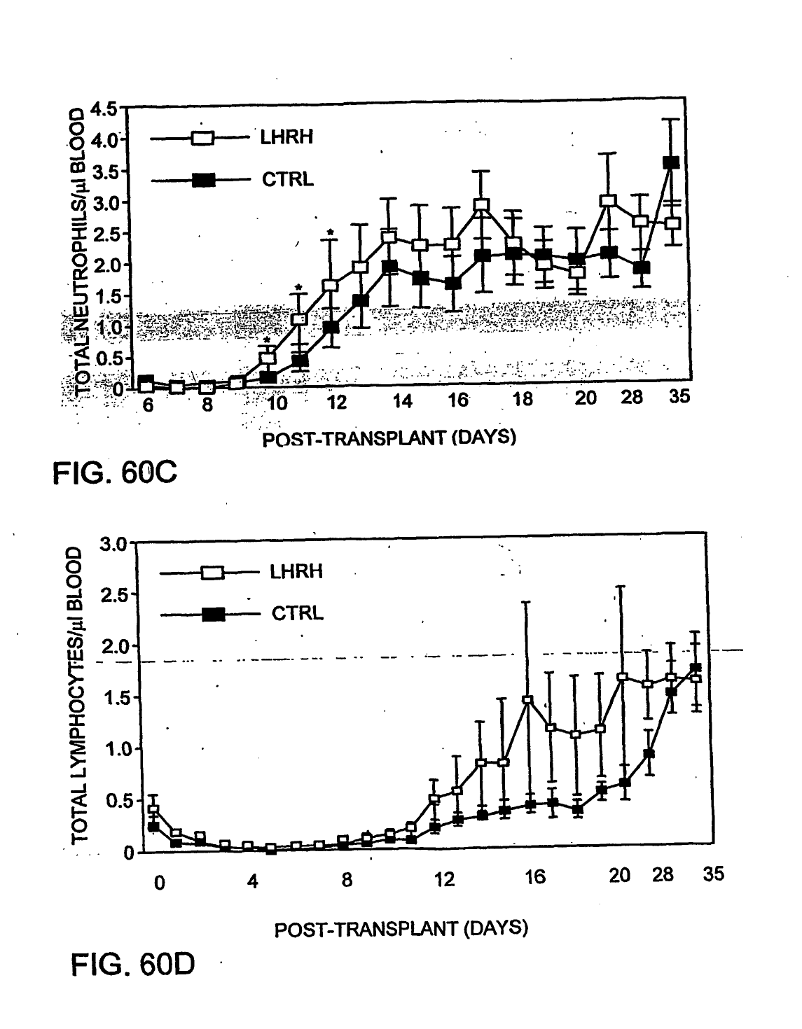

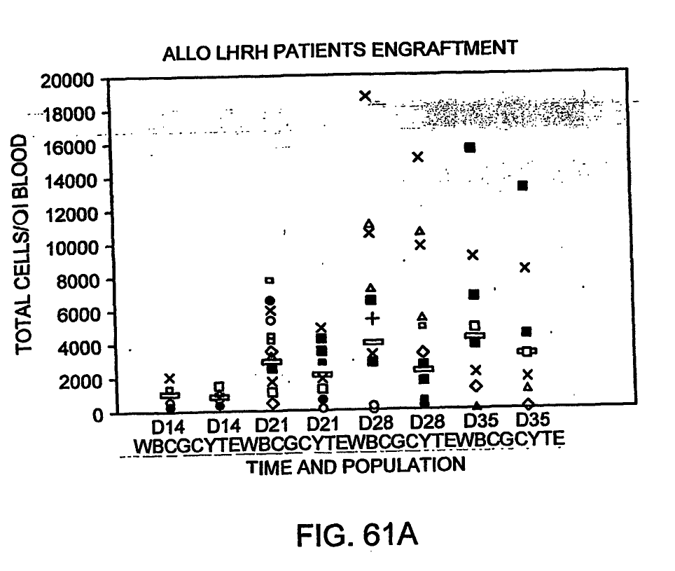

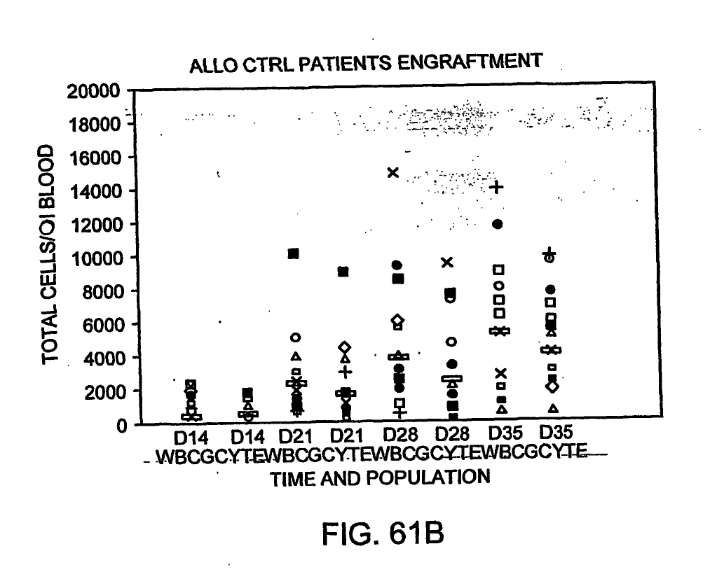

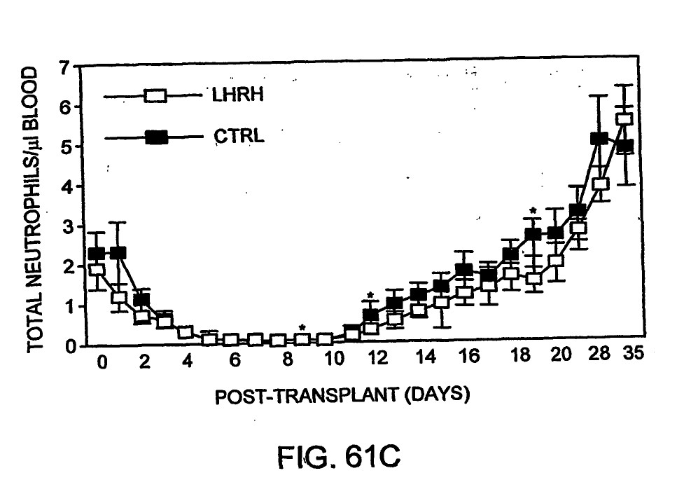

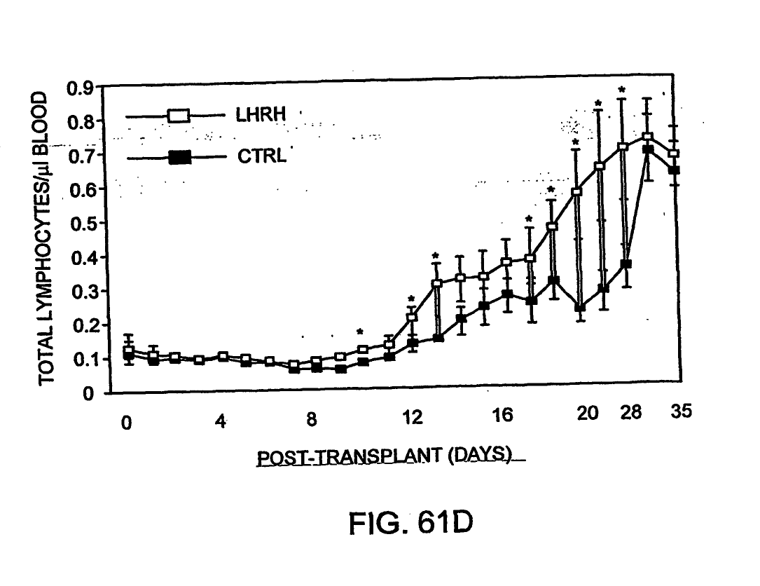

I. Autologous Stem Cell Transplant Patients Undergoing LHRH-A Treatment Prior to Transplant Enhance Rate of Engraftment. Total white blood cell (WBC) counts and granulocyte (G) counts per μl of blood were determined at days 14, 28, and 35 post transplant. As shown in FIGS. 60A-D, autologous patients who were given LHRH-A treatment showed a significantly higher number of WBC at D14 post-transplant compared to controls (FIG. 60B) (p≦0.05), with 87% showing granulocyte engraftment (≧500 cells/PI blood) compared to 45% of controls (p≦0.05) at this time-point. Autologous patients who were given LHRH-A treatment also showed a significantly higher number of neutrophils at D10-12 post-transplant compared to controls (FIG. 60C; data is expressed as mean ±1SEM of 8-20 patients. *=p≦0.05). In addition, although not significant, autologous patients had higher lymphocyte counts throughout the time-points analyzed in LHRH-A treated compared to control group (FIG. 60D). This indicates that LHRH-A therapy significantly increases lymphocyte levels following stem cell transplantation.
I. Allogeneic Stem Cell Transplant Patients Undergoing LHRH-A Treatment Prior to Transplant Enhance Rate of Engraftment. As shown in FIGS. 61A, C and D, allogeneic patients who were given LHRH-A treatment showed a significantly higher number of WBC at D14 post-transplant compared to controls (FIG. 61A) (p≦0.05) with 64% showing granulocyte engraftment (≧500 cells/μl blood) compared to 44% of controls at this time-point. In addition, allogeneic patients who were given LHRH-A treatment showed a significantly higher number of neutrophils at D9, 12 and 19 post-transplantation compared to controls (FIG. 61C; data is expressed as mean ±1SEM of 8-20 patients. *=p≦0.05). Additionally, analysis of patients undergoing peripheral blood stem cell transplantation demonstrated a significant increase in lymphocyte counts when treated with an LHRH-A prior to allogeneic transplantation (p≦0.05 at days 10, 12, 13 and 17-21 post-transplantation) (FIG. 61D).

Conclusion:

In both the allogeneic and autologous transplant models, a significant increase in WBC and granulocyte numbers at D14 post-transplant was observed with LHRH-A treated patients compared to controls (FIGS. 60 and 61). This enhanced rate of engraftment is crucial for the overall patient morbidity with neutropenia (≦200 neutrophils/ml blood) indicative of increased infection rates. As such, an early recovery of WBC and granulocyte numbers demonstrates a better survival rate for LHRH-A treated patients. Inhibition of sex steroids enhances engraftment and reconstitution prior to full thymic regeneration or the release of new T cells as a result of full thymic regeneration.

### Example 23

### Sex Steroid Ablation Increases T Cell Proliferative Responses Within One Week

These studies were conducted to determine if sex steroid ablation was capable of enhancing proliferative responses as early as 3 to 7 days following castration in mice.

Materials and Methods:

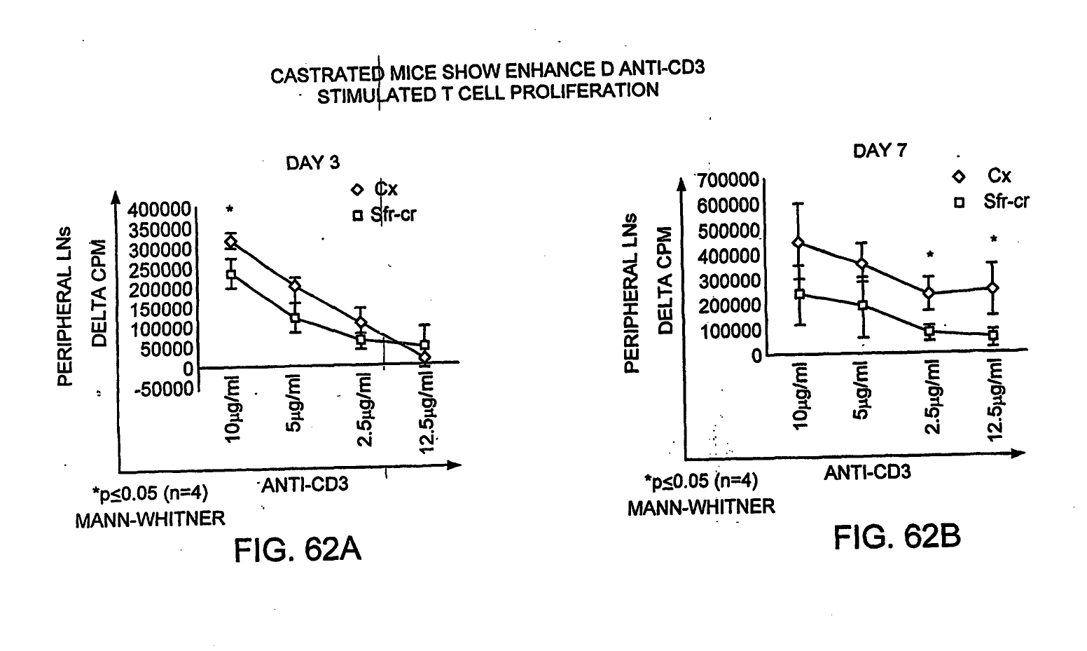

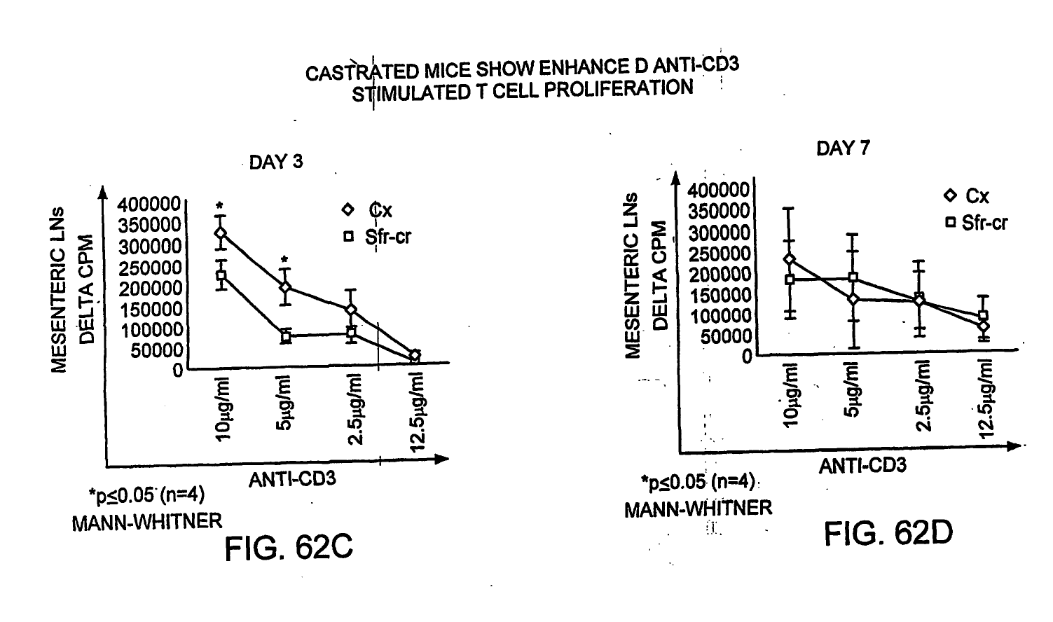

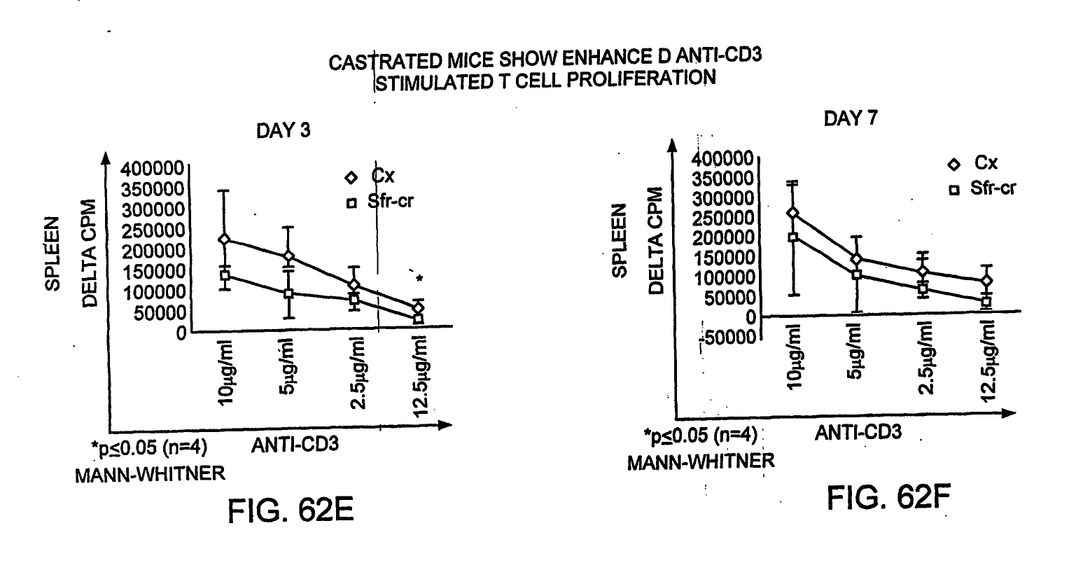

Eight week-old mice were castrated and analyzed for anti-CD3/anti-CD28 stimulated T cell proliferative response 3 days (FIGS. 62A, C, and E) and 7 days (FIGS. 62B, D, and F) after surgery. Peripheral (cervical, axillary, brachial and inguinal) lymph node (FIGS. 62A and B), mesenteric lymph node (FIGS. 62C and D), and spleen cells (FIGS. 62E and F) were stimulated with varying concentrations of anti-CD3 and co-stimulated with anti-CD28 at a constant concentration of 10 μg/ml for 48 hours. Cells were then pulsed with tritiated thymidine for 18 hours and proliferation was measured as ³H-T incorporation. Control mice were sham-castrated, n=4, *p≦0.05 (non-parametric, unpaired, Mann-Whitney statistical test).

Results:

Sex steroid ablated mice show enhanced CD28/CD3-stimulated T cell proliferation at 3 days (FIGS. 62A, C, and E) and 7 days (FIGS. 62 B, D, and F) post-castration. T cells isolated from the peripheral LN showed a significant increase in proliferative responses at 3 days (10 μg/ml anti CD-3) and 7 days (2.5 μg/ml and 1.25 μg/ml anti-CD3) post-castration.

Additionally, T cells isolated from the mesenteric lymph nodes (FIGS. 62C and D) and spleen (FIGS. 62E and F) also showed a significant increase in anti-CD3-stimulated proliferation over sham-castrated mice at 3 days post-castration.

Conclusion:

As early as 3-7 days post-castration (prior to new T cells migrating from the thymus), there is an increase in responsiveness of T cells to stimulation with anti-CD3 and CD28 cross-linking. These data indicate that sex steroid ablation has direct effects on the functionality of the peripheral T cell pool, prior to thymic reactivation.

To determine the extent of direct effects of LHRH-A on peripheral T cells in human patients, studies are conducted. Control patient T cells are incubated with various doses of LHRH-A and are analyzed at varying time-points (D3, D7 and D14) for the level of proliferation compared to control (media alone) samples. This allows the determination of whether the LHRH-A acts directly on the existing T cells by causing their activation, as was observed in the mouse model.

### Example 24

### Sex Steroid Ablation Enhances Haemopoiesis Following Congenic HSCT

Results:

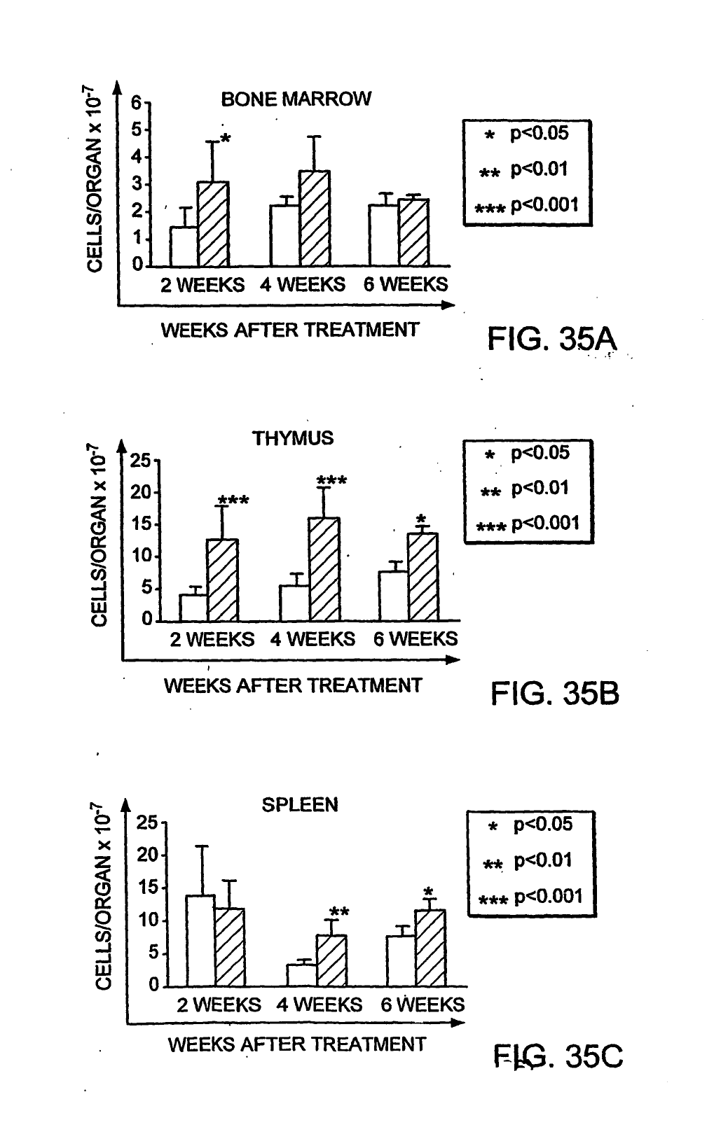

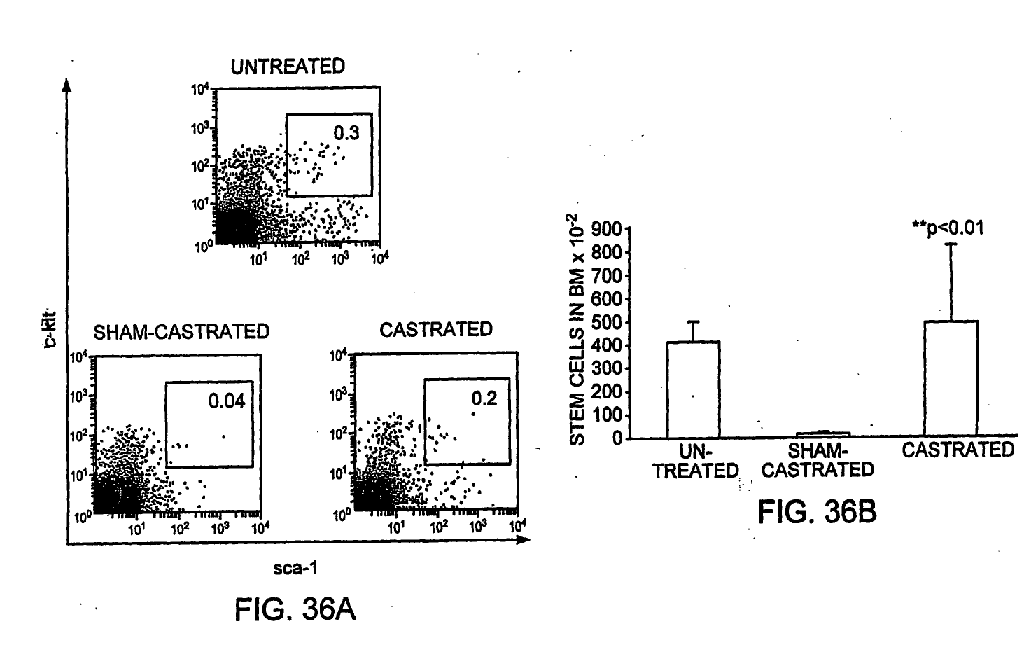

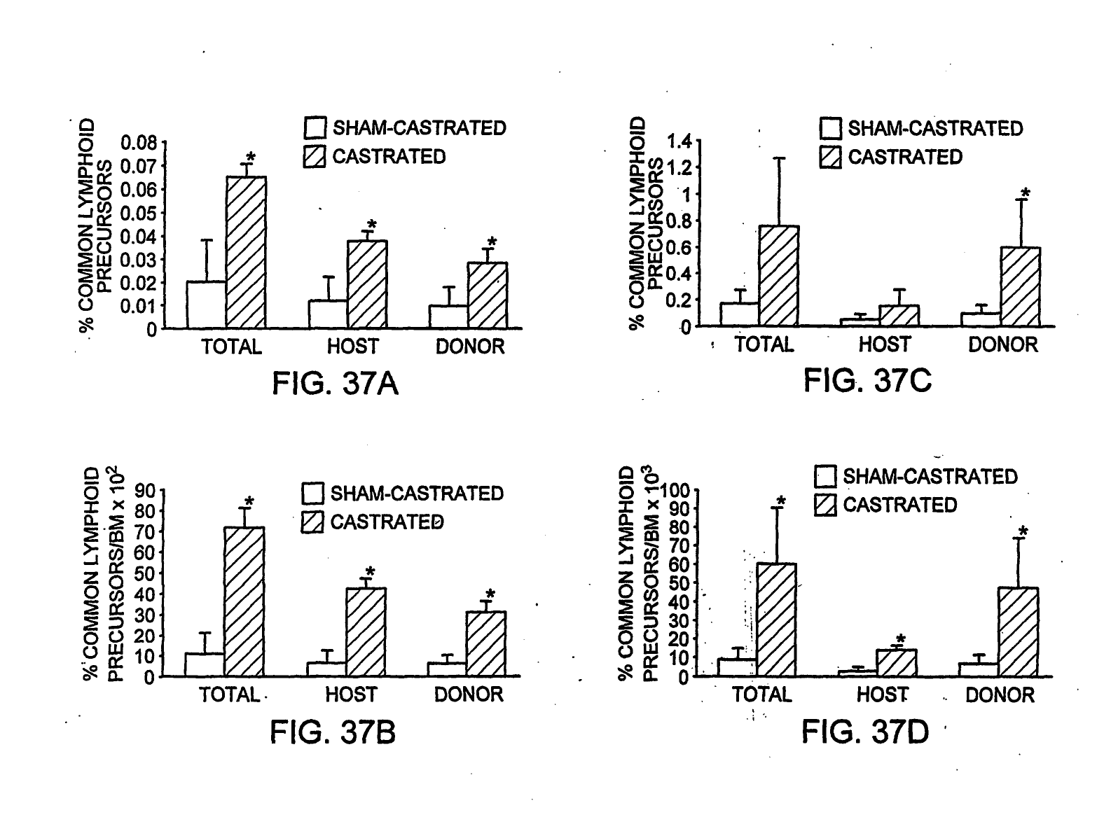

I. Castration Enhances Engraftment in the BM, Thymus, and Spleen Following HSCT. Mice were castrated 1 day before congenic HSCT. 5×10⁶ Ly5.1⁺ BM cells were injected intravenously into irradiated (800 rads) C57/BL6 mice. The BM, spleen and thymus were analyzed by flow cytometry at various time-points (2-6 weeks) post-transplant (FIG. 35). As shown in FIG. 35B, two weeks after castration and HSCT, there are significantly more cells in the BM of castrated mice as compared to sham-castrated controls. Similarly, as shown in FIG. 35C, there is a significant increase in thymic cell number 2, 4 and 6 weeks post-transplant as compared to sham-castrated controls. As shown in FIG. 35C, in the periphery, splenic cell numbers are also significantly higher than controls 4 and 6 weeks post-transplant in the castrated recipients.
II. Castration Enhances Engraftment of HSC in the BM Following Congenic HSCT. Mice were castrated 1 day before congenic HSCT. 5×10⁶ Ly5.1⁺ BM cells were injected intravenously into irradiated (800 rads) C57/BL6 mice. The BM was analyzed for lin-c-kit+sca-1+ HSC by flow cytometry at two weeks post-transplant (FIG. 36). Two weeks after BMT transplantation and castration there are significantly more donor-derived HSCs in the BM of castrated mice compared to sham castrated controls.
III. Castration Enhances Engraftment of HSC in the BM Following Congenic HSCT (2.5×10⁶ cells). Mice were castrated 1 day before congenic HSCT. 2.5×10 Ly5.1⁺ BM cells were injected intravenously into irradiated (800 rads) C57/BL6 mice. The BM was analyzed for lin-c-kit+sca-1+ HSC by flow cytometry at two weeks post-transplant (FIGS. 37A-B). FIG. 37A depicts percent of common lymphoid precursors in the BM. FIG. 37B depicts the number of common lymphoid precursors in the BM. Two weeks after BMT transplantation and castration, there is a significantly increased proportion of donor-derived HSCs in the BM of castrated mice compared to sham-castrated controls.

### IV. Castration Enhances Engraftment of HSC in the BM Following Congenic

HSCT (5×10⁶ cells). 5×10 Ly5.1⁺ BM cells were injected intravenously into irradiated (800 rads) C57/BL6 mice. The BM was analyzed for lin-c-kit+sca-1+ HSC by flow cytometry at two weeks post-transplant (FIG. 37C-D). FIG. 37A depicts percent of common lymphoid precursors in the BM. FIG. 37D depicts the number of common lymphoid precursors in the BM. Two weeks after BMT transplantation and castration, there is a significantly increased proportion of donor-derived HSCs in the BM of castrated mice compared to sham-castrated controls.

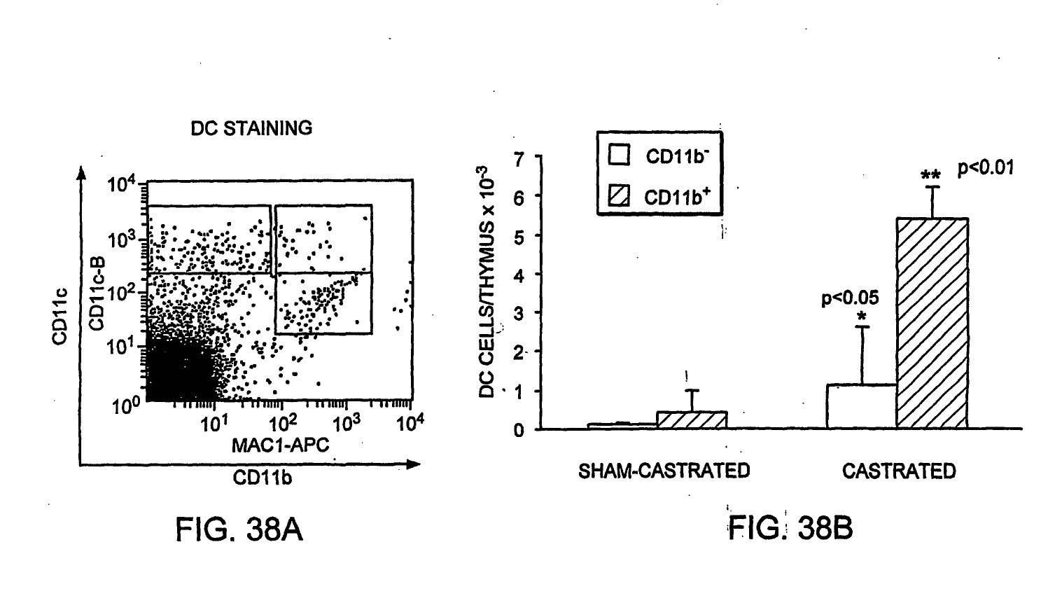

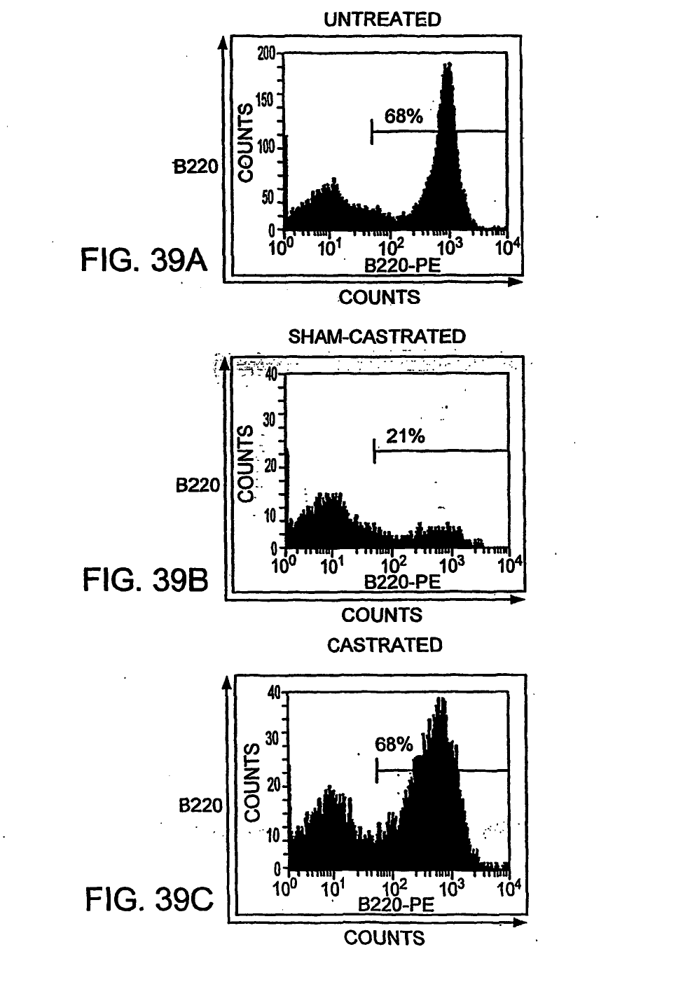

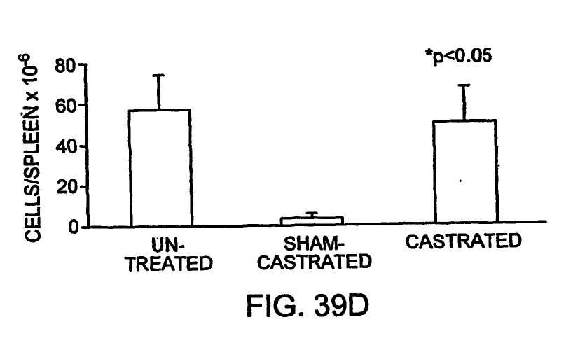

V. Castration Enhances the Rate of Engraftment of Donor-Derived DC in the Thymus Following Congenic HSCT. 5×10⁶ Ly5.1⁺ BM cells were injected intravenously into irradiated (800 rads) C57/BL6 mice. Thymocytes were analyzed by flow cytometry at two weeks post-transplant. Donor-derived DC were defined as CD45.1⁺CD11c⁺MHC class II⁺ CD11b^{+or -}. Donor-derived CD11b⁺ and CD11b⁻ DC are significantly increased in the thymii of castrated mice compared to sham-castrated controls 2 weeks after BMT (FIG. 38).
VI. Castration Enhances the Rate of Engraftment of Donor-Derived B Cells in the Spleen Following Congenic HSCT. 5×10⁶ Ly5.1⁺ BM cells were injected intravenously into irradiated (800 rads) C57/BL6 mice. Splenocytes were analyzed by flow cytometry at two weeks post-transplant. There are significantly more B220⁺ B cells in the spleens of castrated mice, as compared the sham-castrated controls 2 weeks after congenics BMT (FIG. 39).

### Example 25

### Treatment of Cancer Patients with G-CSF and/or GM-CSF Decreases the Incidence of Neutropenia Following Chemotherapy

This example illustrates the use of G-CSF and/or GM-CSF for the increase in neutrophil levels and decrease of incidence of infection in patients receiving chemotherapy.

Materials and Methods:

A randomized double blind, placebo-controlled study is conducted in 100 patients with small cell lung cancer.

G-CSF Administration. Neupogen® (Amgen, Thousand Oaks, Calif.) is administered at a dosage of 4-8 μg/kg/day s.c. from days 4-17 following chemotherapy according to manufacturer's instructions.

GM-CSF Administration. In a first study, patients receive a single SC dose of 6 mg of Neulasta® (Amgen, Thousand Oaks, Calif.) on day 2 of each chemotherapy cycle or Filgrastim® (Amgen, Thousand Oaks, Calif.) at 5 μg/kg/day SC beginning on day 2 of each cycle according to manufacturer's instructions. In a second study, subjects were randomized to receive a single SC injection of Neulasta® at 100 μg/kg on day 2 or Filgrastim® at 5 μg/kg/day SC beginning on day 2 of each cycle of chemotherapy.

Patients. All patients had been diagnosed with small cell lung cancer and were treated with standard cycles of cyclophosphamide, doxorubicin and etoposide. GM-CSF patients are diagnosed with metastatic breast cancer.

Results:

Treatment with G-CSF (Neupogen®) in this manner resulted in a clinically and statistically significant decrease in the incidence of infection as measured by febrile neutropenia, infection rates, in-patient hospitalization and antibiotic use. Numerous other Phase I/II trials report that the use of G-CSF resulted in measurable increases in neutrophils, thereby supporting the clinical use of G-CSF to treat cancer patients receiving immunosuppressive chemotherapy.

Similar results were seen using GM-CSF (Neulasta®) in a randomized, double-blind active control study (using Neupogen®), employing doxorubicin 60 mg/m² and docetaxol 75 mg/m² administered every 21 days for up to 4 cycles in the treatment of metastatic breast cancer.

Duration of neutropenia was chosen as the primary endpoint (obviously with FDA approval). Patients that did not receive Neulasta® had a 100% incidence of severe Neutropenia with a mean duration of 5-7 days and a 30-40% incidence of febrile Neutropenia.

In both studies the patients were administered Neulasta® on day 2 (c.f. Neupogen® where the drug was administered on day 4).

Both studies (one was fixed dose, the other a weight-adjusted dose for Neulasta®) demonstrated no difference in either drug at the primary endpoint of mean days of severe Neutropenia. In both cases this being approximately 1.7 days (c.f. 5-7 days with no therapy). The rates of febrile Neutropenia were comparable for both studies at approximately 10-20%.

### Example 26

### Treatment of Cancer Patients with Sex Steroid Ablation Therapy and G-CSF (and/or GM-CSF) Decreases the Incidence of Neutropenia and Infection Rates Following Chemotherapy

In a randomized double-blind study, 80 patients with small cell lung cancer are randomized into Groups 1-4 that receive G-CSF (Neupogen®) or receive G-CSF and Lupron®. All patients in all groups are monitored daily for the first 4 days of the chemotherapy cycle and thereafter every third day. All patients are monitored for CBC, neutrophil count, haematocrit and differential T cell analysis using techniques well known to those skilled in the art. Additionally, all patients are monitored twice daily for temperature and for any side effects.

Group #1-G-CSF Only

The first group consists of a group of patients (n=20) that receive G-CSF on days 4-17 following standard chemotherapy with cyclophosphamide (1 g/m²/day), doxorubicin (50 mg/m²/day) and etoposide (120 mg/m²/day X 3). All drugs are administered intravenously (I.V.) in accordance with the administration and dosage instructions in the relevant package inserts. All patients receive this therapeutic regimen in 21-day cycles. In this group the dose of G-CSF is 5 μg/kg/day delivered subcutaneously (S.C.) in accordance with the dosage instructions set out on the package insert for Neupogen® (optional range of 0-10 μg/kg/day). Subsequent chemotherapy cycles are optionally given according to the duration and severity of ANC nadir. In accordance with conventional oncology techniques, treating clinicians are also optionally able to decrease the doses used if the ANC increased towards 10,000/mm³, at which point G-CSF is discontinued.

Group #2-G-CSF Plus “High Dose” Lupron®. 21 Days Pre-Chemotherapy

The second group consists of a group of patients (n=20) that are given a “high dose” of Lupron® (3-month sustained-release product, dose of 22.5 mg, S.C.) twenty-one days prior to chemotherapy and are also administered G-CSF (5 μg/kg/day) (optional range of 0-μg/kg/day) in accordance with the aforementioned protocol from days 4-17 following chemotherapy with cyclophosphamide (1 g/m²/day), doxorubicin (50 mg/m²/day) and etoposide (120 mg/m²/day X 3). All patients receive this therapeutic regimen in 21-day cycles. Subsequent chemotherapy cycles are optionally given according to the duration and severity of ANC nadir. In accordance with conventional oncology techniques, treating clinicians are also optionally able to decrease the doses used if the ANC increased towards 10,000/mm³, at which point G-CSF is discontinued. All drugs are administered in accordance with the dosage and administration instructions set out in the respective package inserts.

Group #3-G-CSF Plus “Low Dose” Lupron®, 21 Days Pre-Chemotherapy

The third group consists of a group of patients (n=20) that are give a “low dose” of Lupron® (3 month sustained-release product, dose of 11.25 mg, S.C.) twenty-one days prior to chemotherapy and were administered G-CSF (5 μg/kg/day) from days 4-17 following chemotherapy with cyclophosphamide (1 g/m²/day), doxorubicin (50 mg/m²/day) and etoposide (120 mg/m 2/day X 3). All patients receive this therapeutic regimen in 21-day cycles. Subsequent chemotherapy cycles are optionally given according to the duration and severity of ANC nadir. In accordance with conventional oncology techniques, treating clinicians are also optionally able to decrease the doses used if the ANC increased towards 10,000/mm³, at which point G-CSF is discontinued. The chemotherapy drugs, the G-CSF and Lupron®, are administered in accordance with the package inserts for those drugs.

Group #4-G-CSF Plus “High Dose” Lupron®, 14 Days Pre-Chemotherapy

The fourth group consists of a group of patients (n=20) that are administered a “high dose” of Lupron® (3 month sustained-release product, dose of 22.5 mg, S.C.) 14 days prior to the commencement of chemotherapy. These patients are administered G-CSF (5 μg/kg/day) from days 4-17 following chemotherapy with cyclophosphamide (1 g/m 2/day), doxorubicin (50 mg/m²/day) and etoposide (120 mg/m²/day X 3). All patients received this therapeutic regimen in 21-day cycles. Subsequent chemotherapy cycles are optionally given according to the duration and severity of ANC nadir. In accordance with conventional oncology techniques, treating clinicians are also optionally able to decrease the doses used if the ANC increased towards 10,000/mm³, at which point G-CSF is discontinued. The chemotherapy drugs, the G-CSF and Lupron®, are administered in accordance with the package inserts for those drugs.

Results:

It is expected that treatment with both G-CSF and “high dose” Lupron® (Group 2) will result in a clinically and statistically significant reduction in the incidence of infection—as manifested by infection rates, antibiotic use, WBC counts, mean days of severe neutropenia (ANC <500/mm³) and febrile neutropenia—as compared to the G-CSF treated group (Group 1). It is also expected that treatment with G-CSF and the “low dose” Lupron® (Group 3) will produce results that are not statistically different from those patients treated with the higher Lupron® dosage (Group 2). Finally, it is expected that the administration of Lupron® on day 14 pre-chemotherapy (Group 4) at the 3 month sustained-release, 22.5 mg S.C. dosage, will produce data that are not statistically different from those patients in Group 2. There are expected to be, however, a number of data points that indicate that administration of Lupron® at an earlier stage of the treatment cycle may be of benefit to some patients.

### Example 27

### Treatment of Bone Marrow Transplantation Patients with Sex Steroid Ablation Therapy Reduces WBC Counts, Severe Neutropenia, Febrile Neutropenia, and Infection Rates

In a randomized unblinded study 20 patients are treated with a GnRH analog prior to receiving a BMT. This patient group is to a control group (n=19) who also undergo a BMT, but will not receive a GnRH analog.

All patients receive an allogeneic graft from a matched donor in accordance with accepted medical techniques (Lincz et al., (2001) Leuk. and Lymph. 40: 373).

All patients in the treated group received four (4) monthly doses of a GnRH analog (Lupron® 7.5 mg, S.C.). Lupron® is initially administered 21 days prior to the BMT.

Prior to transplant, the BM from all patients is ablated using standard ablating drugs and techniques (Segeren et al., (1999). Br. J. Haem. 105:127-130).

Blood samples from each patient are drawn at days −21, −2, 0, 1, 2, 3, 7, 10, 14 and 21. The blood samples are then evaluated for WBC, neutrophils, T cells (by FACS analysis, as described in detail above), granulocytes levels, and hematocrit, using the methods described in detail elsewhere herein, as well as methods well known to those skilled in the art.

Additionally, all patients are monitored for infection rates (including viral infection), the use of antibiotics, febrile neutropenia, and mean days of severe neutropenia (ANC<0.5×10⁹/L). These analyses are performed as outlined above, with the patients being followed up at 1 month, 3 months, 6 months and 9 months from the date of transplant.

Results:

It is expected that up to day 21 from transplantation, treatment with GnRH agonist (Lupron®) will result in a clinically and statistically significant reduction in WBC counts, mean days of severe Neutropenia (ANC <500/mm³) and febrile neutropenia, as compared to the non-GnRH agonist treated group.

It is also expected that, due to the small number of patients, the incidence of viral and other infection rates will not be statistically significant between the two groups up to month 1. However, it is expected that after 3, 6 and 9 months from transplant, these infection rates will be statistically different between the 2 groups, wherein the GnRH agonist treated patients will be performing better (i.e., more easily managed and recover faster).

### Example 28

### Treatment of Cancer Patients with Sex Steroid Ablation Therapy Increases Haemopoiesis and Neutrophil Counts

In a randomized, unblinded study, male patients (n=30) with metastatic prostate cancer receive, in the normal course of their treatment, a GnRH agonist (Lupron Depot® injection (7.5 mg s.c. monthly)). All patients also received an anti-androgen drug. Cosudex® may also be given (5 mg/day or 50 mg/day orally) for 1 month.

All patients undergo a Full Blood Analysis (FBA) prior to the commencement of therapy. FBA involves standard pathology analysis of lymphocytes, white blood cells, hematocrit and red blood cell content. In addition, analysis of immunological parameters including T cell stimulation, cytokine production, immune cell subsets and TREC analysis are performed.

Additionally, blood samples from each patient are drawn at days −2, 0, 1, 2, 3, 7, 10, 14, 21, and 28, and monthly thereafter for 6 months. The blood samples are then evaluated for WBC, neutrophils, T cells (by FACS analysis, as described in detail above), granulocytes levels, and hematocrit, using the methods described in detail elsewhere herein, as well as methods well known to those skilled in the art.

It is expected that the majority of patients (approximately 70%) will have an increase in haemopoiesis (as measured by T cell analysis) and neutrophil count, as compared to the baseline levels (days −2 and 0), within the first 14 days of GnRH agonist therapy.

### Example 29

### Treatment of Patients with Sex Steroid Ablation Therapy Increases Influenza Vaccination Efficacy

In a randomized, unblinded study, male patients (n=45) with metastatic prostate cancer received in the normal course of their treatment a GnRH agonist (Lupron Depot® injection (7.5 mg s.c. monthly)). All patients also received an anti-androgen drug. Cosudex® may also be given (5 mg/day or 50 mg/day orally) for 1 month.

All patients underwent a Full Blood Analysis (FBA) prior to the commencement of therapy.

The patients that receive the GnRH agonist and Cosudex® are randomly assigned into one of three groups of 15 patients per group as follows:

Group #1

The first group consists of 15 patients that receives an influenza vaccination (e.g., Fluarix® (GlaxoSmithKline, Australia), 0.5 ml, i.m.) at day 0, according to manufacturer's instructions.

Group #2

The second group consists of 15 patients that receive an influenza vaccination (e.g., Fluarix® (GlaxoSmithKline, Australia), 0.5 ml, i.m.) at day 21, according to manufacturer's instructions.

Group #3

The third group consists of 15 patients that receive an influenza vaccination (e.g., Fluarix® (GlaxoSmithKline, Australia), 0.5 ml, i.m.) at 8 weeks, according to manufacturer's instructions.

Group #4—Control

The fourth group is a control group that consists of an additional group of 15 males of similar age that do not have prostate disease and receive no medication. The control group receives an influenza vaccination (e.g., Fluarix® (GlaxoSmithKline, Australia), 0.5 ml, i.m.) at day 0, according to manufacturer's instructions.

All patients are monitored throughout (on days −2, 0, 2, 3, 5, 7, 14, 21, 28 and monthly thereafter for a further 6 months) for WBC, neutrophils, T cells (by FACS analysis, as described in detail above), granulocytes levels, and hematocrit, using the methods described in detail elsewhere herein, as well as methods well known to those skilled in the art. At each time-point, the patients are also monitored for the presence or absence of Influenza (types A and B) virus, utilizing nasopharyngeal swabs and FLU OIA (Thermo BioStar).

The patients in each group are further monitored for the presence of hemagglutination-inhibition antibody to H1N1 strains of Influenza at day −2, as well as all time-points post-inoculation with the influenza vaccine using techniques well known to those in the art.

Results:

It is expected that no patients will report any infective episodes with influenza in any of Groups 1-3, as compared to the control patients in Group 4 that return a positive swab to influenza.

It is also expected that patients in all groups will have a higher hemagglutination-inhibition antibody titer than prior to GnRH agonist therapy. Patients in Groups 2 and 3 are expected to produce higher antibody titers than those patients in Groups 1 and 4. However, it is expected that all groups will have similar antibody titers from 12 weeks onwards.

Additionally, it is expected that the protection rates (percentage of subjects with hemagglutinin-inhibiters greater than 40 for H1N1 strain of influenza) will be greatest in Group 2 and least in Group 4.

### Example 30

### LHRH-A Treatment Effectively Depletes Serum Testosterone

Materials and Methods:

Detection of sex steroid levels in patient sera was performed using a ¹²⁵I-Testosterone radioimmunoassay (RIA). Prior to the assay, all reagents, samples and controls were brought to room temperature. Control tubes had either buffer alone-non-specific binding (NSB) tube or 0 ng/ml testosterone standard (B₀). Buffer alone, standards (0-10 ng/ml testosterone) or test samples were added to each tube, followed by sex binding globulin inhibitor (SBGI) to limit non-specific binding of the radio-labeled testosterone. The ¹²⁵I-testosterone was added to each tube followed by an anti-testosterone antibody (except for the NSB tubes). Tubes were then incubated at 37° C. for 2 hours. Following this, a secondary antibody was added to all tubes which were vortexed and incubated for a further 60 mins. Tubes were centrifuged (1000_{gmax}) for 15 mins., supernatant removed and the precipitate counted on a Packard Cobra auto-beta counter. Triplicate cpm results were averaged and a standard curve constructed using the formula for percent bound testosterone (B/B₀):

$$\% B / {B}_{0} = \frac{Sample - NSB}{{B}_{0} - NSB}$$

Sample=average cpm of particular test sample
NSB=average cpm of non-specific binding tube
B₀=average cpm of 0 ng/ml standard (total binding tube)

The level of testosterone in each test sample was determined from the standard curve.

Results:

LHRH-A Administration to Prostate Cancer Patients Results in Castrate Levels of Serum Testosterone. In order to determine the efficacy of LHRH-A treatment, serum testosterone levels were analyzed for all patients before treatment and at 4 months of treatment with LHRH-A. Analysis was performed using a radioimmunoassay (RIA) with ¹²⁵I-Testosterone. The concentration of serum testosterone was within the range of 1-3 ng/ml testosterone (mean=2.3 ng/ml) prior to hormonal treatment (FIG. 46A). At 4 months of treatment, patients had essentially no detectable serum testosterone indicating successful abrogation of sex steroid release.

LHRH-A Administration Does Not Affect the Percent of Lymphocyte Subsets Within the Peripheral Blood. Following 4-months of treatment with LHRH-A, no changes in the proportion of any lymphocyte subset was observed compared to pre-treatment values. These values are all within normal ranges (data not shown). Peripheral blood lymphocytes were analyzed by FACS for proportions and cell numbers of T, B, and myeloid-derived (NK and macrophages) cells. No change in proportion of any cell subset was observed following LHRH-A administration. Furthermore, the proportions of all lymphocyte subsets were within normal ranges for this age group (FIG. 84C); Hannet et al., 1992; Xu et al., 1993).

### Example 31

### Thymic Reactivation Following Transient Ablation of Sex Steroids with Goserelin Acetate (Zoladex®) in Patients Undergoing Autologous or Allogeneic Stem Cell Transplantation

In this example, goserelin acetate (Zoladex®) is administered prior to autologous or allogeneic peripheral blood stem cell transplantation (PBSCT). The primary endpoint is thymic reactivation as measured by in vitro assays. Patients will be followed for 6 months post-transplant. Twenty (10 allografts and 10 autografts) patients will be entered into the study. This example investigates the effect of inhibiting sex steroid production at the level of LHRH, using agonists thereof to desensitize the pituitary and hence prevent release of LH and FSH. In turn, this causes a block in the gonadal production of androgens and estrogen which removes the inhibitory effects on thymic function. The groups examined in this trial are patients undergoing high-dose chemoradiotherapy (HDT) and PBSCT.

Goserelin acetate (Zoladex®) is a potent synthetic decapeptide analogue of LHRH. When given acutely, goserelin acetate will release LH from the pituitary gland. However, following chronic administration, goserelin acetate is a potent inhibitor of gonadotrophin production resulting in gonadal suppression and, consequently, sex organ regression. In animals and humans, following an initial stimulation of pituitary, LH secretion and a transient elevation in serum testosterone, chronic administration results in inhibition of gonadotrophin secretion. The result is a sustained suppression of pituitary LH occurring within approximately 3 weeks of initiation of therapy and a reduction in serum testosterone levels in males to a range normally seen in surgically castrated men. This suppression is then maintained as long as therapy is continued.

Patients are male or female, aged 18 years or older that are due to undergo high-dose therapy (HDT) with PBSCT for malignant disease or BM failure. The 10.8 mg implant formulation (for men) of the Zoladex® is dispersed in a cylindrical rod of biodegradable and biocompatible polyglactins and is released continuously over 12 weeks following subcutaneous injection. The 3.6 mg implant (for women) is dispersed in a cylindrical rod of a biodegradable and biocompatible polyglactin and is released continuously over 28 days following subcutaneous injection. The implants are commercially supplied in a purpose-designed applicator with 14-16 gauge needles.

Reduction of sex steroids in the blood to minimal values may take several weeks. Consequently, 21 days prior to PBSC infusion (day 0), patients are injected with the sex steroid ablation therapy in the form of LHRH agonist Zoladex® (implant). For males, 10.8 mg goserelin as a single dose (effective for 3 months) are administered on day −21 with a further injection of 3.6 mg on day 63 (effective for 28 days). For females, 3.6 mg (effective for 28 days) are administered on day −21 and days 7, 35 and 63. This should be effective in reducing sex steroid levels sufficiently to reactivate the thymus (predicted 4 months post PBSCT). Thus, in this example, only 4-6 months of treatment are administered. Other doses may be deemed acceptable as determined readily by those skilled in the art.

PBPCs are infused on day 0. The reactivated thymus takes up the infused precursor cells and convert them into new T lymphocytes and epithelial thymic cells. Maximum sex steroid “ablation” is at the time of PBSC infusion, and hence infused PBSC is able to assist in thymic reconstitution. It is expected that within 3-4 weeks after PBSCT, the first new T cells will be present in the blood stream, but the therapy will be maintained for 3 months post-PBSCT to allow complete normalization of the immune system.

Thymic function is determined by assessment of T cell subsets by flow cytometry, T cell responses in vitro, and production of TRECs.

Prior to the start of the study, routine pre-HDT investigations are performed, and baseline FBE, electrolytes, LFTs documented. Other pre-treatment analyzes include serum β-HCG (women), thymus CT, bone density studies, protein electrophoresis and immunoelectrophoresis, hormone studies: TFTs, FSH, LH, estrogen, progesterone, testosterone. Additionally, various baseline T cell assays will be performed. Leukocytes will be purified from 50 ml of blood and examined as follows:

### (a) Flow Cytometry

Naïve vs. Memory T Cells

### CD27-FITC, CD45RA-PE, CD45RO-PerCP, CD4- or CD8⁻APC

### CD27-FITC, CD45RO-PerCP, CD4/CD8⁻APC, Ki-67-PE

### CD62L, CD45RO-PerCP, CD103, CD4/CD8⁻APC

T Cell Subsets

### CD4-FITC, CD8⁻APC, αβTCR-PE, γδTCR-B/S-PerCP

### CD25-PE, CD69-CyChrome, CD4-FITC, CD8⁻APC

### CD28-CyChrome, αβTCR-PE, CD4-FITC, CD8⁻APC

B Cells/Myeloid Cells

### CD19-FITC, CD3-PerCP, CD56-PE, CD34-APC

CD11b-CyChrome, CD11c-PE, CD4-FITC, CD8⁻FITC

Cytokines

### IL4-PE, IFNg-APC, CD4-FITC, CD8

Other Markers

### CD11a, CD95, HLA-DR, CD2, CD5

All patients serve as internal controls because they are examined pre- and post-treatment. Staining specificity controls will include isotype controls with FITC/PE/APC, etc. and blocking of FcR prior to staining.

### (b) T Cell Function

Blood lymphocytes will be examined for their ability to respond to CD3 cross-linking in vitro.

### (c) TREC Analysis

Naïve T cells will be isolated and probed for the presence of T cell receptor excision circles which are formed as a result of rearrangement of the TCR genes as described above. Their presence is a very strong indication of export from the thymus (being the only source of mainstream T cell production). Because cell division is associated with thymic development post-rearrangement of the TCR genes, TREC levels may be an underestimate of thymic migrants (about 10% of actual levels).

### Example 32

### Treatment of a Patient with Pernicious Anemia

An adult (e.g., 35 years old) human female patient is suffering from pernicious anemia, an autoimmune disease. Her CD34+ hematopoietic stem cells (HSC) are recruited from her blood following 3 days of G-CSF treatment (2 injections/day, for 3 days, 10 g/kg). Her HSC can be purified from her blood using CD34. To collect the CD34⁺ cells, peripheral blood of the donor (i.e., the person who will be donating his/her organ or skin to the recipient) is collected, and CD34⁺ cells isolated from the peripheral blood according to standard methods. One non-limiting method is to incubate the peripheral blood with an antibody that specifically binds to human CD34 (e.g., a murine monoclonal anti-human CD34+ antibody commercially available from Abcam Ltd., Cambridge, UK), secondarily stain the cells with a detectably-labeled anti-murine antibody (e.g., a FITC-labeled goat anti-mouse antibody), and isolate the FITC-labeled CD34⁺ cells through fluorescent-activated cell sorting (FACS). Because of the low number of CD34⁺ cells found in circulating peripheral blood, multiple collection and cell sorting may be required from the donor. The CD34⁺ cells may be cryopreserved until enough are collected for use.

HSC can be transfected by using a variety of techniques including, without limitation, electroporation, viral vectors, laser-based pressure wave technology, lipid-fusion (see, e.g., the methods described in Bonyhadi et al., 1997). In one example, her HSC are transfected with the β chain of the H/K-ATPase proton pump, using the MHC class II promoter for the expression.

To stop the ongoing autoimmune disease, the patient will undergo T cell depletion and/or other immune cell depletion. She will also undergo thymic regeneration to replace these T cells and, hence, overcome the immunodeficiency state. To do this, she will receive 4, once monthly injections of Lupron (7.5 mg) to deplete the sex steroids (by 3 weeks) thereby allowing reactivation of her thymus. This will also allow uptake of the HSC and to establish central tolerance to the autoantigen in question. It is not clear why autoimmune disease starts but cross-reaction to a microorganism is a likely possibility; depleting all T cells will thus remove these cross-reactive cells. If the disease was initiated by such cross-reaction, it may not be necessary to transfect the HSC with the nominal autoantigen. Simply depleting T cells followed by thymic reactivation by disrupting sex steroid signalling may be sufficient. One standard procedure for removing T cells is as follows. The human patient receives anti-T cell antibodies in the form of a daily injection of 15 mg/kg of Atgam (xeno anti-T cell globulin, Pharmacia Upjohn) for a period of 10 days in combination with an inhibitor of T cell activation, cyclosporin A, 3 mg/kg, as a continuous infusion for 3-4 weeks followed by daily tablets at 9 mg/kg as needed. This treatment does not affect early T cell development in the patient's thymus, as the amount of antibody necessary to have such an effect cannot be delivered due to the size and configuration of the human thymus. The treatment is maintained for approximately 4-6 weeks to allow the loss of sex steroids followed by the reconstitution of the thymus. The prevention of T cell reactivity may also be combined with inhibitors of second level signals such as interleukins, accessory molecules (blocking, e.g., CD28), signal transduction molecules or cell adhesion molecules to enhance the T cell ablation or other immune cell depletion.

### Example 33

### Treatment of a Patient with Type I Diabetes

A similar approach to that described in Example 32 is undertaken with a patient with Type I diabetes. The T cells will be removed by broad-based depletion methods (see above), thymic rejuvenation instigated by 4 month Lupron treatment and the patient's immune system recovery enhanced by injection of pre-collected autologous HSC transfected with the pro-insulin gene using the MHC class II promoter. The HSC will enter the thymus, differentiate into DC (and all thymocytes), and present pro-insulin to the developing T cells. All those potentially reactive to the pro-insulin will be killed by apoptosis, leaving a repertoire free to attack foreign infectious agents.

In the case that the autoimmune disease arose as a cross-reaction to an infection or simply “bad luck,” it would be sufficient to use autologous HSC to help boost the thymic regrowth. If there is a genetic predisposition to the disease (family members can often get autoimmune disease), the thymic recovery would be best performed with allogeneic highly-purified HSC to prevent graft versus host reaction through passenger T cells. Umbilical cord blood is also a good source of HSC, and there are generally no or very few alloreactive T cells. Although cord blood does not have high levels of CD34+ HSC, they may be sufficient for establishment of a microchimera—even about 10% of the blood cells being eventually (after 4-6 weeks) sufficient to establish tolerance to the autoantigen with sufficient intrathymic dendritic cells.

### Example 34

### Treatment of a Patient Suffering from Allergies

In the case of allergy, a similar principle as Examples 32 and 33 would be undertaken. The allergic patient would be depleted of T cells as above. In severe cases where there is exacerbation through IgE or IgG producing B cells (plasma cells) it may be necessary to use myeloablation as for chemotherapy. Alternatively, whole body irradiation may be used (e.g., 6 Gy). The entire immune system would be rejuvenated by the use of 34 month GnRH and injection intravenously of the HSC (allogenic or autologous as appropriate). Allogeneic HSC would be used in the case of genetic disposition to allergy but otherwise mobilized autologous HSC would be used.

### Example 35

### Effects of Castration on NOD and NZB Mice

Non-obese diabetic mice (NOD mice) are a very well-characterized model for type I diabetes. Extensive research has confirmed that the pathology of this disease is due to abnormal T cell infiltration of the pancreas and autoimmune destruction of the insulin-producing islet cells. The structure of the thymus in these animals is abnormal—there is ectopic expression of medullary epithelial cells (identified by mAb MTS 10), the presence of large B cell follicles and thymocyte-rich areas which lack the epithelial cells.

To examine the impact of sex steroids on these mice, 20 three week old female NOD mice were surgically ovariectomized and 20 were sham-operated. This stage was chosen because it is prior to disease onset. Blood glucose was monitored from 10 weeks of age. By 21 weeks of age, over 60% of the sham-castrated mice had developed diabetes but <20% of the castrated group had. There was insulitis (infiltration of the pancreas) but no islet destruction. After surgical castration, there was also a normalization of the thymic defects with well-defined cortex and medulla, loss of the B cell follicles, an increase in CD25+ regulatory cells. The increase in regulatory T cells may be very important because they could alter the pathogenic cytokine profile of the emigrating thymocytes. Hence, castration has a dramatic impact on the development and progression of diabetes in NOD mice.

Sixteen (16) ovariectomized and sixteen (16) sham-operated NOD mice were examined for 21 weeks for the development of diabetes (elevated blood glucose levels; BGL) and insulitis. At autopsy they were also examined for the presence of thymic structural abnormalities. As shown in FIG. 45, whereas 60% of the sham-operated NOD mice had diabetes, fewer than 20% of the castrated group had diabetes. This clearly shows a retarding or even prevention of the diabetes.

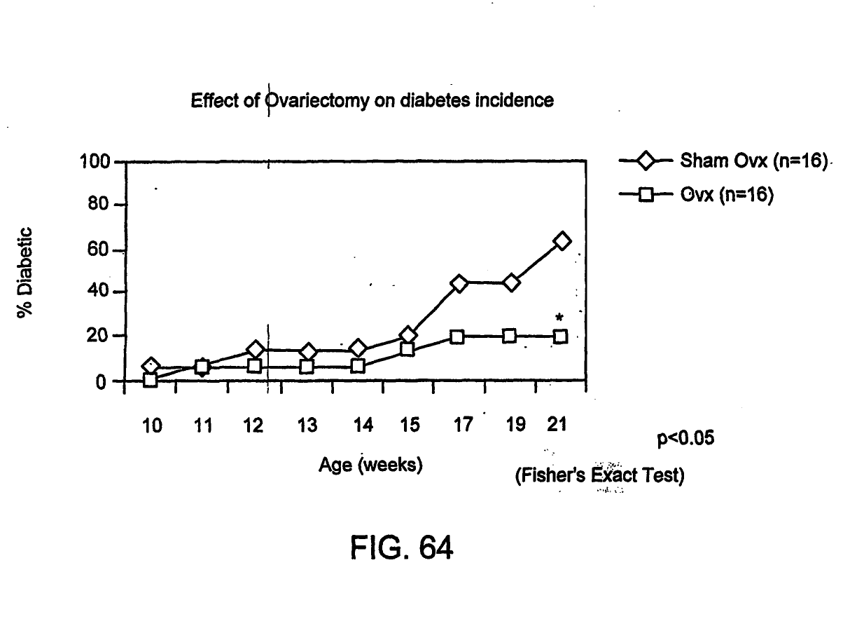

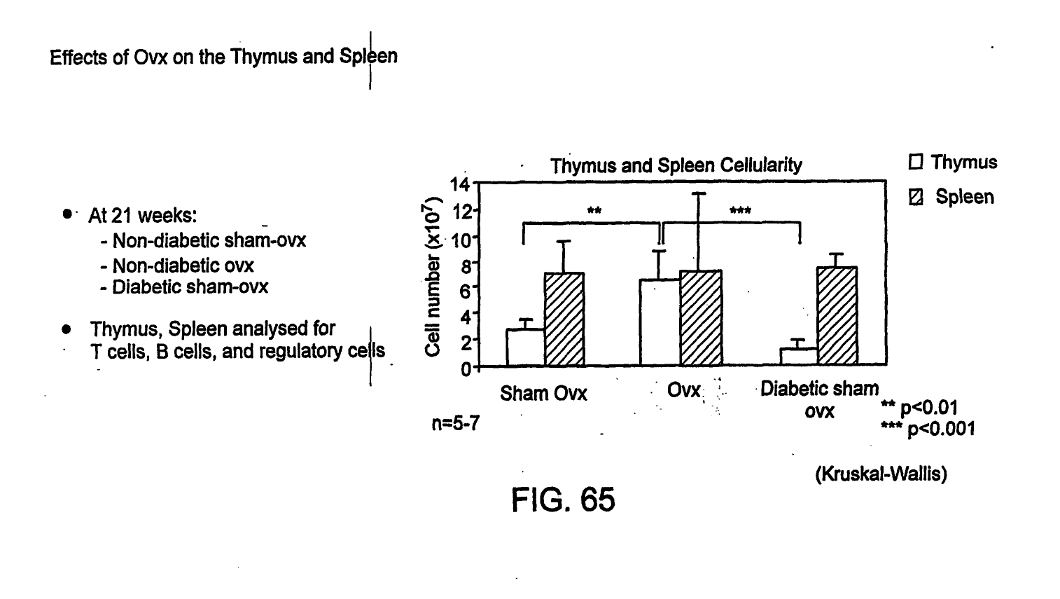

As shown in FIG. 64, castrated NOD mice had a marked increase in total thymocyte number but no differences in total spleen cells. In the diabetic, castrated mice there was a marked decrease in total thymocyte number, which may have pre-disposed these mice to disease and suggests that the diabetes trigger may have occurred before the castration.

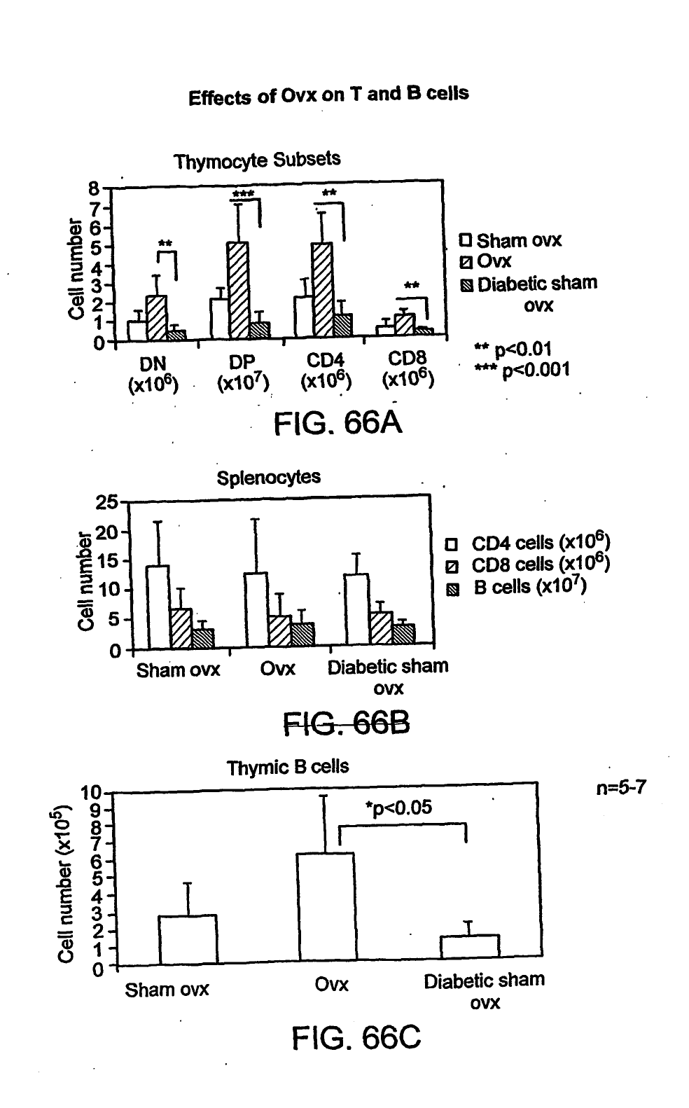

There was a significant increase in all thymocyte subclasses (FIG. 66A) but there was no change in their proportions (data not shown). Interestingly, there was no change in B cells compared to sham-castrated mice (FIG. 66C) nor in the total T or B cells in the spleen (FIG. 66B).

In parallel, with the increase in total thymocytes post-castration, there was a marked increase in CD25+ regulatory cells (data not shown). There was no such change in the spleen.

The effect of castration was also examined on NZB mice, which are a model for systemic lupus erythematosis (SLE). NZB mice have marked abnormalities in the thymus which are manifest before disease onset and are closely associated with disease. These defects include a poorly-defined cortex-medulla demarcation and abnormal clusters of B cells (see Takeoka et al., (1999) Clin. Immunol. 90:388).

Mice were castrated or sham-castrated at 4-7 weeks of age and examined 4 weeks later.

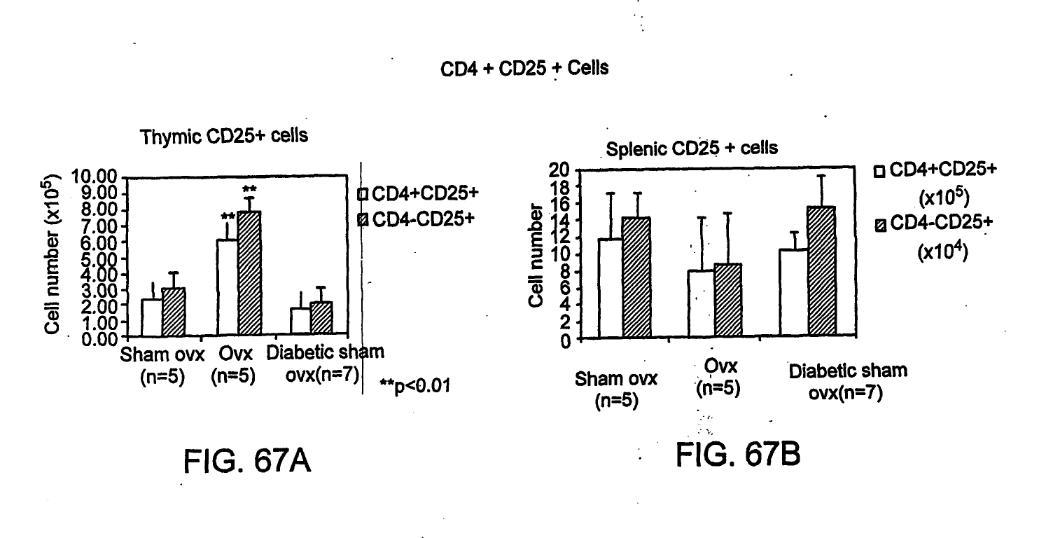

There was a marked increase in total thymocytes (FIG. 67A) and spleen cells (FIG. 67B). There was also a marked increase in thymic regulatory cells (CD25⁺ and NKT cells). The cytokines from these mice may be influencing the effector T cells and modulating their potential pathogenicity. By immunohistology, the castrated mice had a normal thymic architecture and a loss of the B cell follicles (data not shown).

### Example 36

### Effect of Castration on Immunization with Tumor-Specific Antigen

Human Papillomavirus (HPV) infection causes genital herpes, which may lead to cervical cancer in some women. In fact, over 90% of all cervical cancers contain HPV DNA. Papillomaviruses are double-stranded DNA viruses that infect skin and mucosal surfaces. More than 80 types of HPV have been identified to date. HPV16 is one of the major types associated with cervical cancers.

E7 is the major oncogenic protein associated with HPV16-induced cervical cancer. Expression of the E7 open reading frame with activated ras has been shown by other groups to be sufficient to transform primary epithelial cells in culture to a malignant phenotype (Lin et al. (1996) Cancer Res. 56:21). Thus, E7 is an attractive tumor specific antigen for use in immunotherapy and/or vaccination for cervical cancer and precursor lesions. Indeed, other groups have shown that mice immunized with an optimal dose of 50 μg/ml of an E7-GST fusion protein, with Quil A as adjuvant were protected against a subsequent challenge with an HPV16E7-transfected tumor cell line (Fernando et al., (1999) Clin. Exp. Immunol. 115:397.

This experiment was undertaken to determine if castration of mice was able to enhance the efficacy of vaccination (as a prophylactic vaccine) and/or immunotherapy (as an therapeutic vaccine) with a suboptimal dose of HPV16E7.

Materials and Methods

Adult (>9 month old) C57BL/6 mice received a subcutaneous injection of E7-positive syngeneic E7+ TC1 tumor cells derived from primary epithelial cells of C57BL/6 mice co-transformed with HPV-16 E6 and E7 and c-Ha-ras oncogenes. Five days later, the mice were surgically castrated as described above by a scrotal incision, revealing the testes, which were tied with suture and then removed along with surrounding fatty tissue. The wound was then closed using surgical staples. Sham-castrated mice were prepared following the above procedure without removal of the testes and were used as negative controls. Seven days following tumor challenge, castrated or sham-castrated mice were injected with a suboptimal dose (5 μg/ml) of the E7GST fusion protein. Positive control mice received the optimal 50 μg/ml dose of the E7GST fusion protein. Twenty-five days later, mice were analyzed for tumors (visually, and tumor mass), and T cell responsiveness (IFNγ production using standard ELIspot methods and CTL lytic activity by ⁵¹Cr release assays using HPV16E7-pulsed target cells as described above).

### Results

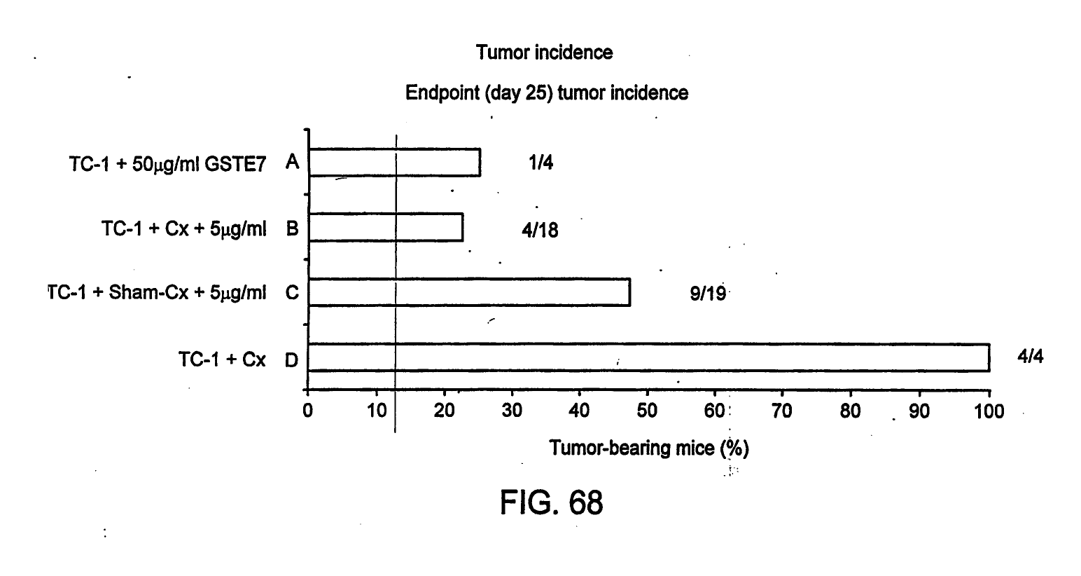

As shown in FIG. 68, only 22% (4/18) of castrated mice receiving the suboptimal dose of E7GST had tumors, as compared to 47% (9/19) tumor occurrence in the sham-castrated suboptimal dose controls. Notably, the proportion of protected, castrated mice receiving the suboptimal dose E7GST vaccine, (78%) was comparable to the proportion of protected, positive control mice, which received a 10-fold higher (optimal) dose of the vaccine (¾, 75%). All of the castrated mice that did not receive the vaccine had tumors.

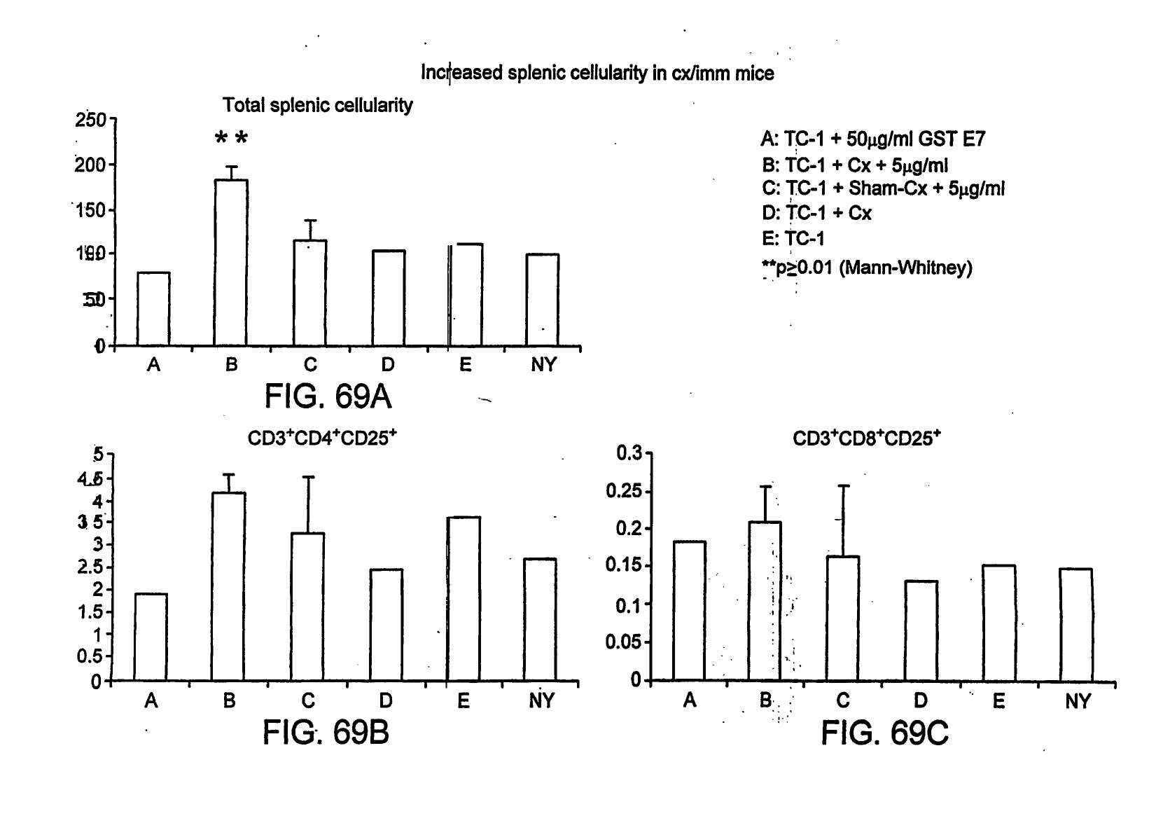

As shown in FIG. 69, castrated mice receiving the suboptimal E7GST dose had a significantly higher number of cells in the spleen as compared to all other groups (FIG. 69A) (p≧0.01). However, the overall number of activated (CD25⁺) CD4⁺ (FIG. 69B) and CD8⁺ (FIG. 50C) T cell in the spleens of the castrated, suboptimal E7GST group remained the same as compared to all the other groups tested.

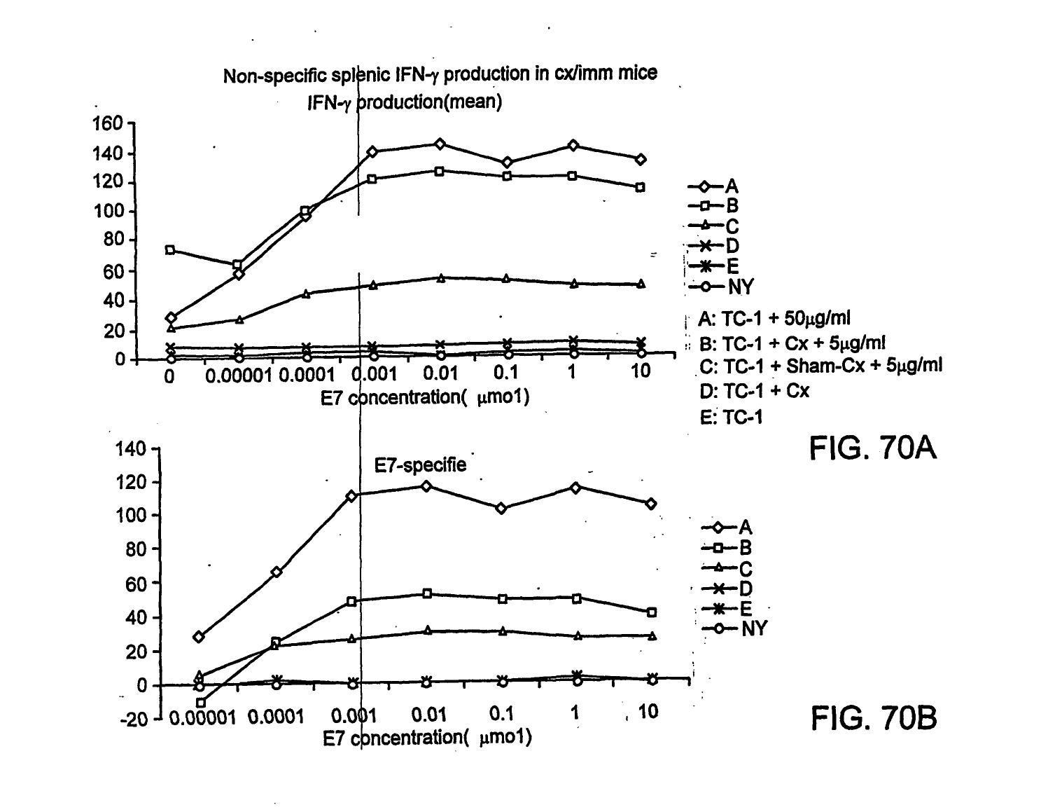

As shown in FIG. 70A, non-specific production of IFNγ in the splenocytes of castrated mice receiving the suboptimal GST-E7 dose was comparable to the positive control mice receiving a 10-fold higher (optimal) dose of the vaccine. Sham-castrated mice receiving the suboptimal dose vaccine had a moderate level of IFNγ production, whereas the negative control animals had little to no non-specific splenocyte production of IFNγ. This data parallels that seen with respect to tumor incidence (see FIG. 68), and indicates that the production of IFNγ by splenocytes is directly proportional to tumor incidence in these animals. This is not surprising given that Th1 cell-mediated responses are primarily responsible for tumor protection.

As shown in FIG. 70B, the level of E7-specific production follows the same trends as discussed above with respect to non-specific production of IFNγ. While the level of E7-specific IFNγ production in castrated mice receiving the suboptimal GST-E7 dose was slightly lower than that of the positive control mice receiving a 10-fold higher (optimal) dose of the vaccine, it was still higher than the levels observed in the sham-castrated mice receiving the suboptimal dose. Once again, the negative control animals had little to no E7-specific splenocyte production of IFNγ.

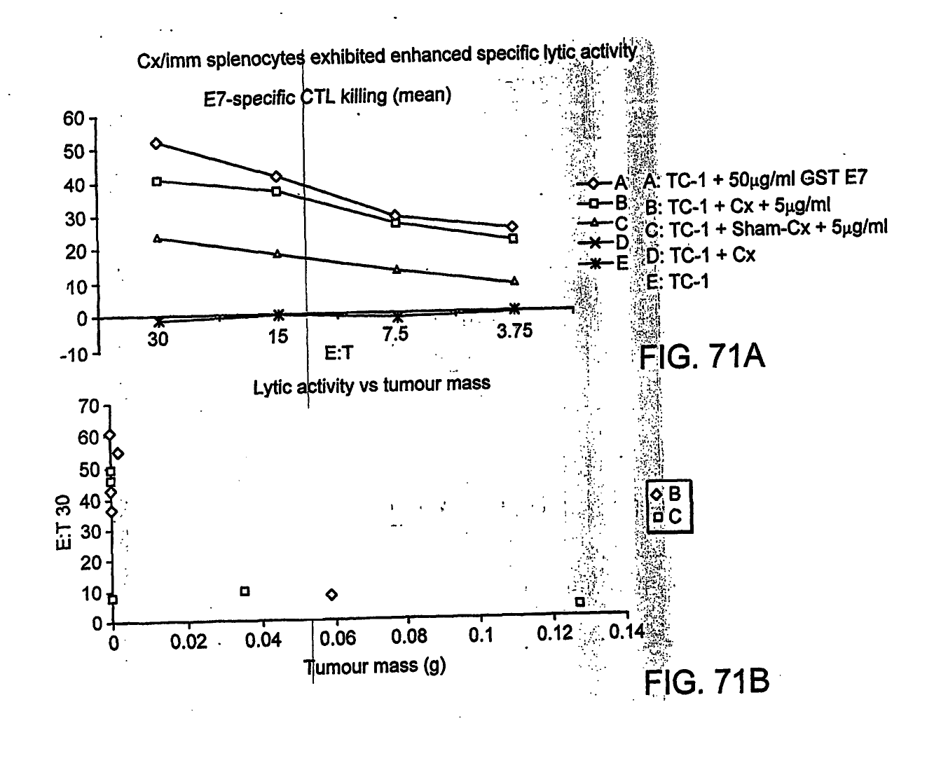

Finally, the mice were analyzed for E7-specific CTL killing of target cells infected with HPV16E7. As shown in FIG. 71A, castrated mice receiving the suboptimal E7GST dose had comparable levels of E7-specific CTL as compared to the positive control mice receiving a 10-fold higher (optimal) dose of the vaccine. Sham-castrated mice receiving the suboptimal dose vaccine had a moderate level of E7-specific CTL responses, whereas the negative control animals had little to no E7-specific CTL. This data parallels that seen with respect to tumor incidence (see FIG. 49) and IFNγ production (see FIG. 70). FIG. 71B shows that E7-specific CTL activity is inversely proportional to tumor mass. That is, mice having the highest levels of CTL activity also had no tumors; whereas, the few mice that did have tumors, were shown to have low E7-specific CTL lytic activity.

Conclusion

These experiments show that castration can potentially increase vaccine efficacy in patients. Equivalent IFNγ production, CTL activity, and protection from tumor challenge is achieved in non-castrated mice receiving a 10-fold higher (optimal) dose of an HPV16-E7 vaccine as compared to castrated mice receiving a suboptimal dose of the vaccine.

A similar experiment could be undertaken to determine the efficacy of castration plus E7 vaccination in the context of an immunotherapeutic vaccination. In this instance, mice would be castrated as indicated above. Mice are injected with TC1 cells at 6 weeks post-castration, and immunized with a GSTE7 vaccination 1 week later. Mice are then analyzed for tumors (visually, and tumor mass), and T cell responsiveness (IFNγ production using standard ELIspot methods and CTL lytic activity by ⁵¹Cr release assays using HPV16E7-pulsed target cells as described above). It is expected that mice receiving this therapeutic vaccination protocol will have reduced tumor mass as compared to sham-castrated controls. Additionally, it is expected that castrated mice will need a lower dose of vaccine than their sham-castrated counterparts.

### Example 37

### Alternative Viral Vaccination Strategies

The methods of the present invention can be used to improve the efficacy of a variety of art-recognized viral vaccines by prior, subsequent, or concurrent administration of an inhibitor of sex steroid signalling, such as a GnRH analog (or other method of castration). In addition to the examples provided above, non-limiting examples of viral vaccines are as follows.

I. Hepatitis B Viral Vaccine.

An example of a viral vaccine and a recombinant DNA vaccine are those developed for Hepatitis B by Glaxo Smith Kline (Engerix B®), and Merck Sharpe and Dohme (HB Vaxil®), respectively. The Engerix B® vaccine preparation is a 20 μg per 1 mL dose administered according to manufacturer's instructions. The HB Vax II® vaccine preparation is a 1 mL 10 μg/mL dose administered according to manufacturer's instructions. A number of pediatric formulations are also available for these and other vaccines. These vaccines may or may not contain preservatives such as Thimerosal (Australian Immunization Handbook, 8^{th} edition). Vaccine doses are typically in the range of 0.5 mL to 1 mL administered by i.m. injection. The usual course of vaccination may vary but usually consists of a single, primary immunization followed by at least one booster immunization at intervals of approximately one or more months.

II. Hepatitis a Viral Vaccine.

Examples of an inactivated viral vaccine are those developed for the treatment of Hepatitis A by Aventis Pasteur (Avaxim®), Glaxo Smith Kline (Havrix 1440® and Havrix Junior®) and others. In the case of Avaxim®, each 0.5 mL dose contains 160 ELISA units of hepatitis A (GBM strain) viral antigens. In the case of Havrix 1440®, each 0.5 mL dose contains 1440 ELISA units of hepatitis A virus (HM 175 strain). Vaccine doses of such monovalent vaccines are in the range of 0.5 mL to 1 mL by IM injection. The usual course of vaccination may vary but usually consists of 3 vaccinations at 6-month intervals.

III. Hepatitis A and Hepatitis B Multivalent Vaccine.

Additionally, polyvalent formulations may be used, which contain more than 1 viral antigen. For instance, Glaxo Smith Kline's Twinrix® contains 720 ELISA units of Hepatitis A viral antigens and 20 μg of recombinant DNA hepatitis B surface antigen protein, and are administered by i.m. injection at 0, 3, and 6 months. Another polyvalent vaccine is Aventis Pasteur's Vivaxim®. Each 1 mL dose contains 160 ELISA units of inactivated Hepatitis A virus antigens and 25 μg purified typhoid capsular polysaccharide. Supplied in a dual chamber syringe, this polyvalent vaccine is administered i.m. in 2 or 3 doses.

IV. Cytomegalovirus Vaccine.

Vical, Inc. has developed an immunotherapeutic DNA-based vaccine against CMV. The vaccine is administered in 3 doses of either 1 or 5 mgs, as provided in the manufacturer's instructions. The DNA plasmid encodes CMV phosphoprotein 65 (pp 65) and glycoprotein B (gB), and the vaccine is formulated with a poloxamer.

V. EBV Vaccine.

MedImmune, GlaxoSmithKline, and Henogen have co-developed a soluble recombinant subunit vaccine against EBV. Live recombinant vaccinia vectors have been used to express EBV gp220/350, and have been shown to confer protection in primates and EBV-negative infants. Additionally, clinical trials of an EBNA-3A peptide have been conducted in Australia (for a review of this virus vaccine and others, see, e.g., THE JORDAN REPORT 2000: ACCELERATED DEVELOPMENT OF VACCINES, published by the Division of Microbiology and Infectious Diseases, National Institute of Allergy and Infectious Diseases, National Institutes of Health.

### Example 38

### Alternative Cancer Vaccination Strategies

The methods of the present invention can be used to improve the efficacy of a variety of art-recognized cancer vaccines by prior, subsequent, or concurrent administration of an inhibitor of sex steroid signalling, such as a GnRH analog (or other method of castration). In addition to the examples provided above, non-limiting examples of cancer vaccines are as follows.

I. Melanoma Vaccine.

The methods of the present invention can be used to improve the efficacy of a melanoma vaccine by prior, subsequent, or concurrent administration of an inhibitor of sex steroid signalling, such as a GnRH analog (or other method of castration). An example of such a melanoma vaccine is the autologous melanoma cellular vaccine developed by AVAX corporation (Philadelphia, Pa.). Metastatic tumors are excised, maintained at 4° C., and delivered to the laboratory within 48 hours of excision. Tumor cells are extracted by enzymatic dissociation with collagenase and DNAse, aliquotted, frozen in a controlled rate freezer, and stored in liquid nitrogen in a medium containing human albumin and 10% dimethylsulfoxide until needed. On the day that a patient is to be treated, an aliquot of cells are thawed, washed, and irradiated to 2500 cGy. The tumor cells are then washed, incubated for 30 mins. with dinitrofluorobenzene (DNFB), and then washed with saline (Miller et al., (1976) J. Immunol. 117:1519). After washing, the cells are counted, suspended in 0.2 ml Hanks solution with human albumin, and maintained at 4° C. until administered.

Vaccine doses are in the range of 0.5−25.0×10⁶ cells. Just prior to injection, 0.1 ml of BCG (Tice, Organon Teknika, Durham, N.C.) may be added to the vaccine. The dose of BCG may be progressively attenuated to produce a local reaction consisting of an inflammatory papule without ulceration. The mixture is injected intradermally into one or more sites, e.g., the upper arm. Multi-dose vaccination is conducted over a variable period (e.g., monthly for 12 months or weekly for 6-12 weeks) and may include the administration of low dose (300 mg/M²) cyclophosphamide, a cytotoxic drug that paradoxically augments cell-mediated immunity when administered at the proper time in relation to immunization.

II. Lung Cancer Vaccine.

The methods of the present invention can also be used to improve the efficacy of a lung cancer vaccine by prior, subsequent, or concurrent administration of an inhibitor of sex steroid signalling, such as a GnRH analog (or other method of castration). An example of such a lung cancer vaccine is the DNA vaccine for non-small cell lung cancer that is made by Corixa Corporation. The vaccine is a recombinant DNA vaccine that is administered with a recombinant adenovirus adjuvant. A Biojector® device (Bioject, Tualatin, Oreg.) is used for vaccine administration and is a reusable needle-free injector powered by a compressed CO₂ cartridge.

Other non-limiting lung vaccines that may be used with the methods of the invention include L523S for non-small cell lung cancer (Wang et al., (2003) Br. J. Cancer 88:887) and BEC-2, GM2, Globo H, fucosyl GM1, and polysialic acid for small cell lung cancers (Krug (2004) Sem. Oncol. 31:112).

III. Prostate Cancer Vaccine.

The methods of the present invention can also be used to improve the efficacy of a prostate cancer vaccine by prior, subsequent, or concurrent administration of an inhibitor of sex steroid signalling, such as a GnRH analog (or other method of castration). One non-limiting example of such a vaccine is Provenge® (Dendreon Corp.), which is a vaccine against androgen independent prostate cancer. This vaccine results in the generation of a T cell immune response to the prostate cancer associated antigen prostatic acid phosphatase (PAP). DC precursors are obtained from the patient by leukopheresis and isolated from other white blood cells using a cell separation device. These cells are then co-cultured with a recombinant PAP fused delivery cassette for about 36 hours to allow the DC to mature. The mature DC are then used in the vaccine. The Provenge vaccine is delivered as 3 30-minute intravenous infusions at 2 week intervals. Other prostate cancer vaccine candidates for use in the invention are known in the art (see, e.g., Shaffer and Scher (2003) Lancet Oncol. 4:407).

IV. Colorectal Cancer Vaccine.

The methods of the present invention can also be used to improve the efficacy of a colorectal cancer vaccine by prior, subsequent, or concurrent administration of an inhibitor of sex steroid signalling, such as a GnRH analog (or other method of castration). One non-limiting example of such a vaccine is Trovax™ (Oxford BioMedica). This vaccine is a solid tumor-associated antigen, 5T4, delivered by modified vaccinia virus Ankara (MVA) via intramuscular injection. It is delivered at weeks 0, 4 and 8, coincident with chemotherapy. Other cytotoxic T-lymphocyte precursor-oriented peptide vaccines for colorectal carcinoma patients are known in the art (see, e.g., Sato et al., (2004) Br. J. Cancer 90:1334).

V. Ovarian Cancer Vaccine.

The methods of the present invention can also be used to improve the efficacy of a ovarian cancer vaccine by prior, subsequent, or concurrent administration of an inhibitor of sex steroid signalling, such as a GnRH analog (or other method of castration). One non-limiting example of such a vaccine is M-FP (CancerVac Pty. Ltd.) for the treatment of metastatic ovarian cancer. It is an autogenic cellular vaccine, whereby DC are injected subcutaneously. A recombinant version of MUC1 is fused to mannan to promote antigen uptake by the patient's purified dendritic precursor cells. Activated DC presenting MUC1 peptides are then injected s.c. into the patient.

### Example 39

### Castration Effect on BM and Spleen in Thymectomized Mice

These preliminary experiments were performed to determine if castration effects were apparent in thymectomized patients (mice). The results indicated that disruption of sex steroid signalling has direct or indirect effects on the immune system (e.g., immune cells in the BM and thymus), irrespective of the presence of a regenerated thymus.

Materials and Methods

Mice were castrated and thymectomized using routine methods known in the art. Mice were divided into the following groups: untreated (i.e., naïve, “untreated”), sham castrated (“sham-cx”), and castrated (“cx”), and each of those three groups was then thymectomized (“tx”) or sham-thymectoized (“shtx”) for a total of 6 groups analyzed. Each of the six groups was analyzed at 2 weeks and 4 weeks following myeloablation and BMT (see methods above).

Results

I. Thymectomy Does Not Impact the Effect of Sex Steroid Inhibition on the BM.

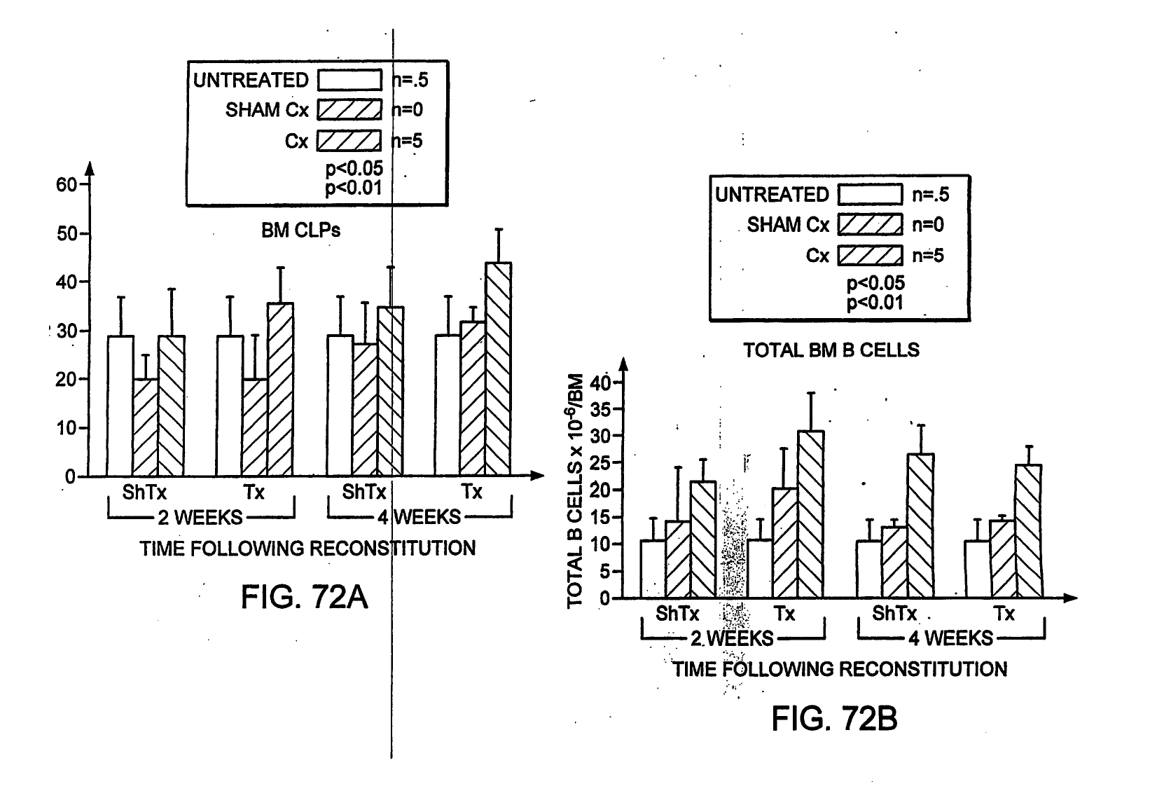

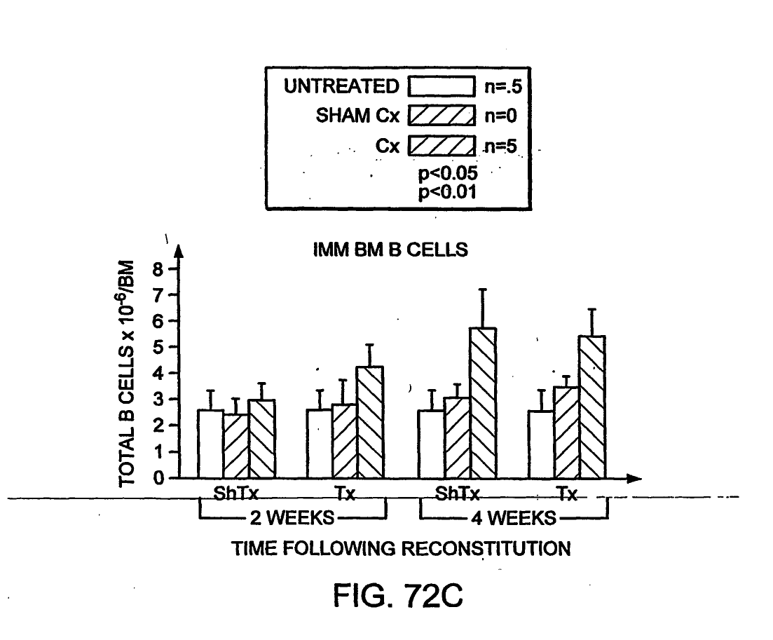

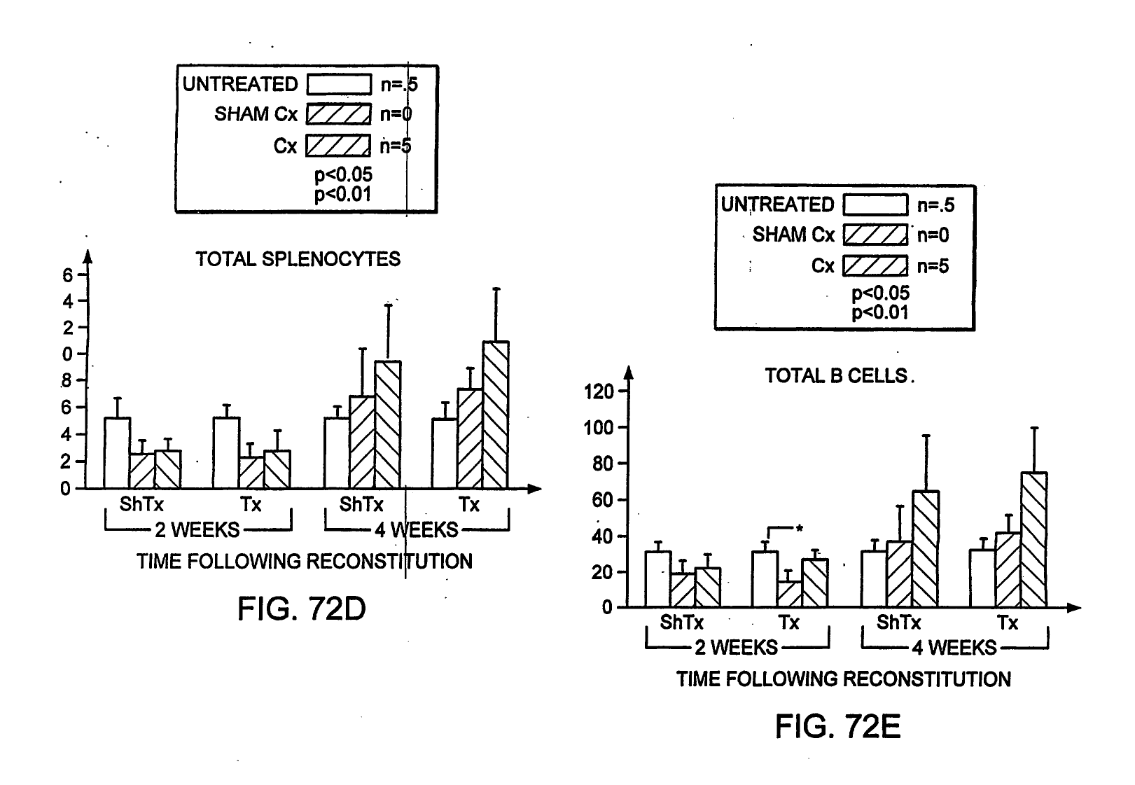

As shown in FIG. 72A, at 4 weeks post-BMT, Tx/Cx mice had an increase in the number of BM common lymphoid progenitors (CLPs), which is comparable to the ShTx/Cx mice.

As shown in FIG. 72B, at 4 weeks post-BMT, Tx/Cx mice have an increase in the total number of B cells in the BM, which is comparable to the ShTx/Cx mice. The Tx/Cx mice and the ShTx/Cx mice also have increased numbers of B cells in the BM, as compared to either the ShamCx/Tx or ShamCx/ShTx controls.

As shown in FIG. 72C, at 4 weeks post-BMT, Tx/Cx mice also have an increase in the total number of immature B cells in the BM, which is comparable to the ShTx/Cx mice. The Tx/Cx mice and the ShTx/Cx mice also have increased numbers of immature B cells in the BM, as compared to either the ShamCx/Tx or ShamCx/ShTx controls.

Thus, the results in FIGS. 72A-C support the conclusion that the effect of castration on increasing the number and functionality of cells in the BM, including increasing engraftment, does not require a reactivated thymus and is, instead, due to direct effects on the BM and other cells of the immune system.

II. Thymectomy Does Not Impact the Effect of Sex Steroid Inhibition on the Spleen.

As shown in FIG. 72D, at 4 weeks post-BMT, Tx/Cx mice also appear to have an increase in the total number of cells in the spleen, which is comparable to the ShTx/Cx mice. The Tx/Cx mice and the ShTx/Cx mice also have increased total numbers of splenocytes, as compared to either the ShamCx/Tx or ShamCx/ShTx controls.

As shown in FIG. 72E, at 4 weeks post-BMT, Tx/Cx mice also appear to have an increase in the total number of B cells in the spleen, which is comparable to the ShTx/Cx mice. The Tx/Cx mice and the ShTx/Cx mice also have increased numbers of B cells in the spleen, as compared to either the ShamCx/Tx or ShamCx/ShTx controls.

Thus, the results in FIGS. 72D-E support the conclusion that the effect of castration on increasing the number and functionality of immune cells in the spleen, including enhanced reconstitution, does not require a reactivated thymus and is, instead, due to direct effects on the BM and other cells of the immune system.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention, as claimed, should not be unduly limited to such specific embodiments. Indeed, various modifications of the described modes for carrying out the invention which are apparent to those skilled in biology or related fields are intended to be within the scope of the following claims.

Those skilled in the art will recognize, or be able to ascertain, using no more than routine experimentation, numerous equivalents to the specific embodiments described specifically herein. Such equivalents are intended to be encompassed in the scope of the following claims.

1-154. (canceled)

155. A method of increasing the functionality of the bone marrow of a patient, comprising disrupting sex steroid-mediated signaling in the patient, wherein the bone marrow functionality is increased without reactivation of the patient's thymus.

156. The method of claim 155, wherein hematopoietic stem cell (HSC) hemopoiesis is increased.

157. The method of claim 155, wherein HSC output from the bone marrow is increased.

158. The method of claim 155, wherein the sex steroid-mediated signaling is disrupted by surgical castration or chemical castration.

159. The method of claim 155, wherein the sex steroid-mediated signaling is disrupted by administration of a pharmaceutical.

160. The method of claim 159, wherein the pharmaceutical is selected from the group consisting of an LHRH agonist, an LHRH antagonist, an anti-LHRH vaccine, an anti-androgen, an anti-estrogen, a SERM, a SARM, a SPRM, an ERD, an aromatase inhibitor, an anti-progestogen, a progestin, an anti-progestin, an adrenal gland blocker, an aldoserone antagonist, a dioxalan derivative, and combinations thereof.

161. The method of claim 160, wherein the LHRH agonist is selected from the group consisting of Goserelin, Leuprolide, Triptorelin, Meterelin, Buserelin, Histrelin, Nafarelin, Lutrelin, Leuprorelin, Deslorelin, Cystorelin, Decapeptyl, Gonadorelin, and combinations thereof.

162. The method of claim 160, wherein the LHRH antagonist is selected from the group consisting of Abarelix, Cetrorelix, acetates, citrates, and other salts thereof, and combinations thereof.

163. The method of claim 160, wherein the anti-androgen is selected from the group consisting of Cosudex®, bicalutamide, cyproterone acetate, liarozole, ketoconazole, flutamide, megestrol acetate, dutasteride, finasteride, Eulexin, and combinations thereof.

164. The method of claim 155, wherein the thymus of the patient is at least in part atrophied.

165. The method of claim 164, wherein the patient has a disease or illness that at least in part atrophied the thymus of the patient.

166. The method of claim 164, wherein the patient has been treated for a disease or illness that at least in part atrophied the thymus of the patient.

167. The method of claim 166, wherein the treatment is immunosuppression, chemotherapy, or radiation treatment.

168. The method of claim 164, wherein the patient is post-pubertal.

169. The method of claim 168, further comprising administering cells to the patient, wherein the cells are stem cells, progenitor cells, dendritic cells, or combinations thereof.

170. The method of claim 169, wherein the stem cells are selected from the group consisting of HSC, epithelial stem cells, and combinations thereof.

171. The method of claim 169, wherein the progenitor cells are selected from the group consisting of lymphoid progenitor cells, myeloid progenitor cells, and combinations thereof.

172. The method of claim 169, wherein the cells are HSC.

173. The method of claim 172, wherein the HSC are CD34⁺.

174. The method of claim 169, wherein the cells are autologous.

175. The method of claim 169, wherein the cells are not autologous.

176. The method of claim 173, wherein the HSC are administered at the time disruption of sex steroid-mediated signaling is begun.

177. A method for treating or diminishing the risk of a disease or illness in a patient in need thereof without thymus reactivation, comprising:
disrupting sex steroid-mediated signaling in the patient;
administering HSC to the patient; and
allowing HSC engraftment in the patient's bone marrow, wherein the HSC engraftment is enhanced without thymus reactivation.

178. The method of claim 177, wherein the disease or illness is caused by an agent selected from the group consisting of viruses, bacteria, fungi, parasites, prions, allergens, asthma-inducing agents, and self proteins and antigens that cause autoimmune disease.

179. The method of claim 178, wherein the agent is a virus.

180. The method of claim 179, wherein the virus is selected from the group consisting of Retroviridae, Picornaviridae, Calciviridae, Togaviridae, Flaviridae, Coronaviridae, Rhabdoviridae, Filoviridae, Paramyxoviridae, Orthomyxoviridae, Bungaviridae, Arenaviridae, Reoviridae, Birnaviridae, Hepadnaviridae, Parvoviridae, Papovaviridae, Adenoviridae, Herpesviridae, Poxyiridae, and Iridoviridae.

181. The method of claim 179, wherein the virus is selected from the group consisting of influenza virus, human immunodeficiency virus, and herpes simplex virus.

182. The method of claim 178, wherein the agent is a bacterium.

183. The method of claim 182, wherein the bacterium is selected from the group consisting of Helicobacter pylori, Borelia burgdorferi, Legionella pneumophilia, Mycobacterium tuberculosis, Mycobacterium avium, Mycobacterium intracellulare, Mycobacterium kansaii, Mycobacterium gordonae, Mycobacteria sporozoites, Staphylococcus aureus, Neisseria gonorrhoeae, Neisseria meningitidis, Listeria monocytogenes, Streptococcus pyogenes, Streptococcus agalactiae, Streptococcus faecalis, Streptococcus bovis, Streptococcus pneumoniae, pathogenic Campylobacter sporozoites, Enterococcus sporozoites, Haemophilus influenzae, Bacillus anthracis, Corynebacterium diphtheriae, Corynebacterium sporozoites, Erysipelothrix rhusiopathiae, Clostridium perfringens, Clostridium tetani, Enterobacter aerogenes, Klebsiella pneumoniae, Pasturella multocida, Bacteroides sporozoites, Fusobacterium nucleatum, Streptobacillus moniliformis, Treponema pallidium, Treponema pertenue, Leptospira, and Actinomyces israelli.

184. The method of claim 182, wherein the bacterium is a mycobacterium.

185. The method of claim 178, wherein the agent is a parasite.

186. The method of claim 185, wherein the parasite is selected from the group consisting of Plasmodium falciparum, Plasmodium yoelli, and Toxoplasma gondii.

187. The method of claim 178, wherein the agent is an infectious fungus.

188. The method of claim 187, wherein the infectious fungus is selected from the group consisting of Cryptococcus neoformans, Histoplasma capsulatum, Coccidioides immitis, Blastomyces dermatitidis, Chlamydia trachomatis, and Candida albicans.

189. The method of claim 177, wherein the illness or disease is a cancer.

190. The method of claim 189, wherein the cancer is selected from the group consisting of a cancer of the brain, a cancer of the lung, a cancer of the ovary, a cancer of the breast, a cancer of the prostate, a cancer of the colon, a cancer of the blood, a cancer of the cervix, a cancer of the uterus, a cancer of the endometrium, a cancer of the bladder, a cancer of the renal organs, a cancer of the gastro-intestinal tract, a cancer of the bone, a cancer of the skin, a cancer of the connective tissue, a carcinoma, a melanoma, and a sarcoma.

191. The method of claim 178, wherein the agent is an allergen.

192. The method of claim 191, wherein the allergen causes an allergic condition selected from the group consisting of eczema, allergic rhinitis, allergic coryza, hay fever, bronchial asthma, urticaria (hives), and food allergies.

193. The method of claim 178, wherein the patient was exposed to the agent prior to the disruption of sex steroid-mediated signaling in the patient.

194. The method of claim 178, wherein the patient was not exposed to the agent prior to the disruption of sex steroid-mediated signaling in the patient.

195. The method of claim 177, further comprising administering to the patient a substance selected from the group consisting of a cytokine, a hematopoietin, a lymphokine, an interleukin, a CSF, a growth factor, and a combination thereof.

196. The method of claim 195, wherein the cytokine is selected from the group consisting of Interleukin 1 (IL-1), Interleukin 2 (IL-2), Interleukin 3 (IL-3), Interleukin 4 (IL-4), Interleukin 5 (IL-5), Interleukin 6 (IL-6), Interleukin 7 (IL-7), Interleukin 8 (IL-8), Interleukin 9 (IL-9), Interleukin 10 (IL-10), Interleukin 11 (IL-11), Interleukin 12 (IL-12), Interleukin 15 (IL-15), Interferon gamma (IFN-γ), and combinations thereof.

197. The method of claim 195, wherein the growth factor is selected from the group consisting of members of the epithelial growth factor family, members of the fibroblast growth factor family, Stem Cell Factor, granulocyte colony stimulating factor (G-CSF), keratinocyte growth factor (KGF), granulocyte-macrophage colony stimulating factor (GM-CSF), insulin-like growth factor-1 (IGF-1), a growth hormone, a thyroid hormone, M-CSF, Meg-CSF, MIF, LIF, TNF, PDGF, B cell growth factor, B cell differentiation factor, eosinophil differentiation factor, and combinations thereof.

198. A method of increasing the functionality of immune cells of a patient, comprising disrupting the sex steroid-mediated signaling in the patient, wherein the immune cell functionality is increased without reactivation of the patient's thymus, compared to the functionality of immune cells of the patient prior to disruption of sex steroid-mediated signaling.

199. The method of claim 198, wherein the functionality of immune cells of the patient is selected from the group consisting of killing of target cells; lymphocyte proliferative response; signaling ability; homing ability; APC activation; levels or activity of receptors, cell adhesion molecules, or co-stimulatory molecules; apoptosis; release of cytokines, interleukins, and other growth factors; levels of antibody in the plasma; increased levels of innate immunity in the blood and body; and combinations thereof.

200. The method of claim 198, wherein the immune cells are selected from the group consisting of T cells, B cells, and dendritic cells.

201. The method of claim 200, wherein the immune cells are T cells.

202. The method of claim 198, wherein the sex steroid-mediated signaling is disrupted by surgical castration or chemical castration.

203. The method of claim 198, wherein the sex steroid-mediated signaling is disrupted by administration of a pharmaceutical.

204. The method of claim 203, wherein the pharmaceutical is selected from the group consisting of an LHRH agonist, an LHRH antagonist, an anti-LHRH vaccine, an anti-androgen, an anti-estrogen, a SERM, a SARM, a SPRM, an ERD, an aromatase inhibitor, an anti-progestogen, a progestins, an anti-progestin, an adrenal gland blocker, an aldoserone antagonist, a dioxalan derivatives, and combinations thereof.

205. The method of claim 204, wherein the LHRH agonist is selected from the group consisting of Goserelin, Leuprolide, Triptorelin, Meterelin, Buserelin, Histrelin, Nafarelin, Lutrelin, Leuprorelin, Deslorelin, Cystorelin, Decapeptyl, Gonadorelin, and combinations thereof.

206. The method of claim 204, wherein the LHRH antagonist is selected from the group consisting of Abarelix, Cetrorelix, acetates, citrates, and other salts thereof, and combinations thereof.

207. The method of claim 204, wherein the anti-androgen is selected from the group consisting of Cosudex®, bicalutamide, cyproterone acetate, liarozole, ketoconazole, flutamide, megestrol acetate, dutasteride, finasteride, Eulexin, and combinations thereof.

208. The method of claim 198, wherein the thymus of the patient is at least in part atrophied.

209. The method of claim 198, wherein the patient has a disease or illness that at least in part atrophied the thymus of the patient.

210. The method of claim 198, wherein the patient has had a treatment of a disease or illness that at least in part atrophied the thymus of the patient.

211. The method of claim 210, wherein the treatment is immunosuppression, chemotherapy, or radiation treatment.

212. The method of claim 198, wherein the patient is post-pubertal.

213. The method of claim 212, further comprising administering to the patient cells selected from the group consisting of stem cells, progenitor cells, dendritic cells, and combinations thereof.

214. The method of claim 213, wherein the stem cells are selected from the group consisting of HSC, epithelial stem cells, and combinations thereof.

215. The method of claim 213, wherein the progenitor cells are selected from the group consisting of lymphoid progenitor cells, myeloid progenitor cells, and combinations thereof.

216. The method of claim 213, wherein the stem cells are HSC.

217. The method of claim 216, wherein the HSC are CD34⁺.

218. The method of claim 213, wherein the cells are autologous.

219. The method of claim 213, wherein the cells are not autologous.

220. The method of claim 216, wherein the HSC are administered at the time disruption of sex steroid-mediated signaling is begun.


SEQUENCE LISTING (11 sequences)

SEQ ID NO: 1 - protein, 9 aa, Artificial Sequence
  Description of Artificial Sequence Synthetic peptide
IYSTVASSL

SEQ ID NO: 2 - protein, 9 aa, Artificial Sequence
  Description of Artificial Sequence Synthetic peptide
TYQRTRALV

SEQ ID NO: 3 - protein, 16 aa, Artificial Sequence
  Description of Artificial Sequence Synthetic peptide
SYVPSAEQILEFVKQI

SEQ ID NO: 4 - DNA, 25 bp, Artificial Sequence
  Description of Artificial Sequence Synthetic primer
ggatggaaaacacagtgtgacatgg

SEQ ID NO: 5 - DNA, 26 bp, Artificial Sequence
  Description of Artificial Sequence Synthetic primer
ctgtcaacaaaggtgatgccacatcc

SEQ ID NO: 6 - DNA, 20 bp, Artificial Sequence
  Description of Artificial Sequence Synthetic oligonucleotide
cacaggactagaacacctgc

SEQ ID NO: 7 - DNA, 19 bp, Artificial Sequence
  Description of Artificial Sequence Synthetic oligonucleotide
gctggtgaaaaggacctct

SEQ ID NO: 8 - DNA, 20 bp, Artificial Sequence
  Description of Artificial Sequence Synthetic oligonucleotide
ctactgcttcagctccacag

SEQ ID NO: 9 - DNA, 19 bp, Artificial Sequence
  Description of Artificial Sequence Synthetic oligonucleotide
tgcacttgcaggagcgcac

SEQ ID NO: 10 - DNA, 20 bp, Artificial Sequence
  Description of Artificial Sequence Synthetic oligonucleotide
gccttgtcacgacctgtttc

SEQ ID NO: 11 - DNA, 22 bp, Artificial Sequence
  Description of Artificial Sequence Synthetic oligonucleotide
agttcacactcgtagccgtttg